(19) United States
(12) Patent Application Publication
(10) Pub. No.: US 2018/0368404 A1
(43) Pub. Date: Dec. 27, 2018

(54) 3-PYRIDYL HETEROBICYCLIC COMPOUND FOR CONTROLLING INVERTEBRATE PESTS

(71) Applicant: BASF SE, Ludwigshafen (DE)

(72) Inventors: Joachim DICKHAUT, Heidelberg (DE); Devendra VYAS, Mumbai (IN); Jean-Yves WACH, Zürich (CH); Arun NARINE, Mannheim (DE); Wolfgang VON DEYN, Neustadt (DE); Martin WEISEL, Mannheim (DE); Raffael KOLLER, Zuerich (CH); Karsten KOERBER, Eppelheim (DE)

(21) Appl. No.: 15/524,848

(22) PCT Filed: Nov. 6, 2015
(86) PCT No.: PCT/EP2015/075941

§ 371 Date: May 5, 2017

Related U.S. Application Data
(60) Provisional application No. 62/075,933, filed on Nov. 6, 2014.

(30) Foreign Application Priority Data
Apr. 24, 2015 (IN) 2116/CHE/2015

(51) Int. Cl. A01N 43/52; C07D 513/04; C07D 498/04; C07D 487/04; C07D 471/04; A01N 43/76; C07D 401/14; C07D 401/04; A01N 43/90; C07D 413/04

(52) U.S. Cl. CPC A01N 43/52; C07D 513/04; C07D 498/04; C07D 487/04; C07D 413/04; A01N 43/76; C07D 401/14; C07D 401/04; A01N 43/90; C07D 471/04

(57) ABSTRACT

The present invention relates to the use of substituted 3-pyridyl heterobicyclic compounds of formula I as agrochemical pesticides. Furthermore, the present invention relates to novel substituted 3-pyridylheterobicyclic compounds of formula I* as well as to processes and intermediates for preparing them, and also to active compound combinations comprising them, to compositions comprising them, and to their use for protecting growing plants from attack or infestation by invertebrate pests. Furthermore, the invention relates to methods of applying the compounds of I, or I*. The present invention also relates to seed comprising such compounds.

The present invention relates to the use of substituted 3-pyridyl heterobicyclic compounds of formula I as agrochemical pesticides. Furthermore, the present invention relates to novel substituted 3-pyridylheterobicyclic compounds of formula I* falling within the scope of formula I, as well as to processes and intermediates for preparing them, and also to active compound combinations comprising them. Moreover, the present invention relates to agricultural or veterinary compositions comprising the compounds I, or I*, and to the use of the compounds I, or I*, or compositions comprising them for combating or controlling invertebrate pests and/or for protecting crops, plants, plant propagation material, and/or growing plants from attack and/or infestation by invertebrate pests. The present invention also relates to methods of applying the compounds I, or I*. Furthermore, the present invention relates to seed comprising compounds according to the invention.

Invertebrate pests and in particular insects, arachnids and nematodes destroy growing and harvested crops and attack wooden dwelling and commercial structures, thereby causing large economic loss to the food supply and to property. Accordingly, there is an ongoing need for new agents for combating invertebrate pests.

It has been found that 5-membered 3-pyridyl heterocycles represent an important class of insecticides. For example, 3-pyridyl thiazoles and in particular tri- and tetra-aryl 3-pyridyl thiazoles have been found to be pesticidally active. In this regard, reference is, e.g., made to WO 2011/128304, WO 2010/129497, WO 2012/061290, WO 2013/062981, WO 2012/102387, WO 2012/108511, JP 2013/256468, WO 2012/168361, WO 2013/000931.

Due to the ability of target pests to develop resistance to pesticidally active agents, there is an ongoing need to identify further compounds, which are suitable for combating invertebrate pests such as insects, arachnids and nematodes. Furthermore, there is a need for new compounds having a high pesticidal activity and showing a broad activity spectrum against a large number of different invertebrate pests, especially against difficult to control insects, arachnids and nematodes.

It is therefore an object of the present invention to identify and provide compounds, which exhibit a high pesticidal activity and have a broad activity spectrum against invertebrate pests.

It has been found that these objects can be achieved by substituted 3-pyridyl heterobicyclic compounds of formula I and formula I*, as depicted and defined below, including their stereoisomers, their salts, in particular their agriculturally or veterinarily acceptable salts, their tautomers and their N-oxides.

The present invention relates to the use of a substituted 3-pyridyl heterobicyclic compound of formula I or a salt, stereoisomer, tautomer, or N-oxide thereof

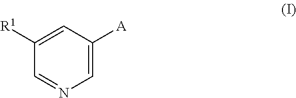

as an agrochemical pesticide,
wherein
R¹ is H, CN or halogen; and
A is a moiety selected from

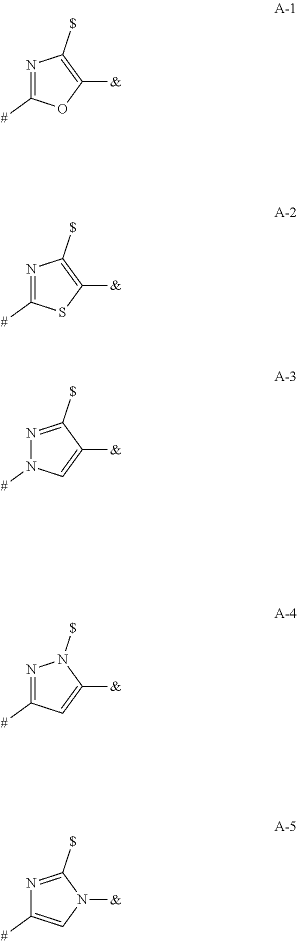

wherein
# denotes the bond to the pyridine ring of formula I; and
$ and & mark the connection to a C₃-C₄-alkylene, or C₃-C₄-alkenylene chain, of which none, one or more C-atoms are independently replaced by a heteroatom O, N, or S, and which forms together with the atoms to which it is bonded an annulated saturated, partially unsaturated, or fully unsaturated carbocyclic, or heterocyclic ring;
   wherein the N- and/or S-atoms of the chain are independently oxidized, or non-oxidized, and wherein the C-atoms and/or the heteroatoms of the chain are unsubstituted, or substituted by one or more, same or different substituents R²;
      wherein

### R² is

(i) halogen, CN, NO₂, C₁-C₁₀-alkyl, C₃-C₈-cycloalkyl, C₂-C₁₀-alkenyl, C₃-C₈-cycloalkenyl, C₂-C₁₀-alkynyl, which aliphatic and cycloaliphatic radicals are in each case unsubstituted or substituted with one or more, same or different substituents R¹¹; OR¹², NR^{13a}R^{13b}, S(O)ₙR¹², S(O)ₙNR^{13a}R^{13b}, Si(R¹⁵)₂R¹⁶;
phenyl, which is unsubstituted, or substituted with one or more, same or different substituents R¹⁴;
or a 3- to 7-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents R¹⁴, and wherein said N- and S-atoms are independently oxidized, or non-oxidized; with the proviso that R² is not halogen, if R² is bonded to a heteroatom; or
(ii) a substituent

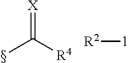

wherein
§ denotes the bond to the atom on which R² is present;
X is NR³, O, or S; and
R⁴ is H, CR⁵R⁶R⁷, NR⁸R⁹, OR¹⁰, or SR¹⁰; or
(iii) two R² present on one C- or S-atom are together ═O, ═S, or ═NR³; and wherein
R³ is H, CN, NO₂, C₁-C₁₀-alkyl, C₃-C₈-cycloalkyl, C₂-C₁₀-alkenyl, C₂-C₁₀-alkynyl, which aliphatic and cycloaliphatic radicals are in each case unsubstituted, or substituted with one or more, same or different substituents R¹¹; OR¹², NR^{13a}R^{13b}, S(O)ₙR¹², S(O)ₙNR^{13a}R^{13b}, C(═O)R¹¹, C(═O)NR^{13a}R^{13b}, C(═O)OR¹², C(═S)R¹¹, C(═S)NR^{13a}R^{13b}, C(═S)OR¹², C(═S)SR¹², C(═NR^{13a})R¹¹, C(═NR^{13a})NR^{13a}R^{13b}; phenyl, which is unsubstituted, or substituted with one or more, same or different substituents R¹⁴;
   or a 3- to 7-membered saturated, partially unsaturated, or fully unsaturated heterocyclic ring wherein said heterocyclic ring comprises one or more, same or different heteroatoms O, N, or S, and is unsubstituted, or substituted with one or more, same or different substituents R¹⁴, and wherein said N- and/or S-atoms, independently of one another, are oxidized, or non-oxidized;
R⁵, R⁶, R⁷ are independently H, halogen, CN, NO₂, —SCN, C₁-C₆-alkyl, C₁-C₆-haloalkyl, C₁-C₆-alkoxy, C₁-C₆-haloalkoxy, C₁-C₆-alkyl-S(O)ₙ—, C₁-C₆-haloalkyl-S(O)ₙ—, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl; Si(R¹⁵)₂R¹⁶, OR¹², OSO₂R¹², S(O)ₙR¹², S(O)ₙNR^{13a}R^{13b}, NR^{13a}R^{13b}, C(═O)NR^{13a}R^{13b}, C(═S)NR^{13a}R^{13b}, C(═O)OR¹²;
   phenyl, which is unsubstituted, or substituted with one or more, same or different substituents R¹⁴;
   a 3- to 7-membered saturated, partially unsaturated, or fully unsaturated heterocyclic ring comprising one or more, same or different heteroatoms O, N or S, wherein said heterocyclic ring is unsubstituted, or substituted with one or more, same or different substituents R¹⁴, and wherein the N- and/or S-atoms of the heterocyclic ring are oxidized, or non-oxidized;
   or R⁵ and R⁶ together form ═O, ═CR¹¹R¹⁷, ═S, ═S(O)ₙR¹²; ═S(O)ₙNR^{13a}R^{13b}, ═NR^{13a}, ═NOR¹², ═NNR^{13a}R^{13b}, and R⁷ is selected from the group above;
   or R⁵ and R⁶ together form a 3- to 8-membered saturated or partially unsaturated carbocyclic or heterocyclic ring together with the C-atom to which they are bonded to, and R⁷ is selected from the group above;
R⁸, R⁹ are independently H, CN, C₁-C₁₀-alkyl, C₃-C₈-cycloalkyl, C₂-C₁₀-alkenyl, C₂-C₁₀-alkynyl, which aliphatic and cycloaliphatic radicals are unsubstituted or substituted with one or more, same or different substituents R¹;
   OR¹², NR^{13a}R^{13b}, S(O)ₙNR^{13a}R^{13b}, C(═O)R¹¹, C(═O)NR^{13a}R^{13b}, C(═O)OR¹², C(═S)R¹¹, C(═S)NR^{13a}R^{13b}, C(═S)OR¹², C(═S)SR¹², C(═NR^{13a})R¹¹, C(═NR^{13a})NR^{13a}R^{13b}, Si(R¹⁵)₂R¹⁶; phenyl, which is unsubstituted or substituted with one or more, same or different substituents R¹⁴;
   a 3- to 7-membered saturated, partially unsaturated, or fully unsaturated heterocyclic ring, wherein said heterocyclic ring comprises one or more, same or different heteroatoms O, N, or S, and is unsubstituted or substituted with one or more, same or different substituents R¹⁴, and wherein said N- and/or S-atoms, independently of one another, are oxidized or non-oxidized; or
   R⁸ and R⁹ together are part of a C₂-C₇-alkylene, C₂-C₇-alkenylene, or C₂-C₇-alkynylene chain and form a 3- to 8-membered saturated, partially unsaturated, or fully unsaturated heterocyclic ring together with the N-atom they are bonded to, wherein none, one or more of the CH₂, CH, or C groups are replaced by one or more, same or different groups C═O, C═S, O, N or NH, and wherein the C-, and/or N-atoms in the C₂-C₇-alkylene, C₂-C₇-alkenylene, or C₂-C₇-alkynylene chain are unsubstituted, or substituted by one or more, same or different substituents selected from halogen, CN, C₁-C₆-alkyl, C₁-C₆-haloalkyl, C₁-C₆-alkoxy, C₁-C₆-haloalkoxy, C₁-C₆-alky-S(O)ₙ—, C₁-C₆-haloalkyl-S(O)ₙ—, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, and phenyl, which groups are unsubstituted, or substituted with one or more, same or different substituents R¹¹, and wherein the S-, and/or N-atoms in the C₂-C₇-alkylene, C₂-C₇-alkenylene or C₂-C₇-alkynylene chain, independently of one another, are oxidized, or non-oxidized;
   or R⁸ and R⁹ together form a ═CHR¹⁷, ═CR¹¹R¹⁷, ═NR^{13a} or ═NOR¹² radical;
R¹⁰ is H, CN, C₁-C₆-alkyl, C₁-C₆-haloalkyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl; Si(R¹⁵)₂R¹⁶, S(O)ₙR¹², S(O)ₙNR^{13a}R^{13b}, NR^{13a}R^{13b}, —N═CR¹¹R¹⁷, C(═O)R¹¹, C(═O)NR^{13a}R^{13b} C(═S)NR^{13a}R^{13b}, C(═O)OR¹²;
   phenyl, which is unsubstituted, or substituted with one or more, same or different substituents R¹⁴;
   or a 3- to 7-membered saturated, partially unsaturated, or fully unsaturated heterocyclic ring comprising one or more, same or different heteroatoms O, N or S, wherein the heterocyclic ring is unsubstituted or substituted with one or more, same or different substituents R¹⁴, and wherein the N- and/or S-atoms of the heterocyclic ring are oxidized, or non-oxidized;
      and wherein
R¹¹ is H, halogen, CN, NO₂, —SCN, C₁-C₆-alkyl, C₁-C₆-haloalkyl, C₁-C₆-alkoxy, C₁-C₆-haloalkoxy, C₁-C₆-alkyl-S(O)ₙ—, C₁-C₆-haloalkyl-S(O)ₙ—, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl; Si(R¹⁵)₂R¹⁶, OR²⁰, OSO₂R²⁰, S(O)ₙR²⁰, S(O)ₙNR^{21a}R^{21b}, NR^{21a}R^{21b}, C(═O)NR^{21a}R^{21b}C(═S)NR^{21a}R^{21b}, C(═O)OR²⁰;
   phenyl, which is unsubstituted, or substituted with one or more, same or different substituents R²²,
   or a 3- to 7-membered saturated, partially unsaturated, or fully unsaturated heterocyclic ring comprising one or more, same or different heteroatoms O, N or S, wherein the heterocyclic ring is unsubstituted, or substituted with one or more, same or different substituents R²², and wherein the N- and/or S-atoms of the heterocyclic ring are oxidized, or non-oxidized;
   or two R¹¹ present on one C-atom together form ═O, ═CR¹⁷R¹⁸, ═S, ═S(O)ₙR²⁰; ═S(O)ₙNR^{21a}R^{21b}, ═NR^{21a}, ═NOR²⁰, ═NNR^{21a};
   or two R¹¹ form a 3- to 8-membered saturated, or partially unsaturated carbocyclic, or heterocyclic ring together with the C-atoms to which the two R¹¹ are bonded to;
R¹² is H, CN;
   C₁-C₆-alkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
   C₁-C₆-haloalkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl; C₂-C₆-alkenyl, C₂-C₆-haloalkenyl, C₂-C₆-alkynyl, or C₂-C₆-haloalkynyl;
   C₃-C₈-cycloalkyl, C₃-C₈-halocycloalkyl, C₃-C₈-cycloalkyl-C₁-C₄-alkyl, or C₃-C₈-halocycloalkyl-C₁-C₄-alkyl, wherein the cyclic moieties are unsubstituted, or substituted with one or more, same or different substituents selected from halogen, CN, C₁-C₄-alkyl, C₁-C₄-haloalkyl, C₁-C₄-alkoxy, C₁-C₄-haloalkoxy, ═O, and acetal groups thereof based on alcohols, or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH;
   Si(R¹⁵)₂R¹⁶, S(O)ₙR²⁰, S(O)ₙNR^{21a}R^{21b}, NR^{21a}R^{21b}, N═CR¹⁷R¹⁸, C(═O)R¹⁹, C(═O)NR^{21a}R^{21b}C(═S)NR^{21a}R^{21b}, C(═O)OR²⁰;
   phenyl, or phenyl-C₁-C₄-alkyl, wherein the aromatic rings are unsubstituted, or substituted with one or more, same or different substituents R²²;
   or a 3- to 7-membered saturated, partially unsaturated, or fully unsaturated heterocyclic ring comprising one or more, same or different heteroatoms O, N or S, wherein the heterocyclic ring is directly bonded to the remainder of the molecule, or bonded to the remainder of the molecule via a C₁-C₄-alkyl group, and wherein the heterocyclic ring is unsubstituted, or substituted with one or more, same or different substituents R²², and wherein the N- and/or S-atoms of the heterocyclic ring are oxidized, or non-oxidized;
R^{13a}, R^{13b} are independently H, C₁-C₆-alkyl, C₁-C₆-haloalkyl, C₁-C₆-alkoxy, C₁-C₆-haloalkoxy, C₁-C₆-alkyl-S—, C₁-C₆-haloalky-S—, C₃-C₈-cycloalkyl, C₃-C₈-cycloalkyl-C₁-C₆-alkyl, C₃-C₈-halocycloalkyl, C₂-C₆-alkenyl, C₂-C₆-haloalkenyl, C₂-C₆-alkynyl, C₂-C₆ haloalkynyl, —S(O)ₙNR^{21a}R^{21b}, C(═O)R¹⁹, C(═O)OR²⁰, C(═O)NR^{21a}R^{21b}, C(═S)R¹⁹, C(═S)SR²⁰, C(═S)NR^{21a}R^{21b}, C(═NR^{21a})R¹⁹;
   phenyl, which is unsubstituted, or substituted with one or more, same or different substituents R²²;
   a 3- to 7-membered saturated, partially unsaturated, or fully unsaturated heterocyclic ring comprising one or more, same or different heteroatoms O, N or S, wherein the heterocyclic ring is unsubstituted, or substituted with one or more, same or different substituents R²², and wherein the N- and/or S-atoms of the heterocyclic ring are oxidized, or non-oxidized;
   or R^{13a} and R^{13b} are together a C₂-C₇ alkylene, or C₂-C₇ alkenylene chain, and form a 3- to 8-membered saturated, partially unsaturated, or fully unsaturated ring together with the N-atom they are bonded to,
   wherein the C₂-C₇ alkylene chain or C₂-C₇ alkenylene chain contains none, one or more, same or different heteratoms O, S or N, and is substituted or unsubstituted with one or more, same or different substituents selected from halogen, C₁-C₆-alkyl, C₁-C₆-haloalkyl, C₁-C₆-alkoxy, C₁-C₆-haloalkoxy, C₁-C₆-alkyl-S—, C₁-C₆-haloalkyl-S, C₃-C₈-cycloalkyl, C₃-C₈-halocycloalkyl, C₂-C₆-alkenyl, C₂-C₆-haloalkenyl, C₂-C₆-alkynyl, and C₂-C₆ haloalkynyl; phenyl, which is unsubstituted, or substituted with one or more, same or different substituents R²²;
   or a 3- to 7-membered saturated, partially unsaturated, or fully unsaturated heterocyclic ring comprising one or more, same or different heteroatoms O, S, or N, and is substituted or unsubstituted with one or more, same or different substituents R²², and wherein the N- and/or S-atoms of the heterocyclic ring are oxidized or non-oxidized;
   or R^{13a} and R^{13b} together form a ═CR¹⁷R¹⁸, ═NR²¹, or ═NOR²⁰ radical;
R¹⁴ is H, halogen, CN, NO₂, SCN, SF₅, C₁-C₁₀-alkyl, C₃-C₈-cycloalkyl, C₂-C₁₀-alkenyl, C₂-C₁₀-alkynyl, wherein the aliphatic and cycloaliphatic radicals are unsubstituted or substituted with one or more, same or different substituents R¹⁹;
   Si(R¹⁵)₂R¹⁶, OR²⁰, OS(O)ₙR²⁰, —S(O)ₙR²⁰, S(O)ₙNR^{21a}R^{21b}, NR^{21a}R^{21b}, C(═O)R¹⁹, C(═O)OR²⁰, —C(═NR^{21a})R¹⁹, C(═O)NR^{21a}R^{21b}, C(═S)NR^{21a}R^{21b};
   phenyl, which is unsubstituted or substituted with one or more, same or different substituents selected from halogen, CN, NO₂, C₁-C₆-alkyl, C₁-C₆-haloalkyl, C₁-C₆-alkoxy, and C₁-C₆-haloalkoxy;
   or a 3- to 7-membered saturated, partially unsaturated, or fully unsaturated, or aromatic heterocyclic ring comprising one or more, same or different heteroatoms O, N, or S, which is unsubstituted or substituted with one or more, same or different substituents selected from halogen, CN, NO₂, C₁-C₆-alkyl, C₁-C₆-haloalkyl, C₁-C₆-alkoxy, and C₁-C₆-haloalkoxy, and wherein the N- and/or S-atoms of the heterocyclic ring are oxidized or non-oxidized; or two R¹⁴ present together on one atom of a partly saturated heterocycle are ═O, ═CR¹⁷R¹⁸, ═NR^{21a}, ═NOR²⁰, or ═NNR^{21a};
   or two R¹⁴ on adjacent C-atoms form a bridge CH₂CH₂CH₂CH₂, CH═CH—CH═CH, N═CHCH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{21a}, CH₂CH═N, CH═CH—NR^{21a}, OCH═N, SCH═N, wherein the bridge is substituted or unsubstituted with one or more, same or different substituents selected from ═O, OH, CH₃, OCH₃, halogen, CN, halomethyl, or halomethoxy;
R¹⁵, R¹⁶ are independently H, C₁-C₆-alkyl, C₁-C₆-haloalkyl, C₁-C₆-alkoxy, C₁-C₄-alkoxy-C₁-C₄-alkyl, C₃-C₈-cycloalkyl, C₃-C₈-halocycloalkyl, C₁-C₄-haloalkoxy-C₁-C₄-alkyl, or phenyl;
R¹⁷, R¹⁸ are independently H, C₁-C₄-alkyl, C₁-C₆-cycloalkyl, C₁-C₂-alkoxy-C₁-C₂-alkyl, phenyl, or benzyl;
R¹⁹ is H, halogen, CN, NO₂, OH, SH, SCN, SF₅, C₁-C₆-alkoxy, C₁-C₆-haloalkoxy, C₁-C₆-alkylS(O)ₙ—, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, C₃-C₈-cycloalkyl, wherein the aliphatic and cyclo-aliphatic radicals are unsubstituted, partially or fully halogenated and/or oxgenated and/or are unsubstituted or substituted by one or more, same or different radicals selected from C₁-C₄-alkoxy;
   phenyl, benzyl, pyridyl, or phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino, and di-(C₁-C₆-alkyl)amino;
   or two R¹⁹ present on the same C-atom are together ═O, ═CH(C₁-C₄-alkyl), ═C(C₁-C₄-alkyl)C₁-C₄-alkyl, ═N(C₁-C₆-alkyl), or ═NO(C₁-C₆-alkyl);
R²⁰ is H, CN, C₁-C₆-alkyl-S(O)ₙ, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, C₃-C₈-cycloalkyl, wherein the aliphatic and cycloaliphatic radicals are unsubstituted, partially or fully halogenated and/or oxygenated and/or substituted by one or more, same or different radicals selected from C₁-C₄-alkoxy;
   phenyl, benzyl, pyridyl, or phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
R^{21a}, R^{21b} are independently H, CN, C₁-C₆-alkoxy, C₁-C₆-haloalkoxy, C₁-C₆-alkyl-S(O)ₙ—, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, C₃-C₈-cycloalkyl, C₃-C₈-cycloalkyl-C₁-C₆-alkyl, C₁-C₆-alkyl-C₃-C₈-cycloalkyl, wherein the aliphatic and cycloaliphatic radicals are unsubstituted, partially or fully halogenated and/or oxygenated and/or substituted by one or more, same or different radicals selected from C₁-C₄-alkoxy;
   phenyl, benzyl, pyridyl, and phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
   or R^{21a} and R^{21b} together represent a C₂-C₆ alkylene chain forming a 3- to 7-membered saturated, partially unsaturated, or fully unsaturated heterocyclic ring together with the N-atom R^{21a} and R^{21b} are bonded to, wherein the alkylene chain contains none, or one or more, same or different heteroatoms O, S, or N, and is unsubstituted or substituted with one or more, same or different substituents selected from halogen, C₁-C₄-haloalkyl, C₁-C₄-alkoxy, and C₁-C₄-haloalkoxy, and wherein the N- and/or S-atoms of the heterocyclic ring are oxidized or non-oxidized;
R²² is H, halogen, NO₂, CN, OH, SH, C₁-C₆-alkoxy, C₁-C₆-haloalkoxy, C₁-C₆-alkyl-S(O)ₙ, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, or C₃-C₈-cycloalkyl, wherein the aliphatic and cyclo-aliphatic radicals are unsubstituted, partially or fully halogenated and/or oxygenated and/or are substituted by one or more, same or different radicals selected from C₁-C₄-alkoxy;
   phenyl, benzyl, pyridyl, or phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
   or two R²² present together on one atom are ═O, ═S, ═N(C₁-C₆-alkyl), ═NO(C₁-C₆-alkyl), ═CH(C₁-C₄-alkyl), or ═C(C₁-C₄-alkyl)C₁-C₄-alkyl;
   or two R²² on two adjacent C-atoms are together a C₂-C₆-alkylene chain or C₂-C₆-alkenylene chain, which form together with the C-atom they are bonded to a 3- to 7-membered saturated, partially unsaturated, or fully unsaturated ring, wherein the alkylene or alkenylene chain contains none, or one or more, same or different heteroatoms selected from O, S, or N, and is unsubstituted or substituted with halogen, C₁-C₄-haloalkyl, C₁-C₄-alkoxy, or C₁-C₄-haloalkoxy, and wherein the N- and/or S-atoms of the heterocyclic ring are oxidized or non-oxidized;
   n is 0, 1, or 2.

The present invention also relates to a substituted 3-pyridyl heterobicyclic compound of formula I*, which corresponds to formula I, or a salt, stereoisomer, tautomer, or N-oxide thereof

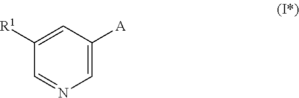

wherein
A is a moiety selected from the group consisting of

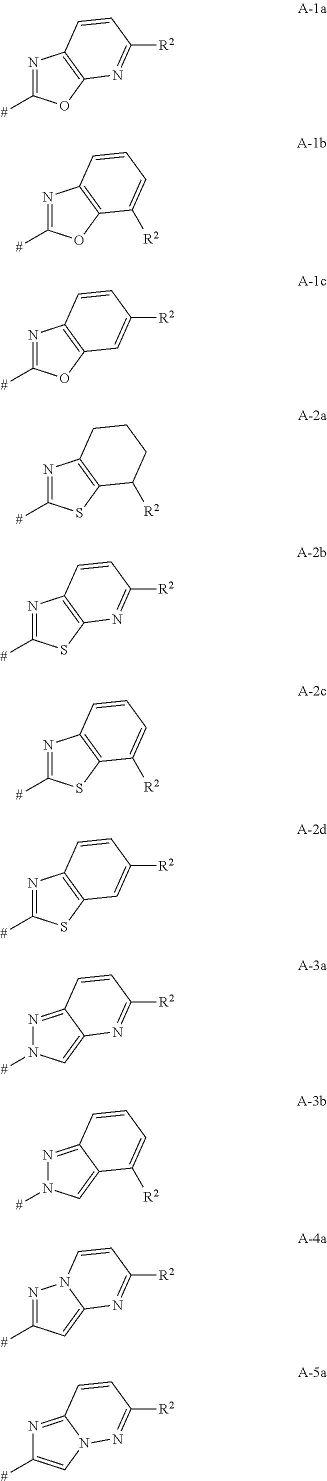

wherein # denotes the bond to the pyridine ring of formula I*, and wherein
R¹ and R² are defined as in formula I above.

The present invention also relates to an agricultural or veterinary composition comprising at least one compound I, or I*; to a method for combating, or controlling invertebrate pests; and to a method for protecting growing plants from attack, or infestation by invertebrate pests. The present invention also relates to the compounds I, or I*, or compositions comprising these compounds for use in treating, or protecting animals against attack, infestation, or infection by parasites. The present invention further relates to seed comprising a compound I, or I*.

Further embodiments of the present invention can be found in the claims, the description and the examples. It is to be understood that the features mentioned above and those still to be illustrated below of the subject matter of the invention can be applied not only in the respective given combination but also in other combinations without leaving the scope of the invention.

In one embodiment, the present invention relates to the use of compounds of formula I, wherein A is a moiety selected from A-1 to A-5.

These compounds correspond to formulae I.A-1, I.A-2, I.A-3, I.A-4 and I.A-5, respectively.

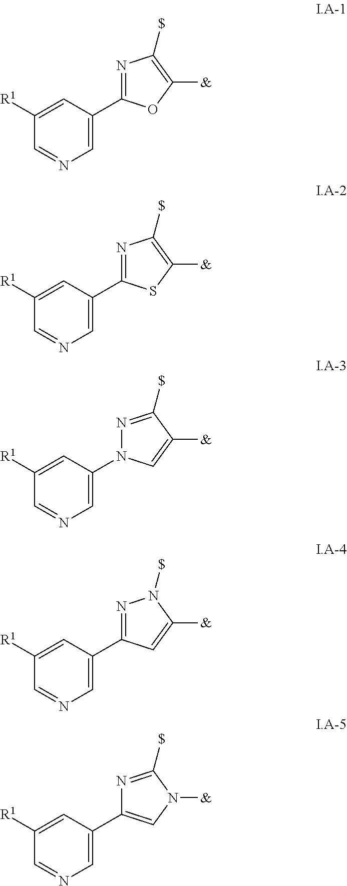

In one embodiment, $ and & mark the connection to a C₄-alkylene or C₄-alkenylene chain, of which the terminal C-atom at the & position may be replaced by O, N, or S. In another embodiment, said terminal C-atom of the C₄-alkylene or C₄-alkenylene chain is not replaced.

In a preferred embodiment, the present invention relates to the use of compounds of formula I.A-1, I.A-2, I.A-3, I.A-4 and I.A-5, wherein $ and & mark the connection to a chain, which is selected from the group consisting of

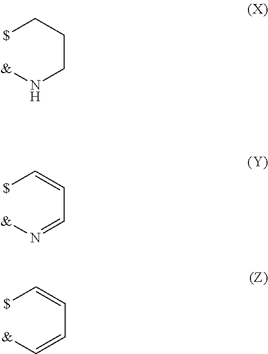

wherein in each case $ marks the connection to the $-position of the moiety A and & marks the connection to the &-position of the moiety A, and wherein the C-atoms and/or the N-atom of the chains may be substituted by 1, 2, or 3 substituents R², said substituents R² being identical or different from one another if more than one substituent R² is present. These compounds correspond to formulae I.A-1X, I.A-1Y, I.A-1Z, I.A-2X, I.A-2Y, I.A-2Z, I.A-3X, I.A-3Y, I.A-3Z, I.A-4X, I.A-4Y, I.A-4Z, I.A-5X, I.A-5Y, and I.A-5Z, respectively.

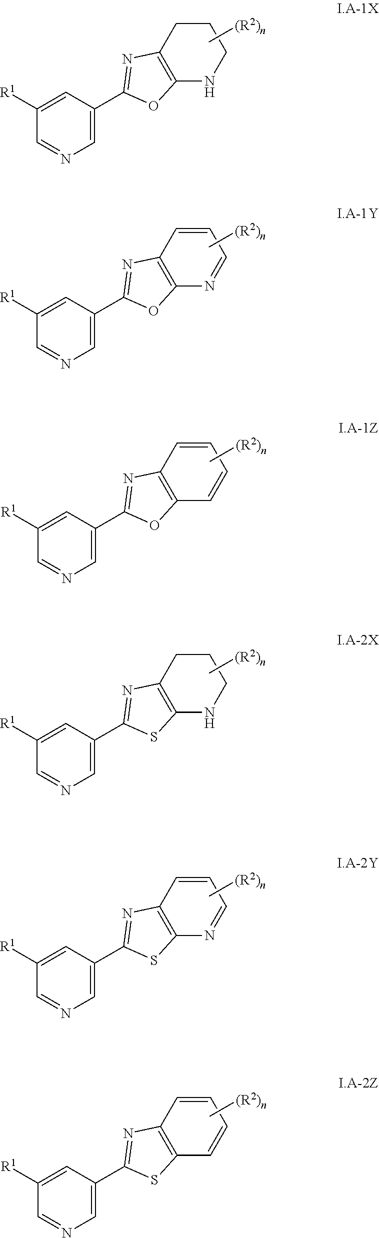

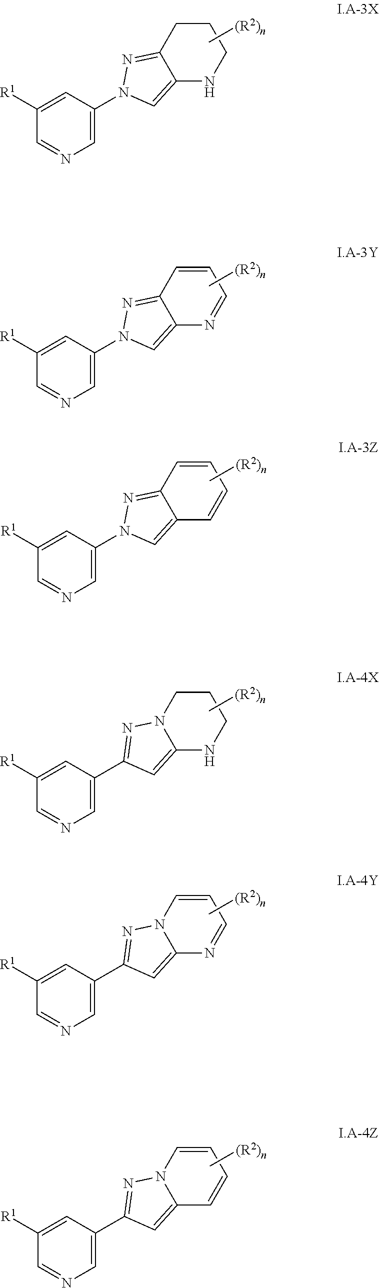

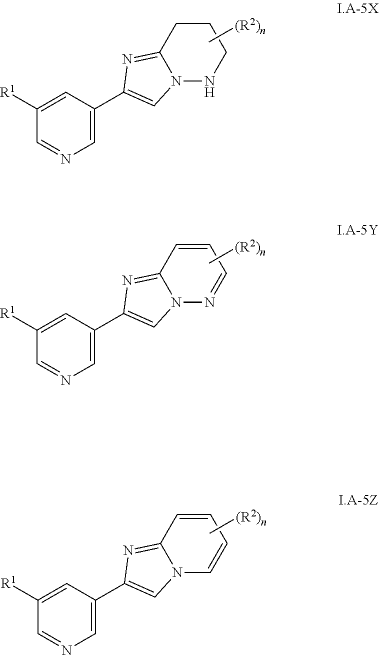

In the compounds depicted above, (R²)ₙ with n being an integer selected from 0, 1, 2, or 3 represents the optional substitution of the C-atoms and/or the N-atom of the chains X, Y, and Z, respectively, with 1, 2, 3 substituents R², said substituents R² being identical or different from one another if more than one substituent R² is present.

In a more preferred embodiment, the present invention relates to the use of compounds of formula I.A-1, I.A-2, I.A-3, I.A-4 and I.A-5, wherein $ and & mark the connection to a chain, which is selected from the group consisting of

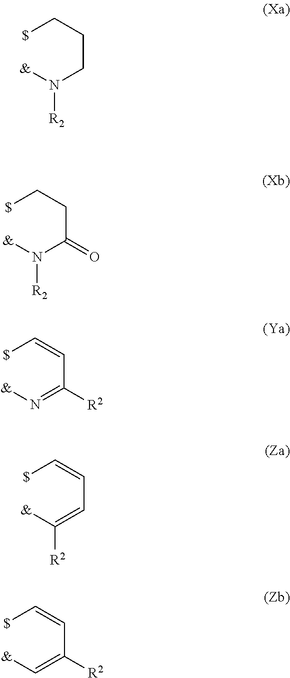

wherein in each case $ marks the connection to the $-position of the moiety A and & marks the connection to the &-position of the moiety A. These compounds correspond to formulae I.A1-Xa, I.A-1-Xb, I.A-1-Ya, I.A-1-Za, I.A-1-Zb, I.A-2-Xa, I.A-2-Xb, I.A-2-Ya, I.A-2-Za, I.A-2-Zb, I.A3-Xa, I.A-3-Xb, I.A-3-Ya, I.A-3-Za, I.A-3-Zb, I.A-4-Xa, I.A-4-Xb, I.A-4-Ya, I.A-4-Za, I.A-4-Zb, I.A5-Xa, I.A-5-Xb, I.A-5-Ya, I.A-5-Za, and I.A-5-Zb.

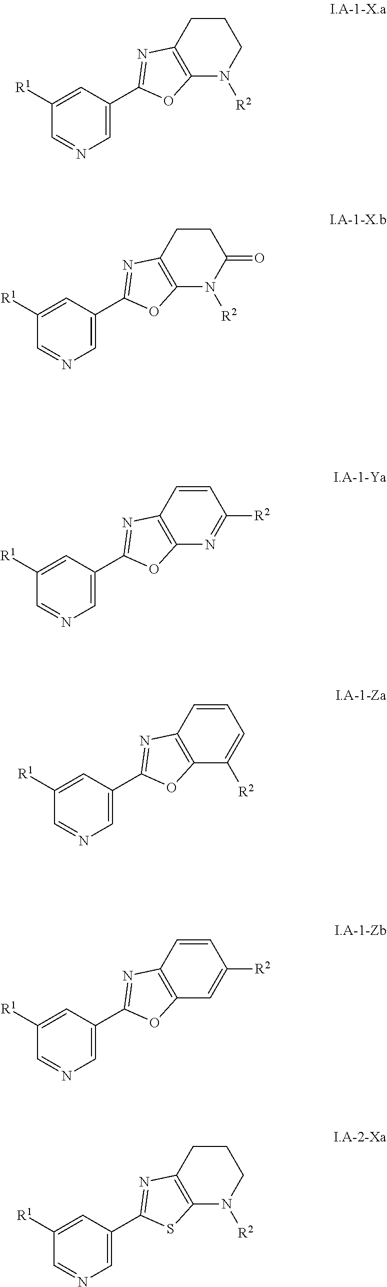

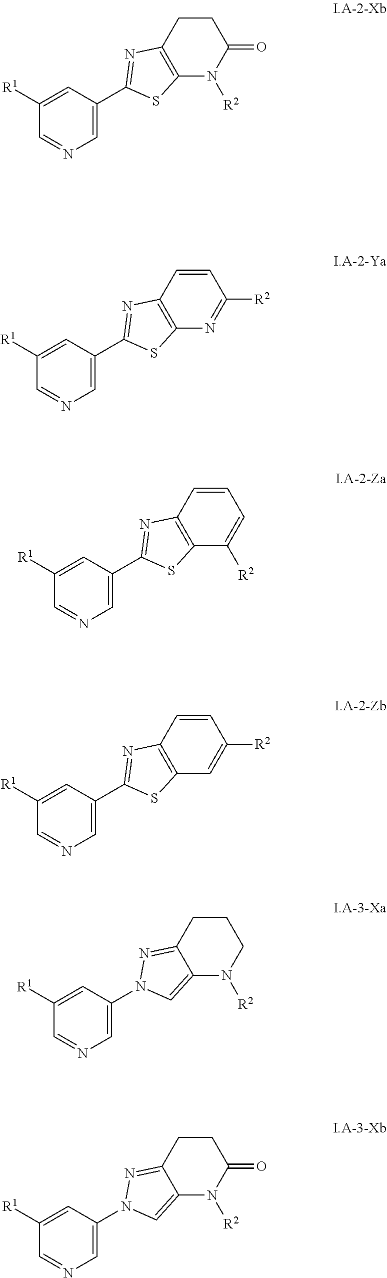

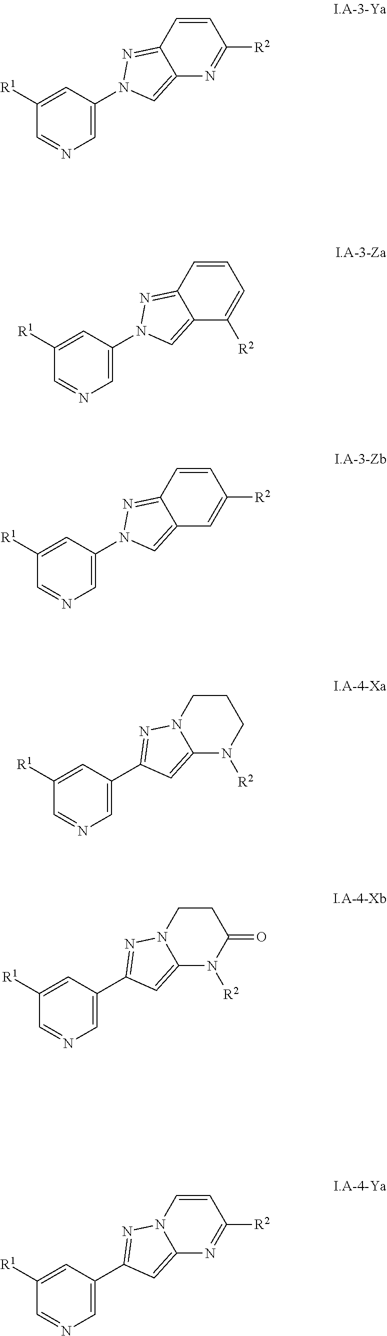

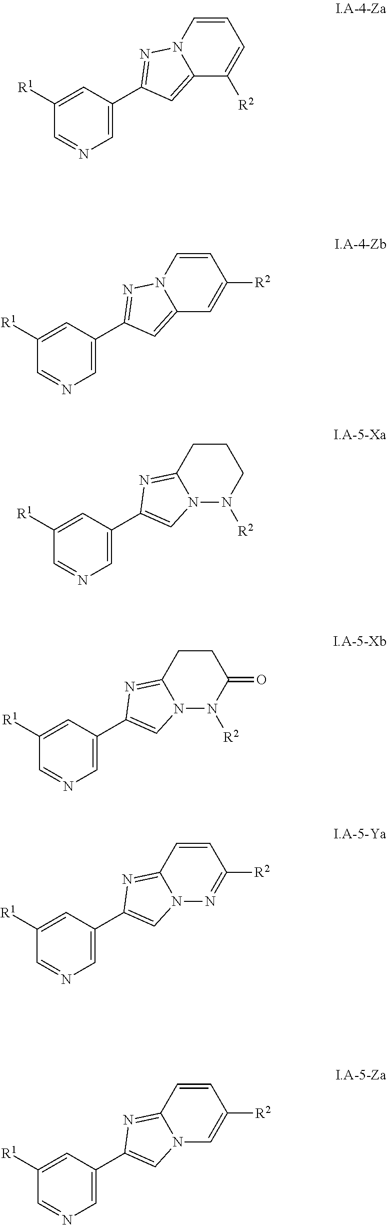

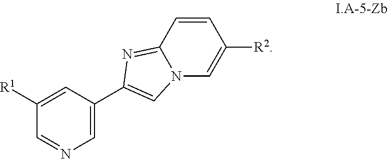

Of these compounds, the compounds I.A-1-Ya, I.A-1-Za, I.A-1-Zb, I.A-2-Xa, I.A-2-Ya, I.A-2-Za, I.A-2-Zb, I.A-3-Ya, I.A-3-Za, I.A-4-Ya, and I.A-5-Ya are particularly preferred. Said compounds are summarized under generic formula I*, and are therefore also referred to as compounds I*.A-1-Ya, I*.A-1-Za, I*.A-1-Zb, I*.A-2-Xa, I*.A-2-Ya, I*.A-2-Za, I*.A-2-Zb, I*.A-3-Ya, I*.A-3-Za, I*.A-4-Ya, and I*.A-5-Ya.

In one embodiment, the present invention relates to these novel compounds I*, i.e. the compounds I*.A-1-Ya, I*.A-1-Za, I*.A-1-Zb, I*.A-2-Xa, I*.A-2-Ya, I*.A-2-Za, I*.A-2-Zb, I*.A-3-Ya, I*.A-3-Za, I*.A-4-Ya, and I*.A-5-Ya, respectively.

In one embodiment, the present invention relates to the use of compounds of formula I, wherein R¹ is H, or F. In a preferred embodiment, R¹ is H. Compounds, wherein R¹ is H correspond to formula I.R¹═H, and compounds wherein R¹ is F correspond to formula I.R¹═F.

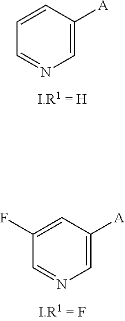

Preferred are compounds I*, wherein R¹ is H, or F, i.e. compounds I*.R¹═H and I*.R¹═F, in particular compounds I*.R¹═H.

In one embodiment, the present invention relates to these novel compounds I*.R¹═H and I*.R¹═F, preferably to compounds I*.R¹═H.

The compounds of the present invention can principally be prepared by metal catalyzed coupling reactions of a pyridine halide or triflate of formula II with a heterobicycle III.A according to standard processes of organic chemistry.

For example, compounds of formula I.A-1 and formula I.A-2 can be prepared analogously to the synthesis routes described in Org. Lett., Vol 11, 2009, pp. 1733-1736, J. Am. Chem. Soc., Vol 132, 2010, pp. 3674-3675, Org. Lett., Vol. 16, 2014, pp. 1984-1987, or WO 2009/027732 according to the following scheme.

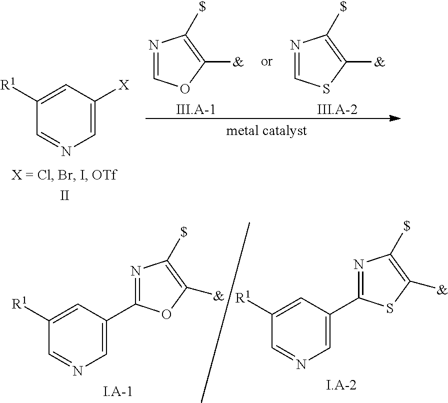

Furthermore, compounds of formula I.A-3 can, e.g., be prepared analogously to the synthesis route described in Tetrahedron, Vol 69, 2013, pp. 7279-7284 according to the following scheme.

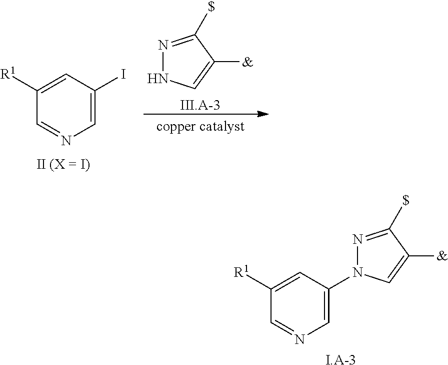

Alternatively, the compounds of the present invention can be prepared by cyclization reactions according to standard processes of organic chemistry. In such reactions, the pyridine moiety is typically introduced upon cyclization of the moiety A.

Compounds of formula I.A-4 and I. A-5 can, e.g., be prepared analogously to the synthesis described in Synth. Commun., Vol. 29, 1999, pp. 311-341. Furthermore, compounds of formula I.A-4 can be prepared analogously to the synthesis described in US 2013/0190290 or WO 2013/043521. For example, compounds of formula I.A-4-Xb can be prepared by reacting compounds of formula IIa with hydrazine to give compounds of formula III-A-4, which are then cyclized and substituted by R² to give compounds of formula I.A-4-Xb, following the scheme below.

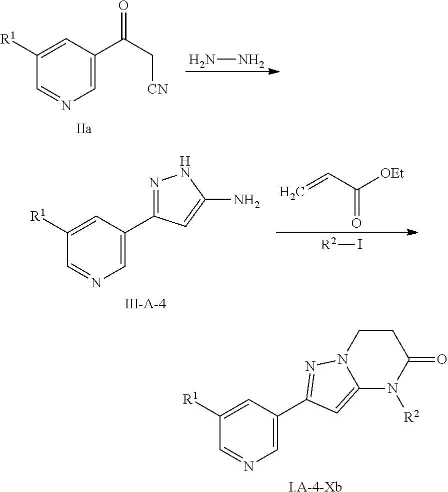

Compounds of formula I.A-5 can also be prepared analogously to the synthesis described in Adv. Synth. Catal., Vol. 356, 2014, pp. 687-691. For example, compounds of formula I.A-5-Za can be prepared by reacting compounds of formula IIb with compounds of formula Ill-Za according to the following scheme.

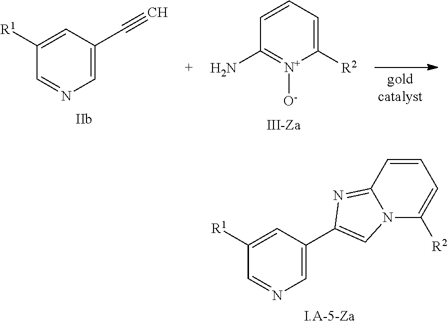

Analogous synthetic routes as described above can be used for the preparation of compounds of formula I*.

N-oxides of the compounds of formula I and I*, can be prepared by oxidation of compounds of formula I or compounds of formula I* according to standard methods of preparing heteroaromatic N-oxides, e.g. by the method described in Journal of Organometallic Chemistry 1989, 370, 17-31.

The formation of an agricultural and veterinary acceptable salt is within the skill of an ordinary organic chemist using standard techniques.

If individual compounds cannot be prepared via the above-described routes, they can be prepared by derivatization of other compounds I or I* or by customary modifications of the synthesis routes described. For example, in individual cases, certain compounds I or I* can advantageously be prepared from other compounds I or I* by ester hydrolysis, amidation, esterification, ether cleavage, olefination, reduction, oxidation and the like.

It is also appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or trituration.

Unless otherwise indicated, the term “compound(s) according to the invention” or “compound(s) of the invention” refers to the compounds of formula I and to the compounds of formula I*.

The term “compound(s) according to the invention”, or “compounds of formula I” and “compounds of formula I*”, or “compounds I” and “compounds I*” comprises the compound(s) as defined herein as well as a stereoisomer, salt, tautomer or N-oxide thereof. The term “compound(s) of the present invention” is to be understood as equivalent to the term “compound(s) according to the invention”, therefore also comprising a stereoisomer, salt, tautomer or N-oxide thereof.

The term “composition(s) according to the invention” or “composition(s) of the present invention” encompasses composition(s) comprising at least one compound of formula I or formula I* according to the invention as defined above. The compositions of the invention are preferably agricultural or veterinary compositions.

Depending on the substitution pattern, the compounds according to the invention may have one or more centres of chirality, in which case they are present as mixtures of enantiomers or diastereomers. The invention provides both the single pure enantiomers or pure diastereomers of the compounds according to the invention, and their mixtures and the use according to the invention of the pure enantiomers or pure diastereomers of the compounds according to the invention or their mixtures. Suitable compounds according to the invention also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one centre of chirality in the molecule, as well as geometrical isomers (cis/trans isomers). The present invention relates to every possible stereoisomer of the compounds of formula I, i.e. to single enantiomers or diastereomers, as well as to mixtures thereof.

The compounds according to the invention may be amorphous or may exist in one or more different crystalline states (polymorphs) which may have different macroscopic properties such as stability or show different biological properties such as activities. The present invention relates to amorphous and crystalline compounds according to the invention, mixtures of different crystalline states of the respective compounds according to the invention, as well as amorphous or crystalline salts thereof.

Salts of the compounds according to the invention are preferably agriculturally and/or veterinary acceptable salts, preferably agriculturally acceptable salts. They can be formed in a customary manner, e.g. by reacting the compound with an acid of the anion in question if the compounds according to the invention have a basic functionality or by reacting acidic compounds according to the invention with a suitable base.

Veterinary and/or agriculturally useful salts of the compounds according to the invention encompass especially the acid addition salts of those acids whose cations and anions, respectively, have no adverse effect on the pesticidal action of the compounds according to the invention.

Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting compounds according to the invention with an acid of the corresponding anion, preferably of hydrochloric acid, hydrobromic acid, sulfuric acid, phosphoric acid or nitric acid.

The term “N-oxide” includes any compound of the present invention which has at least one tertiary nitrogen atom that is oxidized to an N-oxide moiety.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine or bromine.

The term “alkyl” as used herein and in the alkyl moieties of alkylamino, alkylcarbonyl, alkylS(O)ₙ and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein and in the haloalkyl moieties of haloalkylcarbonyl, haloalkoxycarbonyl, haloalkyl-S(O)ₙ, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “cyanoalkyl” as used herein denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with cyano groups. Preferred cyanoalkyl moieties are selected from C₁-C₄-cyanoalkyl, more preferably from C₁-C₃-cyanoalkyl or C₁-C₂-cyanoalkyl, such as CH₂CN, C(CH₃)₂CN, CH₂CH₂CH₂CN, and CH(CN)-i-CH(CH₃)₂.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoromethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2-dichloro-2-fluorethoxy, 2,2,2-trichloroethoxy, pentafluoroethoxy and the like.

The term “alkylamino” as used herein refers to a straight-chain or branched saturated alkyl group having 1 to 10 carbon atoms, preferably 1 to 4 carbon atoms, more preferably 1 to 3 carbon atoms, which is bonded via a nitrogen atom, e.g. an —NH— group.

The term “dialkylamino” as used herein refers to a straight-chain or branched saturated alkyl group having 1 to 10 carbon atoms, preferably 1 to 4 carbon atoms, more preferably 1 to 3 carbon atoms, which is bonded via a nitrogen atom, which is substituted by another straight-chain or branched saturated alkyl group having 1 to 10 carbon atoms, preferably 1 to 4 carbon atoms, more preferably 1 to 3 carbon atoms, e.g. a methylamino or ethylamino group.

The term “alkylthio” (alkylsulfanyl: alkyl-S—)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom. Examples include methylthio, ethylthio, propylthio, isopropylthio, and n-butylthio.

The term “haloalkylthio” as used herein refers to an alkylthio group as mentioned above wherein the hydrogen atoms are partially or fully substituted by fluorine, chlorine, bromine and/or iodine. Examples include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like.

The term “alkylsulfinyl” (alkylsulfoxyl: C₁-C₆-alkyl-S(═O)—), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “alkylsulfonyl” (alkyl-S(═O)₂—) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “alkylcarbonyl” (C₁-C₆—C(═O)—) refers to a straight-chain or branched alkyl group as defined above, which is bonded via the carbon atom of a carbonyl group (C═O) to the remainder of the molecule.

The term “alkoxycarbonyl” refers to an alkylcarbonyl group as defined above, which is bonded via an oxygen atom to the remainder of the molecule.

The term “alkenyl” as used herein denotes in each case a singly unsaturated hydrocarbon radical having usually 2 to 10, frequently 2 to 6, preferably 2 to 4 carbon atoms, wherein the double bond may be present in any position, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radical having usually 2 to 10, frequently 2 to 6, preferably 2 to 4 carbon atoms, wherein the triple bond may be present in any position, e.g. ethynyl, propargyl (2-propyn-1-yl), 1-propyn-1-yl, 1-methyl prop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cycloalkylthio denotes in each case a monocyclic cycloaliphatic radical having usually from 3 to 10 or from 3 to 6 carbon atoms, such as cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “halocycloalkyl” as used herein and in the halocycloalkyl moieties of halocycloalkoxy and halocycloalkylthio denotes in each case a monocyclic cycloaliphatic radical having usually from 3 to 10 C-atoms or 3 to 6 C-atoms, wherein at least one, e.g. 1, 2, 3, 4 or 5 of the hydrogen atoms, are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-, 2- and 3-fluorocyclopentyl, 1,2-, 2,2-, 2,3-, 3,3-, 3,4-, 2,5-difluorocyclopentyl, 1-, 2- and 3-chlorocyclopentyl, 1,2-, 2,2-, 2,3-, 3,3-, 3,4-, 2,5-dichlorocyclopentyl and the like.

The term “cycloalkylalkyl” refers to a cycloalkyl group as defined above which is bonded via an alkyl group, such as a C₁-C₅-alkyl group or a C₁-C₄-alkyl group, in particular a methyl group (=cycloalkylmethyl), to the remainder of the molecule.

The term “halocycloalkylalkyl” refers to a halocycloalkyl group as defined above which is bonded via an alkyl group, such as a C₁-C₅-alkyl group or a C₁-C₄-alkyl group, in particular a methyl group (=cycloalkylmethyl), to the remainder of the molecule.

The term “cycloalkenyl” as used herein and in the cycloalkenyl moieties of cycloalkenyloxy and cycloalkenylthio denotes in each case a monocyclic singly unsaturated non-aromatic radical having usually from 3 to 10, e.g. 3 or 4 or from 5 to 10 carbon atoms, preferably from 3- to 8 carbon atoms. Exemplary cycloalkenyl groups include cyclopropenyl, cycloheptenyl or cyclooctenyl.

The term “carbocycle” or “carbocyclyl” includes, unless otherwise indicated, in general a 3- to 12-membered, preferably a 3- to 8-membered or a 5- to 8-membered, more preferably a 5- or 6-membered mono-cyclic, ring comprising 3 to 12, preferably 3 to 8 or 5 to 8, more preferably 5 or 6 carbon atoms. The heterocyclic radicals may be saturated, partially unsaturated, or fully unsaturated. Preferably, the term “carbocycle” covers cycloalkyl and cycloalkenyl groups as defined above, for example cyclopropane, cyclobutane, cyclopentane and cyclohexane rings.

When it is referred to “fully unsaturated” carbocycles, this term also includes “aromatic” carbocycles. In certain preferred embodiments, a fully unsaturated carbocycle is an aromatic carbocycle as defined below, preferably a 6-membered aromatic carbocycle.

The term “heterocycle” or “heterocyclyl” includes, unless otherwise indicated, in general 3- to 12-membered, preferably 3- to 8-membered, 3- to 7-membered, or 5- to 8-membered, more preferably 5- or 6-membered, in particular 6-membered monocyclic heterocyclic radicals. The heterocyclic radicals may be saturated, partially unsaturated, or fully unsaturated. As used in this context, the term “fully unsaturated” also includes “aromatic”. In a preferred embodiment, a fully unsaturated heterocycles is thus an aromatic heterocycle, preferably a 5- or 6-membered aromatic heterocycle comprising one or more, e.g. 1, 2, 3, or 4, preferably 1, 2, or 3 heteroatoms selected from N, O and S as ring members. Examples of aromatic heterocycles are provided below in connection with the definition of “hetaryl”. Unless otherwise indicated, “hetaryls” are thus covered by the term “heterocycles”. The heterocyclic non-aromatic radicals usually comprise 1, 2, 3, 4 or 5, preferably 1, 2 or 3 heteroatoms selected from N, O and S as ring members, where S-atoms as ring members may be present as S, SO or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S.oxothiopyranyl, Sdioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-onyl and the like.

The term “aryl” or “aromatic carbocycle” or “aromatic carbocyclic ring” includes mono-, bi- or tricyclic aromatic radicals having usually from 6 to 14, preferably 6, 10 or 14 carbon atoms. Exemplary aryl groups include phenyl, naphthyl and anthracenyl. Phenyl is preferred as aryl group.

The term “hetaryl” or “aromatic heterocycle” or “aromatic heterocyclic ring” includes monocyclic 5- or 6-membered heteroaromatic radicals comprising as ring members 1, 2, 3 or 4 heteroatoms selected from N, O and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “hetaryl” also includes bicyclic 8 to 10-membered heteroaromatic radicals comprising as ring members 1, 2 or 3 heteroatoms selected from N, O and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like.

These fused hetaryl radicals may be bonded to the remainder of the molecule via any ring atom of 5- or 6-membered heteroaromatic ring or via a carbon atom of the fused phenyl moiety.

The terms “alkylene”, “alkenylene”, and “alkynylene” refer to alkyl, alkenyl, and alkynyl as defined above, respectively, which are bonded to the remainder of the molecule, via two atoms, preferably via two carbon atoms, of the respective group, so that they represent a linker between two moieties of the molecule. In particular, the term “alkylene” may refer to alkyl chains such as CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂. Similarly, “alkenylene” and “alkynylene” may refer to alkenyl and alkynyl chains, respectively.

When it is referred to an acetal group based on alcohols or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH as a substituent, said substituent is preferably to be understood as a heterocyclic spiro substituent, which is formed by reacting an oxo substituent ═O with a respective alcohol or thiol. An example for such a substituent is realized in the following group B⁵, which may referred to as a C₆-cycloalkyl substituent, which is substituted by an acetyl group based on HOCH₂CH₂OH:

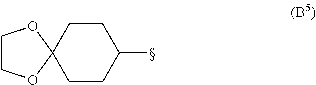

When it is referred to certain radicals being substituted with “one or more” substitutents, the term “one or more” is intended to cover at least one substituent, e.g. 1 to 10 substituents, preferably 1, 2, 3, 4, or 5 substituents, more preferably 1, 2, or 3 substituents, most preferably 1, or 2 substituents.

With respect to the variables, the particularly preferred embodiments of the intermediates correspond to those of the compounds of the formula I and formula I*, respectively.

In a particular embodiment, the variables of the compounds of the formula I and formula I* have the following meanings, these meanings, both on their own and in combination with one another, being particular embodiments of the compounds of the formula I and formula I*:

In a preferred embodiment, the invention relates to the use of compounds of formula I, wherein A is a moiety selected from the group consisting of A-1, A-2, A-3, A-4, and A-5 as depicted above, wherein # denotes the bond to the pyridine ring of formula I, and $ and & mark the connection to a C₄-alkylene or C₄-alkenylene chain of which 1 C-atom may be replaced by N, and which forms together with the atoms to which it is bonded an annulated saturated, partially unsaturated, or aromatic 6-membered carbocyclic or heterocyclic ring, wherein the C-atoms and/or the N-atom of the chain may be substituted by 1, 2, or 3 substituents R², said substituents R² being identical or different from one another if more than one substituent R² is present.

In a more preferred embodiment, the invention relates to the use of compounds of formula I, wherein A is a moiety selected from the group consisting of A-1, A-2, A-3, A-4, and A-5 as depicted above, wherein # denotes the bond to the pyridine ring of formula I; and $ and & mark the connection to a chain, which is selected from the group consisting of X, Y, and Z as depicted above, wherein in each case $ marks the connection to the $-position of the moiety A and & marks the connection to the &-position of the moiety A, and wherein the C-atoms and/or the N-atom of the chains may be substituted by 1, 2, or 3 substituents R², said substituents R² being identical or different from one another if more than one substituent R² is present. These compounds correspond to formulae I.A-1-X, I.A-1-Y, I.A-1-Z, I.A-2-X, I.A-2-Y, I.A-2-Z, I.A-3-X, I.A-3-Y, I.A-3-Z, I.A-4-X, I.A-4-Y, I.A-4-Z, I.A-5-X, I.A-5-Y, and I.A-5-Z, respectively. In one particularly preferred embodiment, the invention relates to the use of compounds selected from compounds of formulae I.A-1-X, I.A-1-Y, and I.A-1-Z. In another particularly preferred embodiment, the invention relates to the use of compounds selected from compounds of formulae I.A-2-X, I.A-2-Y, and I.A-2-Z. In another particularly preferred embodiment, the invention relates to the use of compounds selected from compounds of formulae I.A-3-X, I.A-3-Y, and I.A-3-Z. In yet another particularly preferred embodiment, the present invention relates to the use of compounds selected from compounds of formulae I.A-4-X, I.A-4-Y, and I.A-4-Z. In yet another particularly preferred embodiment, the present invention relates to the use of compounds selected from compounds of formulae I.A-5-X, I.A-5-Y, and I.A-5-Z.

In an even more preferred embodiment, the present invention relates to the use of compounds of formula I, wherein A is a moiety selected from the group consisting of A-1, A-2, A-3, A-4, and A-5 as depicted above, wherein # denotes the bond to the pyridine ring of formula I; and $ and & mark the connection to a chain, which is selected from the group consisting of Xa, Xb, Ya, Za, and Zb as depicted above, wherein in each case $ marks the connection to the $-position of the moiety A and & marks the connection to the &-position of the moiety A. These compounds correspond to formulae I.A-1-Xa, I.A-1-Xb, I.A-1-Ya, I.A-1-Za, I.A-1-Zb, I.A-2-Xa, I.A-2-Xb, I.A-2-Ya, I.A-2-Za, I.A-2-Zb, I.A-3-Xa, I.A-3-Xb, I.A-3-Ya, I.A-3-Za, I.A-3-Zb, I.A-4-Xa, I.A-4-Xb, I.A-4-Ya, I.A-4-Za, I.A-4-Zb, I.A-5-Xa, I.A-5-Xb, I.A-5-Ya, I.A-5-Za, and I.A-5-Zb, respectively.

In one particularly preferred embodiment, the present invention relates to the use of compounds of formula I, wherein A is a moiety selected from the group consisting of

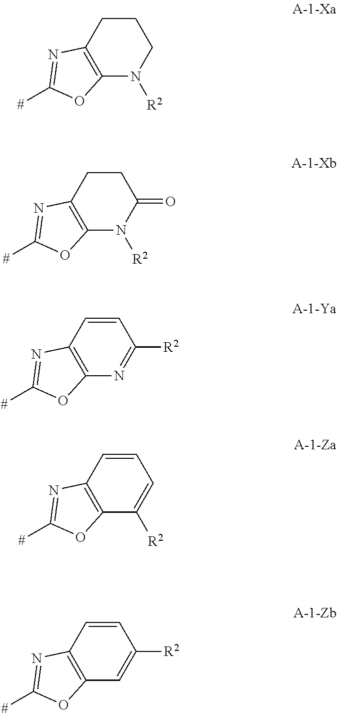

In another particularly preferred embodiment, the present invention relates to the use of compounds of formula I, wherein A is a moiety selected from the group consisting of

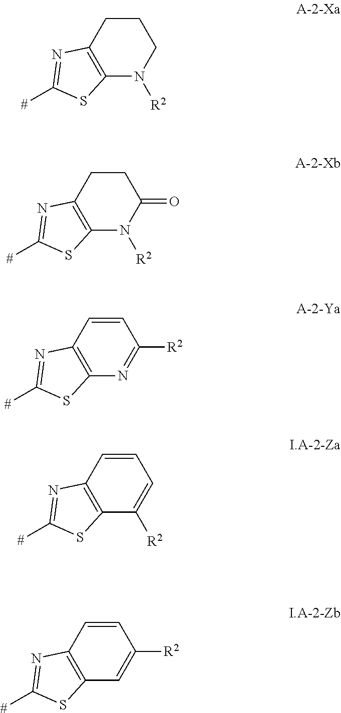

In yet another particularly preferred embodiment, the present invention relates to the use of compounds of formula I, wherein A is a moiety selected from the group consisting of

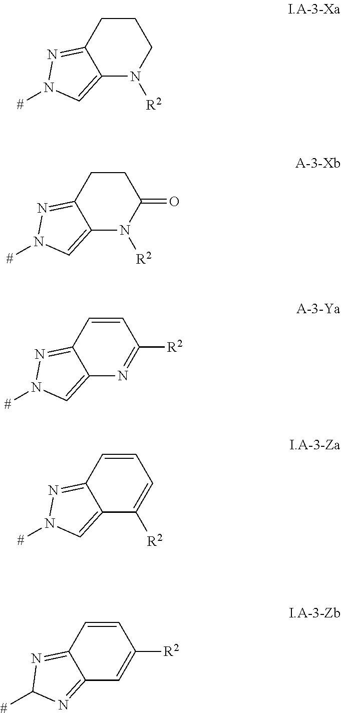

In yet another particularly preferred embodiment, the present invention relates to the use of compounds of formula I, wherein A is a moiety selected from the group consisting of

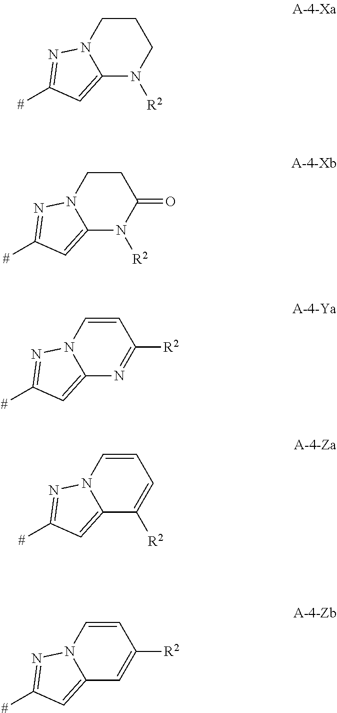

In yet another particularly preferred embodiment, the present invention relates to the use of compounds of formula I, wherein A is a moiety selected from the group consisting of

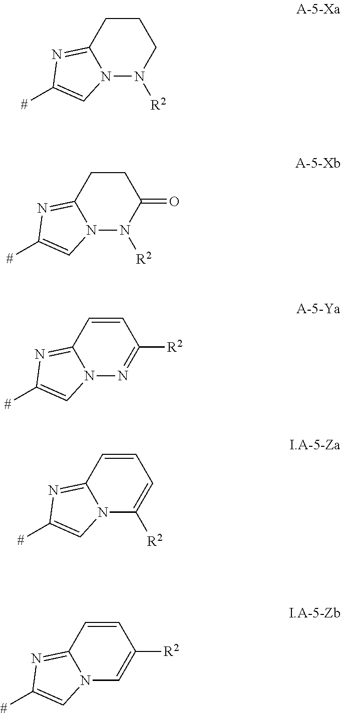

In another particularly preferred embodiment, the present invention relates to the use of compounds selected from compounds of formulae I.A-2-Xa, I.A-2-Xb, I.A-2-Ya, I.A-1-Za, and Thus, in one particularly preferred embodiment, the present invention relates to the use of compounds selected from compounds of formulae I.A-1-Xa, I.A-1-Xb, I.A-1-Ya, I.A-1-Za, and I.A-1-Zb. In another particularly preferred embodiment, the present invention relates to the use of compounds selected from compounds of formulae I.A-2-Xa, I.A-2-Xb, I.A-2-Ya, I.A-2-Za, and I.A-2-Zb. In another particularly preferred embodiment, the present invention relates to the use of compounds selected from compounds of formulae I.A-3-Xa, I.A-3-Xb, I.A-3-Ya, I.A-3-Za, and I.A-3-Zb. In yet another particularly preferred embodiment, the present invention relates to the use of compounds selected from compounds of formulae. I.A-4-Xa, I.A-4-Xb, I.A-4-Ya, I.A-4-Za, and I.A-4-Zb. In yet another particularly preferred embodiment, the present invention relates to the use of compounds selected from compounds of formulae I.A-5-Xa, I.A-5-Xb, I.A-5-Ya, I.A-5-Za, and I.A-5-Zb.

In another particularly preferred embodiment, the present invention relates to the use of compounds selected from compounds of formulae I.A-1-Ya, I.A-1-Za, I.A-1-Zb, I.A-2-Xa, I.A-2-Ya, I.A-2-Za, I.A-2-Zb, I.A-3-Ya, I.A-3-Za, I.A-4-Ya, and I.A-5-Ya, which are summarized under generic formula I*, and are therefore also referred to as compounds of formulae I*.A-1-Ya, I*.A-1-Za, I*.A-1-Zb, I*.A-2-Xa, I*.A-2-Ya, I*.A-2-Za, I*.A-2-Zb, I*.A-3-Ya, I*.A-3-Za, I*.A-4-Ya, and I*.A-5-Ya.

In one preferred embodiment, the present invention relates to the use of compounds of formula I, wherein R² is a substituent

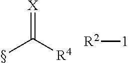

wherein § denotes the bond to the atom on which R² is present; X is NR³, O, or S; and R⁴ is H, CR⁵R⁶R⁷, NR⁸R⁹, OR¹⁰, or SR¹⁰. Preferably, R² is a substituent

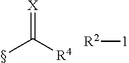

wherein § denotes the bond to the atom on which R² is present; X is O or S; and R⁴ is H, CR⁵R⁶R⁷, NR⁸R⁹, or OR¹⁰, wherein R⁵, R⁶, R⁷, R⁸, R⁹, and R¹⁰ are as defined above.

In view of the above, particularly preferred compounds for use according to the present invention include compounds of formulae I.A-1-Xa.R²-1, I.A-1-Xb.R²-1, I.A-1-Ya.R²-1, I.A-1-Za.R²-1, I.A-1-Z.bR²-1, I.A-2-Xa.R²-1, I.A-2-Xb.R²-1, I.A-2-Ya.R²-1, I.A-2-Za.R²-1, I.A-2-Zb.R²-1, I.A-3-Xa.R²-1, I.A-3-Xb.R²-1, I.A-3-Ya.R²-1, I.A-3-Za.R²-1, I.A-3-Zb.R²-1, I.A-4-Xa.R²-1, I.A-4-Xb.R²-1, I.A-4-Ya.R²-1, I.A-4-Za.R²-1, I.A-4-Zb.R²-1, I.A-5-Xa.R²-1, I.A-5-Xb.R²-1, I.A-5-Ya.R²-1, I.A-5-Za.R²-1, and I.A-5-Zb.R²-1, as depicted below.

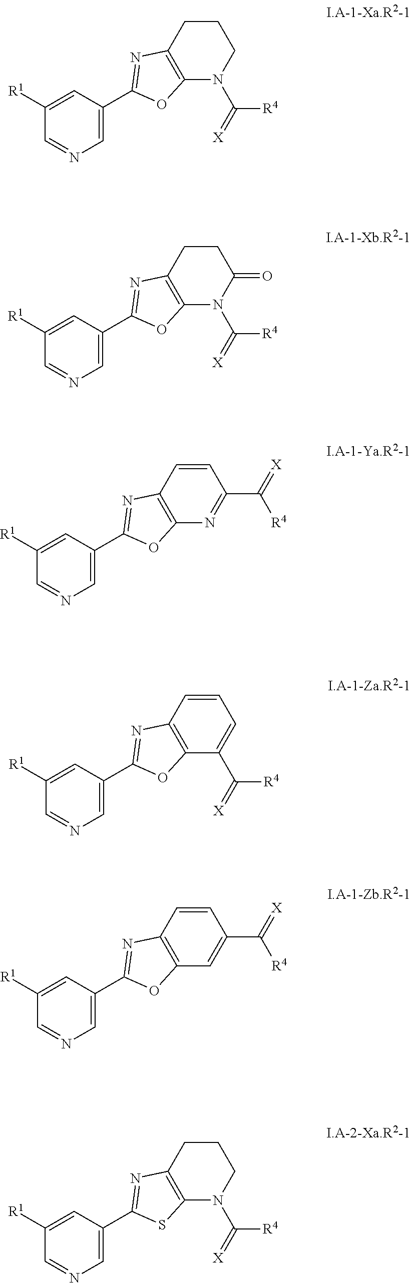

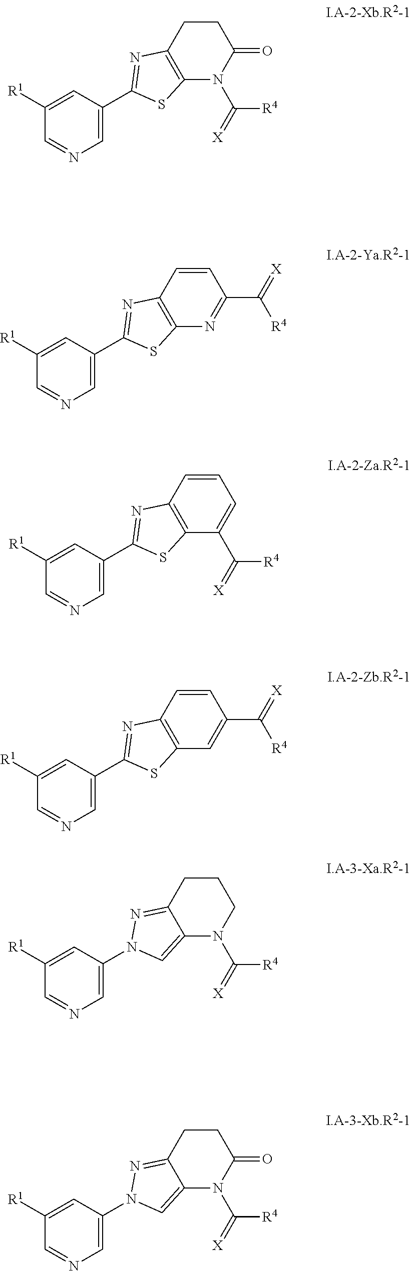

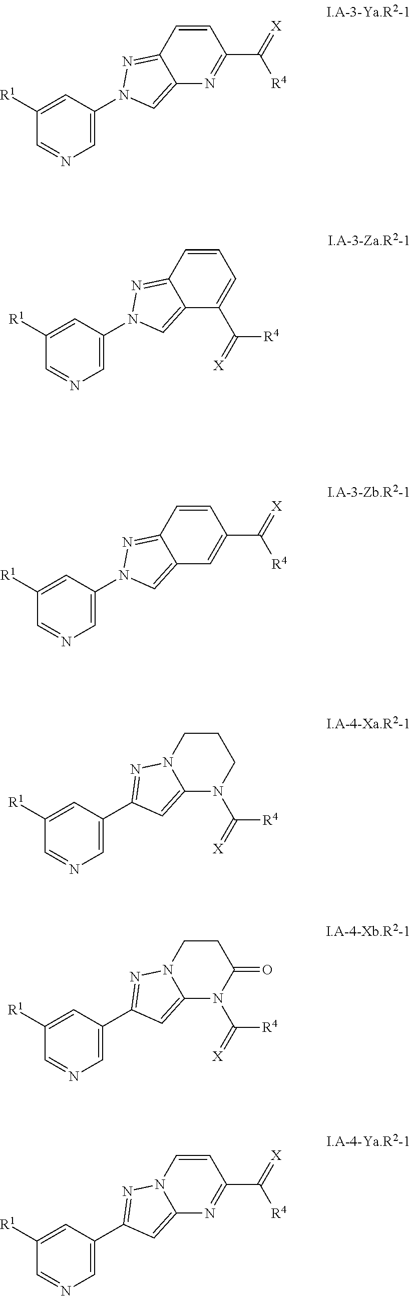

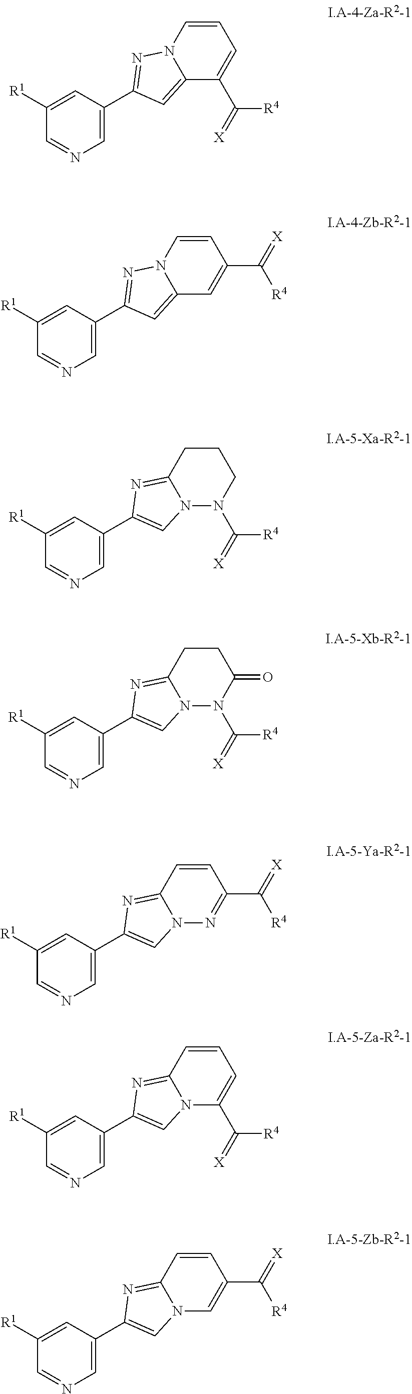

In one particularly preferred embodiment, the present invention relates to the use of compounds selected from any one of the following groups of compounds:

(i) compounds of formulae I.A-1-Xa.R²-1, I.A-1-Xb.R²-1, I.A-1-Ya.R²-1, I.A-1-Za.R²-1, I.A-1-Zb.R²-1, or

(ii) compounds of formulae I.A-2-Xa.R²-1, I.A-2-Xb.R²-1, I.A-2-Ya.R²-1, I.A-2-Za.R²-1, I.A-2-Zb.R²-1, or

(iii) compounds of formulae I.A-3-Xa.R²-1, I.A-3-Xb.R²-1, I.A-3-Ya.R²-1, I.A-3-Za.R²-1, I.A-3-Zb.R²-1, or

(iv) compounds of formulae I.A-4-Xa.R²-1, I.A-4-Xb.R²-1, I.A-4-Ya.R²-1, I.A-4-Za.R²-1, I.A-4-Zb.R²-1,

(v) compounds of formulae I.A-5-Xa.R²-1, I.A-5-Xb.R²-1, I.A-5-Ya.R²-1, I.A-5-Za.R²-1, and I.A-5-Zb.R²-1.

In another particularly preferred embodiment, the present invention relates to the use of compounds selected from compounds of formulae I.A-1-Ya.R²-1, I.A-1-Za.R²-1, I.A-1-Zb.R²-1, I.A-2-Xa.R²-1, I.A-2-Ya.R²-1, I.A-2-Za.R²-1, I.A-2-Zb.R²-1, I.A-3-Ya.R²-1, I.A-3-Za.R²-1, I.A-4-Ya.R²-1, and I.A-5-Ya.R²-1, which can be summarized under generic formula I*.R²-1, and are therefore also referred to as compounds of formulae I*.A-1-Ya.R²-1, I*.A-1-Za.R²-1, I*.A-1-Zb.R²-1, I*.A-2-Xa.R²-1, I*.A-2-Ya.R²-1, I*.A-2-Za.R²-1, I*.A-2-Zb.R²-1, I*.A-3-Ya.R²-1, I*.A-3-Za.R²-1, I*.A-4-Ya.R²-1, and I*.A-5-Ya.R²-1.

In another preferred embodiment, the invention relates to the use of compounds of formula I, wherein R¹ is H or F, i.e. compounds of formula I.R¹═H and formula I.R¹═F as depicted above. Compounds of formula I, wherein R¹ is H, i.e. compounds of formula I.R¹═H, are particularly preferred. Even more preferred is the use of compounds of formula I*, wherein R¹ is H or F, i.e. compounds of formula I*.R¹═H and formula I*.R¹═F, in particular compounds of formula I*.R¹═H. In particular, reference is made to compounds of formulae I*.A-1-Ya.R¹═H, I*.A-1-Za. R¹═H, I*.A-1-Zb.R¹═H, I*.A-2-Xa.R¹═H, I*.A-2-Ya.R¹═H, I*.A-2-Za.R¹═H, I*.A-2-Zb.R¹═H, I*.A-3-Ya.R¹═H, I*.A-3-Za.R¹═H, I*.A-4-Ya.R¹═H, and I*.A-5-Ya.R¹═H.

Most preferred is the use of compounds of formula I*, wherein R¹ is H and R² is R²-1, i.e. compounds of formula I*.R¹═H.R²-1, in particular compounds of formulae I*.A-1-Ya.R¹═H.R²-1, I*.A-1-Za.R¹═H.R²-1, I*.A-1-Zb.R¹═H.R²-1, I*.A-2-Xa.R¹¹═H.R²-1, I*.A-2-Ya.R¹═H.R²-1, I*.A-2-Za.R¹═H.R²-1, I*.A-2-Zb.R¹═H.R²-1, I*.A-3-Ya.R¹═H.R²-1, I*.A-3-Za.R¹═H.R²-1, I*.A-4-Ya.R¹═H.R²-1, and I*.A-5-Ya.R¹═H.R²-1.

In one preferred embodiment, the present invention relates to the use of compounds I as defined above, or to the novel compounds I* as defined above, wherein at least one substituent R² is present, and wherein said at least one substituent
R² is OR¹², S(O)ₙR¹², S(O)ₙNR^{13a}R^{13b}, preferably OR¹² or S(O)ₙR¹²;
and wherein
R¹² is H;
   C₁-C₆-alkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
   C₁-C₆-haloalkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
   C₃-C₈-cycloalkyl, C₃-C₈-cycloalkyl-C₁-C₄-alkyl, C₃-C₈-halocycloalkyl, C₃-C₈-cycloalkyl-C₁-C₄-alkyl, wherein the cyclic moieties are unsubstituted, or substituted with one or more, same or different substituents selected from halogen, CN, C₁-C₄-alkyl, C₁-C₄-haloalkyl, C₁-C₄-alkoxy, C₁-C₄-haloalkoxy, ═O, and acetal groups thereof based on alcohols, or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH;
   C₂-C₆-alkenyl, C₂-C₆-haloalkenyl, C₂-C₆-alkynyl, C₂-C₆-haloalkynyl;
   phenyl, or phenyl-C₁-C₄-alkyl, wherein the aromatic rings are in each case unsubstituted, or substituted with one or more, same or different substituents R²²;
   or a 3- to 7-membered saturated, partially unsaturated, or fully unsaturated heterocyclic ring comprising one or more, same or different heteroatoms O, N or S, wherein the heterocyclic ring is directly bonded to the remainder of the molecule, or bonded to the remainder of the molecule via a C₁-C₄-alkyl group, and wherein the heterocyclic ring is unsubstituted, or substituted with one or more, same or different substituents R²², and wherein the N- and/or S-atoms of the heterocyclic ring are oxidized, or non-oxidized,
R^{13a}, R^{13b} are H, C₁-C₆-alkyl, C₁-C₆-haloalkyl, C₁-C₆-alkoxy, C₁-C₆-haloalkoxy, C₁-C₆-alkyl-S—, C₁-C₆-haloalky-S—, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —S(O)ₙNR^{21a}R^{21b}, C(═O)R¹⁹, C(═O)OR²⁰, C(═O)NR^{21a}R^{21b}, C(═S)R¹⁹, C(═S)SR²⁰, C(═S)NR^{21a}R^{21b}, C(═NR^{21a})R¹⁹; phenyl, which is unsubstituted, or substituted with one or more, same or different substituents R²²;
   a 3- to 7-membered saturated, partially unsaturated or fully unsaturated heterocyclic ring comprising one or more, same or different heteroatoms O, N, or S, wherein the heterocyclic ring is unsubstituted, or substituted with one or more, same or different substituents R²², and wherein the N- and/or S-atoms of the heterocyclic ring are oxidized, or non-oxidized;
   or R^{13a} and R^{13b} are together a C₂-C₇ alkylene, or C₂-C₇ alkenylene chain, and form a 3- to 8-membered saturated, partially unsaturated, or fully unsaturated ring together with the N-atom they are bonded to,
   wherein the C₂-C₇ alkylene chain or C₂-C₇ alkenylene chain contains none, one or more, same or different heteratoms O, S, or N, and is unsubstituted, or substituted with halogen, C₁-C₆-alkyl, C₁-C₆-haloalkyl, C₁-C₆-alkoxy, C₁-C₆-haloalkoxy, C₁-C₆-alkyl-S—, C₁-C₆-haloalkyl-S, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl;
   phenyl, which is unsubstituted, or substituted with one or more, same or different substituents R²²;
   or a 3- to 7-membered saturated, partially unsaturated, or fully unsaturated heterocyclic ring comprising one or more, same or different heteroatoms O, S, or N, wherein the heterocyclic ring is unsubstituted, or substituted with one or more, same or different substituents R²², and wherein the N- and/or S-atoms of the heterocyclic ring are oxidized, or non-oxidized;
and wherein all other substituents have the meanings as defined above.

Preferably, the substituents mentioned in the definition of R¹², R^{13a}, and R^{13b} above have the following meanings:
R¹⁵, R¹⁶ are independently C₁-C₆ alkyl, or phenyl;
R¹⁹ is H, C₁-C₆-alkoxy, C₁-C₆-haloalkoxy, C₁-C₆-alkyl-S(O)ₙ—, C₁-C₆-haloalkyl-S(O)ₙ—, C₁-C₆-alkyl, C₂-C₆-alkenyl, C₂-C₆-alkynyl, C₃-C₈-cycloalkyl, wherein the aliphatic and cycloaliphatic radicals are unsubstituted, partially, or fully halogenated, and/or oxygenated, and/or substituted by one or more, same or different radicals selected from C₁-C₄-alkoxy; phenyl, benzyl, pyridyl, or phenoxy, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino, and di-(C₁-C₆-alkyl)amino;
R²⁰ is H, C₁-C₆-alkyl, C₂-C₆-alkenyl, C₂-C₆-alkynyl, or C₃-C₈-cycloalkyl, wherein the aliphatic and cycloaliphatic radicals are unsubstituted, partially, or fully halogenated, and/or oxygenated, and/or substituted by one or more, same or different substituents selected from C₁-C₄-alkoxy;
   phenyl, benzyl, or pyridyl, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
R^{21a}, R^{21b} are independently H, C₁-C₆-alkoxy, C₁-C₆-haloalkoxy, C₁-C₆-alkyl-S(O)ₙ—, C₁-C₆-haloalkyl-S(O)ₙ—, C₁-C₆-alkyl, C₂-C₆-alkenyl, C₂-C₆-alkynyl, C₃-C₈-cycloalkyl, C₃-C₈-cycloalkyl-C₁-C₆-alkyl, wherein the aliphatic and cycloaliphatic radicals are unsubstituted, partially, or fully halogenated, and/or oxygenated, and/or substituted by one or more, same or different substituents selected from C₁-C₄-alkoxy;
   phenyl, benzyl, pyridyl, and phenoxy, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
   or R^{21a} and R^{21b} are together a C₂-C₆ alkylene chain forming a 3- to 7-membered saturated, partially unsaturated, or fully unsaturated ring together with the N-atom they are bonded to, wherein the alkylene chain contains none, one or more, same or different heteroatoms O, S, or N, and is unsubstituted, or substituted with one or more, same or different substituents selected from halogen, C₁-C₄-haloalkyl, C₁-C₄-alkoxy, and C₁-C₄-haloalkoxy, and wherein the N- and/or S-atoms of the heterocyclic ring are oxidized, or non-oxidized; and
R²² is H, halogen, NO₂, CN, OH, SH, C₁-C₆-alkoxy, C₁-C₆-haloalkoxy, C₁-C₆-alkyl-S(O)ₙ, C₁-C₆-haloalkyl-S(O)ₙ—, C₁-C₆-alkyl, C₂-C₆-alkenyl, C₂-C₆-alkynyl, C₃-C₈-cycloalkyl, wherein the aliphatic and cycloaliphatic radicals are unsubstituted, partially, or fully halogenated, and/or oxygenated, and/or substituted by one or more, same or different substituents selected from C₁-C₄-alkoxy,
   phenyl, benzyl, pyridyl, or phenoxy, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
   or two R²² present together on one atom are ═O, ═S, ═N(C₁-C₆-alkyl), ═NO(C₁-C₆-alkyl), ═CH(C₁-C₄-alkyl), or ═C(C₁-C₄-alkyl)C₁-C₄-alkyl;
   or two R²² on two adjacent C-atoms are together a C₂-C₆ alkylene chain, or C₂-C₆-alkenylene chain, which form together with the C-atom they are bonded to a 3- to 7-membered saturated, partially unsaturated, or fully unsaturated ring, wherein the alkylene, or alkenylene chain contains one or more, same or different heteroatoms O, S, or N, and is unsubstituted, or substituted with one or more, same or different substituents selected from halogen, C₁-C₄-haloalkyl, C₁-C₄-alkoxy, and C₁-C₄-haloalkoxy, and wherein the N- and/or S-atoms of the heterocyclic ring are oxidized, or non-oxidized.

Particularly preferably,
R¹⁵, R¹⁶ are independently selected from C₁-C₆ alkyl;
R¹⁹ is H, C₁-C₆-alkyl, C₂-C₆-alkenyl, C₂-C₆-alkynyl, C₃-C₈-cycloalkyl, wherein the aliphatic and cycloaliphatic radicals are unsubstituted, partially or fully halogenated and/or oxgenated and/or substituted by one or more, same or different radicals selected from C₁-C₄-alkoxy;
R²⁰ is H, C₁-C₆-alkyl, C₂-C₆-alkenyl, C₂-C₆-alkynyl, C₃-C₈-cycloalkyl, wherein the aliphatic and cycloaliphatic radicals are unsubstituted, partially or fully halogenated and/or oxygenated and/or substituted by one or more, same or different radicals selected from C₁-C₄-alkoxy; phenyl, benzyl, or pyridyl, wherein the radicals are unsubstituted, partially or fully halogenated and/or substituted by one or more, same or different radicals selected from halogen, C₁-C₆-alkyl, C₁-C₆-haloalkyl, C₁-C₆-alkoxy, C₁-C₆ haloalkoxy or (C₁-C₆-alkoxy)carbonyl;
R^{21a}, R^{21b} are independently H, C₁-C₆-alkoxy, C₁-C₆-haloalkoxy, C₁-C₆-alkyl-S(O)ₙ—, C₁-C₆-haloalkyl-S(O)ₙ—, C₁-C₆-alkyl, C₂-C₆-alkenyl, C₂-C₆-alkynyl, C₃-C₈-cycloalkyl, or C₃-C₈-cycloalkyl-C₁-C₆-alkyl, wherein the aliphatic and cycloaliphatic radicals are unsubstituted, partially or fully halogenated and/or oxygenated and/or substituted by one or more, same or different radicals selected from C₁-C₄-alkoxy;
   or R^{21a} and R^{21b} are together a C₂-C₆ alkylene chain forming a 3- to 7-membered saturated, partially unsaturated or fully unsaturated ring together with the N-atom R^{21a} and R^{21b} are bonded to, wherein the alkylene chain may contain one or more, same or different heteroatoms O, S, or N, and are unsubstituted or substituted with halogen, C₁-C₄-haloalkyl, C₁-C₄-alkoxy, or C₁-C₄-haloalkoxy, and wherein the N- and/or S-atoms of the heterocyclic ring are oxidized or non-oxidized; and
R²² is H, halogen, NO₂, CN, C₁-C₆-alkoxy, C₁-C₆-haloalkoxy, C₁-C₆-alkyl-S(O)ₙ, C₁-C₆-haloalkyl-S(O)ₙ—, C₁-C₆-alkyl, C₂-C₆-alkenyl, C₂-C₆-alkynyl, or C₃-C₈-cycloalkyl, wherein the aliphatic and cyclo-aliphatic radicals are unsubstituted, partially or fully halogenated, and/or oxygenated, and/or substituted by one or more, same or different radicals selected from C₁-C₄-alkoxy.

It is to be understood that in addition to the above defined at least one substituent R², at least one additional substituent R² may be present, which is preferably halogen, C₁-C₁₀-alkyl, C₁-C₁₀-haloalkyl or CN, and more preferably F, Cl, Br, I, CH₃, CF₃, or CN.

In a preferred embodiment, one substituent R² is present, which is OR¹² or S(O)ₙR¹² with R¹² being as defined above, and one substituent R² is present, which is F, Cl, Br, I, CH₃, CF₃, or CN.

In another preferred embodiment, only one substituent R² is present in the compounds of the present invention, wherein R² is OR¹² or S(O)ₙR¹² with R¹² being as defined above.

In a particularly preferred embodiment, the present invention relates to the use of compounds I as defined above, or the novel compounds I* as defined above, wherein at least one substituent
R² is present, and wherein said at least one substituent
R² is OR¹², S(O)ₙR¹²;
and wherein
R¹² is C₁-C₆-alkyl, which is unsubstituted or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
   C₁-C₆-haloalkyl, which is unsubstituted or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
   C₃-C₈-cycloalkyl, C₃-C₈-cycloalkyl-C₁-C₄-alkyl, C₃-C₈-halocycloalkyl, C₃-C₈-cycloalkyl-C₁-C₄-alkyl, wherein the cyclic moieties are unsubstituted, or substituted with one or more, same or different substituents selected independently from the group consisting of halogen, CN, C₁-C₄-alkyl, C₁-C₄-haloalkyl, C₁-C₄-alkoxy, ═O and acetal groups thereof based on alcohols or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH;
   C₂-C₆-alkenyl, C₂-C₆-haloalkenyl, C₂-C₆-alkynyl, C₂-C₆-haloalkynyl;
   phenyl or phenyl-C₁-C₄-alkyl, wherein the aromatic rings are unsubstituted or substituted with one or more, same or different substituents R²²;
   or a 3- to 7-membered saturated, partially unsaturated, or fully unsaturated heterocyclic ring comprising one or more, same or different heteroatoms O, N, or S, wherein the heterocyclic ring is directly bonded to the remainder of the molecule or bonded to the remainder of the molecule via a C₁-C₄-alkyl group, and wherein the heterocyclic ring is unsubstituted or substituted with one or more, same or different substituents R²², and wherein the N- and/or S-atoms of the heterocyclic ring are oxidized or non-oxidized;
and wherein
R¹⁵, R¹⁶ are independently selected from C₁-C₆-alkyl;
R¹⁹ is H, C₁-C₆-alkyl, or C₃-C₈-cycloalkyl, wherein the aliphatic and cyclo-aliphatic radicals are unsubstituted, partially or fully halogenated;
R²⁰ is H, C₁-C₆-alkyl, C₂-C₆-alkenyl, or C₃-C₈-cycloalkyl, wherein the aliphatic and cycloaliphatic radicals are unsubstituted, or partially or fully halogenated;
   or phenyl, wherein the radical is unsubstituted, or partially or fully halogenated;
R^{21a}, R^{21b} are independently H, or C₁-C₆-alkyl-S(O)ₙ—, and
R²² is halogen, CN, C₁-C₆-alkoxy, or C₁-C₆-alkyl, wherein the aliphatic radicals are unsubstituted, or partially or fully halogenated.

Further preferred embodiments of the invention regarding the substituents R², R¹², R^{13a}, R^{13b}, R^{21a}, and R^{21b} are defined hereinafter.

Preferably, the invention relates to compounds of formula I* and the use of compounds of formula I, wherein
R¹² is H, CN, C₁-C₆-alkyl, C₁-C₆-haloalkyl, C₁-C₆-alkoxy, C₁-C₆-haloalkoxy, C₁-C₆-alkyl-S(O)ₙ, C₁-C₆-haloalky-S, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁵)₂R¹⁶, S(O)ₙR²⁰, S(O)ₙNR^{21a}R^{21b}, NR^{21a}R^{21b}, —N═CR¹⁷R¹⁸, —C(═O)R¹⁹, C(═O)NR^{21a}R^{21b}, C(═S)NR^{21a}R^{21b}, C(═O)OR²⁰,
   phenyl, optionally substituted with one or more substituents R²²; which are selected independently from one another,
   or a 3-, 4-, 5-, 6- or 7-membered saturated, partially unsaturated or fully unsaturated, or aromatic heterocyclic ring comprising 1, 2 or 3 heteroatoms selected from O, N and S, unsubstituted or substituted with one or more, e.g. 1, 2, 3 or 4, substituents R²², selected independently from one another, and wherein the N and/or S atoms of the heterocyclic ring may optionally be oxidized,
R^{13a}, R^{13b} are each independently from one another selected from the group consisting of H, C₁-C₆-alkyl, C₁-C₆-haloalkyl, C₁-C₆-alkoxy, C₁-C₆-haloalkoxy, C₁-C₆-alkyl-S—, C₁-C₆-haloalky-S—, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, —S(O)ₙNR^{21a}R^{21b}, C(═O)R¹⁹, C(═O)OR²⁰, C(═O)NR^{21a}R^{21b}, C(═S)R¹⁹, C(═S)SR²⁰, C(═S)NR^{21a}R^{21b}, C(═NR^{21a})R¹⁹;
   phenyl, optionally substituted with one or more, e.g. 1, 2, 3 or 4, substituents R²², which are selected independently from one another;
   a 3-, 4-, 5-, 6- or 7-membered saturated, partially unsaturated or fully unsaturated, or aromatic heterocyclic ring comprising 1, 2, 3 or 4 heteroatoms selected from O, N and S, optionally substituted with one or more, e.g. 1, 2, 3 or 4, substituents R²², selected independently from one another, and wherein the N and/or S atoms of the heterocyclic ring may optionally be oxidized;
   or R^{13a} and R^{13b} are together a C₂-C₇ alkylene or C₂-C₇ alkenylene chain and form a 3-, 4-, 5-, 6-, 7- or 8-membered saturated, partially unsaturated or fully unsaturated, or aromatic ring together with the N-atom they are bonded to, wherein the alkylene chain or alkenylene chain may contain one or two heteratoms selected from O, S and N, and may optionally be substituted with halogen, C₁-C₆-alkyl, C₁-C₆-haloalkyl, C₁-C₆-alkoxy, C₁-C₆-haloalkoxy, C₁-C₆-alkyl-S—, C₁-C₆-haloalkyl-S, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl;
   phenyl, optionally substituted with one or more substituents R²²; which are selected independently from one another;
   or a 3-, 4-, 5-, 6,- or 7-membered saturated, partially unsaturated or fully unsaturated, or aromatic heterocyclic ring comprising 1, 2 or 3 heteroatoms selected from O, S, and N, optionally substituted with one or more substituents R²², selected independently from one another, and wherein the N and/or S atoms of the heterocyclic ring may optionally be oxidized;
R^{21a}, R^{21b} are each independently from one another selected from the group consisting of H, CN, C₁-C₆-alkoxy, C₁-C₆-haloalkoxy, C₁-C₆-alkyl-S(O)ₙ—, trimethylsilyl, triethylsilyl, tert-butyldimethylsilyl,
   C₁-C₆-alkyl, C₂-C₆-alkenyl, C₂-C₆-alkynyl, C₃-C₈-cycloalkyl, C₃-C₈-cycloalkyl-C₁-C₆-alkyl, C₁-C₆-alkyl-C₃-C₈-cycloalkyl, wherein the aliphatic and cyclo-aliphatic radicals may be unsubstituted, partially or fully halogenated and/or oxygenated and/or may carry 1 or 2 radicals selected from C₁-C₄-alkoxy,
   phenyl, benzyl, pyridyl, and phenoxy, wherein the radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl;
   or R^{21a} and R^{21b} may together be a C₂-C₆ alkylene chain forming a 3- to 7-membered saturated, partly saturated or unsaturated ring together with the N-atom R^{21a} and R^{21b} are bonded to, wherein the alkylene chain may contain 1 or 2 heteroatoms selected from O, S, and N, and may optionally be substituted with halogen, C₁-C₄-haloalkyl, C₁-C₄-alkoxy or C₁-C₄-haloalkoxy, and wherein the N and/or S atoms of the heterocyclic ring may optionally be oxidized;
and wherein all other substituents have the meaning as defined above.

Preferably, the invention relates to compounds of formula I* and the use of compounds of formula I, wherein
R² is OR¹², NR^{13a}R^{13b}, S(O)ₙR¹², S(O)ₙNR^{13a}R^{13b}, Si(R₁₅)₂R₁₆, more preferably from OR¹², S(O)ₙR¹², S(O)ₙNR^{13a}R^{13b};
R¹² is H, CN, C₁-C₆-alkyl, C₁-C₆-haloalkyl, C₁-C₆-alkoxy, C₁-C₆-haloalkoxy, C₁-C₆-alkyl-S(O)ₙ, C₁-C₆-haloalkyl-S, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁵)₂R¹⁶, S(O)ₙR²⁰, S(O)ₙNR^{21a}R^{21b}, NR^{21a}R^{21b}, —N═CR¹⁷R¹⁸, —C(═O)R¹⁹, C(═O)NR^{21a}R^{21b}, C(═S)NR^{21a}R^{21b}C(═O)OR²⁰,
   phenyl, optionally substituted with one or more substituents R²²; which are selected independently from one another,
   or a 3-, 4-, 5-, 6- or 7-membered saturated, partially unsaturated or fully unsaturated, or aromatic heterocyclic ring comprising 1, 2 or 3 heteroatoms selected from O, N and S, unsubstituted or substituted with one or more, e.g. 1, 2, 3 or 4, substituents R²², selected independently from one another, and wherein the N and/or S atoms of the heterocyclic ring may optionally be oxidized,
R^{13a}, R^{13b} are each independently from one another selected from the group consisting of H, C₁-C₆-alkyl, C₁-C₆-haloalkyl, C₁-C₆-alkoxy, C₁-C₆-haloalkoxy, C₁-C₆-alkyl-S—, C₁-C₆-haloalky-S—, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, —S(O)ₙNR^{21a}R^{21b}, C(═O)R¹⁹, C(═O)OR²⁰, C(═O)NR^{21a}R^{21b}, C(═S)R¹⁹, C(═S)SR²⁰, C(═S)NR^{21a}R^{21b}, C(═NR^{21a})R¹⁹;
   phenyl, optionally substituted with one or more, e.g. 1, 2, 3 or 4, substituents R²², which are selected independently from one another;
   a 3-, 4-, 5-, 6- or 7-membered saturated, partially unsaturated or fully unsaturated, or aromatic heterocyclic ring comprising 1, 2, 3 or 4 heteroatoms selected from O, N and S, optionally substituted with one or more, e.g. 1, 2, 3 or 4, substituents R²², selected independently from one another, and wherein the N and/or S atoms of the heterocyclic ring may optionally be oxidized;
   or R^{13a} and R^{13b} are together a C₂-C₇ alkylene or C₂-C₇ alkenylene chain and form a 3-, 4-, 5-, 6-, 7- or 8-membered saturated, partially unsaturated or fully unsaturated, or aromatic ring together with the N-atom they are bonded to,
   wherein the alkylene chain or alkenylene chain may contain one or two heteratoms selected from O, S and N, and may optionally be substituted with halogen, C₁-C₆-alkyl, C₁-C₆-haloalkyl, C₁-C₆-alkoxy, C₁-C₆-haloalkoxy, C₁-C₆-alkyl-S—, C₁-C₆-haloalkyl-S, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl;
   phenyl, optionally substituted with one or more substituents R²²; which are selected independently from one another;
   or a 3-, 4-, 5-, 6,- or 7-membered saturated, partially unsaturated or fully unsaturated, or aromatic heterocyclic ring comprising 1, 2 or 3 heteroatoms selected from O, S, and N, optionally substituted with one or more substituents R²², selected independently from one another, and wherein the N and/or S atoms of the heterocyclic ring may optionally be oxidized;
R^{21a}, R^{21b} are each independently from one another selected from the group consisting of H, CN, C₁-C₆-alkoxy, C₁-C₆-haloalkoxy, C₁-C₆-alkyl-S(O)ₙ—, trimethylsilyl, triethylsilyl, tert-butyldimethylsilyl,
   C₁-C₆-alkyl, C₂-C₆-alkenyl, C₂-C₆-alkynyl, C₃-C₈-cycloalkyl, C₃-C₈-cycloalkyl-C₁-C₆-alkyl, C₁-C₆-alkyl-C₃-C₈-cycloalkyl, wherein the aliphatic and cyclo-aliphatic radicals may be unsubstituted, partially or fully halogenated and/or oxygenated and/or may carry 1 or 2 radicals selected from C₁-C₄-alkoxy,
   phenyl, benzyl, pyridyl, and phenoxy, wherein the radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl;
   or R^{21a} and R^{21b} may together be a C₂-C₆ alkylene chain forming a 3- to 7-membered saturated, partly saturated or unsaturated ring together with the N-atom R^{21a} and R^{21b} are bonded to, wherein the alkylene chain may contain 1 or 2 heteroatoms selected from O, S, and N, and may optionally be substituted with halogen, C₁-C₄-haloalkyl, C₁-C₄-alkoxy or C₁-C₄-haloalkoxy, and wherein the N and/or S atoms of the heterocyclic ring may optionally be oxidized;
and wherein all other substituents have the meaning as defined above.

Preferably, the invention relates to compounds of formula I* and the use of compounds of formula I, wherein
R² is OR¹², S(O)ₙR¹², or S(O)ₙNR^{13a}R^{13b};
R¹² is C₁-C₆-alkyl, which is unsubstituted or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₁-C₆-hetaryl;
   C₁-C₆-haloalkyl, which is unsubstituted or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
   C₃-C₈-cycloalkyl, C₃-C₈-cycloalkyl-C₁-C₄-alkyl, C₃-C₈-halocycloalkyl, C₃-C₈-cycloalkyl-C₁-C₄-alkyl, wherein the cyclic moieties are unsubstituted or substituted with one or more, same or different substituents selected from halogen, CN, C₁-C₄-alkyl, C₁-C₄-haloalkyl, C₁-C₄-alkoxy, ═O, and acetal groups thereof based on alcohols or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH;
   C₂-C₆-alkenyl, C₂-C₆-haloalkenyl, C₂-C₆-alkynyl, C₂-C₆-haloalkynyl;
   phenyl or phenyl-C₁-C₄-alkyl, wherein the aromatic rings are unsubstituted or substituted with one or more, same or different substituents R²²;
   or a 3- to 7-membered saturated, partially unsaturated, or fully unsaturated heterocyclic ring comprising one or more, same or different heteroatoms O, N, or S, wherein the heterocyclic ring are directly bonded to the remainder of the molecule or bonded to the remainder of the molecule via a C₁-C₄-alkyl group, and wherein the heterocyclic ring is unsubstituted or substituted with one or more, same or different substituents R²², and wherein the N- and/or S-atoms of the heterocyclic ring are oxidized or non-oxidized;
R^{13a}, R^{13b} are independently C₁-C₆-alkyl, C₃-C₈-cycloalkyl, C₃-C₈-cycloalkyl-C₁-C₆-alkyl, or C(═O)R¹⁹;
R¹⁵, R¹⁶ are independently selected from C₁-C₆alkyl;
R¹⁹ is H, C₁-C₆-alkyl, or C₃-C₈-cycloalkyl, wherein the aliphatic and cyclo-aliphatic radicals are unsubstituted, or partially or fully halogenated;
R²⁰ is H, C₁-C₆-alkyl, C₂-C₆-alkenyl, or C₃-C₈-cycloalkyl, wherein the aliphatic and cycloaliphatic radicals are unsubstituted, or partially or fully halogenated;
   or phenyl, wherein the radical is unsubstituted, or partially or fully halogenated;
R^{21a}, R^{21b} are independently H, C₁-C₆-alkyl-S(O)ₙ—, C₁-C₆-alkyl, C₃-C₈-cycloalkyl, C₃-C₈-cycloalkyl-C₁-C₆-alkyl, or C₁-C₆-alkyl-C₃-C₈-cycloalkyl, wherein the aliphatic and cyclo-aliphatic radicals are unsubstituted, partially or fully halogenated and/or oxygenated and/or substituted by one or more, same or different radicals selected from C₁-C₄-alkoxy;
R²² is halogen, CN, C₁-C₆-alkoxy, or C₁-C₆-alkyl, wherein the aliphatic radicals are unsubstituted, partially or fully halogenated;
and wherein all other substituents have the meaning as defined above.

Preferably, the invention relates to compounds of formula I* and the use of compounds of formula I, wherein
R² is OR¹², S(O)ₙR¹², S(O)ₙNR^{13a}R^{13b}, more preferably from OR¹²;
R¹² is C₁-C₆-alkyl, which may be substituted with CN, C₁-C₆-alkoxy, C₁-C₆-haloalkoxy, C₁-C₆-alkyl-S(O)ₙ, C₁-C₆-haloalkyl-S(O)ₙ, C₁-C₆-hetaryl;
   C₁-C₆-haloalkyl, which may be substituted with C₁-C₆-alkoxy, C₁-C₆-haloalkoxy, C₁-C₆-alkyl-S(O)ₙ, C₁-C₆-haloalkyl-S(O)ₙ;
   C₃-C₈-cycloalkyl-C₁-C₄-alkyl, which may be substituted with halogen, CN;
   C₁-C₄-alkyl-C₃-C₈-cycloalkyl, C₃-C₈-cycloalkyl, C₃-C₈-halocycloalkyl, C₂-C₆-alkenyl, C₂-C₆-alkynyl, NR^{21a}R^{21b};
R^{13a}, R^{13b} are each independently from one another selected from the group consisting of C₁-C₆-alkyl, C₃-C₈-cycloalkyl, C₃-C₈-cycloalkyl-C₁-C₆-alkyl, C(═O)R¹⁹;
R^{21a}, R^{21b} are each independently from one another selected from the group consisting of H, C₁-C₆-alkyl, C₃-C₈-cycloalkyl, C₃-C₈-cycloalkyl-C₁-C₆-alkyl, C₁-C₆-alkyl-C₃-C₈-cycloalkyl, wherein the aliphatic and cyclo-aliphatic radicals may be unsubstituted, partially or fully halogenated and/or oxygenated and/or may carry 1 or 2 radicals selected from C₁-C₄-alkoxy,
and wherein all other substituents have the meaning as defined above.

Preferably, the invention also relates to compounds of formula I* and the use of compounds of formula I, wherein
R¹² is C₁-C₆-alkyl substituted with one or more, same or different substituents selected from OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, and phenyl;
   C₁-C₆-haloalkyl substituted with one or more, same or different substituents selected from OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, and phenyl;
   C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, wherein the cyclic moieties are unsubstituted or substituted with one or more, same or different substituents selected from C₁-C₄-haloalkyl, C₁-C₄-alkoxy, C₁-C₄-haloalkoxy, ═O and acetal groups thereof based on alcohols or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH;
   phenyl-C₁-C₄-alkyl, wherein the aromatic ring is substituted with one or more, same or different substituents R²²;
   or a 3- to 7-membered saturated, partially unsaturated or fully unsaturated, heterocyclic ring comprising one or more, same or different heteroatoms O, N, or S, wherein the heterocyclic ring is bonded to the remainder of the molecule via a C₁-C₄-alkyl group, and wherein the heterocyclic ring is substituted with one or more, same or different substituents R²², and wherein the N- and/or S-atoms of the heterocyclic ring are oxidized or non-oxidized;
and wherein
R¹⁵, R¹⁶ are independently selected from C₁-C₆-alkyl;
R¹⁹ is H, C₁-C₆-alkyl, or C₃-C₈-cycloalkyl, wherein the aliphatic and cyclo-aliphatic radicals are unsubstituted, or partially or fully halogenated;
R²⁰ is H, C₂-C₆-alkenyl, C₃-C₈-cycloalkyl, wherein the aliphatic and cycloaliphatic radicals are unsubstituted, or partially or fully halogenated; or phenyl, wherein the radical is unsubstituted, or partially or fully halogenated;
R^{21a}, R^{21b} are each independently from one another selected from the group consisting of H, C₁-C₆-alkyl-S(O)ₙ—, and
R²² is halogen, CN, C₁-C₆-alkoxy, C₁-C₆-alkyl, wherein the aliphatic radicals may be unsubstituted, partially or fully halogenated.

Preferably, the invention also relates to compounds of formula I* and the use of compounds of formula I, wherein
R¹² is C₁-C₆-alkyl, substituted with CN, C₁-C₆-alkoxy, C₁-C₆-haloalkoxy, C₁-C₆-alkyl-S(O)ₙ, C₁-C₆-haloalkyl-S(O)ₙ, C₁-C₆-hetaryl;
   C₁-C₆-haloalkyl, substituted with C₁-C₆-alkoxy, C₁-C₆-haloalkoxy, C₁-C₆-alkyl-S(O)ₙ, C₁-C₆-haloalkyl-S(O)ₙ; C₃-C₈-cycloalkyl-C₁-C₄-alkyl, substituted with halogen, CN;
   C₁-C₄-alkyl-C₃-C₈-cycloalkyl,
R^{13a}, R^{13b} are each independently from one another selected from the group consisting of C₁-C₆-alkyl, C₃-C₈-cycloalkyl-C₁-C₆-alkyl;
R^{21a}, R^{21b} are each independently from one another selected from the group consisting of H, C₃-C₈-cycloalkyl-C₁-C₆-alkyl, C₁-C₆-alkyl-C₃-C₈-cycloalkyl;
   and wherein all other substituents have the meaning as defined above.

Formulae I-B to I-K depicted below, together and each alone, form embodiments of formulae I and I*, wherein
Y is O, S, SO, or SO₂;
D is H, F, Cl, Br, I, CH₃, CF₃, or CN, preferably H; and
B is C₁-C₆-alkyl, which is unsubstituted or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₁-C₆-hetaryl;
   C₁-C₆-haloalkyl, which is unsubstituted or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
   C₃-C₈-cycloalkyl, C₃-C₈-cycloalkyl-C₁-C₄-alkyl, C₃-C₈-halocycloalkyl, C₃-C₈-cycloalkyl-C₁-C₄-alkyl, wherein the cyclic moieties are unsubstituted, or substituted with one or more, same or different substituents selected independently from the group consisting of halogen, CN, C₁-C₄-alkyl, C₁-C₄-haloalkyl, C₁-C₄-alkoxy, ═O and acetal groups thereof based on alcohols or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH;
   C₂-C₆-alkenyl, C₂-C₆-haloalkenyl, C₂-C₆-alkynyl, C₂-C₆-haloalkynyl;
   phenyl or phenyl-C₁-C₄-alkyl, wherein the aromatic rings are unsubstituted or substituted with one or more, same or different substituents R²²;
   or a 3- to 7-membered saturated, partially unsaturated, or fully unsaturated heterocyclic ring comprising one or more, same or different heteroatoms O, N, or S, wherein the heterocyclic ring is directly bonded to the remainder of the molecule or bonded to the remainder of the molecule via a C₁-C₄-alkyl group, and wherein the heterocyclic ring is unsubstituted or substituted with one or more, same or different substituents R²², and wherein the N- and/or S-atoms of the heterocyclic ring are oxidized or non-oxidized;
wherein
R¹⁵, R¹⁶ are independently C₁-C₆ alkyl, or phenyl;
R¹⁹ is H, C₁-C₆-alkoxy, C₁-C₆-haloalkoxy, C₁-C₆-alkyl-S(O)ₙ—, C₁-C₆-haloalkyl-S(O)ₙ—, C₁-C₆-alkyl, C₂-C₆-alkenyl, C₂-C₆-alkynyl, C₃-C₈-cycloalkyl, wherein the aliphatic and cycloaliphatic radicals are unsubstituted, partially, or fully halogenated, and/or oxygenated, and/or substituted by one or more, same or different radicals selected from C₁-C₄-alkoxy; phenyl, benzyl, pyridyl, or phenoxy, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino, and di-(C₁-C₆-alkyl)amino;
R²⁰ is H, C₁-C₆-alkyl, C₂-C₆-alkenyl, C₂-C₆-alkynyl, or C₃-C₈-cycloalkyl, wherein the aliphatic and cycloaliphatic radicals are unsubstituted, partially, or fully halogenated, and/or oxygenated, and/or substituted by one or more, same or different substituents selected from C₁-C₄-alkoxy;
   phenyl, benzyl, or pyridyl, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
R^{21a}, R^{21b} are independently H, C₁-C₆-alkoxy, C₁-C₆-haloalkoxy, C₁-C₆-alkyl-S(O)ₙ—, C₁-C₆-haloalkyl-S(O)ₙ—, C₁-C₆-alkyl, C₂-C₆-alkenyl, C₂-C₆-alkynyl, C₃-C₈-cycloalkyl, C₃-C₈-cycloalkyl-C₁-C₆-alkyl, wherein the aliphatic and cycloaliphatic radicals are unsubstituted, partially, or fully halogenated, and/or oxygenated, and/or substituted by one or more, same or different substituents selected from C₁-C₄-alkoxy;
   phenyl, benzyl, pyridyl, and phenoxy, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
   or R^{21a} and R^{21b} are together a C₂-C₆ alkylene chain forming a 3- to 7-membered saturated, partially unsaturated, or fully unsaturated ring together with the N-atom they are bonded to, wherein the alkylene chain contains none, one or more, same or different heteroatoms O, S, or N, and is unsubstituted, or substituted with one or more, same or different substituents selected from halogen, C₁-C₄-haloalkyl, C₁-C₄-alkoxy, and C₁-C₄-haloalkoxy, and wherein the N- and/or S-atoms of the heterocyclic ring are oxidized, or non-oxidized; and
R²² is H, halogen, NO₂, CN, OH, SH, C₁-C₆-alkoxy, C₁-C₆-haloalkoxy, C₁-C₆-alkyl-S(O)ₙ, C₁-C₆-haloalkyl-S(O)ₙ—, C₁-C₆-alkyl, C₂-C₆-alkenyl, C₂-C₆-alkynyl, C₃-C₈-cycloalkyl, wherein the aliphatic and cycloaliphatic radicals are unsubstituted, partially, or fully halogenated, and/or oxygenated, and/or substituted by one or more, same or different substituents selected from C₁-C₄-alkoxy, phenyl, benzyl, pyridyl, or phenoxy, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
   or two R²² present together on one atom are ═O, ═S, ═N(C₁-C₆-alkyl), ═NO(C₁-C₆-alkyl), ═CH(C₁-C₄-alkyl), or ═C(C₁-C₄-alkyl)C₁-C₄-alkyl;
   or two R²² on two adjacent C-atoms are together a C₂-C₆ alkylene chain, or C₂-C₆-alkenylene chain, which form together with the C-atom they are bonded to a 3- to 7-membered saturated, partially unsaturated, or fully unsaturated ring, wherein the alkylene, or alkenylene chain contains one or more, same or different heteroatoms O, S, or N, and is unsubstituted, or substituted with one or more, same or different substituents selected from halogen, C₁-C₄-haloalkyl, C₁-C₄-alkoxy, and C₁-C₄-haloalkoxy, and wherein the N- and/or S-atoms of the heterocyclic ring are oxidized, or non-oxidized.
and wherein preferably
R¹⁵, R¹⁶ are independently selected from C₁-C₆ alkyl;
R¹⁹ is H, C₁-C₆-alkyl, C₂-C₆-alkenyl, C₂-C₆-alkynyl, C₃-C₈-cycloalkyl, wherein the aliphatic and cycloaliphatic radicals are unsubstituted, partially or fully halogenated and/or oxgenated and/or substituted by one or more, same or different radicals selected from C₁-C₄-alkoxy;
R²⁰ is H, C₁-C₆-alkyl, C₂-C₆-alkenyl, C₂-C₆-alkynyl, C₃-C₈-cycloalkyl, wherein the aliphatic and cycloaliphatic radicals are unsubstituted, partially or fully halogenated and/or oxygenated and/or substituted by one or more, same or different radicals selected from C₁-C₄-alkoxy; phenyl, benzyl, or pyridyl, wherein the radicals are unsubstituted, partially or fully halogenated and/or substituted by one or more, same or different radicals selected from halogen, C₁-C₆-alkyl, C₁-C₆-haloalkyl, C₁-C₆-alkoxy, C₁-C₆ haloalkoxy or (C₁-C₆-alkoxy)carbonyl;
R^{21a}, R^{21b} are independently H, C₁-C₆-alkoxy, C₁-C₆-haloalkoxy, C₁-C₆-alkyl-S(O)ₙ—, C₁-C₆-haloalkyl-S(O)ₙ—, C₁-C₆-alkyl, C₂-C₆-alkenyl, C₂-C₆-alkynyl, C₃-C₈-cycloalkyl, or C₃-C₈-cycloalkyl-C₁-C₆-alkyl, wherein the aliphatic and cycloaliphatic radicals are unsubstituted, partially or fully halogenated and/or oxygenated and/or substituted by one or more, same or different radicals selected from C₁-C₄-alkoxy;
   or R^{21a} and R^{21b} are together a C₂-C₆ alkylene chain forming a 3- to 7-membered saturated, partially unsaturated or fully unsaturated ring together with the N-atom R^{21a} and R^{21b} are bonded to, wherein the alkylene chain may contain one or more, same or different heteroatoms O, S, or N, and are unsubstituted or substituted with halogen, C₁-C₄-haloalkyl, C₁-C₄-alkoxy, or C₁-C₄-haloalkoxy, and wherein the N- and/or S-atoms of the heterocyclic ring are oxidized or non-oxidized; and
R²² is H, halogen, NO₂, CN, C₁-C₆-alkoxy, C₁-C₆-haloalkoxy, C₁-C₆-alkyl-S(O)ₙ, C₁-C₆-haloalkyl-S(O)ₙ—, C₁-C₆-alkyl, C₂-C₆-alkenyl, C₂-C₆-alkynyl, or C₃-C₈-cycloalkyl, wherein the aliphatic and cyclo-aliphatic radicals are unsubstituted, partially or fully halogenated, and/or oxygenated, and/or substituted by one or more, same or different radicals selected from C₁-C₄-alkoxy.
and wherein particularly preferably
R¹⁵, R¹⁶ are independently selected from C₁-C₆ alkyl;
R¹⁹ is H, C₁-C₆-alkyl, or C₃-C₈-cycloalkyl, wherein the aliphatic and cyclo-aliphatic radicals are unsubstituted, partially or fully halogenated;
R²⁰ is H, C₁-C₆-alkyl, C₂-C₆-alkenyl, or C₃-C₈-cycloalkyl, wherein the aliphatic and cycloaliphatic radicals are unsubstituted, or partially or fully halogenated;
   or phenyl, wherein the radical is unsubstituted, or partially or fully halogenated;
R^{21a}, R^{21b} are independently H, or C₁-C₆-alkyl-S(O)ₙ—, and
R²² is halogen, CN, C₁-C₆-alkoxy, or C₁-C₆-alkyl, wherein the aliphatic radicals are unsubstituted, or partially or fully halogenated,
or if D is SO, SO₂,
B is one of the above substituents, or (C₁-C₄-alkyl)(C₁-C₄-alkyl)N, (C₃-C₄-cycloalkyl)(C₁-C₄-alkyl)N, (C₃-cycloalkyl-C₁-C₂-alkyl)(C₁-C₄-alkyl)N, (C₁-C₄-alkyl-C₃-cycloalkyl)(C₁-C₄-alkyl)N, C₁-C₃-alkyl-carbonyl-N H, or C₃-cycloalkyl-carbonyl-N H.

In connection with the formulae I-B to I-K, it is further preferred that R¹ is H, CN or F, preferably H.

Formulae I-B to I-K depicted below, together and each alone, form embodiments of formulae I and I*, wherein
Y is O, S, SO, or SO₂;
D is H, F, Cl, Br, I, CH₃, CF₃, or CN; preferably H;
B is C₁-C₄-alkyl, C₃-cycloalkyl-C₁-C₂-alkyl, C₁-C₄-alkyl-C₃-cycloalkyl, C₃-C₄-alkenyl, C₃-C₅-alkinyl, C₁-C₄-haloalkyl, C₄-C₆-halocycloalkyl, C₁-C₄-alkyl-NH, C₃-C₄-cycloalkyl-NH, C₃-cycloalkyl-C₁-C₂-alkyl-N H, C₁-C₄-alkyl-C₃-cycloalkyl-N H;
   C₁-C₄ alkyl, substituted with CN, C₃-halocycloalkyl, C₁-C₃-alkoxy, di-C₁-alkoxy, C₁-haloalkoxy, C₁-C₄-alkyl-S(O)ₙ, C₁-haloalkyl-S, or heteroaromatic 5-membered rings, comprising as ring members one O-atom;
   C₃-cycloalkyl-C₁-C₂-alkyl, substituted with halogen, CN; or
   C₂-haloalkyl, substituted with C₁-alkoxy, C₁-haloalkoxy, C₁-C₄-alkyl-S, C₁-haloalkyl-S;
or if D is SO, SO₂,
B is one of the above substituents, or (C₁-C₄-alkyl)(C₁-C₄-alkyl)N, (C₃-C₄-cycloalkyl)(C₁-C₄-alkyl)N, (C₃-cycloalkyl-C₁-C₂-alkyl)(C₁-C₄-alkyl)N, (C₁-C₄-alkyl-C₃-cycloalkyl)(C₁-C₄-alkyl)N, C₁-C₃-alkyl-carbonyl-N H, C₃-cycloalkyl-carbonyl-NH.

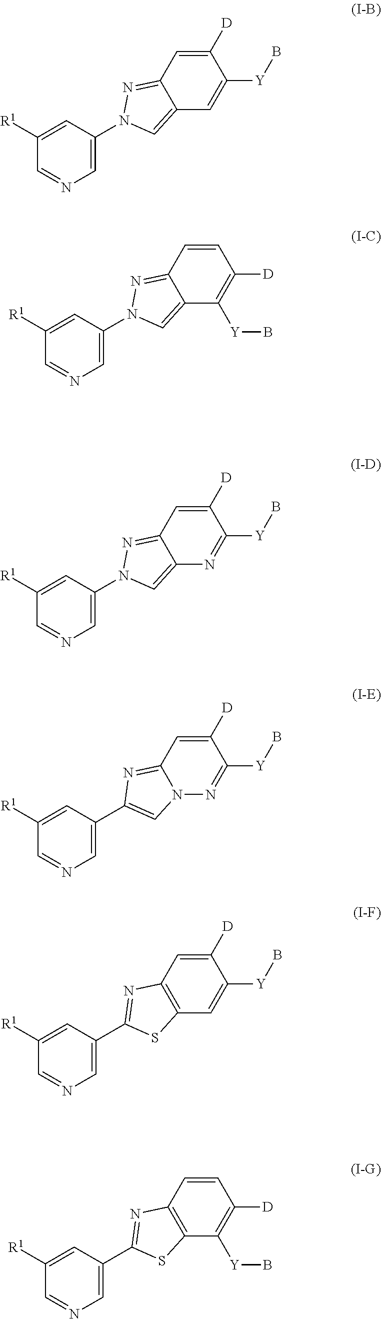

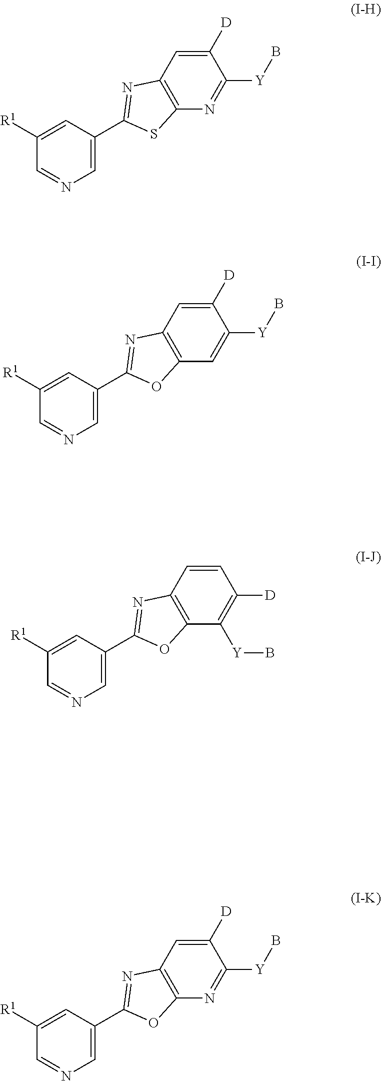

Examples of preferred compounds of formulae I and I* are compiled in tables D to 180 below. Moreover, the meanings mentioned below for the individual variables in the tables are per se, independently of the combination in which they are mentioned, a particularly preferred embodiment of the substituents in question.

Table 1 Compounds of the formula (I-B) in which R¹ is H, Y is O, and the combination of D and B for a compound corresponds in each case to one row of Table A.

Table 2 Compounds of the formula (I-B) in which R¹ is F, Y is O, and the combination of D and B for a compound corresponds in each case to one row of Table A.

Table 3 Compounds of the formula (I-B) in which R¹ is CN, Y is O, and the combination of D and B for a compound corresponds in each case to one row of Table A.

Table 4 Compounds of the formula (I-B) in which R¹ is H, Y is S, and the combination of D and B for a compound corresponds in each case to one row of Table A.

Table 5 Compounds of the formula (I-B) in which R¹ is F, Y is S, and the combination of D and B for a compound corresponds in each case to one row of Table A.

Table 6 Compounds of the formula (I-B) in which R¹ is CN, Y is S, and the combination of D and B for a compound corresponds in each case to one row of Table A.

Table 7 Compounds of the formula (I-B) in which R¹ is H, Y is SO, and the combination of D and B for a compound corresponds in each case to one row of Table A.

Table 8 Compounds of the formula (I-B) in which R¹ is F, Y is SO, and the combination of D and B for a compound corresponds in each case to one row of Table A.

Table 9 Compounds of the formula (I-B) in which R¹ is CN, Y is SO, and the combination of D and B for a compound corresponds in each case to one row of Table A.

Table 10 Compounds of the formula (I-B) in which R¹ is H, Y is SO₂, and the combination of D and B for a compound corresponds in each case to one row of Table A.

Table 11 Compounds of the formula (I-B) in which R¹ is F, Y is SO₂, and the combination of O and B for a compound corresponds in each case to one row of Table A.

Table 12 Compounds of the formula (I-B) in which R¹ is CN, Y is SO₂, and the combination of 0 and B for a compound corresponds in each case to one row of Table A.

Table 13 Compounds of the formula (I-C) in which R¹ is H, Y is O, and the combination of 0 and B for a compound corresponds in each case to one row of Table A.

Table 14 Compounds of the formula (I-C) in which R¹ is F, Y is O, and the combination of D and B for a compound corresponds in each case to one row of Table A.

Table 15 Compounds of the formula (I-C) in which R¹ is CN, Y is O, and the combination of D and B for a compound corresponds in each case to one row of Table A.

Table 16 Compounds of the formula (I-C) in which R¹ is H, Y is S, and the combination of D and B for a compound corresponds in each case to one row of Table A.

Table 17 Compounds of the formula (I-C) in which R¹ is F, Y is S, and the combination of D and B for a compound corresponds in each case to one row of Table A.

Table 18 Compounds of the formula (I-C) in which R¹ is CN, Y is S, and the combination of D and B for a compound corresponds in each case to one row of Table A.

Table 19 Compounds of the formula (I-C) in which R¹ is H, Y is SO, and the combination of D and B for a compound corresponds in each case to one row of Table A.

Table 20 Compounds of the formula (I-C) in which R¹ is F, Y is SO, and the combination of D and B for a compound corresponds in each case to one row of Table A.

Table 21 Compounds of the formula (I-C) in which R¹ is CN, Y is SO, and the combination of D and B for a compound corresponds in each case to one row of Table A.

Table 22 Compounds of the formula (I-C) in which R¹ is H, Y is SO₂, and the combination of D and B for a compound corresponds in each case to one row of Table A.

Table 23 Compounds of the formula (I-C) in which R¹ is F, Y is SO₂, and the combination of D and B for a compound corresponds in each case to one row of Table A.

Table 24 Compounds of the formula (I-C) in which R¹ is CN, Y is SO₂, and the combination of D and B for a compound corresponds in each case to one row of Table A.

Table 25 Compounds of the formula (I-D) in which R¹ is H, Y is O, and the combination of D and B for a compound corresponds in each case to one row of Table A.

Table 26 Compounds of the formula (I-D) in which R¹ is F, Y is O, and the combination of D and B for a compound corresponds in each case to one row of Table A.

Table 27 Compounds of the formula (I-D) in which R¹ is CN, Y is O, and the combination of D and B for a compound corresponds in each case to one row of Table A.

Table 28 Compounds of the formula (I-D) in which R¹ is H, Y is S, and the combination of D and B for a compound corresponds in each case to one row of Table A.

Table 29 Compounds of the formula (I-D) in which R¹ is F, Y is S, and the combination of D and B for a compound corresponds in each case to one row of Table A.

Table 30 Compounds of the formula (I-D) in which R¹ is CN, Y is S, and the combination of D and B for a compound corresponds in each case to one row of Table A.

Table 31 Compounds of the formula (I-D) in which R¹ is H, Y is SO, and the combination of D and B for a compound corresponds in each case to one row of Table A.

Table 32 Compounds of the formula (I-D) in which R¹ is F, Y is SO, and the combination of D and B for a compound corresponds in each case to one row of Table A.

Table 33 Compounds of the formula (I-D) in which R¹ is CN, Y is SO, and the combination of D and B for a compound corresponds in each case to one row of Table A.

Table 34 Compounds of the formula (I-D) in which R¹ is H, Y is SO₂, and the combination of D and B for a compound corresponds in each case to one row of Table A.

Table 35 Compounds of the formula (I-D) in which R¹ is F, Y is SO₂, and the combination of D and B for a compound corresponds in each case to one row of Table A.

Table 36 Compounds of the formula (I-D) in which R¹ is CN, Y is SO₂, and the combination of D and B for a compound corresponds in each case to one row of Table A.

Table 37 Compounds of the formula (I-E) in which R¹ is H, Y is O, and the combination of D and B for a compound corresponds in each case to one row of Table A.

Table 38 Compounds of the formula (I-E) in which R¹ is F, Y is O, and the combination of D and B for a compound corresponds in each case to one row of Table A.

Table 39 Compounds of the formula (I-E) in which R¹ is CN, Y is O, and the combination of D and B for a compound corresponds in each case to one row of Table A.

Table 40 Compounds of the formula (I-E) in which R¹ is H, Y is S, and the combination of D and B for a compound corresponds in each case to one row of Table A.

Table 41 Compounds of the formula (I-E) in which R¹ is F, Y is S, and the combination of D and B for a compound corresponds in each case to one row of Table A.

Table 42 Compounds of the formula (I-E) in which R¹ is CN, Y is S, and the combination of D and B for a compound corresponds in each case to one row of Table A.

Table 43 Compounds of the formula (I-E) in which R¹ is H, Y is SO, and the combination of D and B for a compound corresponds in each case to one row of Table A.

Table 44 Compounds of the formula (I-E) in which R¹ is F, Y is SO, and the combination of D and B for a compound corresponds in each case to one row of Table A.

Table 45 Compounds of the formula (I-E) in which R¹ is CN, Y is SO, and the combination of D and B for a compound corresponds in each case to one row of Table A.

Table 46 Compounds of the formula (I-E) in which R¹ is H, Y is SO₂, and the combination of D and B for a compound corresponds in each case to one row of Table A.

Table 47 Compounds of the formula (I-E) in which R¹ is F, Y is SO₂, and the combination of D and B for a compound corresponds in each case to one row of Table A.

Table 48 Compounds of the formula (I-E) in which R¹ is CN, Y is SO₂, and the combination of D and B for a compound corresponds in each case to one row of Table A.

Table 49 Compounds of the formula (I-F) in which R¹ is H, Y is O, and the combination of D and B for a compound corresponds in each case to one row of Table A.

Table 50 Compounds of the formula (I-F) in which R¹ is F, Y is O, and the combination of D and B for a compound corresponds in each case to one row of Table A.

Table 51 Compounds of the formula (I-F) in which R¹ is CN, Y is O, and the combination of D and B for a compound corresponds in each case to one row of Table A.

Table 52 Compounds of the formula (I-F) in which R¹ is H, Y is S, and the combination of D and B for a compound corresponds in each case to one row of Table A.

Table 53 Compounds of the formula (I-F) in which R¹ is F, Y is S, and the combination of D and B for a compound corresponds in each case to one row of Table A.

Table 54 Compounds of the formula (I-F) in which R¹ is CN, Y is S, and the combination of D and B for a compound corresponds in each case to one row of Table A.

Table 55 Compounds of the formula (I-F) in which R¹ is H, Y is SO, and the combination of D and B for a compound corresponds in each case to one row of Table A.

Table 56 Compounds of the formula (I-F) in which R¹ is F, Y is SO, and the combination of D and B for a compound corresponds in each case to one row of Table A.

Table 57 Compounds of the formula (I-F) in which R¹ is CN, Y is SO, and the combination of D and B for a compound corresponds in each case to one row of Table A.

Table 58 Compounds of the formula (I-F) in which R¹ is H, Y is SO₂, and the combination of D and B for a compound corresponds in each case to one row of Table A.

Table 59 Compounds of the formula (I-F) in which R¹ is F, Y is SO₂, and the combination of D and B for a compound corresponds in each case to one row of Table A.

Table 60 Compounds of the formula (I-F) in which R¹ is CN, Y is SO₂, and the combination of D and B for a compound corresponds in each case to one row of Table A.

Table 61 Compounds of the formula (I-G) in which R¹ is H, Y is O, and the combination of D and B for a compound corresponds in each case to one row of Table A.

Table 62 Compounds of the formula (I-G) in which R¹ is F, Y is O, and the combination of D and B for a compound corresponds in each case to one row of Table A.

Table 63 Compounds of the formula (I-G) in which R¹ is CN, Y is O, and the combination of D and B for a compound corresponds in each case to one row of Table A.

Table 64 Compounds of the formula (I-G) in which R¹ is H, Y is S, and the combination of D and B for a compound corresponds in each case to one row of Table A.

Table 65 Compounds of the formula (I-G) in which R¹ is F, Y is S, and the combination of D and B for a compound corresponds in each case to one row of Table A.

Table 66 Compounds of the formula (I-G) in which R¹ is CN, Y is S, and the combination of D and B for a compound corresponds in each case to one row of Table A.

Table 67 Compounds of the formula (I-G) in which R¹ is H, Y is SO, and the combination of D and B for a compound corresponds in each case to one row of Table A.

Table 68 Compounds of the formula (I-G) in which R¹ is F, Y is SO, and the combination of D and B for a compound corresponds in each case to one row of Table A.

Table 69 Compounds of the formula (I-G) in which R¹ is CN, Y is SO, and the combination of D and B for a compound corresponds in each case to one row of Table A.

Table 70 Compounds of the formula (I-G) in which R¹ is H, Y is SO₂, and the combination of D and B for a compound corresponds in each case to one row of Table A.

Table 71 Compounds of the formula (I-G) in which R¹ is F, Y is SO₂, and the combination of D and B for a compound corresponds in each case to one row of Table A.

Table 72 Compounds of the formula (I-G) in which R¹ is CN, Y is SO₂, and the combination of D and B for a compound corresponds in each case to one row of Table A.

Table 73 Compounds of the formula (I-H) in which R¹ is H, Y is O, and the combination of D and B for a compound corresponds in each case to one row of Table A.

Table 74 Compounds of the formula (I-H) in which R¹ is F, Y is O, and the combination of D and B for a compound corresponds in each case to one row of Table A.

Table 75 Compounds of the formula (I-H) in which R¹ is CN, Y is O, and the combination of D and B for a compound corresponds in each case to one row of Table A.

Table 76 Compounds of the formula (I-H) in which R¹ is H, Y is S, and the combination of D and B for a compound corresponds in each case to one row of Table A.

Table 77 Compounds of the formula (I-H) in which R¹ is F, Y is S, and the combination of D and B for a compound corresponds in each case to one row of Table A.

Table 78 Compounds of the formula (I-H) in which R¹ is CN, Y is S, and the combination of D and B for a compound corresponds in each case to one row of Table A.

Table 79 Compounds of the formula (I-H) in which R¹ is H, Y is SO, and the combination of D and B for a compound corresponds in each case to one row of Table A.

Table 80 Compounds of the formula (I-H) in which R¹ is F, Y is SO, and the combination of D and B for a compound corresponds in each case to one row of Table A.

Table 81 Compounds of the formula (I-H) in which R¹ is CN, Y is SO, and the combination of D and B for a compound corresponds in each case to one row of Table A.

Table 82 Compounds of the formula (I-H) in which R¹ is H, Y is SO₂, and the combination of D and B for a compound corresponds in each case to one row of Table A.

Table 83 Compounds of the formula (I-H) in which R¹ is F, Y is SO₂, and the combination of D and B for a compound corresponds in each case to one row of Table A.

Table 84 Compounds of the formula (I-H) in which R¹ is CN, Y is SO₂, and the combination of D and B for a compound corresponds in each case to one row of Table A.

Table 85 Compounds of the formula (I-I) in which R¹ is H, Y is O, and the combination of D and B for a compound corresponds in each case to one row of Table A.

Table 86 Compounds of the formula (I-I) in which R¹ is F, Y is O, and the combination of D and B for a compound corresponds in each case to one row of Table A.

Table 87 Compounds of the formula (I-I) in which R¹ is CN, Y is O, and the combination of D and B for a compound corresponds in each case to one row of Table A.

Table 88 Compounds of the formula (I-I) in which R¹ is H, Y is S, and the combination of D and B for a compound corresponds in each case to one row of Table A.

Table 89 Compounds of the formula (I-I) in which R¹ is F, Y is S, and the combination of D and B for a compound corresponds in each case to one row of Table A.

Table 90 Compounds of the formula (I-I) in which R¹ is CN, Y is S, and the combination of D and B for a compound corresponds in each case to one row of Table A.

Table 91 Compounds of the formula (I-I) in which R¹ is H, Y is SO, and the combination of D and B for a compound corresponds in each case to one row of Table A.

Table 92 Compounds of the formula (I-I) in which R¹ is F, Y is SO, and the combination of D and B for a compound corresponds in each case to one row of Table A.

Table 93 Compounds of the formula (I-I) in which R¹ is CN, Y is SO, and the combination of D and B for a compound corresponds in each case to one row of Table A.

Table 94 Compounds of the formula (I-I) in which R¹ is H, Y is SO₂, and the combination of D and B for a compound corresponds in each case to one row of Table A.

Table 95 Compounds of the formula (I-I) in which R¹ is F, Y is SO₂, and the combination of D and B for a compound corresponds in each case to one row of Table A.

Table 96 Compounds of the formula (I-I) in which R¹ is CN, Y is SO₂, and the combination of D and B for a compound corresponds in each case to one row of Table A.

Table 97 Compounds of the formula (I-J) in which R¹ is H, Y is O, and the combination of D and B for a compound corresponds in each case to one row of Table A.

Table 98 Compounds of the formula (I-J) in which R¹ is F, Y is O, and the combination of D and B for a compound corresponds in each case to one row of Table A.

Table 99 Compounds of the formula (I-J) in which R¹ is CN, Y is O, and the combination of D and B for a compound corresponds in each case to one row of Table A.

Table 100 Compounds of the formula (I-J) in which R¹ is H, Y is S, and the combination of D and B for a compound corresponds in each case to one row of Table A.

Table 101 Compounds of the formula (I-J) in which R¹ is F, Y is S, and the combination of D and B for a compound corresponds in each case to one row of Table A.

Table 102 Compounds of the formula (I-J) in which R¹ is CN, Y is S, and the combination of D and B for a compound corresponds in each case to one row of Table A.

Table 103 Compounds of the formula (I-J) in which R¹ is H, Y is SO, and the combination of D and B for a compound corresponds in each case to one row of Table A.

Table 104 Compounds of the formula (I-J) in which R¹ is F, Y is SO, and the combination of D and B for a compound corresponds in each case to one row of Table A.

Table 105 Compounds of the formula (I-J) in which R¹ is CN, Y is SO, and the combination of D and B for a compound corresponds in each case to one row of Table A.

Table 106 Compounds of the formula (I-J) in which R¹ is H, Y is SO₂, and the combination of D and B for a compound corresponds in each case to one row of Table A.

Table 107 Compounds of the formula (I-J) in which R¹ is F, Y is SO₂, and the combination of D and B for a compound corresponds in each case to one row of Table A.

Table 108 Compounds of the formula (I-J) in which R¹ is CN, Y is SO₂, and the combination of D and B for a compound corresponds in each case to one row of Table A.

Table 109 Compounds of the formula (I-K) in which R¹ is H, Y is O, and the combination of D and B for a compound corresponds in each case to one row of Table A.

Table 110 Compounds of the formula (I-K) in which R¹ is F, Y is O, and the combination of D and B for a compound corresponds in each case to one row of Table A.

Table 111 Compounds of the formula (I-K) in which R¹ is CN, Y is O, and the combination of D and B for a compound corresponds in each case to one row of Table A.

Table 112 Compounds of the formula (I-K) in which R¹ is H, Y is S, and the combination of D and B for a compound corresponds in each case to one row of Table A.

Table 113 Compounds of the formula (I-K) in which R¹ is F, Y is S, and the combination of D and B for a compound corresponds in each case to one row of Table A.

Table 114 Compounds of the formula (I-K) in which R¹ is CN, Y is S, and the combination of D and B for a compound corresponds in each case to one row of Table A.

Table 115 Compounds of the formula (I-K) in which R¹ is H, Y is SO, and the combination of D and B for a compound corresponds in each case to one row of Table A.

Table 116 Compounds of the formula (I-K) in which R¹ is F, Y is SO, and the combination of D and B for a compound corresponds in each case to one row of Table A.

Table 117 Compounds of the formula (I-K) in which R¹ is CN, Y is SO, and the combination of D and B for a compound corresponds in each case to one row of Table A.

Table 118 Compounds of the formula (I-K) in which R¹ is H, Y is SO₂, and the combination of D and B for a compound corresponds in each case to one row of Table A.

Table 119 Compounds of the formula (I-K) in which R¹ is F, Y is SO₂, and the combination of D and B for a compound corresponds in each case to one row of Table A.

Table 120 Compounds of the formula (I-K) in which R¹ is CN, Y is SO₂, and the combination of D and B for a compound corresponds in each case to one row of Table A.

TABLE A

| | D | B |
|---|---|---|
| A-1 | H | CH₃ |
| A-2 | H | CH₂CH₃ |
| A-3 | H | CH₂CH₂CH₃ |
| A-4 | H | c-C₃H₅ |
| A-5 | H | CH(CH₃)₂ |
| A-6 | H | CH₂—(c-C₃H₅) |
| A-7 | H | CH(CH₃)—(c-C₃H₅) |
| A-8 | H | CH₂CH₂CH₂CH₃ |
| A-9 | H | CH(CH₃)CH₂CH₃ |
| A-10 | H | CH₂CH(CH₃)₂ |
| A-11 | H | C(CH₃)₃ |
| A-12 | H | c-C₄H₇ |
| A-13 | H | 1-CH₃—c-C₃H₄ |
| A-14 | H | CH₂CH═CH₂ |
| A-15 | H | CH(CH₃)CH═CH₂ |
| A-16 | H | CH₂C≡CH |
| A-17 | H | CH(CH₃)C≡CH |
| A-18 | H | C(CH₃)₂C≡CH |
| A-19 | H | CH₂F |
| A-20 | H | CHF₂ |
| A-21 | H | CF₃ |
| A-22 | H | CH₂CH₂F |
| A-23 | H | CH₂CHF₂ |
| A-24 | H | CH₂CF₃ |
| A-25 | H | CH(CH₃)CF₃ |
| A-26 | H | CH₂CH₂CF₃ |
| A-27 | H | CH₂CF₂CF₃ |
| A-28 | H | CH₂CF₂CH₃ |
| A-29 | H | CH₂CH₂CF₂CF₃ |
| A-30 | H | CH(CH(CH₃)₂)CF₃ |
| A-31 | H | CH₂(2,2-FcP) |
| A-32 | H | 3,3-FB |
| A-33 | H | 4,4-FH |
| A-34 | H | CH₂CH₂OCF₃ |
| A-35 | H | CF₂CH₂OCH₃ |
| A-36 | H | CF₂CH₂OCF₃ |
| A-37 | H | CH₂CF₂OCH₃ |
| A-38 | H | CH₂CF₂OCF₃ |
| A-39 | H | CF₂CF₂OCH₃ |
| A-40 | H | CF₂CF₂OCF₃ |
| A-41 | H | CH₂CH₂SCF₃ |
| A-42 | H | CF₂CH₂SCH₃ |
| A-43 | H | CF₂CH₂SCF₃ |
| A-44 | H | CH₂CF₂SCH₃ |
| A-45 | H | CH₂CF₂SCF₃ |
| A-46 | H | CF₂CF₂SCH₃ |
| A-47 | H | CF₂CF₂SCF₃ |
| A-48 | H | CH₂CH₂OCH₃ |
| A-49 | H | CH₂OCH₂CH₃ |
| A-50 | H | C(CH₃)₂OCH₃ |
| A-51 | H | C(CH₃)₂OCH₂CH₃ |
| A-52 | H | CH₂CH₂OCH(CH₃)₂ |
| A-53 | H | CH(CH₃)CH₂OCH₃ |
| A-54 | H | CH(CH₂CH₃)CH₂OCH₃ |
| A-55 | H | CH(CH₃)CH₂OCH₂CH₃ |
| A-56 | H | CH(CH₂CH₃)CH₂OCH₂CH₃ |
| A-57 | H | CH₂CH(CH₃)OCH₃ |
| A-58 | H | CH₂CH(CH₂CH₃)OCH₃ |
| A-59 | H | CH₂CH(CH₃)OCH₂CH₃ |
| A-60 | H | CH₂CH(CH₂CH₃)OCH₂CH₃ |
| A-61 | H | CH₂CH(OCH₃)₂ |
| A-62 | H | CH(CH₃)CH(OCH₃)₂ |
| A-63 | H | CH₂C(CH₃)(OCH₃)₂ |
| A-64 | H | CH₂CH₂CH₂OCH₃ |
| A-65 | H | CH₂CH(CH₃)OCH₃ |
| A-66 | H | CH(CH₂CH₃)CH₂OCH₃ |
| A-67 | H | CH(CH₃)CH₂OCH₃ |
| A-68 | H | CH₂(2-FU) |
| A-69 | H | CH₂(2-HFU) |
| A-70 | H | CH₂(3-FU) |
| A-71 | H | CH₂(3-HFU) |
| A-72 | H | CH₂CH₂SCH₃ |
| A-73 | H | CH₂CH₂S(O)CH₃ |
| A-74 | H | CH₂CH₂S(O)₂CH₃ |
| A-75 | H | CH₂CH₂SCH₂CH₃ |
| A-76 | H | CH₂CH₂S(O)CH₂CH₃ |
| A-77 | H | CH₂CH₂S(O)₂CH₂CH₃ |
| A-78 | H | CH₂CH₂SCH(CH₃)₂ |
| A-79 | H | CH₂CH₂S(O)CH(CH₃)₂ |
| A-80 | H | CH₂CH₂S(O)₂CH(CH₃)₂ |
| A-81 | H | CH₂CH₂SC(CH₃)₃ |
| A-82 | H | CH₂CH₂S(O)C(CH₃)₃ |
| A-83 | H | CH₂CH₂S(O)₂C(CH₃)₃ |
| A-84 | H | CH(CH₃)CH₂SCH₃ |
| A-85 | H | CH(CH₂CH₃)CH₂SCH₃ |
| A-86 | H | CH(CH₃)CH₂S(O)CH₃ |
| A-87 | H | CH(CH₂CH₃)CH₂S(O)CH₃ |
| A-88 | H | CH(CH₃)CH₂S(O)₂CH₃ |
| A-89 | H | CH(CH₂CH₃)CH₂S(O)₂CH₃ |
| A-90 | H | CH₂CH(CH₃)SCH₃ |
| A-91 | H | CH₂CH(CH₂CH₃)SCH₃ |
| A-92 | H | CH₂CH(CH₃)S(O)CH₃ |
| A-93 | H | CH₂CH(CH₂CH₃)S(O)CH₃ |
| A-94 | H | CH₂CH(CH₃)S(O)₂CH₃ |
| A-95 | H | CH₂CH(CH₂CH₃)S(O)₂CH₃ |
| A-96 | H | CH₂CN |
| A-97 | H | CH₂CH₂CN |
| A-98 | H | C(CH₃)₂CN |
| A-99 | H | CH₂CH₂CH₂CN |
| A-100 | H | CH(CN)CH(CH₃)₂ |
| A-101 | H | CH₂CH₂OCH₂CH₃ |
| A-102 | H | CH₂CH₂C(O)OCH₃ |
| A-103 | H | CH₂CH₂CH₂S(O)CH₂CH₂CF₃ |
| A-104 | H | CH₂CH₂CH₂CH₂CH₃ |
| A-105 | H | CH(CH₂OCH₂CF₃)CH₂CH₃ |
| A-106 | H | c-C₅H₉ |
| A-107 | H | CH₂CF₂CF₂H |
| A-108 | H | CH₂CH(OCH₂CH₃)₂ |
| A-109 | H | CH(CH₃)CH(CH₃)₂ |
| A-110 | H | CH₂CH₂CH(CH₃)₂ |
| A-111 | H | CH₂CH₂Si(CH₃)₃ |
| A-112 | H | CH₂CH₂CH₂SCH₂CH₂CF₃ |
| A-113 | H | CH₂CH₂CH₂S(O)CH₃ |
| A-114 | H | CH(CH₃)CH₂OCH═CH₂ |
| A-115 | H | CH(C₆H₅)CH₃ |
| A-116 | H | CH₂CH₂N[C(O)CF₃]S(O)₂CH₃ |
| A-117 | H | CH₂C(CH₃)₂(S(O)₂CH₃) |
| A-118 | H | CH₂CH₂CH₂SCH₃ |
| A-119 | H | CH₂CH(CH₃)CH₂SCH₃ |
| A-120 | H | CH₂CH(CH₃)CH₂S(O)CH₃ |
| A-121 | H | CH₂CH₂CH═CH₂ |
| A-122 | H | CH₂C(O)NH(CH₃) |
| A-123 | H | CH₂C(O)NHS(O)₂CH₃ |
| A-124 | H | CH₂C(CH═NOH)CH₃ |
| A-125 | H | CH₂CH₂CH₂SCH₂CH═CH₂ |
| A-126 | H | CHCH₃C(O)OCH₂CH₃ |
| A-127 | H | CH₂CH₂OH |
| A-128 | H | CH₂CH₂OC(O)c—C₃H₅ |
| A-129 | H | CH₂CH₂C(O)OCH₂CH₃ |
| A-130 | H | B¹ |
| A-131 | H | B² |
| A-132 | H | B³ |
| A-133 | H | B⁴ |
| A-134 | H | B⁵ |
| A-135 | H | B⁶ |
| A-136 | H | B⁷ |
| A-137 | H | B⁸ |
| A-138 | H | B⁹ |
| A-139 | H | B¹⁰ |
| A-140 | H | B¹² |
| A-141 | H | B¹³ |
| A-142 | H | B¹⁴ |
| A-143 | H | B¹⁵ |
| A-144 | H | B¹⁶ |
| A-145 | H | B¹⁸ |
| A-146 | H | B¹⁹ |
| A-147 | H | B²⁰ |
| A-148 | F | CH₃ |
| A-149 | F | CH₂CH₃ |
| A-150 | F | CH₂CH₂CH₃ |
| A-151 | F | c-C₃H₅ |
| A-152 | F | CH(CH₃)₂ |
| A-153 | F | CH₂—(c-C₃H₅) |
| A-154 | F | CH(CH₃)—(c-C₃H₅) |
| A-155 | F | CH₂CH₂CH₂CH₃ |
| A-156 | F | CH(CH₃)CH₂CH₃ |
| A-157 | F | CH₂CH(CH₃)₂ |
| A-158 | F | C(CH₃)₃ |
| A-159 | F | c-C₄H₇ |
| A-160 | F | 1-CH₃—c-C₃H₄ |
| A-161 | F | CH₂CH═CH₂ |
| A-162 | F | CH(CH₃)CH═CH₂ |
| A-163 | F | CH₂C≡CH |
| A-164 | F | CH(CH₃)C≡CH |
| A-165 | F | C(CH₃)₂C≡CH |
| A-166 | F | CH₂F |
| A-167 | F | CHF₂ |
| A-168 | F | CF₃ |
| A-169 | F | CH₂CH₂F |
| A-170 | F | CH₂CHF₂ |
| A-171 | F | CH₂CF₃ |
| A-172 | F | CH(CH₃)CF₃ |
| A-173 | F | CH₂CH₂CF₃ |
| A-174 | F | CH₂CF₂CF₃ |
| A-175 | F | CH₂CF₂CH₃ |
| A-176 | F | CH₂CH₂CF₂CF₃ |
| A-177 | F | CH(CH(CH₃)₂)CF₃ |
| A-178 | F | CH₂(2,2-FcP) |
| A-179 | F | 3,3-FB |
| A-180 | F | 4,4-FH |
| A-181 | F | CH₂CH₂OCF₃ |
| A-182 | F | CF₂CH₂OCH₃ |
| A-183 | F | CF₂CH₂OCF₃ |
| A-184 | F | CH₂CF₂OCH₃ |
| A-185 | F | CH₂CF₂OCF₃ |
| A-186 | F | CF₂CF₂OCH₃ |
| A-187 | F | CF₂CF₂OCF₃ |
| A-188 | F | CH₂CH₂SCF₃ |
| A-189 | F | CF₂CH₂SCH₃ |
| A-190 | F | CF₂CH₂SCF₃ |
| A-191 | F | CH₂CF₂SCH₃ |
| A-192 | F | CH₂CF₂SCF₃ |
| A-193 | F | CF₂CF₂SCH₃ |
| A-194 | F | CF₂CF₂SCF₃ |
| A-195 | F | CH₂CH₂OCH₃ |
| A-196 | F | CH₂OCH₂CH₃ |
| A-197 | F | C(CH₃)₂OCH₃ |
| A-198 | F | C(CH₃)₂OCH₂CH₃ |
| A-199 | F | CH₂CH₂OCH(CH₃)₂ |
| A-200 | F | CH(CH₃)CH₂OCH₃ |
| A-201 | F | CH(CH₂CH₃)CH₂OCH₃ |
| A-202 | F | CH(CH₃)CH₂OCH₂CH₃ |
| A-203 | F | CH(CH₂CH₃)CH₂OCH₂CH₃ |
| A-204 | F | CH₂CH(CH₃)OCH₃ |
| A-205 | F | CH₂CH(CH₂CH₃)OCH₃ |
| A-206 | F | CH₂CH(CH₃)OCH₂CH₃ |
| A-207 | F | CH₂CH(CH₂CH₃)OCH₂CH₃ |
| A-208 | F | CH₂CH(OCH₃)₂ |
| A-209 | F | CH(CH₃)CH(OCH₃)₂ |
| A-210 | F | CH₂C(CH₃)(OCH₃)₂ |
| A-211 | F | CH₂CH₂CH₂OCH₃ |
| A-212 | F | CH₂CH(CH₃)OCH₃ |
| A-213 | F | CH(CH₂CH₃)CH₂OCH₃ |
| A-214 | F | CH(CH₃)CH₂OCH₃ |
| A-215 | F | CH₂(2-FU) |
| A-216 | F | CH₂(2-HFU) |
| A-217 | F | CH₂(3-FU) |
| A-218 | F | CH₂(3-HFU) |
| A-219 | F | CH₂CH₂SCH₃ |
| A-220 | F | CH₂CH₂S(O)CH₃ |
| A-221 | F | CH₂CH₂S(O)₂CH₃ |
| A-222 | F | CH₂CH₂SCH₂CH₃ |
| A-223 | F | CH₂CH₂S(O)CH₂CH₃ |
| A-224 | F | CH₂CH₂S(O)₂CH₂CH₃ |
| A-225 | F | CH₂CH₂SCH(CH₃)₂ |
| A-226 | F | CH₂CH₂S(O)CH(CH₃)₂ |
| A-227 | F | CH₂CH₂S(O)₂CH(CH₃)₂ |
| A-228 | F | CH₂CH₂SC(CH₃)₃ |
| A-229 | F | CH₂CH₂S(O)C(CH₃)₃ |
| A-230 | F | CH₂CH₂S(O)₂C(CH₃)₃ |
| A-231 | F | CH(CH₃)CH₂SCH₃ |
| A-232 | F | CH(CH₂CH₃)CH₂SCH₃ |
| A-233 | F | CH(CH₃)CH₂S(O)CH₃ |
| A-234 | F | CH(CH₂CH₃)CH₂S(O)CH₃ |
| A-235 | F | CH(CH₃)CH₂S(O)₂CH₃ |
| A-236 | F | CH(CH₂CH₃)CH₂S(O)₂CH₃ |
| A-237 | F | CH₂CH(CH₃)SCH₃ |
| A-238 | F | CH₂CH(CH₂CH₃)SCH₃ |
| A-239 | F | CH₂CH(CH₃)S(O)CH₃ |
| A-240 | F | CH₂CH(CH₂CH₃)S(O)CH₃ |
| A-241 | F | CH₂CH(CH₃)S(O)₂CH₃ |
| A-242 | F | CH₂CH(CH₂CH₃)S(O)₂CH₃ |
| A-243 | F | CH₂CN |
| A-244 | F | CH₂CH₂CN |
| A-245 | F | C(CH₃)₂CN |
| A-246 | F | CH₂CH₂CH₂CN |
| A-247 | F | CH(CN)CH(CH₃)₂ |
| A-248 | F | CH₂CH₂OCH₂CH₃ |
| A-249 | F | CH₂CH₂C(O)OCH₃ |
| A-250 | F | CH₂CH₂CH₂S(O)CH₂CH₂CF₃ |
| A-251 | F | CH₂CH₂CH₂CH₂CH₃ |
| A-252 | F | CH(CH₂OCH₂CF₃)CH₂CH₃ |
| A-253 | F | c-C₅H₉ |
| A-254 | F | CH₂CF₂CF₂H |
| A-255 | F | CH₂CH(OCH₂CH₃)₂ |
| A-256 | F | CH(CH₃)CH(CH₃)₂ |
| A-257 | F | CH₂CH₂CH(CH₃)₂ |
| A-258 | F | CH₂CH₂Si(CH₃)₃ |
| A-259 | F | CH₂CH₂CH₂SCH₂CH₂CF₃ |
| A-260 | F | CH₂CH₂CH₂S(O)CH₃ |
| A-261 | F | CH(CH₃)CH₂OCH═CH₂ |
| A-262 | F | CH(C₆H₅)CH₃ |
| A-263 | F | CH₂CH₂N[C(O)CF₃]S(O)₂CH₃ |
| A-264 | F | CH₂C(CH₃)₂(S(O)₂CH₃) |
| A-265 | F | CH₂CH₂CH₂SCH₃ |
| A-266 | F | CH₂CH(CH₃)CH₂SCH₃ |
| A-267 | F | CH₂CH(CH₃)CH₂S(O)CH₃ |
| A-268 | F | CH₂CH₂CH═CH₂ |
| A-269 | F | CH₂C(O)NH(CH₃) |
| A-270 | F | CH₂C(O)NHS(O)₂CH₃ |
| A-271 | F | CH₂C(CH═NOH)CH₃ |
| A-272 | F | CH₂CH₂CH₂SCH₂CH═CH₂ |
| A-273 | F | CHCH₃C(O)OCH₂CH₃ |
| A-274 | F | CH₂CH₂OH |
| A-275 | F | CH₂CH₂OC(O)c—C₃H₅ |
| A-276 | F | CH₂CH₂C(O)OCH₂CH₃ |
| A-277 | F | B¹ |
| A-278 | F | B² |
| A-279 | F | B³ |
| A-280 | F | B⁴ |
| A-281 | F | B⁵ |
| A-282 | F | B⁶ |
| A-283 | F | B⁷ |
| A-284 | F | B⁸ |
| A-285 | F | B⁹ |
| A-286 | F | B¹⁰ |
| A-287 | F | B¹² |
| A-288 | F | B¹³ |
| A-289 | F | B¹⁴ |
| A-290 | F | B¹⁵ |
| A-291 | F | B¹⁶ |
| A-292 | F | B¹⁸ |
| A-293 | F | B¹⁹ |
| A-294 | F | B²⁰ |
| A-295 | Cl | CH₃ |
| A-296 | Cl | CH₂CH₃ |
| A-297 | Cl | CH₂CH₂CH₃ |
| A-298 | Cl | c-C₃H₅ |
| A-299 | Cl | CH(CH₃)₂ |
| A-300 | Cl | CH₂—(c-C₃H₅) |
| A-301 | Cl | CH(CH₃)—(c-C₃H₅) |
| A-302 | Cl | CH₂CH₂CH₂CH₃ |
| A-303 | Cl | CH(CH₃)CH₂CH₃ |
| A-304 | Cl | CH₂CH(CH₃)₂ |
| A-305 | Cl | C(CH₃)₃ |
| A-306 | Cl | c-C₄H₇ |
| A-307 | Cl | 1-CH₃—c-C₃H₄ |
| A-308 | Cl | CH₂CH═CH₂ |
| A-309 | Cl | CH(CH₃)CH═CH₂ |
| A-310 | Cl | CH₂C≡CH |
| A-311 | Cl | CH(CH₃)C≡CH |
| A-312 | Cl | C(CH₃)₂C≡CH |
| A-313 | Cl | CH₂F |
| A-314 | Cl | CHF₂ |
| A-315 | Cl | CF₃ |
| A-316 | Cl | CH₂CH₂F |
| A-317 | Cl | CH₂CHF₂ |
| A-318 | Cl | CH₂CF₃ |
| A-319 | Cl | CH(CH₃)CF₃ |
| A-320 | Cl | CH₂CH₂CF₃ |
| A-321 | Cl | CH₂CF₂CF₃ |
| A-322 | Cl | CH₂CF₂CH₃ |
| A-323 | Cl | CH₂CH₂CF₂CF₃ |
| A-324 | Cl | CH(CH(CH₃)₂)CF₃ |
| A-325 | Cl | CH₂(2,2-FcP) |
| A-326 | Cl | 3,3-FB |
| A-327 | Cl | 4,4-FH |
| A-328 | Cl | CH₂CH₂OCF₃ |
| A-329 | Cl | CF₂CH₂OCH₃ |
| A-330 | Cl | CF₂CH₂OCF₃ |
| A-331 | Cl | CH₂CF₂OCH₃ |
| A-332 | Cl | CH₂CF₂OCF₃ |
| A-333 | Cl | CF₂CF₂OCH₃ |
| A-334 | Cl | CF₂CF₂OCF₃ |
| A-335 | Cl | CH₂CH₂SCF₃ |
| A-336 | Cl | CF₂CH₂SCH₃ |
| A-337 | Cl | CF₂CH₂SCF₃ |
| A-338 | Cl | CH₂CF₂SCH₃ |
| A-339 | Cl | CH₂CF₂SCF₃ |
| A-340 | Cl | CF₂CF₂SCH₃ |
| A-341 | Cl | CF₂CF₂SCF₃ |
| A-342 | Cl | CH₂CH₂OCH₃ |
| A-343 | Cl | CH₂OCH₂CH₃ |
| A-344 | Cl | C(CH₃)₂OCH₃ |
| A-345 | Cl | C(CH₃)₂OCH₂CH₃ |
| A-346 | Cl | CH₂CH₂OCH(CH₃)₂ |
| A-347 | Cl | CH(CH₃)CH₂OCH₃ |
| A-348 | Cl | CH(CH₂CH₃)CH₂OCH₃ |
| A-349 | Cl | CH(CH₃)CH₂OCH₂CH₃ |
| A-350 | Cl | CH(CH₂CH₃)CH₂OCH₂CH₃ |
| A-351 | Cl | CH₂CH(CH₃)OCH₃ |
| A-352 | Cl | CH₂CH(CH₂CH₃)OCH₃ |
| A-353 | Cl | CH₂CH(CH₃)OCH₂CH₃ |
| A-354 | Cl | CH₂CH(CH₂CH₃)OCH₂CH₃ |
| A-355 | Cl | CH₂CH(OCH₃)₂ |
| A-356 | Cl | CH(CH₃)CH(OCH₃)₂ |
| A-357 | Cl | CH₂C(CH₃)(OCH₃)₂ |
| A-358 | Cl | CH₂CH₂CH₂OCH₃ |
| A-359 | Cl | CH₂CH(CH₃)OCH₃ |
| A-360 | Cl | CH(CH₂CH₃)CH₂OCH₃ |
| A-361 | Cl | CH(CH₃)CH₂OCH₃ |
| A-362 | Cl | CH₂(2-FU) |
| A-363 | Cl | CH₂(2-HFU) |
| A-364 | Cl | CH₂(3-FU) |
| A-365 | Cl | CH₂(3-HFU) |
| A-366 | Cl | CH₂CH₂SCH₃ |
| A-367 | Cl | CH₂CH₂S(O)CH₃ |
| A-368 | Cl | CH₂CH₂S(O)₂CH₃ |
| A-369 | Cl | CH₂CH₂SCH₂CH₃ |
| A-370 | Cl | CH₂CH₂S(O)CH₂CH₃ |
| A-371 | Cl | CH₂CH₂S(O)₂CH₂CH₃ |
| A-372 | Cl | CH₂CH₂SCH(CH₃)₂ |
| A-373 | Cl | CH₂CH₂S(O)CH(CH₃)₂ |
| A-374 | Cl | CH₂CH₂S(O)₂CH(CH₃)₂ |
| A-375 | Cl | CH₂CH₂SC(CH₃)₃ |
| A-376 | Cl | CH₂CH₂S(O)C(CH₃)₃ |
| A-377 | Cl | CH₂CH₂S(O)₂C(CH₃)₃ |
| A-378 | Cl | CH(CH₃)CH₂SCH₃ |
| A-379 | Cl | CH(CH₂CH₃)CH₂SCH₃ |
| A-380 | Cl | CH(CH₃)CH₂S(O)CH₃ |
| A-381 | Cl | CH(CH₂CH₃)CH₂S(O)CH₃ |
| A-382 | Cl | CH(CH₃)CH₂S(O)₂CH₃ |
| A-383 | Cl | CH(CH₂CH₃)CH₂S(O)₂CH₃ |
| A-384 | Cl | CH₂CH(CH₃)SCH₃ |
| A-385 | Cl | CH₂CH(CH₂CH₃)SCH₃ |
| A-386 | Cl | CH₂CH(CH₃)S(O)CH₃ |
| A-387 | Cl | CH₂CH(CH₂CH₃)S(O)CH₃ |
| A-388 | Cl | CH₂CH(CH₃)S(O)₂CH₃ |
| A-389 | Cl | CH₂CH(CH₂CH₃)S(O)₂CH₃ |
| A-390 | Cl | CH₂CN |
| A-391 | Cl | CH₂CH₂CN |
| A-392 | Cl | C(CH₃)₂CN |
| A-393 | Cl | CH₂CH₂CH₂CN |
| A-394 | Cl | CH(CN)CH(CH₃)₂ |
| A-395 | Cl | CH₂CH₂OCH₂CH₃ |
| A-396 | Cl | CH₂CH₂C(O)OCH₃ |
| A-397 | Cl | CH₂CH₂CH₂S(O)CH₂CH₂CF₃ |
| A-398 | Cl | CH₂CH₂CH₂CH₂CH₃ |
| A-399 | Cl | CH(CH₂OCH₂CF₃)CH₂CH₃ |
| A-400 | Cl | c-C₅H₉ |
| A-401 | Cl | CH₂CF₂CF₂H |
| A-402 | Cl | CH₂CH(OCH₂CH₃)₂ |
| A-403 | Cl | CH(CH₃)CH(CH₃)₂ |
| A-404 | Cl | CH₂CH₂CH(CH₃)₂ |
| A-405 | Cl | CH₂CH₂Si(CH₃)₃ |
| A-406 | Cl | CH₂CH₂CH₂SCH₂CH₂CF₃ |
| A-407 | Cl | CH₂CH₂CH₂S(O)CH₃ |
| A-408 | Cl | CH(CH₃)CH₂OCH═CH₂ |
| A-409 | Cl | CH(C₆H₅)CH₃ |
| A-410 | Cl | CH₂CH₂N[C(O)CF₃]S(O)₂CH₃ |
| A-411 | Cl | CH₂C(CH₃)₂(S(O)₂CH₃) |
| A-412 | Cl | CH₂CH₂CH₂SCH₃ |
| A-413 | Cl | CH₂CH(CH₃)CH₂SCH₃ |
| A-414 | Cl | CH₂CH(CH₃)CH₂S(O)CH₃ |
| A-415 | Cl | CH₂CH₂CH═CH₂ |
| A-416 | Cl | CH₂C(O)NH(CH₃) |
| A-417 | Cl | CH₂C(O)NHS(O)₂CH₃ |
| A-418 | Cl | CH₂C(CH═NOH)CH₃ |
| A-419 | Cl | CH₂CH₂CH₂SCH₂CH═CH₂ |
| A-420 | Cl | CHCH₃C(O)OCH₂CH₃ |
| A-421 | Cl | CH₂CH₂OH |
| A-422 | Cl | CH₂CH₂OC(O)c—C₃H₅ |
| A-423 | Cl | CH₂CH₂C(O)OCH₂CH₃ |
| A-424 | Cl | B¹ |
| A-425 | Cl | B² |
| A-426 | Cl | B³ |
| A-427 | Cl | B⁴ |
| A-428 | Cl | B⁵ |
| A-429 | Cl | B⁶ |
| A-430 | Cl | B⁷ |
| A-431 | Cl | B⁸ |
| A-432 | Cl | B⁹ |
| A-433 | Cl | B¹⁰ |
| A-434 | Cl | B¹² |
| A-435 | Cl | B¹³ |
| A-436 | Cl | B¹⁴ |
| A-437 | Cl | B¹⁵ |
| A-438 | Cl | B¹⁶ |
| A-439 | Cl | B¹⁸ |
| A-440 | Cl | B¹⁹ |
| A-441 | Cl | B²⁰ |
| A-442 | Br | CH₃ |
| A-443 | Br | CH₂CH₃ |
| A-444 | Br | CH₂CH₂CH₃ |
| A-445 | Br | c-C₃H₅ |
| A-446 | Br | CH(CH₃)₂ |
| A-447 | Br | CH₂—(c-C₃H₅) |
| A-448 | Br | CH(CH₃)—(c-C₃H₅) |
| A-449 | Br | CH₂CH₂CH₂CH₃ |
| A-450 | Br | CH(CH₃)CH₂CH₃ |
| A-451 | Br | CH₂CH(CH₃)₂ |
| A-452 | Br | C(CH₃)₃ |
| A-453 | Br | c-C₄H₇ |
| A-454 | Br | 1-CH₃—c-C₃H₄ |
| A-455 | Br | CH₂CH═CH₂ |
| A-456 | Br | CH(CH₃)CH═CH₂ |
| A-457 | Br | CH₂C≡CH |
| A-458 | Br | CH(CH₃)C≡CH |
| A-459 | Br | C(CH₃)₂C≡CH |
| A-460 | Br | CH₂F |
| A-461 | Br | CHF₂ |
| A-462 | Br | CF₃ |
| A-463 | Br | CH₂CH₂F |
| A-464 | Br | CH₂CHF₂ |
| A-465 | Br | CH₂CF₃ |
| A-466 | Br | CH(CH₃)CF₃ |
| A-467 | Br | CH₂CH₂CF₃ |
| A-468 | Br | CH₂CF₂CF₃ |
| A-469 | Br | CH₂CF₂CH₃ |
| A-470 | Br | CH₂CH₂CF₂CF₃ |
| A-471 | Br | CH(CH(CH₃)₂)CF₃ |
| A-472 | Br | CH₂(2,2-FcP) |
| A-473 | Br | 3,3-FB |
| A-474 | Br | 4,4-FH |
| A-475 | Br | CH₂CH₂OCF₃ |
| A-476 | Br | CF₂CH₂OCH₃ |
| A-477 | Br | CF₂CH₂OCF₃ |
| A-478 | Br | CH₂CF₂OCH₃ |
| A-479 | Br | CH₂CF₂OCF₃ |
| A-480 | Br | CF₂CF₂OCH₃ |
| A-481 | Br | CF₂CF₂OCF₃ |
| A-482 | Br | CH₂CH₂SCF₃ |
| A-483 | Br | CF₂CH₂SCH₃ |
| A-484 | Br | CF₂CH₂SCF₃ |
| A-485 | Br | CH₂CF₂SCH₃ |
| A-486 | Br | CH₂CF₂SCF₃ |
| A-487 | Br | CF₂CF₂SCH₃ |
| A-488 | Br | CF₂CF₂SCF₃ |
| A-489 | Br | CH₂CH₂OCH₃ |
| A-490 | Br | CH₂OCH₂CH₃ |
| A-491 | Br | C(CH₃)₂OCH₃ |
| A-492 | Br | C(CH₃)₂OCH₂CH₃ |
| A-493 | Br | CH₂CH₂OCH(CH₃)₂ |
| A-494 | Br | CH(CH₃)CH₂OCH₃ |
| A-495 | Br | CH(CH₂CH₃)CH₂OCH₃ |
| A-496 | Br | CH(CH₃)CH₂OCH₂CH₃ |
| A-497 | Br | CH(CH₂CH₃)CH₂OCH₂CH₃ |
| A-498 | Br | CH₂CH(CH₃)OCH₃ |
| A-499 | Br | CH₂CH(CH₂CH₃)OCH₃ |
| A-500 | Br | CH₂CH(CH₃)OCH₂CH₃ |
| A-501 | Br | CH₂CH(CH₂CH₃)OCH₂CH₃ |
| A-502 | Br | CH₂CH(OCH₃)₂ |
| A-503 | Br | CH(CH₃)CH(OCH₃)₂ |
| A-504 | Br | CH₂C(CH₃)(OCH₃)₂ |
| A-505 | Br | CH₂CH₂CH₂OCH₃ |
| A-506 | Br | CH₂CH(CH₃)OCH₃ |
| A-507 | Br | CH(CH₂CH₃)CH₂OCH₃ |
| A-508 | Br | CH(CH₃)CH₂OCH₃ |
| A-509 | Br | CH₂(2-FU) |
| A-510 | Br | CH₂(2-HFU) |
| A-511 | Br | CH₂(2-FU) |
| A-512 | Br | CH₂(2-HFU) |
| A-513 | Br | CH₂CH₂SCH₃ |
| A-514 | Br | CH₂CH₂S(O)CH₃ |
| A-515 | Br | CH₂CH₂S(O)₂CH₃ |
| A-516 | Br | CH₂CH₂SCH₂CH₃ |
| A-517 | Br | CH₂CH₂S(O)CH₂CH₃ |
| A-518 | Br | CH₂CH₂S(O)₂CH₂CH₃ |
| A-519 | Br | CH₂CH₂SCH(CH₃)₂ |
| A-520 | Br | CH₂CH₂S(O)CH(CH₃)₂ |
| A-521 | Br | CH₂CH₂S(O)₂CH(CH₃)₂ |
| A-522 | Br | CH₂CH₂SC(CH₃)₃ |
| A-523 | Br | CH₂CH₂S(O)C(CH₃)₃ |
| A-524 | Br | CH₂CH₂S(O)₂C(CH₃)₃ |
| A-525 | Br | CH(CH₃)CH₂SCH₃ |
| A-526 | Br | CH(CH₂CH₃)CH₂SCH₃ |
| A-527 | Br | CH(CH₃)CH₂S(O)CH₃ |
| A-528 | Br | CH(CH₂CH₃)CH₂S(O)CH₃ |
| A-529 | Br | CH(CH₃)CH₂S(O)₂CH₃ |
| A-530 | Br | CH(CH₂CH₃)CH₂S(O)₂CH₃ |
| A-531 | Br | CH₂CH(CH₃)SCH₃ |
| A-532 | Br | CH₂CH(CH₂CH₃)SCH₃ |
| A-533 | Br | CH₂CH(CH₃)S(O)CH₃ |
| A-534 | Br | CH₂CH(CH₂CH₃)S(O)CH₃ |
| A-535 | Br | CH₂CH(CH₃)S(O)₂CH₃ |
| A-536 | Br | CH₂CH(CH₂CH₃)S(O)₂CH₃ |
| A-537 | Br | CH₂CN |
| A-538 | Br | CH₂CH₂CN |
| A-539 | Br | C(CH₃)₂CN |
| A-540 | Br | CH₂CH₂CH₂CN |
| A-541 | Br | CH(CN)CH(CH₃)₂ |
| A-542 | Br | CH₂CH₂OCH₂CH₃ |
| A-543 | Br | CH₂CH₂C(O)OCH₃ |
| A-544 | Br | CH₂CH₂CH₂S(O)CH₂CH₂CF₃ |
| A-545 | Br | CH₂CH₂CH₂CH₂CH₃ |
| A-546 | Br | CH(CH₂OCH₂CF₃)CH₂CH₃ |
| A-547 | Br | c-C₅H₉ |
| A-548 | Br | CH₂CF₂CF₂H |
| A-549 | Br | CH₂CH(OCH₂CH₃)₂ |
| A-550 | Br | CH(CH₃)CH(CH₃)₂ |
| A-551 | Br | CH₂CH₂CH(CH₃)₂ |
| A-552 | Br | CH₂CH₂Si(CH₃)₃ |
| A-553 | Br | CH₂CH₂CH₂SCH₂CH₂CF₃ |
| A-554 | Br | CH₂CH₂CH₂S(O)CH₃ |
| A-555 | Br | CH(CH₃)CH₂OCH═CH₂ |
| A-556 | Br | CH(C₆H₅)CH₃ |
| A-557 | Br | CH₂CH₂N[C(O)CF₃]S(O)₂CH₃ |
| A-558 | Br | CH₂C(CH₃)₂(S(O)₂CH₃) |
| A-559 | Br | CH₂CH₂CH₂SCH₃ |
| A-560 | Br | CH₂CH(CH₃)CH₂SCH₃ |
| A-561 | Br | CH₂CH(CH₃)CH₂S(O)CH₃ |
| A-562 | Br | CH₂CH₂CH═CH₂ |
| A-563 | Br | CH₂C(O)NH(CH₃) |
| A-564 | Br | CH₂C(O)NHS(O)₂CH₃ |
| A-565 | Br | CH₂C(CH═NOH)CH₃ |
| A-566 | Br | CH₂CH₂CH₂SCH₂CH═CH₂ |
| A-567 | Br | CHCH₃C(O)OCH₂CH₃ |
| A-568 | Br | CH₂CH₂OH |
| A-569 | Br | CH₂CH₂OC(O)c—C₃H₅ |
| A-570 | Br | CH₂CH₂C(O)OCH₂CH₃ |
| A-571 | Br | B¹ |
| A-572 | Br | B² |
| A-573 | Br | B³ |
| A-574 | Br | B⁴ |
| A-575 | Br | B⁵ |
| A-576 | Br | B⁶ |
| A-577 | Br | B⁷ |
| A-578 | Br | B⁸ |
| A-579 | Br | B⁹ |
| A-580 | Br | B¹⁰ |
| A-581 | Br | B¹² |
| A-582 | Br | B¹³ |
| A-583 | Br | B¹⁴ |
| A-584 | Br | B¹⁵ |
| A-585 | Br | B¹⁶ |
| A-586 | Br | B¹⁸ |
| A-587 | Br | B¹⁹ |
| A-588 | Br | B²⁰ |
| A-589 | I | CH₃ |
| A-590 | I | CH₂CH₃ |
| A-591 | I | CH₂CH₂CH₃ |
| A-592 | I | c-C₃H₅ |
| A-593 | I | CH(CH₃)₂ |
| A-594 | I | CH₂—(c-C₃H₅) |
| A-595 | I | CH(CH₃)—(c-C₃H₅) |
| A-596 | I | CH₂CH₂CH₂CH₃ |
| A-597 | I | CH(CH₃)CH₂CH₃ |
| A-598 | I | CH₂CH(CH₃)₂ |
| A-599 | I | C(CH₃)₃ |
| A-600 | I | c-C₄H₇ |
| A-601 | I | 1-CH₃—c-C₃H₄ |
| A-602 | I | CH₂CH═CH₂ |
| A-603 | I | CH(CH₃)CH═CH₂ |
| A-604 | I | CH₂C≡CH |
| A-605 | I | CH(CH₃)C≡CH |
| A-606 | I | C(CH₃)₂C≡CH |
| A-607 | I | CH₂F |
| A-608 | I | CHF₂ |
| A-609 | I | CF₃ |
| A-610 | I | CH₂CH₂F |
| A-611 | I | CH₂CHF₂ |
| A-612 | I | CH₂CF₃ |
| A-613 | I | CH(CH₃)CF₃ |
| A-614 | I | CH₂CH₂CF₃ |
| A-615 | I | CH₂CF₂CF₃ |
| A-616 | I | CH₂CF₂CH₃ |
| A-617 | I | CH₂CH₂CF₂CF₃ |
| A-618 | I | CH(CH(CH₃)₂)CF₃ |
| A-619 | I | CH₂(2,2-FcP) |
| A-620 | I | 3,3-FB |
| A-621 | I | 4,4-FH |
| A-622 | I | CH₂CH₂OCF₃ |
| A-623 | I | CF₂CH₂OCH₃ |
| A-624 | I | CF₂CH₂OCF₃ |
| A-625 | I | CH₂CF₂OCH₃ |
| A-626 | I | CH₂CF₂OCF₃ |
| A-627 | I | CF₂CF₂OCH₃ |
| A-628 | I | CF₂CF₂OCF₃ |
| A-629 | I | CH₂CH₂SCF₃ |
| A-630 | I | CF₂CH₂SCH₃ |
| A-631 | I | CF₂CH₂SCF₃ |
| A-632 | I | CH₂CF₂SCH₃ |
| A-633 | I | CH₂CF₂SCF₃ |
| A-634 | I | CF₂CF₂SCH₃ |
| A-635 | I | CF₂CF₂SCF₃ |
| A-636 | I | CH₂CH₂OCH₃ |
| A-637 | I | CH₂OCH₂CH₃ |
| A-638 | I | C(CH₃)₂OCH₃ |
| A-639 | I | C(CH₃)₂OCH₂CH₃ |
| A-640 | I | CH₂CH₂OCH(CH₃)₂ |
| A-641 | I | CH(CH₃)CH₂OCH₃ |
| A-642 | I | CH(CH₂CH₃)CH₂OCH₃ |
| A-643 | I | CH(CH₃)CH₂OCH₂CH₃ |
| A-644 | I | CH(CH₂CH₃)CH₂OCH₂CH₃ |
| A-645 | I | CH₂CH(CH₃)OCH₃ |
| A-646 | I | CH₂CH(CH₂CH₃)OCH₃ |
| A-647 | I | CH₂CH(CH₃)OCH₂CH₃ |
| A-648 | I | CH₂CH(CH₂CH₃)OCH₂CH₃ |
| A-649 | I | CH₂CH(OCH₃)₂ |
| A-650 | I | CH(CH₃)CH(OCH₃)₂ |
| A-651 | I | CH₂C(CH₃)(OCH₃)₂ |
| A-652 | I | CH₂CH₂CH₂OCH₃ |
| A-653 | I | CH₂CH(CH₃)OCH₃ |
| A-654 | I | CH(CH₂CH₃)CH₂OCH₃ |
| A-655 | I | CH(CH₃)CH₂OCH₃ |
| A-656 | I | CH₂(2-FU) |
| A-657 | I | CH₂(2-HFU) |
| A-658 | I | CH₂(3-FU) |
| A-659 | I | CH₂(3-HFU) |
| A-660 | I | CH₂CH₂SCH₃ |
| A-661 | I | CH₂CH₂S(O)CH₃ |
| A-662 | I | CH₂CH₂S(O)₂CH₃ |
| A-663 | I | CH₂CH₂SCH₂CH₃ |
| A-664 | I | CH₂CH₂S(O)CH₂CH₃ |
| A-665 | I | CH₂CH₂S(O)₂CH₂CH₃ |
| A-666 | I | CH₂CH₂SCH(CH₃)₂ |
| A-667 | I | CH₂CH₂S(O)CH(CH₃)₂ |
| A-668 | I | CH₂CH₂S(O)₂CH(CH₃)₂ |
| A-669 | I | CH₂CH₂SC(CH₃)₃ |
| A-670 | I | CH₂CH₂S(O)C(CH₃)₃ |
| A-671 | I | CH₂CH₂S(O)₂C(CH₃)₃ |
| A-672 | I | CH(CH₃)CH₂SCH₃ |
| A-673 | I | CH(CH₂CH₃)CH₂SCH₃ |
| A-674 | I | CH(CH₃)CH₂S(O)CH₃ |
| A-675 | I | CH(CH₂CH₃)CH₂S(O)CH₃ |
| A-676 | I | CH(CH₃)CH₂S(O)₂CH₃ |
| A-677 | I | CH(CH₂CH₃)CH₂S(O)₂CH₃ |
| A-678 | I | CH₂CH(CH₃)SCH₃ |
| A-679 | I | CH₂CH(CH₂CH₃)SCH₃ |
| A-680 | I | CH₂CH(CH₃)S(O)CH₃ |
| A-681 | I | CH₂CH(CH₂CH₃)S(O)CH₃ |
| A-682 | I | CH₂CH(CH₃)S(O)₂CH₃ |
| A-683 | I | CH₂CH(CH₂CH₃)S(O)₂CH₃ |
| A-684 | I | CH₂CN |
| A-685 | I | CH₂CH₂CN |
| A-686 | I | C(CH₃)₂CN |
| A-687 | I | CH₂CH₂CH₂CN |
| A-688 | I | CH(CN)CH(CH₃)₂ |
| A-689 | I | CH₂CH₂OCH₂CH₃ |
| A-690 | I | CH₂CH₂C(O)OCH₃ |
| A-691 | I | CH₂CH₂CH₂S(O)CH₂CH₂CF₃ |
| A-692 | I | CH₂CH₂CH₂CH₂CH₃ |
| A-693 | I | CH(CH₂OCH₂CF₃)CH₂CH₃ |
| A-694 | I | c-C₅H₉ |
| A-695 | I | CH₂CF₂CF₂H |
| A-696 | I | CH₂CH(OCH₂CH₃)₂ |
| A-697 | I | CH(CH₃)CH(CH₃)₂ |
| A-698 | I | CH₂CH₂CH(CH₃)₂ |
| A-699 | I | CH₂CH₂Si(CH₃)₃ |
| A-700 | I | CH₂CH₂CH₂SCH₂CH₂CF₃ |
| A-701 | I | CH₂CH₂CH₂S(O)CH₃ |
| A-702 | I | CH(CH₃)CH₂OCH═CH₂ |
| A-703 | I | CH(C₆H₅)CH₃ |
| A-704 | I | CH₂CH₂N[C(O)CF₃]S(O)₂CH₃ |
| A-705 | I | CH₂C(CH₃)₂(S(O)₂CH₃) |
| A-706 | I | CH₂CH₂CH₂SCH₃ |
| A-707 | I | CH₂CH(CH₃)CH₂SCH₃ |
| A-708 | I | CH₂CH(CH₃)CH₂S(O)CH₃ |
| A-709 | I | CH₂CH₂CH═CH₂ |
| A-710 | I | CH₂C(O)NH(CH₃) |
| A-711 | I | CH₂C(O)NHS(O)₂CH₃ |
| A-712 | I | CH₂C(CH═NOH)CH₃ |
| A-713 | I | CH₂CH₂CH₂SCH₂CH═CH₂ |
| A-714 | I | CHCH₃C(O)OCH₂CH₃ |
| A-715 | I | CH₂CH₂OH |
| A-716 | I | CH₂CH₂OC(O)c—C₃H₅ |
| A-717 | I | CH₂CH₂C(O)OCH₂CH₃ |
| A-718 | I | B¹ |
| A-719 | I | B² |
| A-720 | I | B³ |
| A-721 | I | B⁴ |
| A-722 | I | B⁵ |
| A-723 | I | B⁶ |
| A-724 | I | B⁷ |
| A-725 | I | B⁸ |
| A-726 | I | B⁹ |
| A-727 | I | B¹⁰ |
| A-728 | I | B¹² |
| A-729 | I | B¹³ |
| A-730 | I | B¹⁴ |
| A-731 | I | B¹⁵ |
| A-732 | I | B¹⁶ |
| A-733 | I | B¹⁸ |
| A-734 | I | B¹⁹ |
| A-735 | I | B²⁰ |
| A-736 | CH₃ | CH₃ |
| A-737 | CH₃ | CH₂CH₃ |
| A-738 | CH₃ | CH₂CH₂CH₃ |
| A-739 | CH₃ | c-C₃H₅ |
| A-740 | CH₃ | CH(CH₃)₂ |
| A-741 | CH₃ | CH₂—(c-C₃H₅) |
| A-742 | CH₃ | CH(CH₃)—(c-C₃H₅) |
| A-743 | CH₃ | CH₂CH₂CH₂CH₃ |
| A-744 | CH₃ | CH(CH₃)CH₂CH₃ |
| A-745 | CH₃ | CH₂CH(CH₃)₂ |
| A-746 | CH₃ | C(CH₃)₃ |
| A-747 | CH₃ | c-C₄H₇ |
| A-748 | CH₃ | 1-CH₃—c-C₃H₄ |
| A-749 | CH₃ | CH₂CH═CH₂ |
| A-750 | CH₃ | CH(CH₃)CH═CH₂ |
| A-751 | CH₃ | CH₂C≡CH |
| A-752 | CH₃ | CH(CH₃)C≡CH |
| A-753 | CH₃ | C(CH₃)₂C≡CH |
| A-754 | CH₃ | CH₂F |
| A-755 | CH₃ | CHF₂ |
| A-756 | CH₃ | CF₃ |
| A-757 | CH₃ | CH₂CH₂F |
| A-758 | CH₃ | CH₂CHF₂ |
| A-759 | CH₃ | CH₂CF₃ |
| A-760 | CH₃ | CH(CH₃)CF₃ |
| A-761 | CH₃ | CH₂CH₂CF₃ |
| A-762 | CH₃ | CH₂CF₂CF₃ |
| A-763 | CH₃ | CH₂CF₂CH₃ |
| A-764 | CH₃ | CH₂CH₂CF₂CF₃ |
| A-765 | CH₃ | CH(CH(CH₃)₂)CF₃ |
| A-766 | CH₃ | CH₂(2,2-FcP) |
| A-767 | CH₃ | 3,3-FB |
| A-768 | CH₃ | 4,4-FH |
| A-769 | CH₃ | CH₂CH₂OCF₃ |
| A-770 | CH₃ | CF₂CH₂OCH₃ |
| A-771 | CH₃ | CF₂CH₂OCF₃ |
| A-772 | CH₃ | CH₂CF₂OCH₃ |
| A-773 | CH₃ | CH₂CF₂OCF₃ |
| A-774 | CH₃ | CF₂CF₂OCH₃ |
| A-775 | CH₃ | CF₂CF₂OCF₃ |
| A-776 | CH₃ | CH₂CH₂SCF₃ |
| A-777 | CH₃ | CF₂CH₂SCH₃ |
| A-778 | CH₃ | CF₂CH₂SCF₃ |
| A-779 | CH₃ | CH₂CF₂SCH₃ |
| A-780 | CH₃ | CH₂CF₂SCF₃ |
| A-781 | CH₃ | CF₂CF₂SCH₃ |
| A-782 | CH₃ | CF₂CF₂SCF₃ |
| A-783 | CH₃ | CH₂CH₂OCH₃ |
| A-784 | CH₃ | CH₂OCH₂CH₃ |
| A-785 | CH₃ | C(CH₃)₂OCH₃ |
| A-786 | CH₃ | C(CH₃)₂OCH₂CH₃ |
| A-787 | CH₃ | CH₂CH₂OCH(CH₃)₂ |
| A-788 | CH₃ | CH(CH₃)CH₂OCH₃ |
| A-789 | CH₃ | CH(CH₂CH₃)CH₂OCH₃ |
| A-790 | CH₃ | CH(CH₃)CH₂OCH₂CH₃ |
| A-791 | CH₃ | CH(CH₂CH₃)CH₂OCH₂CH₃ |
| A-792 | CH₃ | CH₂CH(CH₃)OCH₃ |
| A-793 | CH₃ | CH₂CH(CH₂CH₃)OCH₃ |
| A-794 | CH₃ | CH₂CH(CH₃)OCH₂CH₃ |
| A-795 | CH₃ | CH₂CH(CH₂CH₃)OCH₂CH₃ |
| A-796 | CH₃ | CH₂CH(OCH₃)₂ |
| A-797 | CH₃ | CH(CH₃)CH(OCH₃)₂ |
| A-798 | CH₃ | CH₂C(CH₃)(OCH₃)₂ |
| A-799 | CH₃ | CH₂CH₂CH₂OCH₃ |
| A-800 | CH₃ | CH₂CH(CH₃)OCH₃ |
| A-801 | CH₃ | CH(CH₂CH₃)CH₂OCH₃ |
| A-802 | CH₃ | CH(CH₃)CH₂OCH₃ |
| A-803 | CH₃ | CH₂(2-FU) |
| A-804 | CH₃ | CH₂(2-HFU) |
| A-805 | CH₃ | CH₂(3-FU) |
| A-806 | CH₃ | CH₂(3-HFU) |
| A-807 | CH₃ | CH₂CH₂SCH₃ |
| A-808 | CH₃ | CH₂CH₂S(O)CH₃ |
| A-809 | CH₃ | CH₂CH₂S(O)₂CH₃ |
| A-810 | CH₃ | CH₂CH₂SCH₂CH₃ |
| A-811 | CH₃ | CH₂CH₂S(O)CH₂CH₃ |
| A-812 | CH₃ | CH₂CH₂S(O)₂CH₂CH₃ |
| A-813 | CH₃ | CH₂CH₂SCH(CH₃)₂ |
| A-814 | CH₃ | CH₂CH₂S(O)CH(CH₃)₂ |
| A-815 | CH₃ | CH₂CH₂S(O)₂CH(CH₃)₂ |
| A-816 | CH₃ | CH₂CH₂SC(CH₃)₃ |
| A-817 | CH₃ | CH₂CH₂S(O)C(CH₃)₃ |
| A-818 | CH₃ | CH₂CH₂S(O)₂C(CH₃)₃ |
| A-819 | CH₃ | CH(CH₃)CH₂SCH₃ |
| A-820 | CH₃ | CH(CH₂CH₃)CH₂SCH₃ |
| A-821 | CH₃ | CH(CH₃)CH₂S(O)CH₃ |
| A-822 | CH₃ | CH(CH₂CH₃)CH₂S(O)CH₃ |
| A-823 | CH₃ | CH(CH₃)CH₂S(O)₂CH₃ |
| A-824 | CH₃ | CH(CH₂CH₃)CH₂S(O)₂CH₃ |
| A-825 | CH₃ | CH₂CH(CH₃)SCH₃ |
| A-826 | CH₃ | CH₂CH(CH₂CH₃)SCH₃ |
| A-827 | CH₃ | CH₂CH(CH₃)S(O)CH₃ |
| A-828 | CH₃ | CH₂CH(CH₂CH₃)S(O)CH₃ |
| A-829 | CH₃ | CH₂CH(CH₃)S(O)₂CH₃ |
| A-830 | CH₃ | CH₂CH(CH₂CH₃)S(O)₂CH₃ |
| A-831 | CH₃ | CH₂CN |
| A-832 | CH₃ | CH₂CH₂CN |
| A-833 | CH₃ | C(CH₃)₂CN |
| A-834 | CH₃ | CH₂CH₂CH₂CN |
| A-835 | CH₃ | CH(CN)CH(CH₃)₂ |
| A-836 | CH₃ | CH₂CH₂OCH₂CH₃ |
| A-837 | CH₃ | CH₂CH₂C(O)OCH₃ |
| A-838 | CH₃ | CH₂CH₂CH₂S(O)CH₂CH₂CF₃ |
| A-839 | CH₃ | CH₂CH₂CH₂CH₂CH₃ |
| A-840 | CH₃ | CH(CH₂OCH₂CF₃)CH₂CH₃ |
| A-841 | CH₃ | c-C₅H₉ |
| A-842 | CH₃ | CH₂CF₂CF₂H |
| A-843 | CH₃ | CH₂CH(OCH₂CH₃)₂ |
| A-844 | CH₃ | CH(CH₃)CH(CH₃)₂ |
| A-845 | CH₃ | CH₂CH₂CH(CH₃)₂ |
| A-846 | CH₃ | CH₂CH₂Si(CH₃)₃ |
| A-847 | CH₃ | CH₂CH₂CH₂SCH₂CH₂CF₃ |
| A-848 | CH₃ | CH₂CH₂CH₂S(O)CH₃ |
| A-849 | CH₃ | CH(CH₃)CH₂OCH═CH₂ |
| A-850 | CH₃ | CH(C₆H₅)CH₃ |
| A-851 | CH₃ | CH₂CH₂N[C(O)CF₃]S(O)₂CH₃ |
| A-852 | CH₃ | CH₂C(CH₃)₂(S(O)₂CH₃) |
| A-853 | CH₃ | CH₂CH₂CH₂SCH₃ |
| A-854 | CH₃ | CH₂CH(CH₃)CH₂SCH₃ |
| A-855 | CH₃ | CH₂CH(CH₃)CH₂S(O)CH₃ |
| A-856 | CH₃ | CH₂CH₂CH═CH₂ |
| A-857 | CH₃ | CH₂C(O)NH(CH₃) |
| A-858 | CH₃ | CH₂C(O)NHS(O)₂CH₃ |
| A-859 | CH₃ | CH₂C(CH═NOH)CH₃ |
| A-860 | CH₃ | CH₂CH₂CH₂SCH₂CH═CH₂ |
| A-861 | CH₃ | CHCH₃C(O)OCH₂CH₃ |
| A-862 | CH₃ | CH₂CH₂OH |
| A-863 | CH₃ | CH₂CH₂OC(O)c—C₃H₅ |
| A-864 | CH₃ | CH₂CH₂C(O)OCH₂CH₃ |
| A-865 | CH₃ | B¹ |
| A-866 | CH₃ | B² |
| A-867 | CH₃ | B³ |
| A-868 | CH₃ | B⁴ |
| A-869 | CH₃ | B⁵ |
| A-870 | CH₃ | B⁶ |
| A-871 | CH₃ | B⁷ |
| A-872 | CH₃ | B⁸ |
| A-873 | CH₃ | B⁹ |
| A-874 | CH₃ | B¹⁰ |
| A-875 | CH₃ | B¹² |
| A-876 | CH₃ | B¹³ |
| A-877 | CH₃ | B¹⁴ |
| A-878 | CH₃ | B¹⁵ |
| A-879 | CH₃ | B¹⁶ |
| A-880 | CH₃ | B¹⁸ |
| A-881 | CH₃ | B¹⁹ |
| A-882 | CH₃ | B²⁰ |
| A-883 | CF₃ | CH₃ |
| A-884 | CF₃ | CH₂CH₃ |
| A-885 | CF₃ | CH₂CH₂CH₃ |
| A-886 | CF₃ | c-C₃H₅ |
| A-887 | CF₃ | CH(CH₃)₂ |
| A-888 | CF₃ | CH₂—(c-C₃H₅₎ |
| A-889 | CF₃ | CH(CH₃)—(c-C₃H₅) |
| A-890 | CF₃ | CH₂CH₂CH₂CH₃ |
| A-891 | CF₃ | CH(CH₃)CH₂CH₃ |
| A-892 | CF₃ | CH₂CH(CH₃)₂ |
| A-893 | CF₃ | C(CH₃)₃ |
| A-894 | CF₃ | c-C₄H₇ |
| A-895 | CF₃ | 1-CH₃—c-C₃H₄ |
| A-896 | CF₃ | CH₂CH═CH₂ |
| A-897 | CF₃ | CH(CH₃)CH═CH₂ |
| A-898 | CF₃ | CH₂C≡CH |
| A-899 | CF₃ | CH(CH₃)C≡CH |
| A-900 | CF₃ | C(CH₃)₂C≡CH |
| A-901 | CF₃ | CH₂F |
| A-902 | CF₃ | CHF₂ |
| A-903 | CF₃ | CF₃ |
| A-904 | CF₃ | CH₂CH₂F |
| A-905 | CF₃ | CH₂CHF₂ |
| A-906 | CF₃ | CH₂CF₃ |
| A-907 | CF₃ | CH(CH₃)CF₃ |
| A-908 | CF₃ | CH₂CH₂CF₃ |
| A-909 | CF₃ | CH₂CF₂CF₃ |
| A-910 | CF₃ | CH₂CF₂CH₃ |
| A-911 | CF₃ | CH₂CH₂CF₂CF₃ |
| A-912 | CF₃ | CH(CH(CH₃)₂)CF₃ |
| A-913 | CF₃ | CH₂(2,2-FcP) |
| A-914 | CF₃ | 3,3-FB |
| A-915 | CF₃ | 4,4-FH |
| A-916 | CF₃ | CH₂CH₂OCF₃ |
| A-917 | CF₃ | CF₂CH₂OCH₃ |
| A-918 | CF₃ | CF₂CH₂OCF₃ |
| A-919 | CF₃ | CH₂CF₂OCH₃ |
| A-920 | CF₃ | CH₂CF₂OCF₃ |
| A-921 | CF₃ | CF₂CF₂OCH₃ |
| A-922 | CF₃ | CF₂CF₂OCF₃ |
| A-923 | CF₃ | CH₂CH₂SCF₃ |
| A-924 | CF₃ | CF₂CH₂SCH₃ |
| A-925 | CF₃ | CF₂CH₂SCF₃ |
| A-926 | CF₃ | CH₂CF₂SCH₃ |
| A-927 | CF₃ | CH₂CF₂SCF₃ |
| A-928 | CF₃ | CF₂CF₂SCH₃ |
| A-929 | CF₃ | CF₂CF₂SCF₃ |
| A-930 | CF₃ | CH₂CH₂OCH₃ |
| A-931 | CF₃ | CH₂OCH₂CH₃ |
| A-932 | CF₃ | C(CH₃)₂OCH₃ |
| A-933 | CF₃ | C(CH₃)₂OCH₂CH₃ |
| A-934 | CF₃ | CH₂CH₂OCH(CH₃)₂ |
| A-935 | CF₃ | CH(CH₃)CH₂OCH₃ |
| A-936 | CF₃ | CH(CH₂CH₃)CH₂OCH₃ |
| A-937 | CF₃ | CH(CH₃)CH₂OCH₂CH₃ |
| A-938 | CF₃ | CH(CH₂CH₃)CH₂OCH₂CH₃ |
| A-939 | CF₃ | CH₂CH(CH₃)OCH₃ |
| A-940 | CF₃ | CH₂CH(CH₂CH₃)OCH₃ |
| A-941 | CF₃ | CH₂CH(CH₃)OCH₂CH₃ |
| A-942 | CF₃ | CH₂CH(CH₂CH₃)OCH₂CH₃ |
| A-943 | CF₃ | CH₂CH(OCH₃)₂ |
| A-944 | CF₃ | CH(CH₃)CH(OCH₃)₂ |
| A-945 | CF₃ | CH₂C(CH₃)(OCH₃)₂ |
| A-946 | CF₃ | CH₂CH₂CH₂OCH₃ |
| A-947 | CF₃ | CH₂CH(CH₃)OCH₃ |
| A-948 | CF₃ | CH(CH₂CH₃)CH₂OCH₃ |
| A-949 | CF₃ | CH(CH₃)CH₂OCH₃ |
| A-950 | CF₃ | CH₂(2-FU) |
| A-951 | CF₃ | CH₂(2-HFU) |
| A-952 | CF₃ | CH₂(3-FU) |
| A-953 | CF₃ | CH₂(3-HFU) |
| A-954 | CF₃ | CH₂CH₂SCH₃ |
| A-955 | CF₃ | CH₂CH₂S(O)CH₃ |
| A-956 | CF₃ | CH₂CH₂S(O)₂CH₃ |
| A-957 | CF₃ | CH₂CH₂SCH₂CH₃ |
| A-958 | CF₃ | CH₂CH₂S(O)CH₂CH₃ |
| A-959 | CF₃ | CH₂CH₂S(O)₂CH₂CH₃ |
| A-960 | CF₃ | CH₂CH₂SCH(CH₃)₂ |
| A-961 | CF₃ | CH₂CH₂S(O)CH(CH₃)₂ |
| A-962 | CF₃ | CH₂CH₂S(O)₂CH(CH₃)₂ |
| A-963 | CF₃ | CH₂CH₂SC(CH₃)₃ |
| A-964 | CF₃ | CH₂CH₂S(O)C(CH₃)₃ |
| A-965 | CF₃ | CH₂CH₂S(O)₂C(CH₃)₃ |
| A-966 | CF₃ | CH(CH₃)CH₂SCH₃ |
| A-967 | CF₃ | CH(CH₂CH₃)CH₂SCH₃ |
| A-968 | CF₃ | CH(CH₃)CH₂S(O)CH₃ |
| A-969 | CF₃ | CH(CH₂CH₃)CH₂S(O)CH₃ |
| A-970 | CF₃ | CH(CH₃)CH₂S(O)₂CH₃ |
| A-971 | CF₃ | CH(CH₂CH₃)CH₂S(O)₂CH₃ |
| A-972 | CF₃ | CH₂CH(CH₃)SCH₃ |
| A-973 | CF₃ | CH₂CH(CH₂CH₃)SCH₃ |
| A-974 | CF₃ | CH₂CH(CH₃)S(O)CH₃ |
| A-975 | CF₃ | CH₂CH(CH₂CH₃)S(O)CH₃ |
| A-976 | CF₃ | CH₂CH(CH₃)S(O)₂CH₃ |
| A-977 | CF₃ | CH₂CH(CH₂CH₃)S(O)₂CH₃ |
| A-978 | CF₃ | CH₂CN |
| A-979 | CF₃ | CH₂CH₂CN |
| A-980 | CF₃ | C(CH₃)₂CN |
| A-981 | CF₃ | CH₂CH₂CH₂CN |
| A-982 | CF₃ | CH(CN)CH (CH₃)₂ |
| A-983 | CF₃ | CH₂CH₂OCH₂CH₃ |
| A-984 | CF₃ | CH₂CH₂C(O)OCH₃ |
| A-985 | CF₃ | CH₂CH₂CH₂S(O)CH₂CH₂CF₃ |
| A-986 | CF₃ | CH₂CH₂CH₂CH₂CH₃ |
| A-987 | CF₃ | CH(CH₂OCH₂CF₃)CH₂CH₃ |
| A-988 | CF₃ | c-C₅H₉ |
| A-989 | CF₃ | CH₂CF₂CF₂H |
| A-990 | CF₃ | CH₂CH(OCH₂CH₃)₂ |
| A-991 | CF₃ | CH(CH₃)CH(CH₃)₂ |
| A-992 | CF₃ | CH₂CH₂CH(CH₃)₂ |
| A-993 | CF₃ | CH₂CH₂Si(CH₃)₃ |
| A-994 | CF₃ | CH₂CH₂CH₂SCH₂CH₂CF₃ |
| A-995 | CF₃ | CH₂CH₂CH₂S(O)CH₃ |
| A-996 | CF₃ | CH(CH₃)CH₂OCH═CH₂ |
| A-997 | CF₃ | CH(C₆H₅)CH₃ |
| A-998 | CF₃ | CH₂CH₂N[C(O)CF₃]S(O)₂CH₃ |
| A-999 | CF₃ | CH₂C(CH₃)₂(S(O)₂CH₃) |
| A-1000 | CF₃ | CH₂CH₂CH₂SCH₃ |
| A-1001 | CF₃ | CH₂CH(CH₃)CH₂SCH₃ |
| A-1002 | CF₃ | CH₂CH(CH₃)CH₂S(O)CH₃ |
| A-1003 | CF₃ | CH₂CH₂CH═CH₂ |
| A-1004 | CF₃ | CH₂C(O)NH (CH₃) |
| A-1005 | CF₃ | CH₂C(O)NHS(O)₂CH₃ |
| A-1006 | CF₃ | CH₂C(CH═NOH)CH₃ |
| A-1007 | CF₃ | CH₂CH₂CH₂SCH₂CH═CH₂ |
| A-1008 | CF₃ | CHCH₃C(O)OCH₂CH₃ |
| A-1009 | CF₃ | CH₂CH₂OH |
| A-1010 | CF₃ | CH₂CH₂OC(O)c—C₃H₅ |
| A-1011 | CF₃ | CH₂CH₂C(O)OCH₂CH₃ |
| A-1012 | CF₃ | B¹ |
| A-1013 | CF₃ | B² |
| A-1014 | CF₃ | B³ |
| A-1015 | CF₃ | B⁴ |
| A-1016 | CF₃ | B⁵ |
| A-1017 | CF₃ | B⁶ |
| A-1018 | CF₃ | B⁷ |
| A-1019 | CF₃ | B⁸ |
| A-1020 | CF₃ | B⁹ |
| A-1021 | CF₃ | B¹⁰ |
| A-1022 | CF₃ | B¹² |
| A-1023 | CF₃ | B¹³ |
| A-1024 | CF₃ | B¹⁴ |
| A-1025 | CF₃ | B¹⁵ |
| A-1026 | CF₃ | B¹⁶ |
| A-1027 | CF₃ | B¹⁸ |
| A-1028 | CF₃ | B¹⁹ |
| A-1029 | CF₃ | B²⁰ |
| A-1030 | CN | CH₃ |
| A-1031 | CN | CH₂CH₃ |
| A-1032 | CN | CH₂CH₂CH₃ |
| A-1033 | CN | c-C₃H₅ |
| A-1034 | CN | CH(CH₃)₂ |
| A-1035 | CN | CH₂—(c-C₃H₅) |
| A-1036 | CN | CH(CH₃)—(c-C₃H₅) |
| A-1037 | CN | CH₂CH₂CH₂CH₃ |
| A-1038 | CN | CH(CH₃)CH₂CH₃ |
| A-1039 | CN | CH₂CH(CH₃)₂ |
| A-1040 | CN | C(CH₃)₃ |
| A-1041 | CN | c-C₄H₇ |
| A-1042 | CN | 1-CH₃—c-C₃H₄ |
| A-1043 | CN | CH₂CH═CH₂ |
| A-1044 | CN | CH(CH₃)CH═CH₂ |
| A-1045 | CN | CH₂C≡CH |
| A-1046 | CN | CH(CH₃)C≡CH |
| A-1047 | CN | C(CH₃)₂C≡CH |
| A-1048 | CN | CH₂F |
| A-1049 | CN | CHF₂ |
| A-1050 | CN | CF₃ |
| A-1051 | CN | CH₂CH₂F |
| A-1052 | CN | CH₂CHF₂ |
| A-1053 | CN | CH₂CF₃ |
| A-1054 | CN | CH(CH₃)CF₃ |
| A-1055 | CN | CH₂CH₂CF₃ |
| A-1056 | CN | CH₂CF₂CF₃ |
| A-1057 | CN | CH₂CF₂CH₃ |
| A-1058 | CN | CH₂CH₂CF₂CF₃ |
| A-1059 | CN | CH(CH(CH₃)₂)CF₃ |
| A-1060 | CN | CH₂(2,2-FcP) |
| A-1061 | CN | 3,3-FB |
| A-1062 | CN | 4,4-FH |
| A-1063 | CN | CH₂CH₂OCF₃ |
| A-1064 | CN | CF₂CH₂OCH₃ |
| A-1065 | CN | CF₂CH₂OCF₃ |
| A-1066 | CN | CH₂CF₂OCH₃ |
| A-1067 | CN | CH₂CF₂OCF₃ |
| A-1068 | CN | CF₂CF₂OCH₃ |
| A-1069 | CN | CF₂CF₂OCF₃ |
| A-1070 | CN | CH₂CH₂SCF₃ |
| A-1071 | CN | CF₂CH₂SCH₃ |
| A-1072 | CN | CF₂CH₂SCF₃ |
| A-1073 | CN | CH₂CF₂SCH₃ |
| A-1074 | CN | CH₂CF₂SCF₃ |
| A-1075 | CN | CF₂CF₂SCH₃ |
| A-1076 | CN | CF₂CF₂SCF₃ |
| A-1077 | CN | CH₂CH₂OCH₃ |
| A-1078 | CN | CH₂OCH₂CH₃ |
| A-1079 | CN | C(CH₃)₂OCH₃ |
| A-1080 | CN | C(CH₃)₂OCH₂CH₃ |
| A-1081 | CN | CH₂CH₂OCH(CH₃)₂ |
| A-1082 | CN | CH(CH₃)CH₂OCH₃ |
| A-1083 | CN | CH(CH₂CH₃)CH₂OCH₃ |
| A-1084 | CN | CH(CH₃)CH₂OCH₂CH₃ |
| A-1085 | CN | CH(CH₂CH₃)CH₂OCH₂CH₃ |
| A-1086 | CN | CH₂CH(CH₃)OCH₃ |
| A-1087 | CN | CH₂CH(CH₂CH₃)OCH₃ |
| A-1088 | CN | CH₂CH(CH₃)OCH₂CH₃ |
| A-1089 | CN | CH₂CH(CH₂CH₃)OCH₂CH₃ |
| A-1090 | CN | CH₂CH(OCH₃)₂ |
| A-1091 | CN | CH(CH₃)CH(OCH₃)₂ |
| A-1092 | CN | CH₂C(CH₃)(OCH₃)₂ |
| A-1093 | CN | CH₂CH₂CH₂OCH₃ |
| A-1094 | CN | CH₂CH(CH₃)OCH₃ |
| A-1095 | CN | CH(CH₂CH₃)CH₂OCH₃ |
| A-1096 | CN | CH(CH₃)CH₂OCH₃ |
| A-1097 | CN | CH₂(2-FU) |
| A-1098 | CN | CH₂(2-HFU) |
| A-1099 | CN | CH₂(3-FU) |
| A-1100 | CN | CH₂(3-HFU) |
| A-1101 | CN | CH₂CH₂SCH₃ |
| A-1102 | CN | CH₂CH₂S(O)CH₃ |
| A-1103 | CN | CH₂CH₂S(O)₂CH₃ |
| A-1104 | CN | CH₂CH₂SCH₂CH₃ |
| A-1105 | CN | CH₂CH₂S(O)CH₂CH₃ |
| A-1106 | CN | CH₂CH₂S(O)₂CH₂CH₃ |
| A-1107 | CN | CH₂CH₂SCH(CH₃)₂ |
| A-1108 | CN | CH₂CH₂S(O)CH(CH₃)₂ |
| A-1109 | CN | CH₂CH₂S(O)₂CH(CH₃)₂ |
| A-1110 | CN | CH₂CH₂SC(CH₃)₃ |
| A-1111 | CN | CH₂CH₂S(O)C(CH₃)₃ |
| A-1112 | CN | CH₂CH₂S(O)₂C(CH₃)₃ |
| A-1113 | CN | CH(CH₃)CH₂SCH₃ |
| A-1114 | CN | CH(CH₂CH₃)CH₂SCH₃ |
| A-1115 | CN | CH(CH₃)CH₂S(O)CH₃ |
| A-1116 | CN | CH(CH₂CH₃)CH₂S(O)CH₃ |
| A-1117 | CN | CH(CH₃)CH₂S(O)₂CH₃ |
| A-1118 | CN | CH(CH₂CH₃)CH₂S(O)₂CH₃ |
| A-1119 | CN | CH₂CH(CH₃)SCH₃ |
| A-1120 | CN | CH₂CH(CH₂CH₃)SCH₃ |
| A-1121 | CN | CH₂CH(CH₃)S(O)CH₃ |
| A-1122 | CN | CH₂CH(CH₂CH₃)S(O)CH₃ |
| A-1123 | CN | CH₂CH(CH₃)S(O)₂CH₃ |
| A-1124 | CN | CH₂CH(CH₂CH₃)S(O)₂CH₃ |
| A-1125 | CN | CH₂CN |
| A-1126 | CN | CH₂CH₂CN |
| A-1127 | CN | C(CH₃)₂CN |
| A-1128 | CN | CH₂CH₂CH₂CN |
| A-1129 | CN | CH(CN)CH(CH₃)₂ |
| A-1130 | CN | CH₂CH₂OCH₂CH₃ |
| A-1131 | CN | CH₂CH₂C(O)OCH₃ |
| A-1132 | CN | CH₂CH₂CH₂S(O)CH₂CH₂CF₃ |
| A-1133 | CN | CH₂CH₂CH₂CH₂CH₃ |
| A-1134 | CN | CH(CH₂OCH₂CF₃)CH₂CH₃ |
| A-1135 | CN | c-C₅H₉ |
| A-1136 | CN | CH₂CF₂CF2H |
| A-1137 | CN | CH₂CH(OCH₂CH₃)₂ |
| A-1138 | CN | CH(CH₃)CH(CH₃)₂ |
| A-1139 | CN | CH₂CH₂CH(CH₃)₂ |
| A-1140 | CN | CH₂CH₂Si(CH₃)₃ |
| A-1141 | CN | CH₂CH₂CH₂SCH₂CH₂CF₃ |
| A-1142 | CN | CH₂CH₂CH₂S(O)CH₃ |
| A-1143 | CN | CH(CH₃)CH₂OCH═CH₂ |
| A-1144 | CN | CH(C₆H₅)CH₃ |
| A-1145 | CN | CH₂CH₂N[C(O)CF₃]S(O)₂CH₃ |
| A-1146 | CN | CH₂C(CH₃)₂(S(O)₂CH₃) |
| A-1147 | CN | CH₂CH₂CH₂SCH₃ |
| A-1148 | CN | CH₂CH(CH₃)CH₂SCH₃ |
| A-1149 | CN | CH₂CH(CH₃)CH₂S(O)CH₃ |
| A-1150 | CN | CH₂CH₂CH═CH₂ |
| A-1151 | CN | CH₂C(O)NH(CH₃) |
| A-1152 | CN | CH₂C(O)NHS(O)₂CH₃ |
| A-1153 | CN | CH₂C(CH═NOH)CH₃ |
| A-1154 | CN | CH₂CH₂CH₂SCH₂CH═CH₂ |
| A-1155 | CN | CHCH₃C(O)OCH₂CH₃ |
| A-1156 | CN | CH₂CH₂OH |
| A-1157 | CN | CH₂CH₂OC(O)c—C₃H₅ |
| A-1158 | CN | CH₂CH₂C(O)OCH₂CH₃ |
| A-1159 | CN | B¹ |
| A-1160 | CN | B² |
| A-1161 | CN | B³ |
| A-1162 | CN | B⁴ |
| A-1163 | CN | B⁵ |
| A-1164 | CN | B⁶ |
| A-1165 | CN | B⁷ |
| A-1166 | CN | B⁸ |
| A-1167 | CN | B⁹ |
| A-1168 | CN | B¹⁰ |
| A-1169 | CN | B¹² |
| A-1170 | CN | B¹³ |
| A-1171 | CN | B¹⁴ |
| A-1172 | CN | B¹⁵ |
| A-1173 | CN | B¹⁶ |
| A-1174 | CN | B¹⁸ |
| A-1175 | CN | B¹⁹ |
| A-1176 | CN | B²⁰ |

Table 121 Compounds of the formula (I-B) in which R¹ is H, Y is SO, and the combination of D and B for a compound corresponds in each case to one row of Table B.

Table 122 Compounds of the formula (I-B) in which R¹ is F, Y is SO, and the combination of D and B for a compound corresponds in each case to one row of Table B.

Table 123 Compounds of the formula (I-B) in which R¹ is CN, Y is SO, and the combination of D and B for a compound corresponds in each case to one row of Table B.

Table 124 Compounds of the formula (I-B) in which R¹ is H, Y is SO₂, and the combination of D and B for a compound corresponds in each case to one row of Table B.

Table 125 Compounds of the formula (I-B) in which R¹ is F, Y is SO₂, and the combination of D and B for a compound corresponds in each case to one row of Table B.

Table 126 Compounds of the formula (I-B) in which R¹ is CN, Y is SO₂, and the combination of D and B for a compound corresponds in each case to one row of Table B.

Table 127 Compounds of the formula (I-C) in which R¹ is H, Y is SO, and the combination of D and B for a compound corresponds in each case to one row of Table B.

Table 128 Compounds of the formula (I-C) in which R¹ is F, Y is SO, and the combination of D and B for a compound corresponds in each case to one row of Table B.

Table 129 Compounds of the formula (I-C) in which R¹ is CN, Y is SO, and the combination of D and B for a compound corresponds in each case to one row of Table B.

Table 130 Compounds of the formula (I-C) in which R¹ is H, Y is SO₂, and the combination of D and B for a compound corresponds in each case to one row of Table B.

Table 131 Compounds of the formula (I-C) in which R¹ is F, Y is SO₂, and the combination of D and B for a compound corresponds in each case to one row of Table B.

Table 132 Compounds of the formula (I-C) in which R¹ is CN, Y is SO₂, and the combination of D and B for a compound corresponds in each case to one row of Table B.

Table 133 Compounds of the formula (I-D) in which R¹ is H, Y is SO, and the combination of D and B for a compound corresponds in each case to one row of Table B.

Table 134 Compounds of the formula (I-D) in which R¹ is F, Y is SO, and the combination of D and B for a compound corresponds in each case to one row of Table B.

Table 135 Compounds of the formula (I-D) in which R¹ is CN, Y is SO, and the combination of D and B for a compound corresponds in each case to one row of Table B.

Table 136 Compounds of the formula (I-D) in which R¹ is H, Y is SO₂, and the combination of D and B for a compound corresponds in each case to one row of Table B.

Table 137 Compounds of the formula (I-D) in which R¹ is F, Y is SO₂, and the combination of D and B for a compound corresponds in each case to one row of Table B.

Table 138 Compounds of the formula (I-D) in which R¹ is CN, Y is SO₂, and the combination of D and B for a compound corresponds in each case to one row of Table B.

Table 139 Compounds of the formula (I-E) in which R¹ is H, Y is SO, and the combination of D and B for a compound corresponds in each case to one row of Table B.

Table 140 Compounds of the formula (I-E) in which R¹ is F, Y is SO, and the combination of D and B for a compound corresponds in each case to one row of Table B.

Table 141 Compounds of the formula (I-E) in which R¹ is CN, Y is SO, and the combination of D and B for a compound corresponds in each case to one row of Table B.

Table 142 Compounds of the formula (I-E) in which R¹ is H, Y is SO₂, and the combination of D and B for a compound corresponds in each case to one row of Table B.

Table 143 Compounds of the formula (I-E) in which R¹ is F, Y is SO₂, and the combination of D and B for a compound corresponds in each case to one row of Table B.

Table 144 Compounds of the formula (I-E) in which R¹ is CN, Y is SO₂, and the combination of D and B for a compound corresponds in each case to one row of Table B.

Table 145 Compounds of the formula (I-F) in which R¹ is H, Y is SO, and the combination of D and B for a compound corresponds in each case to one row of Table B.

Table 146 Compounds of the formula (I-F) in which R¹ is F, Y is SO, and the combination of D and B for a compound corresponds in each case to one row of Table B.

Table 147 Compounds of the formula (I-F) in which R¹ is CN, Y is SO, and the combination of D and B for a compound corresponds in each case to one row of Table B.

Table 148 Compounds of the formula (I-F) in which R¹ is H, Y is SO₂, and the combination of D and B for a compound corresponds in each case to one row of Table B.

Table 149 Compounds of the formula (I-F) in which R¹ is F, Y is SO₂, and the combination of D and B for a compound corresponds in each case to one row of Table B.

Table 150 Compounds of the formula (I-F) in which R¹ is CN, Y is SO₂, and the combination of D and B for a compound corresponds in each case to one row of Table B.

Table 151 Compounds of the formula (I-G) in which R¹ is H, Y is SO, and the combination of D and B for a compound corresponds in each case to one row of Table B.

Table 152 Compounds of the formula (I-G) in which R¹ is F, Y is SO, and the combination of D and B for a compound corresponds in each case to one row of Table B.

Table 153 Compounds of the formula (I-G) in which R¹ is CN, Y is SO, and the combination of D and B for a compound corresponds in each case to one row of Table B.

Table 154 Compounds of the formula (I-G) in which R¹ is H, Y is SO₂, and the combination of D and B for a compound corresponds in each case to one row of Table B.

Table 155 Compounds of the formula (I-G) in which R¹ is F, Y is SO₂, and the combination of D and B for a compound corresponds in each case to one row of Table B.

Table 156 Compounds of the formula (I-G) in which R¹ is CN, Y is SO₂, and the combination of D and B for a compound corresponds in each case to one row of Table B.

Table 157 Compounds of the formula (I-H) in which R¹ is H, Y is SO, and the combination of D and B for a compound corresponds in each case to one row of Table B.

Table 158 Compounds of the formula (I-H) in which R¹ is F, Y is SO, and the combination of D and B for a compound corresponds in each case to one row of Table B.

Table 159 Compounds of the formula (I-H) in which R¹ is CN, Y is SO, and the combination of D and B for a compound corresponds in each case to one row of Table B.

Table 160 Compounds of the formula (I-H) in which R¹ is H, Y is SO₂, and the combination of D and B for a compound corresponds in each case to one row of Table B.

Table 161 Compounds of the formula (I-H) in which R¹ is F, Y is SO₂, and the combination of D and B for a compound corresponds in each case to one row of Table B.

Table 162 Compounds of the formula (I-H) in which R¹ is CN, Y is SO₂, and the combination of D and B for a compound corresponds in each case to one row of Table B.

Table 163 Compounds of the formula (I-I) in which R¹ is H, Y is SO, and the combination of D and B for a compound corresponds in each case to one row of Table B.

Table 164 Compounds of the formula (I-I) in which R¹ is F, Y is SO, and the combination of D and B for a compound corresponds in each case to one row of Table B.

Table 165 Compounds of the formula (I-I) in which R¹ is CN, Y is SO, and the combination of D and B for a compound corresponds in each case to one row of Table B.

Table 166 Compounds of the formula (I-I) in which R¹ is H, Y is SO₂, and the combination of D and B for a compound corresponds in each case to one row of Table B.

Table 167 Compounds of the formula (I-I) in which R¹ is F, Y is SO₂, and the combination of D and B for a compound corresponds in each case to one row of Table B.

Table 168 Compounds of the formula (I-I) in which R¹ is CN, Y is SO₂, and the combination of D and B for a compound corresponds in each case to one row of Table B.

Table 169 Compounds of the formula (I-J) in which R¹ is H, Y is SO, and the combination of D and B for a compound corresponds in each case to one row of Table B.

Table 170 Compounds of the formula (I-J) in which R¹ is F, Y is SO, and the combination of D and B for a compound corresponds in each case to one row of Table B.

Table 171 Compounds of the formula (I-J) in which R¹ is CN, Y is SO, and the combination of D and B for a compound corresponds in each case to one row of Table B.

Table 172 Compounds of the formula (I-J) in which R¹ is H, Y is SO₂, and the combination of D and B for a compound corresponds in each case to one row of Table B.

Table 173 Compounds of the formula (I-J) in which R¹ is F, Y is SO₂, and the combination of D and B for a compound corresponds in each case to one row of Table B.

Table 174 Compounds of the formula (I-J) in which R¹ is CN, Y is SO₂, and the combination of D and B for a compound corresponds in each case to one row of Table B.

Table 175 Compounds of the formula (I-K) in which R¹ is H, Y is SO, and the combination of D and B for a compound corresponds in each case to one row of Table B.

Table 176 Compounds of the formula (I-K) in which R¹ is F, Y is SO, and the combination of D and B for a compound corresponds in each case to one row of Table B.

Table 177 Compounds of the formula (I-K) in which R¹ is CN, Y is SO, and the combination of D and B for a compound corresponds in each case to one row of Table B.

Table 178 Compounds of the formula (I-K) in which R¹ is H, Y is SO₂, and the combination of D and B for a compound corresponds in each case to one row of Table B.

Table 179 Compounds of the formula (I-K) in which R¹ is F, Y is SO₂, and the combination of D and B for a compound corresponds in each case to one row of Table B.

Table 180 Compounds of the formula (I-K) in which R¹ is CN, Y is SO₂, and the combination of D and B for a compound corresponds in each case to one row of Table B.

TABLE B

| | D | B |
|---|---|---|
| B-1 | H | N(CH₃)₂ |
| B-2 | H | N(CH₃)CH₂CH₃ |
| B-3 | H | N(CH₃)CH₂CH₂CH₃ |
| B-4 | H | N(CH₃)—c-C₃H₅ |
| B-5 | H | N(CH₃)CH(CH₃)₂ |
| B-6 | H | N(CH₃)CH₂CH(CH₃)₂ |
| B-7 | H | N(CH₃)CH(CH₃)—c-C₃H₅ |
| B-8 | H | N(CH₃)CH₂CH₂CH₂CH₃ |
| B-9 | H | N(CH₃)CH(CH₃)CH₂CH₃ |
| B-10 | H | N(CH₃)(CH₂CH(CH₃)₂ |
| B-11 | H | N(CH₃)C(CH₃)₃ |
| B-12 | H | N(CH₃)—c-C₄H₇ |
| B-13 | H | N(CH₃)-1-CH₂—c-C₃H₅ |
| B-14 | H | NH(CO)CH₃ |
| B-15 | H | NH(CO)CH₂CH₃ |
| B-16 | H | NH(CO)CH(CH₃)₂ |
| B-17 | H | NH(CO)—c-C₃H₅ |
| B-18 | F | N(CH₃)₂ |
| B-19 | F | N(CH₃)CH₂CH₃ |
| B-20 | F | N(CH₃)CH₂CH₂CH₃ |
| B-21 | F | N(CH₃)—c-C₃H₅ |
| B-22 | F | N(CH₃)CH(CH₃)₂ |
| B-23 | F | N(CH₃)CH₂CH(CH₃)₂ |
| B-24 | F | N(CH₃)CH(CH₃)—c-C₃H₅ |
| B-25 | F | N(CH₃)CH₂CH₂CH₂CH₃ |
| B-26 | F | N(CH₃)CH(CH₃)CH₂CH₃ |
| B-27 | F | N(CH₃)(CH₂CH(CH₃)₂ |
| B-28 | F | N(CH₃)C(CH₃)₃ |
| B-29 | F | N(CH₃)—c-C₄H₇ |
| B-30 | F | N(CH₃)-1-CH₂—c-C₃H₅ |
| B-31 | F | NH(CO)CH₃ |
| B-32 | F | NH(CO)CH₂CH₃ |
| B-33 | F | NH(CO)CH(CH₃)₂ |
| B-34 | F | NH(CO)—c-C₃H₅ |
| B-35 | Cl | N(CH₃)₂ |
| B-36 | Cl | N(CH₃)CH₂CH₃ |
| B-37 | Cl | N(CH₃)CH₂CH₂CH₃ |
| B-38 | Cl | N(CH₃)—c-C₃H₅ |
| B-39 | Cl | N(CH₃)CH(CH₃)₂ |
| B-40 | Cl | N(CH₃)CH₂CH(CH₃)₂ |
| B-41 | Cl | N(CH₃)CH(CH₃)—c-C₃H₅ |
| B-42 | Cl | N(CH₃)CH₂CH₂CH₂CH₃ |
| B-43 | Cl | N(CH₃)CH(CH₃)CH₂CH₃ |
| B-44 | Cl | N(CH₃)(CH₂CH(CH₃)₂ |
| B-45 | Cl | N(CH₃)C(CH₃)₃ |
| B-46 | Cl | N(CH₃)—c-C₄H₇ |
| B-47 | Cl | N(CH₃)-1-CH₂—c-C₃H₅ |
| B-48 | Cl | NH(CO)CH₃ |
| B-49 | Cl | NH(CO)CH₂CH₃ |
| B-50 | Cl | NH(CO)CH(CH₃)₂ |
| B-51 | Cl | NH(CO)—c-C₃H₅ |
| B-52 | Br | N(CH₃)₂ |
| B-53 | Br | N(CH₃)CH₂CH₃ |
| B-54 | Br | N(CH₃)CH₂CH₂CH₃ |
| B-55 | Br | N(CH₃)—c-C₃H₅ |
| B-56 | Br | N(CH₃)CH(CH₃)₂ |
| B-57 | Br | N(CH₃)CH₂CH(CH₃)₂ |
| B-58 | Br | N(CH₃)CH(CH₃)—c-C₃H₅ |
| B-59 | Br | N(CH₃)CH₂CH₂CH₂CH₃ |
| B-60 | Br | N(CH₃)CH(CH₃)CH₂CH₃ |
| B-61 | Br | N(CH₃)(CH₂CH(CH₃)₂ |
| B-62 | Br | N(CH₃)C(CH₃)₃ |
| B-63 | Br | N(CH₃)—c-C₄H₇ |
| B-64 | Br | N(CH₃)-1-CH₂—c-C₃H₅ |
| B-65 | Br | NH(CO)CH₃ |
| B-66 | Br | NH(CO)CH₂CH₃ |
| B-67 | Br | NH(CO)CH(CH₃)₂ |
| B-68 | Br | NH(CO)—c-C₃H₅ |
| B-69 | I | N(CH₃)₂ |
| B-70 | I | N(CH₃)CH₂CH₃ |
| B-71 | I | N(CH₃)CH₂CH₂CH₃ |
| B-72 | I | N(CH₃)—c-C₃H₅ |
| B-73 | I | N(CH₃)CH(CH₃)₂ |
| B-74 | I | N(CH₃)CH₂CH(CH₃)₂ |
| B-75 | I | N(CH₃)CH(CH₃)—c-C₃H₅ |
| B-76 | I | N(CH₃)CH₂CH₂CH₂CH₃ |
| B-77 | I | N(CH₃)CH(CH₃)CH₂CH₃ |
| B-78 | I | N(CH₃)(CH₂CH(CH₃)₂ |
| B-79 | I | N(CH₃)C(CH₃)₃ |
| B-80 | I | N(CH₃)—c-C₄H₇ |
| B-81 | I | N(CH₃)-1-CH₂—c-C₃H₅ |
| B-82 | I | NH(CO)CH₃ |
| B-83 | I | NH(CO)CH₂CH₃ |
| B-84 | I | NH(CO)CH(CH₃)₂ |
| B-85 | I | NH(CO)—c-C₃H₅ |
| B-86 | CH₃ | N(CH₃)₂ |
| B-87 | CH₃ | N(CH₃)CH₂CH₃ |
| B-88 | CH₃ | N(CH₃)CH₂CH₂CH₃ |
| B-89 | CH₃ | N(CH₃)—c-C₃H₅ |
| B-90 | CH₃ | N(CH₃)CH(CH₃)₂ |
| B-91 | CH₃ | N(CH₃)CH₂CH(CH₃)₂ |
| B-92 | CH₃ | N(CH₃)CH(CH₃)—c-C₃H₅ |
| B-93 | CH₃ | N(CH₃)CH₂CH₂CH₂CH₃ |
| B-94 | CH₃ | N(CH₃)CH(CH₃)CH₂CH₃ |
| B-95 | CH₃ | N(CH₃)(CH₂CH(CH₃)₂ |
| B-96 | CH₃ | N(CH₃)C(CH₃)₃ |
| B-97 | CH₃ | N(CH₃)—c-C₄H₇ |
| B-98 | CH₃ | N(CH₃)-1-CH₂—c-C₃H₅ |
| B-99 | CH₃ | NH(CO)CH₃ |
| B-100 | CH₃ | NH(CO)CH₂CH₃ |
| B-101 | CH₃ | NH(CO)CH(CH₃)₂ |
| B-102 | CH₃ | NH(CO)—c-C₃H₅ |
| B-103 | CF₃ | N(CH₃)₂ |
| B-104 | CF₃ | N(CH₃)CH₂CH₃ |
| B-105 | CF₃ | N(CH₃)CH₂CH₂CH₃ |
| B-106 | CF₃ | N(CH₃)—c-C₃H₅ |
| B-107 | CF₃ | N(CH₃)CH(CH₃)₂ |
| B-108 | CF₃ | N(CH₃)CH₂CH(CH₃)₂ |
| B-109 | CF₃ | N(CH₃)CH(CH₃)—c-C₃H₅ |
| B-110 | CF₃ | N(CH₃)CH₂CH₂CH₂CH₃ |
| B-111 | CF₃ | N(CH₃)CH(CH₃)CH₂CH₃ |
| B-112 | CF₃ | N(CH₃)(CH₂CH(CH₃)₂ |
| B-113 | CF₃ | N(CH₃)C(CH₃)₃ |
| B-114 | CF₃ | N(CH₃)—c-C₄H₇ |
| B-115 | CF₃ | N(CH₃)-1-CH₂—c-C₃H₅ |
| B-116 | CF₃ | NH(CO)CH₃ |
| B-117 | CF₃ | NH(CO)CH₂CH₃ |
| B-118 | CF₃ | NH(CO)CH(CH₃)₂ |
| B-119 | CF₃ | NH(CO)—c-C₃H₅ |
| B-120 | CN | N(CH₃)₂ |
| B-121 | CN | N(CH₃)CH₂CH₃ |
| B-122 | CN | N(CH₃)CH₂CH₂CH₃ |
| B-123 | CN | N(CH₃)—c-C₃H₅ |
| B-124 | CN | N(CH₃)CH(CH₃)₂ |
| B-125 | CN | N(CH₃)CH₂CH(CH₃)₂ |
| B-126 | CN | N(CH₃)CH(CH₃)—c-C₃H₅ |
| B-127 | CN | N(CH₃)CH₂CH₂CH₂CH₃ |
| B-128 | CN | N(CH₃)CH(CH₃)CH₂CH₃ |
| B-129 | CN | N(CH₃)(CH₂CH(CH₃)₂ |
| B-130 | CN | N(CH₃)C(CH₃)₃ |
| B-131 | CN | N(CH₃)—c-C₄H₇ |
| B-132 | CN | N(CH₃)-1-CH₂—c-C₃H₅ |
| B-133 | CN | NH(CO)CH₃ |
| B-134 | CN | NH(CO)CH₂CH₃ |
| B-135 | CN | NH(CO)CH(CH₃)₂ |
| B-136 | CN | NH(CO)—c-C₃H₅ |

In both Table A and Table B, the following abbreviations have the following meanings: 1-CH₃-c-C₃H₄ is 1-methyl-cyclopropyl; 2,2-FcP is 2,2-difluoro-cyclopropyl; 3,3-FB is 3,3-difluoro-cyclobutyl; 4,4-FH is 4,4-difluoro-cyclohexyl; 2-FU is 2-furanyl; 3-FU is 3-furanyl; 2-HFU is tetrahydro-2-furanyl; 3-HFU is tetrahydro-3-furanyl; c-C₃H₅ is cyclopropyl; c-C₄H₇ is cyclobutyl; c-C₅H₉ is cyclopentyl; CH₂-(c-C₃H₅), which may also be referred to as CH₂-c-C₃H₅, is cyclopropylmethyl; CH₂(2,2-FcP) corresponds to B¹¹ as defined below and CH₂(2-HFU) corresponds to B¹⁷ as defined below; C₆H₅ is phenyl.

The groups B¹ to B²⁰ have the following meanings, wherein § designates the bond to the Y-group of the compounds of formulae I-B, I-C, I-D, I-E, I-F, I-G, I-H, I-I, I-J, and I-K, respectively.

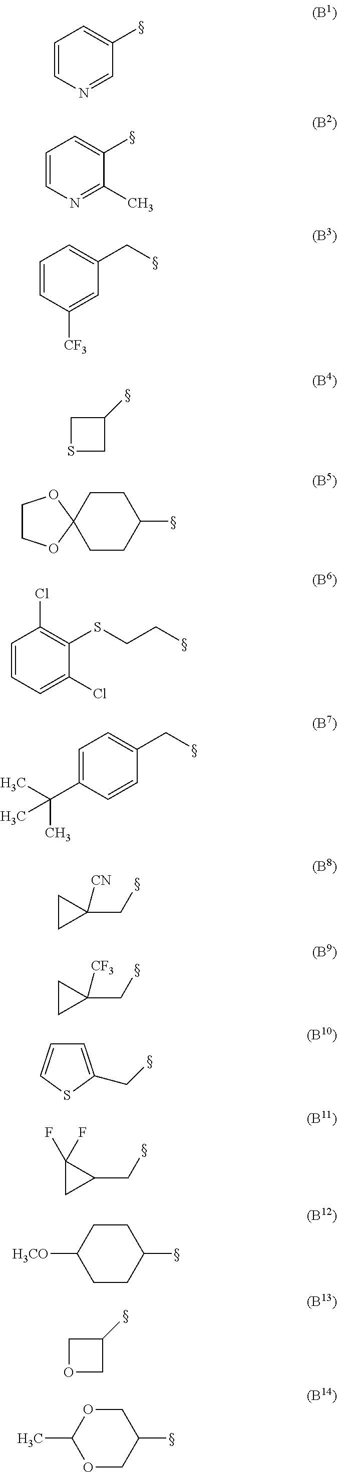

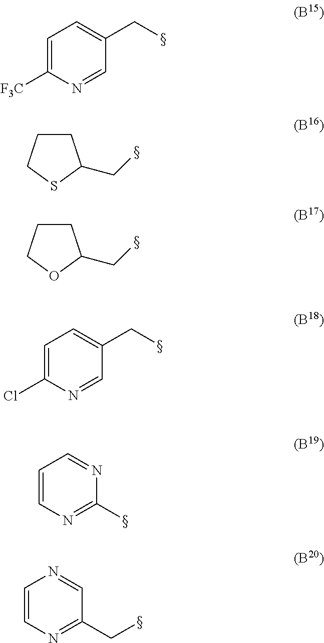

Especially preferred compounds of formula I in the methods and uses and preferred compounds I* according to the invention are those of formula I-B, I-C, I-D, and I-E, and in particular those of Table 1-3, 13-15, 25-27, and 37-39.

Due to their excellent activity, the compounds of the present invention may be used for controlling invertebrate pests. Preferences in this regard, in particular with regard to suitable mixtures, formulations, application methods, pests and animal health, are provided hereinafter.

As used hereinafter, the term “compound(s) of the present invention” or “compound(s) according to the invention” refers to the compound(s) of formula (I) or (I*) as defined above, including their salts, tautomers, stereoisomers, and N-oxides, and including the preferred compounds defined above and the compounds listed in Tables 1 to 180.

The present invention also relates to a mixture of at least one compound of the present invention with at least one mixing partner as defined herein after. Preferred are binary mixtures of one compound of the present invention as component I with one mixing partner as defined herein after as component II. Preferred weight ratios for such binary mixtures are from 5000:1 to 1:5000, preferably from 1000:1 to 1:1000, more preferably from 100:1 to 1:100, particularly preferably from 10:1 to 1:10. In such binary mixtures, components I and II may be used in equal amounts, or an excess of component I, or an excess of component II may be used.

Mixing partners can be selected from pesticides, in particular insecticides, nematicides, and acaricides, fungicides, herbicides, plant growth regulators, fertilizers, and the like. Preferred mixing partners are insecticides, nematicides and fungicides.

The following list M of pesticides, grouped and numbered according the Mode of Action Classification of the Insecticide Resistance Action Committee (IRAC), together with which the compounds of the present invention can be used and with which potential synergistic effects might be produced, is intended to illustrate the possible combinations, but not to impose any limitation: M.1 Acetylcholine esterase (AChE) inhibitors from the class of: M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;

M.2. GABA-gated chloride channel antagonists such as: M.2A cyclodiene organochlorine compounds, as for example endosulfan or chlordane; or M.2B fiproles (phenylpyrazoles), as for example ethiprole, fipronil, flufiprole, pyrafluprole and pyriprole;

M.3 Sodium channel modulators from the class of M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, heptafluthrin, imiprothrin, meperfluthrin, metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or M.3B sodium channel modulators such as DDT or methoxychlor;

M.4 Nicotinic acetylcholine receptor agonists (nAChR) from the class of M.4A neonicotinoids, for example acetamiprid, clothianidin, cycloxaprid, dinotefuran, imidacloprid, nitenpyram, thiacloprid and thiamethoxam; or the compounds M.4A.2: (2E-)-1-[(6-Chloropyridin-3-yl)methyl]-N′-nitro-2-pentylidenehydrazinecarboximidamide; or M4.A.3: 1-[(6-Chloropyridin-3-yl)methyl]-7-methyl-8-nitro-5-propoxy-1,2,3,5,6,7-hexahydroimidazo[1,2-a]pyridine; or from the class M.4B nicotine;

M.5 Nicotinic acetylcholine receptor allosteric activators from the class of spinosyns, for example spinosad or spinetoram;

M.6 Chloride channel activators from the class of avermectins and milbemycins, for example abamectin, emamectin benzoate, ivermectin, lepimectin or milbemectin;

M.7 Juvenile hormone mimics, such as M.7A juvenile hormone analogues as hydroprene, kinoprene and methoprene; or others as M.7B fenoxycarb or M.7C pyriproxyfen;

M.8 miscellaneous non-specific (multi-site) inhibitors, for example M.8A alkyl halides as methyl bromide and other alkyl halides, or M.8B chloropicrin, or M.8C sulfuryl fluoride, or M.8D borax, or M.8E tartar emetic;

M.9 Selective homopteran feeding blockers, for example M.9B pymetrozine, or M.9C flonicamid;

M.10 Mite growth inhibitors, for example M.10A clofentezine, hexythiazox and diflovidazin, or M.10B etoxazole;

M.11 Microbial disruptors of insect midgut membranes, for example bacillus thuringiensis or bacillus sphaericus and the insecticdal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;

M.12 Inhibitors of mitochondrial ATP synthase, for example M.12A diafenthiuron, or M.12B organotin miticides such as azocyclotin, cyhexatin or fenbutatin oxide, or M.12C propargite, or M.12D tetradifon;

M.13 Uncouplers of oxidative phosphorylation via disruption of the proton gradient, for example chlorfenapyr, DNOC or sulfluramid;

M.14 Nicotinic acetylcholine receptor (nAChR) channel blockers, for example nereistoxin analogues as bensultap, cartap hydrochloride, thiocyclam or thiosultap sodium;

M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;

M.16 Inhibitors of the chitin biosynthesis type 1, as for example buprofezin;

M.17 Moulting disruptors, Dipteran, as for example cyromazine;

M.18 Ecdyson receptor agonists such as diacylhydrazines, for example methoxyfenozide, tebufenozide, halofenozide, fufenozide or chromafenozide;

M.19 Octopamin receptor agonists, as for example amitraz;

M.20 Mitochondrial complex III electron transport inhibitors, for example M.20A hydramethylnon, or M.20B acequinocyl, or M.20C fluacrypyrim;

M.21 Mitochondrial complex I electron transport inhibitors, for example M.21A METI acaricides and insecticides such as fenazaquin, fenpyroximate, pyrimidifen, pyridaben, tebufenpyrad or tolfenpyrad, or M.21B rotenone;

M.22 Voltage-dependent sodium channel blockers, for example M.22A indoxacarb, or M.22B metaflumizone, or M.22B.1: 2-[2-(4-Cyanophenyl)-1-[3-(trifluoromethyl)phenyl]ethylidene]-N-[4-(difluoromethoxy)phenyl]-hydrazinecarboxamide or M.22B.2: N-(3-Chloro-2-methylphenyl)-2-[(4-chloro-phenyl)[4-[methyl(methylsulfonyl)amino]phenyl]methylene]-hydrazinecarboxamide;

M.23 Inhibitors of the of acetyl CoA carboxylase, such as Tetronic and Tetramic acid derivatives, for example spirodiclofen, spiromesifen or spirotetramat;

M.24 Mitochondrial complex IV electron transport inhibitors, for example M.24A phosphine such as aluminium phosphide, calcium phosphide, phosphine or zinc phosphide, or M.24B cyanide;

M.25 Mitochondrial complex II electron transport inhibitors, such as beta-ketonitrile derivatives, for example cyenopyrafen or cyflumetofen;

M.28 Ryanodine receptor-modulators from the class of diamides, as for example flubendiamide, chlorantraniliprole (Rynaxypyr®), cyantraniliprole (Cyazypyr®), tetraniliprole, or the phthalamide compounds M.28.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and M.28.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a compound selected from M.28.5a) to M.28.5d) and M.28.5h) to M.28.5I): M.28.5a)N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide; M.28.5b)N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide; M.28.5c)N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide; M.28.5d)N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide; M.28.5h)N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide; M.28.5i)N-[2-(5-Amino-1,3,4-thiadiazol-2-yl)-4-chloro-6-methylphenyl]-3-bromo-1-(3-chloro-2-pyridinyl)-1H-pyrazole-5-carboxamide; M.28.5j) 3-Chloro-1-(3-chloro-2-pyridinyl)-N-[2,4-dichloro-6-[[(1-cyano-1-methylethyl)amino]carbonyl]phenyl]-1H-pyrazole-5-carboxamide; M.28.5k) 3-Bromo-N-[2,4-dichloro-6-(methylcarbamoyl)phenyl]-1-(3,5-dichloro-2-pyridyl)-1H-pyrazole-5-carboxamide; M.28.5I)N-[4-Chloro-2-[[(1,1-dimethylethyl)amino]carbonyl]-6-methylphenyl]-1-(3-chloro-2-pyridinyl)-3-(fluoromethoxy)-1H-pyrazole-5-carboxamide; or

M.28.6: cyhalodiamide; or;

M.29. insecticidal active compounds of unknown or uncertain mode of action, as for example afidopyropen, afoxolaner, azadirachtin, amidoflumet, benzoximate, bifenazate, broflanilide, bromopropylate, chinomethionat, cryolite, dicloromezotiaz, dicofol, flufenerim, flometoquin, fluensulfone, fluhexafon, fluopyram, flupyradifurone, fluralaner, metoxadiazone, piperonyl butoxide, pyflubumide, pyridalyl, pyrifluquinazon, sulfoxaflor, tioxazafen, triflumezopyrim, or the compounds

M.29.3: 11-(4-chloro-2,6-dimethylphenyl)-12-hydroxy-1,4-dioxa-9-azadispiro[4.2.4.2]-tetradec-11-en-10-one, or the compound
M.29.4: 3-(4′-fluoro-2,4-dimethylbiphenyl-3-yl)-4-hydroxy-8-oxa-1-azaspiro[4.5]dec-3-en-2-one, or the compound
M.29.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, 1-1582); or

a compound selected from the group of M.29.6, wherein the compound is selected from M.29.6a) to M.29.6k): M.29.6a) (E/Z)-N-[1-[(6-chloro-3-pyridyl)methyl]-2-pyridylidene]-2,2,2-trifluoro-acetamide; M.29.6b) (E/Z)-N-[1-[(6-chloro-5-fluoro-3-pyridyl)methyl]-2-pyridylidene]-2,2,2-trifluoro-acetamide; M.29.6c) (E/Z)-2,2,2-trifluoro-N-[1-[(6-fluoro-3-pyridyl)methyl]-2-pyridylidene]acetamide; M.29.6d) (E/Z)-N-[1-[(6-bromo-3-pyridyl)methyl]-2-pyridylidene]-2,2,2-trifluoroacetamide; M.29.6e) (E/Z)-N-[1-[1-(6-chloro-3-pyridyl)ethyl]-2-pyridylidene]-2,2,2-trifluoro-acetamide; M.29.6f) (E/Z)-N-[1-[(6-chloro-3-pyridyl)methyl]-2-pyridylidene]-2,2-difluoro-acetamide; M.29.6g) (E/Z)-2-chloro-N-[1-[(6-chloro-3-pyridyl)methyl]-2-pyridylidene]-2,2-difluoro-acetamide; M.29.6h) (E/Z)-N-[1-[(2-chloropyrimidin-5-yl)methyl]-2-pyridylidene]-2,2,2-trifluoro-acetamide; M.29.6i) (E/Z)-N-[1-[(6-chloro-3-pyridyl)methyl]-2-pyridylidene]-2,2,3,3,3-pentafluoro-propanamide.); M.29.6j)N-[1-[(6-chloro-3-pyridyl)methyl]-2-pyridylidene]-2,2,2-trifluoro-thioacetamide; or M.29.6k)N-[1-[(6-chloro-3-pyridyl)methyl]-2-pyridylidene]-2,2,2-trifluoro-N′-isopropyl-acetamidine; or the compounds

M.29.8: fluazaindolizine; or the compounds

M.29.9.a): 4-[5-(3,5-dichlorophenyl)-5-(trifluoromethyl)-4H-isoxazol-3-yl]-2-methyl-N-(1-oxothietan-3-yl)benzamide; or M.29.9.b): fluxametamide; or

M.29.10: 5-[3-[2,6-dichloro-4-(3,3-dichloroallyloxy)phenoxy]propoxy]-1H-pyrazole; or

a compound selected from the group of M.29.11, wherein the compound is selected from M.29.11b) to M.29.11p): M.29.11.b) 3-(benzoylmethylamino)-N-[2-bromo-4-[1,2,2,3,3,3-hexafluoro-1-(trifluoromethyl)propyl]-6-(trifluoromethyl)phenyl]-2-fluoro-benzamide; M.29.11.c) 3-(benzoylmethylamino)-2-fluoro-N-[2-iodo-4-[1,2,2,2-tetrafluoro-1-(trifluoromethyl)ethyl]-6-(trifluoromethyl)phenyl]-benzamide; M.29.11.d)N-[3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; M.29.11.e)N-[3-[[[2-bromo-4-[1,2,2,2-tetrafluoro-1-(trifluoromethyl)ethyl]-6-(trifluoromethyl)phenyl]amino]carbonyl]-2-fluorophenyl]-4-fluoro-N-methyl-benzamide; M.29.11.f) 4-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; M.29.11.g) 3-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; M.29.11.h) 2-chloro-N-[3-[[[2-iodo-4-[1,2,2,2-tetrafluoro-1-(trifluoromethyl)ethyl]-6-(trifluoromethyl)phenyl]amino]carbonyl]phenyl]-3-pyridinecarboxamide; M.29.11.i) 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide; M.29.11.j) 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide; M.29.11.k)N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide; M.29.11.l)N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; M.29.11.m)N-[5-[[2-bromo-6-chloro-4-[1,2,2,3,3,3-hexafluoro-1-(trifluoromethyl)propyl]phenyl]carbamoyl]-2-cyano-phenyl]-4-cyano-2-methyl-benzamide; M.29.11.n) 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide; M.29.11.o) 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide; M.29.11.p)N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide; or

a compound selected from the group of M.29.12, wherein the compound is selected from M.29.12a) to M.29.12m): M.29.12.a) 2-(1,3-Dioxan-2-yl)-6-[2-(3-pyridinyl)-5-thiazolyl]-pyridine; M.29.12.b) 2-[6-[2-(5-Fluoro-3-pyridinyl)-5-thiazolyl]-2-pyridinyl]-pyrimidine; M.29.12.c) 2-[6-[2-(3-Pyridinyl)-5-thiazolyl]-2-pyridinyl]-pyrimidine; M.29.12.d)N-Methylsulfonyl-6-[2-(3-pyridyl)thiazol-5-yl]pyridine-2-carboxamide; M.29.12.e)N-Methylsulfonyl-6-[2-(3-pyridyl)thiazol-5-yl]pyridine-2-carboxamide; M.29.12.f)N-Ethyl-N-[4-methyl-2-(3-pyridyl)thiazol-5-yl]-3-methylthio-propanamide; M.29.12.g)N-M ethyl-N-[4-methyl-2-(3-pyridyl)thiazol-5-yl]-3-methylthio-propanamide; M.29.12.h) N,2-Dimethyl-N-[4-methyl-2-(3-pyridyl)thiazol-5-yl]-3-methylthio-propanamide; M.29.12.i)N-Ethyl-2-methyl-N-[4-methyl-2-(3-pyridyl)thiazol-5-yl]-3-methylthio-propanamide; M.29.12.j)N-[4-Chloro-2-(3-pyridyl)thiazol-5-yl]-N-ethyl-2-methyl-3-methylthio-propanamide; M.29.12.k)N-[4-Chloro-2-(3-pyridyl)thiazol-5-yl]-N,2-dimethyl-3-methylthio-propanamide; M.29.12.l)N-[4-Chloro-2-(3-pyridyl)thiazol-5-yl]-N-methyl-3-methylthio-propanamide; M.29.12.m)N-[4-Chloro-2-(3-pyridyl)thiazol-5-yl]-N-ethyl-3-methylthio-propanamide; or the compounds

M.29.14a) 1-[(6-Chloro-3-pyridinyl)methyl]-1,2,3,5,6,7-hexahydro-5-methoxy-7-methyl-8-nitroimidazo[1,2-a]pyridine; or M.29.14b) 1-[(6-Chloropyridin-3-yl)methyl]-7-methyl-8-nitro-1,2,3,5,6,7-hexahydroimidazo[1,2-a]pyridin-5-ol; or the compounds M.29.16a) 1-isopropyl-N,5-dimethyl-N-pyridazin-4-yl-pyrazole-4-carboxamide; or M.29.16b) 1-(1,2-dimethylpropyl)-N-ethyl-5-methyl-N-pyridazin-4-yl-pyrazole-4-carboxamide; M.29.16c) N,5-dimethyl-N-pyridazin-4-yl-1-(2,2,2-trifluoro-1-methyl-ethyl)pyrazole-4-carboxamide; M.29.16d) 1-[1-(1-cyanocyclopropyl)ethyl]-N-ethyl-5-methyl-N-pyridazin-4-yl-pyrazole-4-carboxamide; M.29.16e)N-ethyl-1-(2-fluoro-1-methyl-propyl)-5-methyl-N-pyridazin-4-yl-pyrazole-4-carboxamide; M.29.16f) 1-(1,2-dimethylpropyl)-N,5-dimethyl-N-pyridazin-4-yl-pyrazole-4-carboxamide; M.29.16g) 1-[1-(1-cyanocyclopropyl)ethyl]-N,5-dimethyl-N-pyridazin-4-yl-pyrazole-4-carboxamide; M.29.16h)N-methyl-1-(2-fluoro-1-methyl-propyl]-5-methyl-N-pyridazin-4-yl-pyrazole-4-carboxamide; M.29.16i) 1-(4,4-difluorocyclohexyl)-N-ethyl-5-methyl-N-pyridazin-4-yl-pyrazole-4-carboxamide; or M.29.16j) 1-(4,4-difluorocyclohexyl)-N,5-dimethyl-N-pyridazin-4-yl-pyrazole-4-carboxamide, or

M.29.17 a compound selected from the compounds M.29.17a) to M.29.17j): M.29.17a)N-(1-methylethyl)-2-(3-pyridinyl)-2H-indazole-4-carboxamide; M.29.17b)N-cyclopropyl-2-(3-pyridinyl)-2H-indazole-4-carboxamide; M.29.17c)N-cyclohexyl-2-(3-pyridinyl)-2H-indazole-4-carboxamide; M.29.17d) 2-(3-pyridinyl)-N-(2,2,2-trifluoroethyl)-2H-indazole-4-carboxamide; M.29.17e) 2-(3-pyridinyl)-N-[(tetrahydro-2-furanyl)methyl]-2H-indazole-5-carboxamide; M.29.17f) methyl 2-[[2-(3-pyridinyl)-2H-indazol-5-yl]carbonyl]hydrazinecarboxylate; M.29.17g)N-[(2,2-difluorocyclopropyl)methyl]-2-(3-pyridinyl)-2H-indazole-5-carboxamide; M.29.17h)N-(2,2-difluoropropyl)-2-(3-pyridinyl)-2H-indazole-5-carboxamide; M.29.17i) 2-(3-pyridinyl)-N-(2-pyrimidinylmethyl)-2H-indazole-5-carboxamide; M.29.17j)N-[(5-methyl-2-pyrazinyl)methyl]-2-(3-pyridinyl)-2H-indazole-5-carboxamide, or

M.29.18 a compound selected from the compounds M.29.18a) to M.29.18d): M.29.18a)N-[3-chloro-1-(3-pyridyl)pyrazol-4-yl]-N-ethyl-3-(3,3,3-trifluoropropylsulfanyl)propanamide; M.29.18b) N-[3-chloro-1-(3-pyridyl)pyrazol-4-yl]-N-ethyl-3-(3,3,3-trifluoropropylsulfinyl)propanamide; M.29.18c)N-[3-chloro-1-(3-pyridyl)pyrazol-4-yl]-3-[(2,2-difluorocyclopropyl)methylsulfanyl]-N-ethyl-propanamide; M.29.18d)N-[3-chloro-1-(3-pyridyl)pyrazol-4-yl]-3-[(2,2-difluorocyclopropyl)methylsulfinyl]-N-ethyl-propanamide; or the compound

M.29.19 sarolaner, or the compound

M.29.20 lotilaner.

The commercially available compounds of the group M listed above may be found in The Pesticide Manual, 16th Edition, C. MacBean, British Crop Protection Council (2013) among other publications. The online Pesticide Manual is updated regularly and is accessible through http://bcpcdata.com/pesticide-manual.html.

Another online data base for pesticides providing the ISO common names is http://www.alanwood. net/pesticides.

The M.4 neonicotinoid cycloxaprid is known from WO2010/069266 and WO2011/069456, the neonicotinoid M.4A.2, sometimes also to be named as guadipyr, is known from WO2013/003977, and the neonicotinoid M.4A.3 (approved as paichongding in China) is known from WO2007/101369. The metaflumizone analogue M.22B.1 is described in CN10171577 and the analogue M.22B.2 in CN102126994. The phthalamides M.28.1 and M.28.2 are both known from WO2007/101540. The anthranilamide M.28.3 is described in WO2005/077934. The hydrazide compound M.28.4 is described in WO2007/043677. The anthranilamides M.28.5a) to M.28.5d) and M.28.5h) are described in WO 2007/006670, WO2013/024009 and WO2013/024010, the anthranilamide M.28.5i) is described in WO2011/085575, M.28.5j) in WO2008/134969, M.28.5k) in US2011/046186 and M.28.5I) in WO2012/034403. The diamide compound M.28.6 can be found in WO2012/034472. The spiroketal-substituted cyclic ketoenol derivative M.29.3 is known from WO2006/089633 and the biphenyl-substituted spirocyclic ketoenol derivative M.29.4 from WO2008/067911. The triazoylphenylsulfide M.29.5 is described in WO2006/043635, and biological control agents on the basis of bacillus firmus are described in WO2009/124707. The compounds M.29.6a) to M.29.6i) listed under M.29.6 are described in WO2012/029672, and M.29.6j) and M.29.6k) in WO2013/129688. The nematicide M.29.8 is known from WO2013/055584. The isoxazoline M.29.9.a) is described in WO2013/050317. The isoxazoline M.29.9.b) is described in WO2014/126208. The pyridalyl-type analogue M.29.10 is known from WO2010/060379. The carboxamides broflanilide and M.29.11.b) to M.29.11.h) are described in WO2010/018714, and the carboxamides M.29.11i) to M.29.11.p) in WO2010/127926. The pyridylthiazoles M.29.12.a) to M.29.12.c) are known from WO2010/006713, M.29.12.d) and M.29.12.e) are known from WO2012/000896, and M.29.12.f) to M.29.12.m) from WO2010/129497. The compounds M.29.14a) and M.29.14b) are known from WO2007/101369. The pyrazoles M.29.16.a) to M.29.16h) are described in WO2010/034737, WO2012/084670, and WO2012/143317, respectively, and the pyrazoles M.29.16i) and M.29.16j) are described in U.S. 61/891,437. The pyridinylindazoles M.29.17a) to M.29.17.j) are described in WO2015/038503. The pyridylpyrazoles M.29.18a) to M.29.18d) are described in US2014/0213448. The isoxazoline M.29.19 is described in WO2014/036056. The isoxazoline M.29.20 is known from WO2014/090918.

The following list of fungicides, in conjunction with which the compounds of the present invention can be used, is intended to illustrate the possible combinations but does not limit them:

A) Respiration inhibitors
Inhibitors of complex III at Qₒ site (e.g. strobilurins): azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxy.strobin (A. 1.13), pyraclostrobin (A.1.14), pyrametostrobin (A.1.15), pyraoxystrobin (A. 1.16), trifloxystrobin (A.1.17), 2-(2-(3-(2,6-dichlorophenyl)-1-methyl-allylideneami nooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenyl-pyrazol-3-yl)oxylmethyl]phenyl]-N-methoxy-carbamate (A.1.22), 1-[3-chloro-2-[[1-(4-chlorophenyl)-1H-pyrazol-3-yl]oxymethyl]phenyl]-4-methyl-tetrazol-5-one (A. 1.23), 1-[3-bromo-2-[[1-(4-chlorophenyl)pyrazol-3-yl]oxymethyl]phenyl]-4-methyl-tetrazol-5-one (A. 1.24), 1-[2-[[1-(4-chlorophenyl)pyrazol-3-yl]oxymethyl]-3-methyl-phenyl]-4-methyl-tetrazol-5-one (A.1.25), 1-[2-[[1-(4-chlorophenyl)pyrazol-3-yl]oxymethyl]-3-fluoro-phenyl]-4-methyl-tetrazol-5-one (A.1.26), 1-[2-[[1-(2,4-dichlorophenyl)pyrazol-3-yl]oxymethyl]-3-fluoro-phenyl]-4-methyl-tetrazol-5-one (A.1.27), 1-[2-[[4-(4-chlorophenyl)thiazol-2-yl]oxymethyl]-3-methyl-phenyl]-4-methyl-tetrazol-5-one (A.1.28), 1-[3-chloro-2-[[4-(p-tolyl)thiazol-2-yl]oxymethyl]phenyl]-4-methyl-tetrazol-5-one (A.1.29), 1-[3-cyclopropyl-2-[[2-methyl-4-(1-methylpyrazol-3-yl)phenoxy]methyl]phenyl]-4-methyl-tetrazol-5-one (A.1.30), 1-[3-(difluoromethoxy)-2-[[2-methyl-4-(1-methylpyrazol-3-yl)phenoxy]methyl]phenyl]-4-methyl-tetrazol-5-one (A.1.31), 1-methyl-4-[3-methyl-2-[[2-methyl-4-(1-methylpyrazol-3-yl)phenoxy]methyl]phenyl]tetrazol-5-one (A.1.32), 1-methyl-4-[3-methyl-2-[[1-[3-(trifluoromethyl)phenyl]-ethylideneamino]oxymethyl]phenyl]tetrazol-5-one (A.1.33), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethyl-pent-3-enamide (A.1.35), (Z,2E)-5-[1-(4-chloro-2-fluoro-phenyl)pyrazol-3-yl]oxy-2-methoxyimino-N,3-dimethyl-pent-3-enamide (A.1.36),
inhibitors of complex III at Qi site: cyazofamid (A.2.1), amisulbrom (A.2.2), [(3S,6S,7R,8R)-8-benzyl-3-[(3-acetoxy-4-methoxy-pyridine-2-carbonyl)amino]-6-methyl-4,9-dioxo-1,5-dioxonan-7-yl] 2-methylpropanoate (A.2.3), [(3S,6S,7R,8R)-8-benzyl-3-[[3-(acetoxymethoxy)-4-methoxypyridine-2-carbonyl]amino]-6-methyl-4,9-dioxo-1,5-dioxonan-7-yl] 2-methylpropanoate (A.2.4), [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl] 2-methylpropanoate (A.2.5), [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl] 2-methylpropanoate (A.2.6); (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate (A.2.7), (3S,6S,7R,8R)-8-benzyl-3-[3-[(isobutyryloxy)methoxy]-4-methoxypicolinamido]-6-methyl-4,9-dioxo-1,5-dioxonan-7-yl isobutyrate (A.2.8);
inhibitors of complex II (e.g. carboxamides): benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.14), penthiopyrad (A.3.15), sedaxane (A.3.16), tecloftalam (A.3.17), thifluzamide (A.3.18), N-(4′-trifluoromethylthiobiphenyl-2-yl)-3-difluoromethyl-1-methyl-1H-pyrazole-4-carboxamide (A.3.19), N-(2-(1,3,3-trimethyl-butyl)-phenyl)-1,3-dimethyl-5-fluoro-1H-pyrazole-4-carboxamide (A.3.20), 3-(difluoromethyl)-1-methyl-N-(1,1,3-trimethylindan-4-yl)pyrazole-4-carboxamide (A.3.21), 3-(trifluoromethyl)-1-methyl-N-(1,1,3-trimethylindan-4-yl)pyrazole-4-carboxamide (A.3.22), 1,3-dimethyl-N-(1,1,3-trimethylindan-4-yl)pyrazole-4-carboxamide (A.3.23), 3-(trifluoromethyl)-1,5-dimethyl-N-(1,1,3-trimethylindan-4-yl)pyrazole-4-carboxamide (A.3.24), 1,3,5-trimethyl-N-(1,1,3-trimethylindan-4-yl)pyrazole-4-carboxamide (A.3.25), N-(7-fluoro-1,1,3-trimethyl-indan-4-yl)-1,3-dimethyl-pyrazole-4-carboxamide (A.3.26), N-[2-(2,4-dichlorophenyl)-2-methoxy-1-methyl-ethyl]-3-(difluoromethyl)-1-methyl-pyrazole-4-carboxamide (A.3.27);
other respiration inhibitors (e.g. complex I, uncouplers): diflumetorim (A.4.1), (5,8-difluoroquinazolin-4-yl)-{2-[2-fluoro-4-(4-trifluoromethylpyridin-2-yloxy)-phenyl]-ethyl}-amine (A.4.2); nitrophenyl derivates: binapacryl (A.4.3), dinobuton (A.4.4), dinocap (A.4.5), fluazinam (A.4.6); ferimzone (A.4.7); organometal compounds: fentin salts, such as fentin-acetate (A.4.8), fentin chloride (A.4.9) or fentin hydroxide (A.4.10); ametoctradin (A.4.11); and silthiofam (A.4.12);

B) Sterol biosynthesis inhibitors (SBI fungicides)
C14 demethylase inhibitors (DMI fungicides): triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 1-[rel-(2S,3R)-3-(2-chlorophenyl)-2-(2,4-difluorophenyl)-oxiranylmethyl]-5-thiocyanato-1H-[1,2,4]triazolo (B.1.31), 2-[rel-(2S;3R)-3-(2-chlorophenyl)-2-(2,4-difluorophenyl)-oxiranylmethyl]-2H-[1,2,4]triazole-3-thiol (B.1.32), 2-[2-chloro-4-(4-chlorophenoxy)phenyl]-1-(1,2,4-triazol-1-yl)pentan-2-ol (B.1.33), 1-[4-(4-chlorophenoxy)-2-(trifluoromethyl)phenyl]-1-cyclopropyl-2-(1,2,4-triazol-1-yl)ethanol (B.1.34), 2-[4-(4-chlorophenoxy)-2-(trifluoromethyl)phenyl]-1-(1,2,4-triazol-1-yl)butan-2-ol (B.1.35), 2-[2-chloro-4-(4-chlorophenoxy)phenyl]-1-(1,2,4-triazol-1-yl)butan-2-ol (B.1.36), 2-[4-(4-chlorophenoxy)-2-(trifluoromethyl)phenyl]-3-methyl-1-(1,2,4-triazol-1-yl)butan-2-ol (B.1.37), 2-[4-(4-chlorophenoxy)-2-(trifluoromethyl)phenyl]-1-(1,2,4-triazol-1-yl)propan-2-ol (B.1.38), 2-[2-chloro-4-(4-chlorophenoxy)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol (B.1.41), 2-[2-chloro-4-(4-chlorophenoxy)phenyl]-1-(1,2,4-triazol-1-yl)pent-3-yn-2-ol (B.1.51); imidazoles: imazalil (B.1.42), pefurazoate (B.1.43), prochloraz (B.1.44), triflumizol (B.1.45); pyrimidines, pyridines and piperazines: fenarimol (B.1.46), nuarimol (B.1.47), pyrifenox (B.1.48), triforine (B.1.49), [3-(4-chloro-2-fluoro-phenyl)-5-(2,4-difluorophenyl)isoxazol-4-yl]-(3-pyridyl)methanol (B.1.50);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);

C) Nucleic acid synthesis inhibitors
phenylamides or acyl amino acid fungicides: benalaxyl (C.1.1), benalaxyl-M (C.1.2), kiralaxyl (C.1.3), metalaxyl (C.1.4), metalaxyl-M (mefenoxam, C.1.5), ofurace (C.1.6), oxadixyl (C.1.7);
others: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7);

D) Inhibitors of cell division and cytoskeleton
tubulin inhibitors, such as benzimidazoles, thiophanates: benomyl (D1.1), carbendazim (D1.2), fuberidazole (D1.3), thiabendazole (D1.4), thiophanate-methyl (D1.5); triazolopyrimidines: 5-chloro-7-(4-methylpiperidin-1-yl)-6-(2,4,6-trifluorophenyl)-[1,2,4]triazolo[1,5-a]pyrimidine (D1.6);
other cell division inhibitors: diethofencarb (D2.1), ethaboxam (D2.2), pencycuron (D2.3), fluopicolide (D2.4), zoxamide (D2.5), metrafenone (D2.6), pyriofenone (D2.7);

E) Inhibitors of amino acid and protein synthesis
methionine synthesis inhibitors (anilino-pyrimidines): cyprodinil (E.1.1), mepanipyrim (E.1.2), pyrimethanil (E.1.3);
protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6), polyoxine (E.2.7), validamycin A (E.2.8);

F) Signal transduction inhibitors
MAP/histidine kinase inhibitors: fluoroimid (F.1.1), iprodione (F.1.2), procymidone (F.1.3), vinclozolin (F.1.4), fenpiclonil (F.1.5), fludioxonil (F.1.6);
G protein inhibitors: quinoxyfen (F.2.1);

G) Lipid and membrane synthesis inhibitors
Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7) and N-(1-(1-(4-cyano-phenyl)ethanesulfonyl)-but-2-yl) carbamic acid-(4-fluorophenyl) ester (G.3.8);
compounds affecting cell membrane permeability and fatty acides: propamocarb (G.4.1);
fatty acid amide hydrolase inhibitors: oxathiapiprolin (G.5.1), 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3);

H) Inhibitors with Multi Site Action
inorganic active substances: Bordeaux mixture (H.1.1), copper acetate (H.1.2), copper hydroxide (H.1.3), copper oxychloride (H.1.4), basic copper sulfate (H.1.5), sulfur (H.1.6);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds (e.g. phthalimides, sulfamides, chloronitriles): anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11), N-(4-chloro-2-nitro-phenyl)-N-ethyl-4-methylbenzenesulfonamide (H.3.12);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H[1,4]dithiino[2,3-c:5,6-c′]dipyrrole-1,3,5,7(2H,6H)-tetraone (H.4.10);

I) Cell wall synthesis inhibitors
inhibitors of glucan synthesis: validamycin (1.1.1), polyoxin B (1.1.2);
melanin synthesis inhibitors: pyroquilon (1.2.1), tricyclazole (1.2.2), carpropamid (1.2.3), dicyclomet (1.2.4), fenoxanil (1.2.5);

J) Plant defence inducers
acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), potassium or sodium bicarbonate (J.1.9);

K) Unknown mode of action
bronopol (K.1.1), chinomethionat (K.1.2), cyflufenamid (K.1.3), cymoxanil (K.1.4), dazomet (K. 1.5), debacarb (K. 1.6), diclomezine (K. 1.7), difenzoquat (K. 1.8), difenzoquat-methylsulfate (K.1.9), diphenylamin (K.1.10), fenpyrazamine (K.1.11), flumetover (K.1.12), flusulfamide (K.1.13), flutianil (K.1.14), methasulfocarb (K.1.15), nitrapyrin (K.1.16), nitrothal-isopropyl (K.1.18), oxathiapiprolin (K.1.19), tolprocarb (K.1.20), oxin-copper (K.1.21), proquinazid (K.1.22), tebufloquin (K.1.23), tecloftalam (K.1.24), triazoxide (K.1.25), 2-butoxy-6-iodo-3-propylchromen-4-one (K.1.26), 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone (K. 1.27), 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone (K.1.28), 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone (K.1.29), N-(cyclopropylmethoxyimino-(6-difluoro-methoxy-2,3-difluoro-phenyl)-methyl)-2-phenyl acetamide (K.1.30), N′-(4-(4-chloro-3-trifluoromethyl-phenoxy)-2,5-dimethyl-phenyl)-N-ethyl-N-methyl formamidine (K.1.31), N′-(4-(4-fluoro-3-trifluoromethyl-phenoxy)-2,5-dimethyl-phenyl)-N-ethyl-N-methyl formamidine (K.1.32), N′-(2-methyl-5-trifluoromethyl-4-(3-trimethylsilanyl-propoxy)-phenyl)-N-ethyl-N-methyl formamidine (K.1.33), N′-(5-difluoromethyl-2-methyl-4-(3-trimethylsilanyl-propoxy)-phenyl)-N-ethyl-N-methyl formamidine (K.1.34), methoxy-acetic acid 6-tert-butyl-8-fluoro-2,3-dimethylquinolin-4-yl ester (K.1.35), 3-[5-(4-methylphenyl)-2,3-dimethyl-isoxazolidin-3-yl]-pyridine (K.1.36), 3-[5-(4-chloro-phenyl)-2,3-dimethyl-isoxazolidin-3-yl]-pyridine (pyrisoxazole) (K.1.37), N-(6-methoxy-pyridin-3-yl) cyclopropanecarboxylic acid amide (K.1.38), 5-chloro-1-(4,6-dimethoxy-pyrimidin-2-yl)-2-methyl-1H-benzoimidazole (K.1.39), 2-(4-chloro-phenyl)-N-[4-(3,4-dimethoxy-phenyl)-isoxazol-5-yl]-2-prop-2-ynyloxy-acetamide, ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), 2-[2-[(7,8-difluoro-2-methyl-3-quinolyl)oxy]-6-fluoro-phenyl]propan-2-ol (K.1.43), 2-[2-fluoro-6-[(8-fluoro-2-methyl-3-quinolyl)oxy]phen-yl]propan-2-ol (K.1.44), 3-(5-fluoro-3,3,4,4-tetramethyl-3,4-dihydroisoquinolin-1-yl)quinoline (K.1.45), 3-(4,4-difluoro-3,3-dimethyl-3,4-dihydroisoquinolin-1-yl)quinoline (K.1.46), 3-(4,4,5-trifluoro-3,3-dimethyl-3,4-dihydroisoquinolin-1-yl)quinoline (K.1.47), 9-fluoro-2,2-dimethyl-5-(3-quinolyl)-3H-1,4-benzoxazepine (K.1.48).

The fungicides described by common names, their preparation and their activity e.g. against harmful fungi is known (cf.: http://www.alanwood.net/pesticides/); these substances are commercially available.

The fungicides described by IUPAC nomenclature, their preparation and their pesticidal activity is also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657, WO2012/168188, WO 2007/006670, WO 2011/77514; WO13/047749, WO 10/069882, WO 13/047441, WO 03/16303, WO 09/90181, WO 13/007767, WO 13/010862, WO 13/127704, WO 13/024009, WO 13/024010 and WO 13/047441, WO 13/162072, WO 13/092224, WO 11/135833).

In one embodiment, the present invention relates to mixtures of a compound of the present invention with a compound of the class of fipronils including fipronil, and ethiprole.

In one embodiment, the present invention relates to mixtures of a compound of the present invention with a compound of the class of pyrethroids including cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, permethrin, phenothrin, and pyrethrin (pyrethrum).

In one embodiment, the present invention relates to mixtures of a compound of the present invention with a compound of the class of neonicotinoids including acetamiprid, chlothianidin, dinotefuran, imidacloprid, nitenpyram, thiacloprid, and thiamethoxam

In one embodiment, the present invention relates to mixtures of a compound of the present invention with a compound of the class of spinosyns including spinosad, and spinetoram

In one embodiment, the present invention relates to mixtures of a compound of the present invention with a compound of the class of avermectins and milbemycins including abamectin, and emamectin

In one embodiment, the present invention relates to mixtures of a compound of the present invention with a compound of the class of uncouplers of oxidative phosphorylation including chlorfenapyr.

In one embodiment, the present invention relates to mixtures of a compound of the present invention with a compound of the class of mitochondrial complex I electron transport inhibitors including pyridaben, tebufenpyrad, and tolfenpyrad.

In one embodiment, the present invention relates to mixtures of a compound of the present invention with a compound of the class of voltage-dependent sodium channel blockers including indoxacarb, and metaflumizone.

In one embodiment, the present invention relates to mixtures of a compound of the present invention with a compound of the class of mitochondrial complex II electron transport inhibitors including cyenopyrafen, and cyflumetofen.

In one embodiment, the present invention relates to mixtures of a compound of the present invention with a compound of the class of diamides including flubendiamide, chlorantraniliprole, cyantraniliprole, cyclaniliprole, and tetraniliprole.

In one embodiment, the present invention relates to mixtures of a compound of the present invention with a compound of the class of inhibitors of complex III at Qₒ site including azoxystrobin, coumethoxystrobin, coumoxystrobin, dimoxystrobin, enestroburin, fenaminstrobin, fenoxystrobin/flufenoxystrobin, fluoxastrobin, kresoxim-methyl, mandestrobin, metominostrobin, orysastrobin, picoxystrobin, pyraclostrobin, pyrametostrobin, pyraoxystrobin, trifloxystrobin, pyribencarb, triclopyricarb/chlorodincarb, famoxadone, and fenamidone.

In one embodiment, the present invention relates to mixtures of a compound of the present invention with a compound of the class of inhibitors of complex II including benodanil, benzovindiflupyr, bixafen, boscalid, carboxin, fenfuram, fluopyram, flutolanil, fluxapyroxad, furametpyr, isofetamid, isopyrazam, mepronil, oxycarboxin, penflufen, penthiopyrad, sedaxane, tecloftalam, and thifluzamide.

In one embodiment, the present invention relates to mixtures of a compound of the present invention with other respiration inhibitors including diflumetorim, binapacryl, dinobuton, dinocap, fluazinam, ferimzone, fentin-acetate, fentin chloride, fentin hydroxide, ametoctradin, and silthiofam.

In one embodiment, the present invention relates to mixtures of a compound of the present invention with a compound of the class of C14 demethylase inhibitors including triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, imazalil, pefurazoate, prochloraz, triflumizol, fenarimol, nuarimol, pyrifenox, and triforine.

In one embodiment, the present invention relates to mixtures of a compound of the present invention with a compound of the class of delta14-reductase inhibitors including aldimorph, dodemorph, dodemorph-acetate, fenpropimorph, tridemorph, fenpropidin, piperalin, and spiroxamine.

In one embodiment, the present invention relates to mixtures of a compound of the present invention with a compound of the class of inhibitors of 3-keto reductase including fenhexamid.

In one embodiment, the present invention relates to mixtures of a compound of the present invention with a compound of the class of phenylamides or acyl amino acid fungicides including benalaxyl, benalaxyl-M, kiralaxyl, metalaxyl, metalaxyl-M (mefenoxam), ofurace, and oxadixyl.

In one embodiment, the present invention relates to mixtures of a compound of the present invention with a compound of the class of tubulin inhibitors from the class of benzimidazoles, and thiophanates: benomyl, carbendazim, fuberidazole, thiabendazole, thiophanatemethyl.

In one embodiment, the present invention relates to mixtures of a compound of the present invention with a compound of the class of methionine synthesis inhibitors from the class of anilino-pyrimidines: cyprodinil, mepanipyrim, and pyrimethanil.

In one embodiment, the present invention relates to mixtures of a compound of the present invention with a compound of the class of MAP/histidine kinase inhibitors including fluoroimid, iprodione, procymidone, vinclozolin, fenpiclonil, and fludioxonil.

In one embodiment, the present invention relates to mixtures of a compound of the present invention with a compound of the class of thio- and dithiocarbamates including ferbam, mancozeb, maneb, metam, metiram, propineb, thiram, zineb, and ziram.

In one embodiment, the present invention relates to mixtures of a compound of the present invention with a compound of the class of organochlorine compounds from the class of phthalimides, sulfamides, and chloronitriles: anilazine, chlorothalonil, captafol, captan, folpet, dichlofluanid, dichlorophen, hexachlorobenzene, pentachlorphenole and its salts, and phthalide.

In one embodiment, the present invention relates to mixtures of a compound of the present invention with a compound of the class of melanin synthesis inhibitors including pyroquilon, tricyclazole, carpropamid, dicyclomet, and fenoxanil.

The invention also relates to agrochemical compositions comprising an auxiliary and at least one compound of the present invention or a mixture thereof.

An agrochemical composition comprises a pesticidally effective amount of a compound of the present invention or a mixture thereof. The term “pesticidally effective amount” is defined below.

The compounds of the present invention or the mixtures thereof can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Examples for suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclo-hexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl-sulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl-naphthalenes, sulfosuccinates or sulfosuccinamates. Examples of sulfates are sulfates of fatty acids and oils, of ethoxylated alkylphenols, of alcohols, of ethox-ylated alcohols, or of fatty acid esters. Examples of phosphates are phosphate esters. Exam-ples of carboxylates are alkyl carboxylates, and carboxylated alcohol or alkylphenol eth-oxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-subsititued fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compounds of the present invention on the target. Examples are surfactants, mineral or vegetable oils, and other auxilaries.

Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

i) Water-soluble concentrates (SL, LS) 10-60 wt % of a compound according to the invention and 5-15 wt % wetting agent (e.g. alcohol alkoxylates) are dissolved in water and/or in a water-soluble solvent (e.g. alcohols) up to 100 wt %. The active substance dissolves upon dilution with water.

ii) Dispersible concentrates (DC)

5-25 wt % of a compound according to the invention and 1-10 wt % dispersant (e.g. polyvinylpyrrolidone) are dissolved in up to 100 wt % organic solvent (e.g. cyclohexanone). Dilution with water gives a dispersion.

iii) Emulsifiable concentrates (EC)

15-70 wt % of a compound according to the invention and 5-10 wt % emulsifiers (e.g. calcium dodecylbenzenesulfonate and castor oil ethoxylate) are dissolved in up to 100 wt % water-insoluble organic solvent (e.g. aromatic hydrocarbon). Dilution with water gives an emulsion.

iv) Emulsions (EW, EO, ES)

5-40 wt % of a compound according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into up to 100 wt % water by means of an emulsifying machine and made into a homogeneous emulsion. Dilution with water gives an emulsion.

v) Suspensions (SC, OD, FS)

In an agitated ball mill, 20-60 wt % of a compound according to the invention are comminuted with addition of 2-10 wt % dispersants and wetting agents (e.g. sodium lignosulfonate and alcohol ethoxylate), 0.1-2 wt % thickener (e.g. xanthan gum) and up to 100 wt % water to give a fine active substance suspension. Dilution with water gives a stable suspension of the active substance. For FS type composition up to 40 wt % binder (e.g. polyvinylalcohol) is added.

vi) Water-dispersible granules and water-soluble granules (WG, SG) 50-80 wt % of a compound according to the invention are ground finely with addition of up to 100 wt % dispersants and wetting agents (e.g. sodium lignosulfonate and alcohol ethoxylate) and prepared as water-dispersible or water-soluble granules by means of technical appliances (e.g. extrusion, spray tower, fluidized bed). Dilution with water gives a stable dispersion or solution of the active substance.

vii) Water-dispersible powders and water-soluble powders (WP, SP, WS)

50-80 wt % of a compound according to the invention are ground in a rotor-stator mill with addition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dispersion or solution of the active substance.

viii) Gel (GW, GF)

In an agitated ball mill, 5-25 wt % of a compound according to the invention are comminuted with addition of 3-10 wt % dispersants (e.g. sodium lignosulfonate), 1-5 wt % thickener (e.g. carboxymethylcellulose) and up to 100 wt % water to give a fine suspension of the active substance. Dilution with water gives a stable suspension of the active substance.

ix) Microemulsion (ME)

5-20 wt % of a compound according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water up to 100%. This mixture is stirred for 1 h to produce spontaneously a thermodynamically stable microemulsion.

x) Microcapsules (CS)

An oil phase comprising 5-50 wt % of a compound according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radi-cal initiator results in the formation of poly(meth)acrylate microcapsules. Alternatively, an oil phase comprising 5-50 wt % of a compound according to the invention, 0-40 wt % water insolu-ble organic solvent (e.g. aromatic hydrocarbon), and an isocyanate monomer (e.g. diphenylme-thene-4,4′-diisocyanatae) are dispersed into an aqueous solution of a protective colloid (e.g. polyvinyl alcohol). The addition of a polyamine (e.g. hexamethylenediamine) results in the for-mation of a polyurea microcapsule. The monomers amount to 1-10 wt %. The wt % relate to the total CS composition.

xi) Dustable powders (DP, DS)

1-10 wt % of a compound according to the invention are ground finely and mixed intimately with up to 100 wt % solid carrier, e.g. finely divided kaolin.

xii) Granules (GR, FG)

0.5-30 wt % of a compound according to the invention is ground finely and associated with up to 100 wt % solid carrier (e.g. silicate). Granulation is achieved by extrusion, spray-drying or the fluidized bed.

xiii) Ultra-low volume liquids (UL)

1-50 wt % of a compound according to the invention are dissolved in up to 100 wt % organic solvent, e.g. aromatic hydrocarbon.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and most preferably between 0.5 and 75%, by weight of active sub-stance. The active substances are employed in a purity of from 90% to 100%, preferably from 95% to 100% (according to NMR spectrum).

Various types of oils, wetters, adjuvants, fertilizer, or micronutrients, and other pesticides (e.g. herbicides, insecticides, fungicides, growth regulators, safeners) may be added to the active substances or the compositions com-prising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage de-vice, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e.g. components comprising compounds of the present invention and/or mixing partners as defined above, may be mixed by the user in a spray tank and further auxiliaries and additives may be added, if appropriate.

In a further embodiment, either individual components of the composition according to the invention or partially premixed components, e.g. components comprising compounds of the present invention and/or mixing partners as defined above, can be applied jointly (e.g. after tank mix) or consecutively.

The compounds of the present invention are suitable for use in protecting crops, plants, plant propagation materials, such as seeds, or soil or water, in which the plants are growing, from attack or infestation by animal pests. Therefore, the present invention also relates to a plant protection method, which comprises contacting crops, plants, plant propagation materials, such as seeds, or soil or water, in which the plants are growing, to be protected from attack or infestation by animal pests, with a pesticidally effective amount of a compound of the present invention.

The compounds of the present invention are also suitable for use in combating or controlling animal pests. Therefore, the present invention also relates to a method of combating or controlling animal pests, which comprises contacting the animal pests, their habitat, breeding ground, or food supply, or the crops, plants, plant propagation materials, such as seeds, or soil, or the area, material or environment in which the animal pests are growing or may grow, with a pesticidally effective amount of a compound of the present invention.

The compounds of the present invention are effective through both contact and ingestion. Furthermore, the compounds of the present invention can be applied to any and all developmental stages, such as egg, larva, pupa, and adult.

The compounds of the present invention can be applied as such or in form of compositions comprising them as defined above. Furthermore, the compounds of the present invention can be applied together with a mixing partner as defined above or in form of compositions comprising said mixtures as defined above. The components of said mixture can be applied simultaneously, jointly or separately, or in succession, that is immediately one after another and thereby creating the mixture “in situ” on the desired location, e.g. the plant, the sequence, in the case of separate application, generally not having any effect on the result of the control measures.

The application can be carried out both before and after the infestation of the crops, plants, plant propagation materials, such as seeds, soil, or the area, material or environment by the pests.

Suitable application methods include inter alia soil treatment, seed treatment, in furrow application, and foliar application. Soil treatment methods include drenching the soil, drip irrigation (drip application onto the soil), dipping roots, tubers or bulbs, or soil injection. Seed treatment techniques include seed dressing, seed coating, seed dusting, seed soaking, and seed pelleting. In furrow applications typically include the steps of making a furrow in cultivated land, seeding the furrow with seeds, applying the pesticidally active compound to the furrow, and closing the furrow. Foliar application refers to the application of the pesticidally active compound to plant foliage, e.g. through spray equipment. For foliar applications, it can be advantageous to modify the behavior of the pests by use of pheromones in combination with the compounds of the present invention. Suitable pheromones for specific crops and pests are known to a skilled person and publicly available from databases of pheromones and semiochemicals, such as http://www.pherobase.com.

As used herein, the term “contacting” includes both direct contact (applying the compounds/compositions directly on the animal pest or plant—typically to the foliage, stem or roots of the plant) and indirect contact (applying the compounds/compositions to the locus, i.e. habitat, breeding ground, plant, seed, soil, area, material or environment in which a pest is growing or may grow, of the animal pest or plant).

The term “animal pest” includes arthropods, gastropods, and nematodes. Preferred animal pests according to the invention are arthropods, preferably insects and arachnids, in particular insects. Insects, which are of particular relevance for crops, are typically referred to as crop insect pests.

The term “crop” refers to both, growing and harvested crops.

The term “plant” includes cereals, e.g. durum and other wheat, rye, barley, triticale, oats, rice, or maize (fodder maize and sugar maize/sweet and field corn); beet, e.g. sugar beet or fodder beet; fruits, such as pomes, stone fruits or soft fruits, e.g. apples, pears, plums, peaches, nectarines, almonds, cherries, papayas, strawberries, raspberries, blackberries or gooseberries; leguminous plants, such as beans, lentils, peas, alfalfa or soybeans; oil plants, such as rapeseed (oilseed rape), turnip rape, mustard, olives, sunflowers, coconut, cocoa beans, castor oil plants, oil palms, ground nuts or soybeans; cucurbits, such as squashes, pumpkins, cucumber or melons; fiber plants, such as cotton, flax, hemp or jute; citrus fruit, such as oranges, lemons, grapefruits or mandarins; vegetables, such as eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, such as avocados, cinnamon or camphor; energy and raw material plants, such as corn, soybean, rapeseed, sugar cane or oil palm; tobacco; nuts, e.g. walnuts; pistachios; coffee; tea; bananas; vines (table grapes and grape juice grape vines); hop; sweet leaf (also called Stevia); natural rubber plants or ornamental and forestry plants, such as flowers (e.g. carnation, petunias, geranium/pelargoniums, pansies and impatiens), shrubs, broad-leaved trees (e.g. poplar) or evergreens, e.g. conifers; eucalyptus; turf; lawn; grass such as grass for animal feed or ornamental uses. Preferred plants include potatoes sugar beets, tobacco, wheat, rye, barley, oats, rice, corn, cotton, soybeans, rapeseed, legumes, sunflowers, coffee or sugar cane; fruits; vines; ornamentals; or vegetables, such as cucumbers, tomatoes, beans or squashes.

The term “plant” is to be understood as including wild type plants and plants, which have been modified by either conventional breeding, or mutagenesis or genetic engineering, or by a combination thereof.

Plants, which have been modified by mutagenesis or genetic engineering, and are of particular commercial importance, include alfalfa, rapeseed (e.g. oilseed rape), bean, carnation, chicory, cotton, eggplant, eucalyptus, flax, lentil, maize, melon, papaya, petunia, plum, poplar, potato, rice, soybean, squash, sugar beet, sugarcane, sunflower, sweet pepper, tobacco, tomato, and cereals (e.g. wheat), in particular maize, soybean, cotton, wheat, and rice. In plants, which have been modified by mutagenesis or genetic engineering, one or more genes have been mutagenized or integrated into the genetic material of the plant. The one or more mutagenized or integrated genes are preferably selected from pat, epsps, crylAb, bar, cry1Fa2, cry1Ac, cry34Ab1, cry35AB1, cry3A, cryF, cry1F, mcry3a, cry2Ab2, cry3Bbl, cry1A.105, dfr, barnase, vip3Aa20, barstar, als, bxn, bp40, asn1, and ppo5. The mutagenesis or integration of the one or more genes is performed in order to improve certain properties of the plant. Such properties, also known as traits, include abiotic stress tolerance, altered growth/yield, disease resistance, herbicide tolerance, insect resistance, modified product quality, and pollination control. Of these properties, herbicide tolerance, e.g. imidazolinone tolerance, glyphosate tolerance, or glufosinate tolerance, is of particular importance. Several plants have been rendered tolerant to herbicides by mutagenesis, for example Clearfield® oilseed rape being tolerant to imidazolinones, e.g. imazamox. Alternatively, genetic engineering methods have been used to render plants, such as soybean, cotton, corn, beets and oil seed rape, tolerant to herbicides, such as glyphosate and glufosinate, some of which are commercially available under the trade names RoundupReady® (glyphosate) and LibertyLink® (glufosinate). Furthermore, insect resistance is of importance, in particular lepidopteran insect resistance and coleopteran insect resistance. Insect resistance is typically achieved by modifying plants by integrating cry and/or vip genes, which were isolated from Bacillus thuringiensis (Bt), and code for the respective Bt toxins. Genetically modified plants with insect resistance are commercially available under trade names including WideStrike®, Bollgard®, Agrisure®, Herculex®, YieldGard®, Genuity®, and Intacta®. Plants may be modified by mutagenesis or genetic engineering either in terms of one property (singular traits) or in terms of a combination of properties (stacked traits). Stacked traits, e.g. the combination of herbicide tolerance and insect resistance, are of increasing importance. In general, all relevant modified plants in connection with singular or stacked traits as well as detailed information as to the mutagenized or integrated genes and the respective events are available from websites of the organizations “International Service for the Acquisition of Agri-biotech Applications (ISAAA)” (http://www.isaaa.org/gmapprovaldatabase) and “Center for Environmental Risk Assessment (CERA)” (http://cera-gmc.org/GMCropDatabase).

It has surprisingly been found that the pesticidal activity of the compounds of the present invention may be enhanced by the insecticidal trait of a modified plant. Furthermore, it has been found that the compounds of the present invention are suitable for preventing insects to become resistant to the insecticidal trait or for combating pests, which already have become resistant to the insecticidal trait of a modified plant. Moreover, the compounds of the present invention are suitable for combating pests, against which the insecticidal trait is not effective, so that a complementary insecticidal activity can advantageously be used.

The term “plant propagation material” refers to all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “seed” embraces seeds and plant propagules of all kinds including but not limited to true seeds, seed pieces, suckers, corms, bulbs, fruit, tubers, grains, cuttings, cut shoots and the like, and means in a preferred embodiment true seeds.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment, in furrow application or of application to the pests dwelling place or nest, the quantity of active ingredient ranges from 0.0001 to 500 g per 100 m², preferably from 0.001 to 20 g per 100 m².

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare, more desirably from 10 g to 50 g per hectare, e.g., 10 to 20 g per hectare, 20 to 30 g per hectare, 30 to 40 g per hectare, or 40 to 50 g per hectare.

The compounds of the present invention are particularly suitable for use in the treatment of seeds in order to protect the seeds from insect pests, in particular from soil-living insect pests, and the resulting seedling's roots and shoots against soil pests and foliar insects. The present invention therefore also relates to a method for the protection of seeds from insects, in particular from soil insects, and of the seedling's roots and shoots from insects, in particular from soil and foliar insects, said method comprising treating the seeds before sowing and/or after pregermination with a compound of the present invention. The protection of the seedling's roots and shoots is preferred. More preferred is the protection of seedling's shoots from piercing and sucking insects, chewing insects and nematodes.

The term “seed treatment” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The present invention also comprises seeds coated with or containing the active compound. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is for example seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which have been modified by mutagenisis or genetic engineering, and which e.g. tolerate the action of herbicides or fungicides or insecticides. Such modified plants have been described in detail above.

Conventional seed treatment formulations include for example flowable concentrates FS, solutions LS, suspoemulsions (SE), powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter. Preferably, the formulations are applied such that germination is not included.

The active substance concentrations in ready-to-use formulations, which may be obtained after two-to-tenfold dilution, are preferably from 0.01 to 60% by weight, more preferably from 0.1 to 40% by weight.

In a preferred embodiment a FS formulation is used for seed treatment. Typically, a FS formulation may comprise 1-800 g/l of active ingredient, 1-200 g/I Surfactant, 0 to 200 g/I antifreezing agent, 0 to 400 g/l of binder, 0 to 200 g/l of a pigment and up to 1 liter of a solvent, preferably water.

Especially preferred FS formulations of the compounds of the present invention for seed treatment usually comprise from 0.1 to 80% by weight (1 to 800 g/I) of the active ingredient, from 0.1 to 20% by weight (1 to 200 g/I) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

In the treatment of seed, the application rates of the compounds of the invention are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed, e.g. from 1 g to 100 g or from 5 g to 100 g per 100 kg of seed.

The invention therefore also relates to seed comprising a compound of the present invention, or an agriculturally useful salt thereof, as defined herein. The amount of the compound of the present invention or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

The compounds of the present invention may also be used for improving the health of a plant. Therefore, the present invention also relates to a method for improving plant health by treating a plant, plant propagation material and/or the locus where the plant is growing or is to grow with an effective and non-phytotoxic amount of a compound of the present invention.

As used herein “an effective and non-phytotoxic amount” means that the compound is used in a quantity which allows to obtain the desired effect but which does not give rise to any phytotoxic symptom on the treated plant or on the plant grown from the treated propagule or treated soil.

The terms “plant” and “plant propagation material” are defined above.

“Plant health” is defined as a condition of the plant and/or its products which is determined by several aspects alone or in combination with each other such as yield (for example increased biomass and/or increased content of valuable ingredients), quality (for example improved content or composition of certain ingredients or shelf life), plant vigour (for example improved plant growth and/or greener leaves (“greening effect”), tolerance to abiotic (for example drought) and/or biotic stress (for example disease) and production efficiency (for example, harvesting efficiency, processability).

The above identified indicators for the health condition of a plant may be interdependent and may result from each other. Each indicator is defined in the art and can be determined by methods known to a skilled person.

The compounds of the invention are also suitable for use against non-crop insect pests. For use against said non-crop pests, compounds of the present invention can be used as bait composition, gel, general insect spray, aerosol, as ultra-low volume application and bed net (impregnated or surface applied). Furthermore, drenching and rodding methods can be used.

As used herein, the term “non-crop insect pest” refers to pests, which are particularly relevant for non-crop targets, such as ants, termites, wasps, flies, ticks, mosquitos, crickets, or cockroaches.

The bait can be a liquid, a solid or a semisolid preparation (e.g. a gel). The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature (e.g. http://www.pherobase.com), and are known to those skilled in the art.

For use in bait compositions, the typical content of active ingredient is from 0.001 weight % to 15 weight %, desirably from 0.001 weight % to 5% weight % of active compound.

Formulations of the compounds of the present invention as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents, furthermore auxiliaries such as emulsifiers, perfume oils, if appropriate stabilizers, and, if required, propellants.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of the present invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the present invention and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder.

The compounds of the present invention and its compositions can be used for protecting wooden materials such as trees, board fences, sleepers, frames, artistic artifacts, etc. and buildings, but also construction materials, furniture, leathers, fibers, vinyl articles, electric wires and cables etc. from ants and/or termites, and for controlling ants and termites from doing harm to crops or human being (e.g. when the pests invade into houses and public facilities).

Customary application rates in the protection of materials are, for example, from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

The compounds of the the present invention are especially suitable for efficiently combating animal pests such as arthropods, gastropods and nematodes including but not limited to: insects from the order of Lepidoptera, for example Achroia grisella, Acleris sspp. such as A. fimbriana, A. gloverana, A. variana; Acrolepiopstis assectella, Acronicta major, Adoxophyes spp. such as A. cyrtosema, A. orana; Aedia leucomelas, Agrotis spp. such as A. exclamationis, A. fucosa, A. ipsilon, A. orthogoma, A. segetum, A. subterranea; Alabama arglllacea, Aleurodicus dispersus, Alsophila pometaria, Ampelophaga rubiginosa, Amyelotis transitella, Anacampstis sarcitella, Anagasta kuehniella, Anarsia lineatella, Antisota senatoria, Antheraea pemyi, Anticarsia (=Thermesia) spp. such as A. gemmatalis; Apamea spp., Aproaerema modicella, Archips spp. such as A. argyrospila, A. fuscocupreanus, A. rosana, A. xyloseanus; Argyresthia conjugella, Argyroploce spp., Argyrotaenia spp. such as A. velutinana; Athetis mindara, Austroasca viridignisea, Autographa gamma, Autographa nigrisigna, Barathra brassicae, Bedellia spp., Bonagota salubricola, Borbo cinnara, Bucculatrix thurberiella, Bupalus piniarius, Busseola spp., Cacoecia spp. such as C. murinana, C. podana; CactoblastIS cactorum, Cadra cautella, Calingo braziliensis, Calopas theivora, Capua reticulana, Carposina spp. such as C. niponensis, C. sasakii; Cephus spp., Chaetocnema aridula, Cheimatobia brumata, Chilospp. such as C. Indicus, C. suppressalis, C. partellus; Choreutis pariana, Choristoneura spp. such as C. conflictana, C. fumiferana, C. longicellana, C. murinana, C. occidentalis, C. rosaceana; Chrysodeixis (=Pseudoplusia) spp. such as C. eriosoma, C. includens; Cirphtis unipuncta, Clysia ambiguella, Cnaphalocerus spp., Cnaphalocroctis medinalis, Cnephasia spp., Cochyilis hospes, Coleophora spp., Colias eurytheme, Conopomorpha spp., Conotrachelus spp., Copitarsia spp., Corcyra cephalonica, Crambus caliginosellus, Crambus teterrellus, Crocidosema (=Epinotia) aporema, Cydalima (=Diaphania) perspectalis, Cydia (=Carpocapsa) spp. such as C. pomonella, C. latiferreana; Dalaca noctuides, Datana integerrima, Dasychira pinicola, Dendrolimus spp. such as D. pini, D. spectabilis, D. sibiricus; Desmia funeralis, Diaphania spp. such as D. nitidalis, D. hyalinata; Diatraea grandiosella, Diatraea saccharalis, Diphthera festiva, Earias spp. such as E. insulana, E. vittella; Ecdytolopha aurantianu, Egira (=Xylomyges) curiails, Elasmopalpus lignosellus, Eldana saccharina, Endopiza viteana, Ennomos subsignaria, Eoreuma loftini, Ephestia spp. such as E. cautella, E. elutella, E. kuehniella; Epinotia aporema, Epiphyas postvittana, Eranntis tiliaria, Erionota thrax, Etiella spp., Eulia spp., Eupoecilia ambiguella, Euproctis chrysorrhoea, Euxoa spp., Evetria bouliana, Faronta albilinea, Feltia spp. such as F. subterranean; Galleria mellonella, Gracillaria spp., Grapholita spp. such as G. funebrana, G. molesta, G. inopinata; Halysidota spp., Harrisina americana, Hedylepta spp., Helicoverpa spp. such as H. armigera (=HeliothiS armigera), H. zea (=HeliothiS zea); Heliothis spp. such as H. assulta, H. subtlexa, H. virescens; Hellula spp. such as H. undalis, H. rogatails; Helocoverpa gelotopoeon, Hemileuca oliviae, Herpetogramma licarsisalis, Hibernia defoliaria, Hofmannophila pseudospretella, Homoeosoma electellum, Homona magnanima, Hypena scabra, Hyphantria cunea, Hyponomeuta padella, Hyponomeuta malinellus, Kakivoria flavofasciata, Keiferia lycopersicella, Lambdina fiscellaria, fiscellaria, Lambdina fiscellaria lugubrosa, Lamprosema indicata, Laspeyresia molesta, Leguminivora glycinivorella, Lerodea eufala, Leucinodes orbonalis, Leucoma salicis, Leucoptera spp. such as L. coffeella, L. scitella; Leuminivora lycinivorella, Lithocollegs blancardella, Lithophane antennata, Llattia octo (=Amyna axis), Lobesia botrana, Lophocampa spp., Loxagrogs albicosta, Loxostege spp. such as L. sticticalis, L. cereralis; Lymantria spp. such as L. dispar, L. monacha; Lyonetia clerkella, Lyonetia prunifoliella, Malacosoma spp. such as M. americanum, M. californicum, M. constrictum, M. neustria; Mamestra spp. such as M. brassicae, M. configurata; Mamstra brassicae, Manduca spp. such as M. quinquemaculata, M. sexta; Marasmia spp, Marmara spp., Maruca testulalis, Megalopyge lanata, Melanchra picta, Melanitis leda, Mocis spp. such as M. lapites, M. repanda; Mocis latipes, Monochroa fragariae, Mythirnna separata, Nemapogon cloacella, Neoleucinodes elegantalis, Nepytia spp., Nymphula spp., Oiketicus spp., Omiodes indicata, Omphisa anastomosalis, Operophtera brumata, Orgyia pseudotsugata, Oria spp., Orthaga thyrisalis, Ostrinia spp. such as O. nubilalis; Oulema oryzae, Paleacrita vernata, Panolos fiammea, Parnara spp., Papaipema nebnis, Papilio cresphontes, ParamyelotS transitella, Paranthrene regalis, Paysandisia archon, Pectinophora spp. such as P. gossypiella; Peridroma saucia, Perileucoptera spp., such as P. coffeella; Phalera bucephala, Phoganidia californica, Phthorirnaea spp. such as P. operculella; Phyllocnitis citrella, Phyllonoycter spp. such as P. blancardella, P. crataegella, P. issikii, P. ringoniella; Pieris spp. such as P. brassicae, P. rapae, P. napi; Pilocrocis tripunctata, Plathypena scabra, Platynota spp. such as P. flavedana, P. idaeusalis, P. stultana; Platyptilia carduidactyla, Plebejus argus, Plodia interpunctella, Plusia spp, Plutella maculipennis, Plutella xylostella, Pontia protodica, Prays spp., Prodenia spp., Proxenus lepigone, Pseudaletia spp. such as P. sequax, P. unipuncta; Pyrausta nubilalis, Rachiplusia nu, Richia albicosta, Rhizobius ventralis, Rhyacionia frustrana, Sabulodes aegrotata, Schizura concinna, Schoenobius spp., Schreckensteinia festaliella, Scirpophaga spp. such as S. incertulas, S. innotata; Scotia segetum, Sesamia spp. such as S. inferens, Seudyra subfiava, Sitotroga cerealella, Sparganothis pilleriana, Spilonota lechriaspis, S. ocellana, Spodoptera (=Lamphygma) spp. such as S. cosmoides, S. eridania, S. exigua, S. frugiperda, S. lagsfascia, S. littoralis, S. litura, S. omithogalli; Stigmella spp., Stomopteryx subsecivella, Strymon bazochii, Sylepta derogata, Synanthedon spp. such as S. exitiosa, Tecia solanivora, Telehin licus, Thaumatopoea pityocampa, Thaumatotibia (=Ctyptophlebia) leucotreta, Thaumetopoea pityocampa, Thecla spp., Theresimima ampelophaga, Thyrinteina spp, Tildenia inconspicuella, Tinea spp. such as T. cloacella, T. pellionella; Tineola bisselliella, Tortrix spp. such as T. viridana; Trichophaga tapetzella, Trichoplusia spp. such as T. ni; Tuta (=Scrobipalpula) absoluta, Udea spp. such as U. rubigalis, U. rubigalis; Virachola spp., Yponomeuta padella, and Zeiraphera canadensis;

insects from the order of Coleoptera, for example Acalymma vittatum, Acanthoscehdes obtectus, Adoretus spp., Agelastica alni, Agrilus spp. such as A. anxius, A. planipennis, A. sonuatus; Agriotes spp. such as A. fuscicollis, A. lineatus, A. obscurus; Alphitobius diaperinus, Amphirnallus solstitialis, Anisandrus dispar, Antisoplia austriaca, Anobium punctatum, Anomala corpulenta, Anomala rufocuprea, Anoplophora spp. such as A. glabripennis; Anthonomus spp. such as A. eugenit, A. grandis, A. pomorum; Anthrenus spp., Aphthona euphoridae, Apion spp., Apogonia spp., Athous haemorrhoidalis, Atomaria spp. such as A. linearis; Attagenus spp., Aulacophora Femoralis, Blastophagus piniperda, Blitophaga undata, Bruchidius obtectus, Bruchus spp. such as B. lentis, B. pisorum, B. rufimanus; Byctiscus betulae, Callidiellum rutipenne, Callopistria floridensis, Callosobruchus chinensis, Cameraria ohndella, Cassida nebulosa, Cerotoma trifurcata, Cetonia aurata, Ceuthorhynchus spp. such as C. assimilis, C. napi Chaetocnema tibialis, Cleonus mendicus, Conoderus spp. such as C. vespertinus; Conotrachelus nenuphar, Cosmopolites spp., Costelytra zealandica, Criocenis asparagi, Ctyptolestes ferrugineus, Ctyptorhynchus lapathi, Ctenicera spp. such as C. destructor; Curculio spp., Cylindrocopturus spp., Cyclocephala spp., Dactylispa balyi, Dectes texanus, Dermestes spp., Diabrotica Spp. Such as D. indecimpunctata, D. speciosa, D. longicornis, D. semipunctata, D. virgifera; Diaprepes abbreviates, Dichocroctis spp., Dicladispa armigera, Diloboderus abderus, Diocalandra frumenti (Diocalandra stigmaticollis), Enaphalodes rufulus, Epilachna spp. such as E. varivestis, E. vigintioctomaculata; Epitrix spp. such as E. hirtipennis, E. similaris; Eutheola humilis, Eutinobothrus brasiliensis, Faustinus cubae, Gibbium psylloides, Gnathocerus cornutus, Hellula undalis, Heteronychus arator, Hylamorpha elegans, Hylobius abietis, Hylotrupes bajulus, Hypera spp. such as H. brunneipennis, H. postica; Hypomeces squamosus, Hypothenemus spp., Ips typographus, Lachnosterna consanguinea, Lasioderma serricome, Latheticus oryzae, Lathridius spp., Lema spp. such as L. bilineata, L. melanopus; Leptinotarsa spp. such as L. decemlineata; Leptispa pygmaea, Limonius californicus, Lissorhoptrus oyzophilus, Lixus spp., Luperodes spp., Lyctus spp. such as L. bruneus; Liogenys fuscus, Macrodactylus spp. such as M. subspinosus; Maladera matrida, Megaplatypus mutates, Megascelis spp., Melanotus communis, Meligethes spp. such as M. aeneus; Melolontha spp. such as M. hippocastani, M. melolontha; Metamasius hemipterus, Microtheca spp., Migdolus spp. such as M. fryanus, Monochamus spp. such as M. alternatus; Naupactus xanthographus, Niptus hololeucus, Oberia brevis, Oemona hirta, Oryctes rhinoceros, Oryzaephilus surinamensis, Oryzaphagus oryzae, Otiorrhynchus sulcatus, Otiorrhynchus ovatus, Otiorrhynchussulcatus, Oulema melanopus, Oulema oryzae, Oxycetonia jucunda, Phaedon spp. such as P. brassicae, P. cochleariae; Phoracantha recurva, Phyllobius pyri, Phyllopertha horticola, Phyllophaga spp. such as P. helleri, Phyllotreta spp. such as P. chrysocephala, P. nemorum, P. striolata, P. vittula; Phyllopertha horticola, Popillia japonica, Premnotrypes spp., Psacothea hilaris, Psylliodes chrysocephala, Prostephanus truncates, Psylliodes spp., Ptinus spp., Pulga saltona, Rhizopertha dominica, Rhynchophorus spp. such as R. billineatus, R. ferrugineus, R. palmarum, R. phoenicis, R. vulneratus; Saperda candida, Scolytus schevyrewi, Scyphophorus acupunctatus, Sitona lineatus, Sitophllus spp. such as S. granaria, S. oryzae, S. zeamais; Sphenophorus spp. such as S. levis; Stegobium paniceum, Sternechus spp. such as S. subsignatus; Strophomorphus ctenotus, Symphyletes spp., Tanymecus spp., Tenebrio molitor, Tenebrioides mauretanicus, Tribolium spp. such as T. castaneum; Trogoderma spp., Tychius spp., Xylotrechus spp. such as X. pyrrhoderus; and, Zabrus spp. such as Z. tenebrioides;

insects from the order of Diptera for example Aedes spp. such asA. aegypti, A. albopictus, A. vexans; Anastrepha ludens, Anopheles spp. such as A. albirnanus, A. crucians, A. freeborn, A. gambiae, A. leucosphyrus, A. maculipennis, A. minimus, A. quadrimaculatus, A. sinensis; Bactrocera invadens, Bibio hortulanus, Calliphora erythrocephala, Calliphora vicina, Ceratitis capitata, Chrysomyia spp. such as C. bezziana, C. hominivorax, C. macellaria; Chrysops atlanticus, Chrysops discalis, Chrysops silacea, Cochliomyia spp. such as C. hominivorax; Contarinia spp. such as C. sorghicola; Cordylobia anthropophaga, Culex spp. such as C. nigripalpus, C. pipiens, C. quinquefasciatus, C. tarsalis, C. tritaeniorhynchus; Culicoides furens, Culiseta inornata, Culiseta melanura, Cuterebra spp., Dacus cucurbitae, Dacus oleae, Dasineura brassicae, Dasineura oxycoccana, Delia spp. such as D. antique, D. coarctata, D. platura, D. radicum; Dermatobia hominis, Drosophila spp. such as D. suzukit, Fannia spp. such as F. canicularis; Gastraphilus spp. such as G. intestinalis; Geomyza tipunctata, Glossina spp. such as G. fuscipes, G. morsitans, G. palpalis, G. tachinoides; Haematobia irratans, Haplodiplostis equestris, Hippelates spp., Hylemyia spp. such as H. platura; Hypoderma spp. such as H. lineata; Hyppobosca spp., Hydrellia philippina, Leptoconops torrens, Liriomyza spp. such as L. sativae, L. trifolii; Lucilia spp. such as L. caprin, L. cuprina, L. sericata; Lycoria pectoralis, Mansonia titillanus, Mayetiola spp. such as M. destructor; Musca spp. such as M. autumnalis, M. dourestica; Muscina stabulans, Oestrus spp. such as O. ovis; Opomyza fiorum, Oscinella spp. such as O. frit; Orseolia oryzae, Pegomya hysocyami, Phlebotomus argentipes, Phorbia spp. such as P. antiqua, P. brassicae, P. coarctata; Phytomyza gymnostoma, Prosirnulium mixtum, Psila rosae, Psorophora columbiae, Psorophora discolor, Rhagolettis spp. such as R. cerasi, R. cingulate, R. indifferens, R. mendax, R. pornonella; Rivellia quadrifasciata, Sarcophaga spp. such as S. haemorrhoidalis; Simulium vittatum, Sitodiplostis mosellana, Stomoxys spp. such as S. calcitrans; Tabanus spp. such as T. atratus, T. bovinus, T. lineola, T. similis; Tannia spp., Thecodiplostis japonensis, Tipula oleracea, Tipula paludosa, and Wohlfahrtia spp;

insects from the order of Thysanoptera for example, Baliothrips biformis, Dichromothrips corbetti, Dichromothrips ssp., Echinothrips americanus, Enneothrips flavens, Frankliniella spp. such as F. fusca, F. occidentalis, F. tritici; Heliothrips spp., Hercinothrips femoralis, Kakothrips spp., Microcephalothrips abdominalis, Neohydatothrips samayunkur, Pezothrips kellyanus, Rhoiphorothrips cruentatus, Scirtothrips spp. such as S. citri S. dorsalis, S. perseae; Stenchaetothrips spp, Taeniothrips cardamoni, Taeniothrips inconsequens, Thrips spp. such as T. imagines, T. hawaiiensis, T. oryzae, T. palmi, T. parvispinus, T. tabaci;

insects from the order of Hemiptera for example, Acizzia jamatonica, Acrostemum spp. such as A. hilare; Acyrthosipon spp. such as A. onobrychis, A. pisum; Adelges laricis, Adelges tsugae, Adelphoconis spp., such as A. rapidus, A. superbus; Aeneolamia spp., Agonoscena spp., Aulacorthum solani, Aleurocanthus woglumi, Aleurodes spp., Aleurodicus disperses, Aleurolobus barodensis, Aleurothrbas spp., Amrasca spp., Anasa tristis, Antestiopstis spp., Anuraphis cardui, Aonidiella spp., Aphanostigma piri, Aphidula nasturtit, Aphis spp. such as A. craccivora, A. fabae, A. forbesi, A. gossypit, A. grossulariae, A. maidiradicis, A. pouri, A. sambuci, A. schneideri, A. spiraecola; Arboridia apicalis, Arilus critatus, Aspidiella spp., Aspidiotus spp., Atanus spp., Aulacaspis yasumatsui, Aulacorthum solani, Bactericera cockerelli (Paratrioza cockerelli), Bemisia spp. such as B. argentifolii, B. tabaci (Aleurodes tabaci); Blissus spp. such as B. leucopterus; Brachycaudus spp. such as B. cardui, B. helichrysi, B. persicae, B. prunicola; Brachycolus spp., Brachycorynella asparagi, Brevicoryne brassicae, Cacopsylla spp. such as C. fulguralis, C. pyricola (Psylla piri); Calligypona marginata, Caloconis spp., Campylomma livida, Capitophorus horni, Cameocephala fulgida, Cavelerius spp., Ceraplastes spp., Ceratovacuna lanigera, Ceroplastes ceriferus, Cerosipha gossypit, Chaetosiphon fragaefolii, Chionaspis tegalensis, Chlorita onukit, Chromaphil juglandicola, Chtysomphalus ficus, Cicadulina mbila, Cirnex spp. such as C. hemipterus, C. lectularius; Coccomytilus halli, Coccus spp. such as C. hesperidum, C. pseudomagnoliarum; Corythucha arcuata, Creontiades dilutus, Cryptomyzus ribis, Chtysomphalus aonidum, Cryptomyzus ribis, Ctenarytaina spatulata, Cyrtopeltis notatus, Dalbulus spp., Dasynus piperis, Dialeurodes spp. such as D. citrifolii; Dalbulus maidis, Diaphorina spp. such as D. citri; Diaspis spp. such as D. bromeliae; Dichelops furcatus, Diconoconis hewetti, Doralis spp., Dreyfusia nordmannianae, Dreyfusia piceae, Drosicha spp., Dysaphtis spp. such as D. plantaginea, D. pyri, D. radicola; Dysaulacorthum pseudosolani, Dysdercus spp. such as D. cingulatus, D. intermedius; Dysmicoccus spp., Edessa spp., Geoconis spp., Empoasca spp. such as E. fabae, E. solana; Epidiaspis leperii, Eriosoma spp. such as E. lanigerum, E. pyricola; Erythroneura spp., Eutygaster spp. such as E. integriceps; Euscelis bllobatus, Euschtistus spp. such as E. heros, E. impictiventris, E. serous Fiorinia theae, Geococcus coffeae, Glycaspis brimblecombei, Halyomorpha spp. such as H. halys; Heliopelttis spp., Homalodtisca vitripenntis (=H. coagulata), Horcias nobilellus, Hyalopterus pruni, Hyperomyzus lactucae, Icelya spp. such as I. purchase; Idiocerus spp., Idioscopus spp., Laodelphax striatellus, Lecanium spp., Lecanoideus floccissimus, Lepidosaphes spp. such as L. ulmi; Leptocorisa spp., Leptoglossus phyllopus, Lipaphis Lygus spp. such as L. hesperus, L. lineolaris, L. pratensis; Maconellicoccus hirsutus, Marchalina hellenica, Macropes excavatus, Macrosiphum spp. such as M. rosae, M. avenae, M. euphorbiae; Macrosteles quadfilineatus, Mahanarva fimbriolata, Megacopta cribraria, Megoura viciae, Melanaphis pyrarius, Melanaphis sacchan, Melanocallis (=Tinocallis) caryaefoliae, Metcafiella spp., Metopolophium dirhodum, Monellia costalis, Monelliopstis pecanis, Myzocallis coryli, Murgantia spp., Myzus spp. such as M. ascalonicus, M. cerasi, M. nicotianae, M. persicae, M. varians; Nasonovia ribis nigri, Neotoxoptera Formosan, Neomegalotomus spp, Nephotettix spp. such as N. malayanus, N. nigropictus, N. parvus, N. virescens; Nezara spp. such as N. viridula; Nilaparvata lugens, Nysius huttoni, Oebalus spp. such as O. pugnax; Oncometopia spp., Orthezia praeionga, Oxycaraenus hyalinipennis, Parabemisia myricae, Parlatoria spp., Parthenolecanium spp. such as P. corni, P. persicae; Pemphigus spp. such as P. bursarius, P. populivenae; Peregrinus maidis, Perkinsiella saccharicida, Phenacoccus spp. such as P. aceris, P. gossypii; Phloeomyzus passerinit, Phorodon humuli, Phylloxera spp. such as P. devastatrix, Piesma quadrata, Piezodorus spp. such as P. guildinii; Pinnaspis aspidtstrae, Planococcus spp. such as P. citri, P. ficus; Prosapia bicincta, Protopulvinaria pyriformis, Psallus seriatus, Pseudacysta persea, Pseudaulacaspis pentagons, Pseudococcus spp. such as P. comstocki, Psylla spp. such as P. mali; Pteromalus spp., Pulvinaria amygdali, Pyrilla spp., Quadraspidiotus spp., such as Q. perniciosus; Quesada gigas, Rastrococcus spp., Reduvius senilis, Rhizoecus americanus, Rhodnius spp., Rhopalomyzus ascalonicus, Rhopalosiphum spp. such as R. pseudobrassicas, R. insertum, R. maidis, R. padi; Sagatodes spp., Sahlbergella singularis, Saissetia spp., Sappaphis mala, Sappaphis malt, Scaptocoris spp., Scaphoides titanus, Schizaphtis graminum, Schizoneura lanuginosa, Scotinophora spp., Selenaspidus articulatus, Sitobion avenae, Sogata spp., Sogatella furcifera, Solubea insularis, Spissistilus festinus (=Stictocephala festina), Stephanitis nashi, Stephanitis pyrioides, Stephanitis takeyai, Tenalaphara malayensis, Tetraleurodes perseae, Therioaphis maculate, Thyanta spp. such as T. accerra, T. perditor; Tibraca spp., Tomasapis spp., Toxoptera spp. such as T. aurantitii; Trialeurodes spp. such as T. abutilonea, T. ricini, T. vaporariorum; Triatoma spp., Trioza spp., Typhlocyba spp., Unaspis spp. such as U. citri, U. yanonensis; and Viteus vitifolii,

Insects from the order Hymenoptera for example Acanthomyops interjectus, Athalia rosae, Atta spp. such as A. capiguara, A. cephalotes, A. cephalotes, A. laevigata, A. robusta, A. sexdens, A. texana, Bombus spp., Brachymyrmex spp., Camponotus spp. such as C. floridanus, C. pennsylvanicus, C. modoc; Cardiocondyla nuda, Chalibion sp, Crematogaster spp., Dasymutilla occidentalis, Diprion spp., Dolichovespula maculata, Doymyrmex spp., Dryocosmus kuriphilus, Formica spp., Hoplocampa spp. such as H. minuta, H. testudinea; Indomyrmex humilis, Lasius spp. such as L. niger, Linepithema humile, Liometopum spp., Leptocybe invasa, Monomorium spp. such as M. pharaonis, Monomorium, Nylandria fulva, Pachycondyla chinensis, Paratrechina longicornis, Paravespula spp., such as P. germanica, P. pennsylvanica, P. vulgaris; Pheidole spp. such as P. megacephala; Pogonomyrmex spp. such as P. barbatus, P. californicus, Polistes rubiginosa, Prenoleptis impairs, Pseudomyrmex gracflis, Schelipron spp., Sirex cyaneus, Solenopstis spp. such as S. geminata, S. invicta, S. molesta, S. richteri, S. xyloni, Sphecius speciosus, Sphex spp., Tapinoma spp. such as T. melanocephalum, T. sessile; Tetramorium spp. such as T. caespitum, T. bicarinatum, Vespa spp. such as V. crabro; Vespula spp. such as V. squamosal; Wasmannia auropunctata, Xylocopa sp;

Insects from the order Orthoptera for example Acheta domesticus, Calliptamus itaiicus, Chortoicetes terminifera, Ceuthophilus spp., Diastrammena asynamora, Dociostaurus maroccanus, Gyllotalpa spp. such as G. africana, G. gtyllotalpa; Gryllus spp., Hieroglyphus daganensis, Kraussaria angulifera, Locusta spp. such as L. migratoria, L. pardalina; Melanoplus spp. such as M. bivittatus, M. femurrubrum, M. mexicanus, M. sanguinipes, M. spretus; Nomadacris septemfasciata, Oedaleus senegalensis, Scaptenscus spp., Schtistocerca spp. such as S. americana, S. gregaria, Stemopelmatus spp., Tachycines asynamorus, and Zonozerus variegatus;

Pests from the Class Arachnida for example Acari, e.g. of the families Argasidae, Ixodidae and Sarcoptidae, such as Amblyomma spp. (e.g. A. americanum, A. variegatum, A. maculatum), Argas spp. such as A. persicu), Boophilus spp. such as B. annulatus, B. decoloratus, B. microplus, Dermacentor spp. such as D. silvarum, D. andersoni, D. variabflis, Hyalomma spp. such as H. truncatum, Ixodes spp. such as I. ricinus, I. rubicundus, I. scapularis, I. holocyclus, I. pacificus, Rhipicephalus sanguineus, Ornithodorus spp. such as O. moubata, O. hermsi, O. turicata, Ornithonyssus bacoti, Otobius megnini, Dermanyssus gallinae, Psoroptes spp. such as P. ovis, Rhipicephalus spp. such as R. sanguineus, R. appendiculatus, Rhipicephalus evertsi; Rhizoglyphus spp., Sarcoptes spp. such as S. Scabiei; and Family Eriophyidae including Aceria spp. such as A. sheldoni, A. anthocoptes, Acallitus spp., Aculops spp. such as A. lycopersici, A. pelekassi; Aculus spp. such as A. schlechtendali; Colomerus vitis, Epitrirnerus pyri, Phyllocoptruta oleivora; Eriophytes ribis and Eriophyes spp. such as Eriophyes sheldoni; Family Tarsonemidae including Hemitarsonemus spp., Phytonemus pallidus and Polyphagotarsonemus latus, Stenotarsonemus spp. Steneotarsonemus spinki; Family Tenuipalpidae including Brevipalpus spp. such as B. phoenictis; Family Tetranychidae including Eotetranychus spp., Eutetranychus spp., Oligonychus spp., Petrobia latens, Tetranychus spp. such as T. cinnabarinus, T. evansi, T. kanzawai, T, pacificus, T. phaseulus, T. telarius and T. urticae; Blyobia praetiosa; Panonychus spp. such as P. ulmi, P. citri; Metatetranychus spp. and Oligonychus spp. such as O. pratensis, O. perseae, Vasates lycopersici; Raoiella indica, Family Carpoglyphidae including Carpoglyphus spp.; Penthaleidae spp. such as Halotydeus destructot; Family Demodicidae with species such as Demodex Spp.; Family Trombicidea including Trombicula spp.; Family Macronyssidae including Ornothonyssus spp.; Family Pyemotidae including Pyemotes tritici; Tyrophagus putrescentiae; Family Acaridae including Acarus siro; Family Araneida including Latrodectus mactans, Tegenaria agrestis, Chiracanthium sp, Lycosa sp Achaearanea tepidariorum and Loxosceles reclusa;

Pests from the Phylum Nematoda, for example, plant parasitic nematodes such as root-knot nematodes, Meloidogyne spp. such as M. hapla, M. incognita, M. javanica; cyst-forming nematodes, Globodera spp. such as G. rostochiensis; Heterodera spp. such as H. avenae, H. glycines, H. Schachtii, H. trifolii; Seed gall nematodes, Anguina spp.; Stem and foliar nematodes, Aphelenchoides spp. such as A. besseyii; Sting nematodes, Belonolaimus spp. such as B. longi caudatus; Pine nematodes, Bursaphelenchus spp. such as B. lignicolus, B. xylophilus; Ring nematodes, Criconema spp., Criconemella spp. such as C. xenoplax and C. ornata; and, Criconemoides spp. such as Criconemoides informis; Mesocriconema spp.; Stem and bulb nematodes, Ditylenchus spp. such as D. destructor, D. dipsaci; Awl nematodes, Dolichodorus spp.; Spiral nematodes, Heliocotylenchus multicinctus; Sheath and sheathoid nematodes, Hemicycliophora spp. and Hemicriconemoides spp.; Hirshmanniella spp.; Lance nematodes, Hoploairnus spp.; False rootknot nematodes, Nacobbus spp.; Needle nematodes, Longidorus spp. such as L. etongatus; Lesion nematodes, Pratylenchus spp. such as P. brachyurus, P. neglectus, P. penetrans, P. curvitatus, P. goodeyi; Burrowing nematodes, Radopholus spp. such as R. sirnilis; Rhadopholus spp.; Rhodopholus spp.; Reniform nematodes, Rotylenchus spp. such as R. robustus, R. reniformis; Scutellonema spp.; Stubby-root nematode, Trichodorus spp. such as T. obtusus, T. prirnitivus; Paratrichodorus spp. such as P. minor; Stunt nematodes, Tylenchorhynchus spp. such as T. claytoni, T. dubius; Citrus nematodes, Tylenchulus spp. such as T. semipenetrans; Dagger nematodes, Xiphinema spp.; and other plant parasitic nematode species;

Insects from the order Isoptera for example Calotermes fiavicollis, Coptotermes spp. such as C. formosanus, C. gestroi, C. acinaciformis; Cornitermes cumulans, Cryptotermes spp. such as C. brevis, C. cavifrons; Globitermes sulfureus, Heterotermes spp. such as H. aureus, H. longiceps, H. tenuis; Leucotermes flavipes, Odontotermes spp., Incisitermes spp. such as I. minor, I. Snyder; Marginitermes hubbardi, Mastotermes spp. such as M. darwinienstis Neocapritermes spp. such as N. opacus, N. parvus; Neotermes spp., Procornitermes spp., Zootermopstis spp. such as Z. angusticolltS, Z. nevadenstis, Reticulitermes spp. such as R. hesperus, R. tibialis, R. speratus, R. flavipes, R. grassei, R. lucifugus, R. santonensis, R. virginicus; Termes natalensis,

Insects from the order Blattaria for example Blatta spp. such as B. orientalis, B. lateralis; Blattella spp. such as B. asahinae, B. germanica; Leucophaea maderae, Panchlora nivea, Periplaneta spp. such as P. americana, P. australasiae, P. brunnea, P. fuligginosa, P. japonica; Supella longipala, Parcoblatta pennsylvanica, Eutycotis floridana, Pycnoscelus surinamensis,

Insects from the order Siphonoptera for example Cediopsylla simples, Ceratophyllus spp., Ctenocephalides spp. such as C. felts, C. canis, Xenopsylla cheopis, Pulex irritans, Trichodectes cants, Tunga penetrans, and Nosopsyllus fasciatus,

Insects from the order Thysanura for example Lepisma saccharin, Ctenolepisma urban, and Thermobia domestica,

Pests from the class Chilopoda for example Geophilus spp., Scutigera spp. such as Scutigera coleoptrata;

Pests from the class Diplopoda for example Blaniulus guttulatus, Julus spp., Narceus spp.,

Pests from the class Symphyla for example Scutigerella immaculata,

Insects from the order Dermaptera, for example Forficula auricularia,

Insects from the order Collembola, for example Onychiurus spp., such as Onychiurus armatus,

Pests from the order Isopoda for example, Armadillidium vulgare, Oniscus asellus, Porcellio scaber,

Insects from the order Phthiraptera, for example Damalinia spp., Pediculus spp. such as Pediculus humanus capitis, Pediculus humanus corporis, Pediculus humanus humanus; Pthirus pubis, Haematopinus spp. such as Haematopinus eurysternus, Haematopinus suns; Linognathus spp. such as Linognathus vitulti; Bovicola bovis, Menopon gallinae, Menacanthus stramineus and Solenopotes captilatus, Trichodectes spp.,

Examples of further pest species which may be controlled by compounds of fomula (I) include: from the Phylum Mollusca, class Bivalvia, for example, Dreissena spp.; class Gastropoda, for example, Arion spp., Biomphaiaria spp., Bulinus spp., Deroceras spp., Galba spp., Lymnaea spp., Oncomelania spp., Pomacea canaliclata, Succinea spp.; from the class of the helminths, for example, Ancylostoma duodenaie, Ancylostoma ceylanicum, Acylostoma braziliensis, Ancylostoma spp., Ascaris lumbricoides, Ascaris spp., Brugia malayi, Brugia tirnori, Bunostomum spp., Chabertia spp., Clonorchtis spp., Cooperia spp., Dicrocoelium spp., Dictyocaulus Nana, Diphyllobothrium latum, Dracunculus medinensis, Echinococcus granulosus, Echinococcus multilocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britow, Trichinella nelson, Trichinella pseudopsiralis, Trichostrongulus spp., Trichuris trichiura, Wuchereria bancrofti.

The compounds of the present invention are suitable for use in treating or protecting animals against infestation or infection by parasites. Therefore, the present invention also relates to the use of a compound of the present invention for the manufacture of a medicament for the treatment or protection of animals against infestation or infection by parasites. Furthermore, the present invention relates to a method of treating or protecting animals against infestation and infection by parasites, which comprises orally, topically or parenterally administering or applying to the animals a parasiticidally effective amount of a compound of the present invention.

The present invention also relates to the non-therapeutic use of compounds of the present invention for treating or protecting animals against infestation and infection by parasites. Moreover, the present invention relates to a non-therapeutic method of treating or protecting animals against infestation and infection by parasites, which comprises applying to a locus a parasiticidally effective amount of a compound of the present invention.

The compounds of the present invention are further suitable for use in combating or controlling parasites in and on animals. Furthermore, the present invention relates to a method of combating or controlling parasites in and on animals, which comprises contacting the parasites with a parasitically effective amount of a compound of the present invention.

The present invention also relates to the non-therapeutic use of compounds of the present invention for controlling or combating parasites. Moreover, the present invention relates to a nontherapeutic method of combating or controlling parasites, which comprises applying to a locus a parasiticidally effective amount of a compound of the present invention.

The compounds of the present invention can be effective through both contact (via soil, glass, wall, bed net, carpet, blankets or animal parts) and ingestion (e.g. baits). Furthermore, the compounds of the present invention can be applied to any and all developmental stages.

The compounds of the present invention can be applied as such or in form of compositions comprising the compounds of the present invention.

The compounds of the present invention can also be applied together with a mixing partner, which acts against pathogenic parasites, e.g. with synthetic coccidiosis compounds, polyetherantibiotics such as Amprolium, Robenidin, Toltrazuril, Monensin, Salinomycin, Maduramicin, Lasalocid, Narasin or Semduramicin, or with other mixing partners as defined above, or in form of compositions comprising said mixtures.

The compounds of the present invention and compositions comprising them can be applied orally, parenterally or topically, e.g. dermally. The compounds of the present invention can be systemically or non-systemically effective.

The application can be carried out prophylactically, therapeutically or non-therapeutically. Furthermore, the application can be carried out preventively to places at which occurrence of the parasites is expected.

As used herein, the term “contacting” includes both direct contact (applying the compounds/compositions directly on the parasite, including the application directly on the animal or excluding the application directly on the animal, e.g. at it's locus for the latter) and indirect contact (applying the compounds/compositions to the locus of the parasite). The contact of the parasite through application to its locus is an example of a non-therapeutic use of the compounds of the present invention.

The term “locus” means the habitat, food supply, breeding ground, area, material or environment in which a parasite is growing or may grow outside of the animal.

As used herein, the term “parasites” includes endo- and ectoparasites. In some embodiments of the present invention, endoparasites can be preferred. In other embodiments, ectoparasites can be preferred. Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention are especially useful for combating parasites of the following orders and species, respectively:

fleas (Siphonaptera), e.g. Ctenocephalides felis, Ctenocephalides canis, Xenopsylla cheoptis, Pulex irritans, Tunga penetrans, and Nosopsyllus fasciatus; cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis, flies, mosquitoes (Diptera), e.g. Aedes aegypti, Aedes albopictus, Aedes vexans, Anastrepha ludens, Anopheles maculipennis, Anopheles crucians, Anopheles albirnanus, Anopheles gambiae, Anopheles freeborni, Anopheles leucosphyrus, Anopheles minimus, Anopheles quadrirnaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplostis equestris, Hippelates spp., Hypoderma lineata, Leptoconops torrens, Lucilia caprin, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosirnulium mixtum, Sarcophaga haemorrhoidaltis, Sarcophaga sp., Simulium vittatum, Stomoxys calcitrans, Tabanus bovinus, Tabanus atratus, Tabanus lineola, and Tabanus simils; lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus; ticks and parasitic mites (Parasitiformes): ticks (Ixodida), e.g. Ixodes scapularis, Ixodes holocyclus, Ixodes paciticus, Rhiphicephalus sanguineus, Dermacentor andersoni, Dermacentor variabills, Amblyomma americanum, Amblyomma maculatum, Ornithodorus hermsi, Ornithodorus turicata and parasitic mites (Mesostigmata), e.g. Ornithonyssus bacoti and Dermanyssus gallinae; Actinedida (Prostigmata) and Acaridida (Astigmata), e.g. Acaraptis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp; Bugs (Heteropterida): Cimex lectularius, Cimex hemipterus, Reduvius senilis, Triatoma spp., Rhodnius ssp., Panstrongylus ssp., and Arilus critatus; Anoplurida, e.g. Haematopinus spp., Linognathus spp., Pediculus spp., Phtirus spp., and Solenopotes spp.; Mallophagida (suborders Arnblycerina and Ischnocerina), e.g. Trimenopon spp., Menopon spp., Trinoton spp., Bovicola spp., Wemeckiella spp., Lepikentron spp., Trichodectes spp., and Felicola spp.; Roundworms Nematoda: Wipeworms and Trichinosis (Trichosyringida), e.g. Trichinellidae (Trichinella spp.), (Trichuridae) Trichunis spp., Capillaria spp.; Rhabditida, e.g. Rhabditis spp., Strongyloides spp., Helicephalobus spp.; Strongylida, e.g. Strongylus spp., Ancylostoma spp., Necator americanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp., Aleurostrongylus abstrusus, and Dioctophyma renale; Intestinal roundworms (Ascaridida), e.g. Ascaris lumbricoides, Ascaris suum, Ascaridia galli, Parascanis equorum, Enterobius vermicularis (Threadworm), Toxocara canis, Toxascanis leonine, Skrjabinema spp., and Oxyuris equi; Camallanida, e.g. Dracunculus medinenstis (guinea worm); Spirurida, e.g. Thelazia spp., Wuchereria spp., Brugia spp., Onchocerca spp., Dirofilari spp., Dipetalonema spp., Setaria spp., Elaeophora spp., Spirocerca lupi, and Habronema spp.; Thorny headed worms (Acanthocephala), e.g. Acanthocephalus spp., Macracanthorhynchus hirudinaceus and Oncicola spp.; Planarians (Plathelminthes): Flukes (Trematoda), e.g. Faciola spp., Fascioloides magna, Paragonimus spp., Dicrocoelium spp., Fasciolopstis buski, Clonorchis sinensis, Schistosoma spp., Trichobilharzia spp., Alaria alata, Paragonimus spp., and Nanocyetes spp.; Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolegs spp., Moniezia spp., Anoplocephala spp., Sirometra spp., Anoplocephala spp., and Hymenoiepis spp.

As used herein, the term “animal” includes warm-blooded animals (including humans) and fish.

Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

Generally, it is favorable to apply the compounds of the present invention in total amounts of 0.5 mg/kg to 100 mg/kg per day, preferably 1 mg/kg to 50 mg/kg per day.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-inwater or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further auxiliaries such as acids, bases, buffer salts, preservatives, and solubilizers. Suitable auxiliaries for injection solutions are known in the art. The solutions are filtered and filled sterile.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Gels are applied to or spread on the skin or introduced into body cavities. Gels are prepared by treating solutions which have been prepared as described in the case of the injection solutions with sufficient thickener that a clear material having an ointment-like consistency results. Suitable thickeners are known in the art.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added. Suitable such auxiliaries are known in the art.

Emulsions can be administered orally, dermally or as injections. Emulsions are either of the water-in-oil type or of the oil-in-water type. They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances. Suitable hydrophobic phases (oils), suitable hydrophilic phases, suitable emulsifiers, and suitable further auxiliaries for emulsions are known in the art.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers. Suitable suspending agents, and suitable other auxiliaries for suspensions including wetting agents are known in the art.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form. Suitable auxiliaries for this purpose are known in the art.

The compositions which can be used in the invention can comprise generally from about 0.001 to 95% of the compound of the present invention.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

Topical application may be conducted with compound-containing shaped articles such as collars, medallions, ear tags, bands for fixing at body parts, and adhesive strips and foils.

Generally it is favorable to apply solid formulations which release compounds of the present invention in total amounts of 10 mg/kg to 300 mg/kg, preferably 20 mg/kg to 200 mg/kg, most preferably 25 mg/kg to 160 mg/kg body weight of the treated animal in the course of three weeks.

The present invention is further illustrated by the following examples.

### EXAMPLES

### A. Preparation Examples

With appropriate modification of the starting materials, the procedures as described in the synthesis examples below were used to obtain further compounds of formula I-B, I-C, I-D, I-E, I-H, I-I, or I-K. The compounds obtained in this manner are listed in the tables that follow, together with physical data.

The products shown below were characterized by melting point determination, by NMR spectroscopy or by the masses ([m/z]) or retention time (RT; [min.]) via HPLC-MS or HPLC spectrometry.

HPLC-MS=high performance liquid chromatography-coupled mass spectrometry;

Method A: Phenomenex Kinetex 1.7 μm XB-C18 100A; 50×2.1 mm; mobile phase: A: water+0.1% trifluoroacetic acid (TFA); B: acetonitrile+0.1% TFA; gradient: 5-100% B in 1.50 minutes; 100% B 0.20 min; flow: 0.8-1.0 ml/min in 1.50 minutes at 60° C. MS: quadrupole electrospray ionization, 80 V (positive mode).

Method B: YMC-PACK ODS-A; 50×3.0 mm; mobile phase: A: water+0.1% formic acid; B: acetonitrile+0.1% formic acid; gradient: 10-100% B in 1.50 minutes; 100% B 2.00 minutes min; flow: 1.2 ml/min in 1.50 minutes at 40° C. MS: quadrupole electrospray ionization, 80 V (positive mode).

### A.1 Preparation Examples for Compounds of Formula I-B

### Example 1: Preparation of 5-methoxy-2-(3-pyridyl)indazole I-B4

To a solution of 2-methoxy-5-nitrobenzaldehyde (500 mg, 2.76 mmol) in i-PrOH (12.5 mL) was added 3-aminopyridine (290 mg, 3.04 mmol) in one portion and the resulting solution was heated to 80° C. under N₂ for 4h. The mixture was cooled to 20 to 25° C. and tri-n-butylphosphine (1.68 g, 2.07 mL, 8.28 mmol) was added in one portion followed by stirring at 80° C. under N₂ for 16h. The mixture was cooled to 20 to 25° C. and diluted with EtOAc (50 mL). The organics were washed with ammonium chloride (30 mL), brine (30 ml), dried over MgSO₄, filtered, and concentrated in vacuo. The residue was purified by silica gel chromatography (heptane/EtOAc) to afford 5-methoxy-2-(3-pyridyl)indazole (280 mg, 45%) as a white solid (HPLC/MS: Rt=0.751 min; m/z=225.9).

### Example 2.1: Preparation of 5-bromo-2-(3-pyridyl)indazole

To a solution of 5-bromo-2-nitrobenzaldehyde (5.00 g, 21.74 mmol) in i-PrOH (75 mL) was added 3-aminopyridine (2.25 g, 23.91 mmol) in one portion and the resulting solution was heated to 80° C. under N₂ for 4h. The mixture was cooled to 20 to 25° C. and tri-n-butylphosphine (13.19 g, 16.29 mL, 65.21 mmol) was added in one portion followed by stirring at 80° C. under N₂ for 16h. The mixture was cooled to 20 to 25° C. and diluted with EtOAc (100 mL). The organics were washed with ammonium chloride (100 mL), brine (100 ml), dried over MgSO₄, filtered, and concentrated in vacuo. The residue was purified by silica gel chromatography (heptane/EtOAc) to afford 5-bromo-2-(3-pyridyl)indazole (4.40 g, 74%) as a white solid (HPLC/MS: Rt=0.966 min; m/z=275.9).

### Example 2.2: Preparation of 5-(2-ethoxyethylsulfanyl)-2-(3-pyridyl)indazole I-B5

To a solution of 5-bromo-2-(3-pyridyl)indazole (274 mg, 1.0 mmol) in dioxane (2 mL) under N2 was added Hünig's base (0.34 mL, 2.0 mmol), Pd₂(dba)₃ (23 mg, 0.03 mmol), Xantphos (29 mg, 0.05 mmol) and 2-ethoxyethanethiol (106 mg, 0.120 mL, 1.0 mmol). The mixture was refluxed for 15h. The reaction mixture was filtered over Celite and concentrated. The residue was purified by silica gel chromatography (Cyclohexane/EtOAc) to afford 5-(2-ethoxyethylsulfanyl)-2-(3-pyridyl)indazole (201 mg, 67%) as a off-white solid (HPLC/MS: Rt=0.998 min; m/z=299.9).

A.2 Preparation Examples for Compounds of Formula I-C

### Example 3.1: Preparation of 4-bromo-2-(3-pyridyl)indazole

To a solution of 6-bromo-2-nitrobenzaldehyde (5.00 g, 21.74 mmol) in i-PrOH (75 mL) was added 3-aminopyridine (2.25 g, 23.91 mmol) in one portion and the resulting solution was heated to 80° C. under N₂ for 16h. The mixture was cooled to 20 to 25° C. and tri-n-butylphosphine (13.19 g, 16.29 mL, 65.21 mmol) was added in one portion followed by stirring at 80° C. under N₂ for 16h. The mixture was cooled to 20 to 25° C. and diluted with EtOAc (100 mL). The organics were washed with ammonium chloride (100 mL), brine (100 ml), dried over MgSO₄, filtered, and concentrated in vacuo. The residue was purified by silica gel chromatography (heptane/EtOAc) to afford 5-bromo-2-(3-pyridyl)indazole (1.30 g, 22%) as a white solid (HPLC/MS: Rt=0.960 min; m/z=275.9).

### Example 3.2: Preparation of 4-(2-ethoxyethylsulfanyl)-2-(3-pyridyl)indazole I-C1

To a solution of 4-bromo-2-(3-pyridyl)indazole (500 mg, 1.82 mmol) in dioxane (4 mL) under N₂ was added Hünig's base (0.62 mL, 3.64 mmol), Pd₂(dba)₃ (50 mg, 0.06 mmol), Xantphos (53 mg, 0.09 mmol) and 2-ethoxyethanethiol (193 mg, 0.920 mL, 1.8 mmol). The mixture was refluxed for 15h. The reaction mixture was filtered over Celite and concentrated. The residue was purified by silica gel chromatography (Cyclohexane/EtOAc) to afford 4-(2-ethoxyethylsulfanyl)-2-(3-pyridyl)indazole (460 mg, 84%) as an off-white solid (HPLC/MS: Rt=1.025 min; m/z=299.8).

### A.3 Preparation Examples for Compounds of Formula I-D

### Example 4: Preparation of 5-methoxy-2-(3-pyridyl)pyrazolo[4,3-b]pyridine I-D57

A suspension of 5-methoxy-1H-pyrazolo[4,3-b]pyridine (1.50 g, 10.0 mmol), 3-pyridylboronic acid (1.30 g, 11.0 mmol) and Cu(OAc)₂ (320 mg, 2.0 mmol) in pyridine (50 mL) was refluxed under N₂ for 24h. The reaction mixture was concentrated under reduced pressure. The residue was diluted with EtOAc (50 mL), washed with sat. ammonium chloride (2×20 mL) and brine (20 mL). The organic layer was dried over MgSO₄, filtered, and concentrated in vacuo. The residue was purified by silica gel chromatography (heptane/EtOAc) to afford 5-methoxy-2-(3-pyridyl)pyrazolo[4,3-b]pyridine (723 mg, 32%) as a beige solid (HPLC/MS: Rt=0.772 min; m/z=227.0).

### Example 5.1: Preparation of 2-(3-pyridyl)-4H-pyrazolo[4,3-b]pyridin-5-one hydrochloride

To a solution of 5-methoxy-2-(3-pyridyl)pyrazolo[4,3-b]pyridine (10.0 g, 44.2 mmol) in TH F (100 mL) was added conc. HCl (37%, 20 mL). The mixture was stirred at RT for 15h. The precipitate was filtered and dried to give 2-(3-pyridyl)-4H-pyrazolo[4,3-b]pyridin-5-one hydrochloride (9.7 g, 39.1 mmol) as a white solid which was used in the next step without further purification (HPLC/MS: Rt=0.485 min; m/z=213.0).

### Example 5.2: Preparation 5-chloro-2-(3-pyridyl)pyrazolo[4,3-b]pyridine hydrochloride

2-(3-pyridyl)-4H-pyrazolo[4,3-b]pyridin-5-one hydrochloride (4.30 g, 18.292 mmol) was dissolved in POCl₃ (50 mL) and stirred at 130° C. for 5h and at RT for 16h. The remaining of POCl₃ was removed by distillation. The residue was suspended in H₂O (70 mL) at RT and stirred for 1h. The solid was filtered, washed with water and dried to give 5-chloro-2-(3-pyridyl)pyrazolo[4,3-b]pyridine hydrochloride (4.00 g, 83%) as a yellow solid (HPLC/MS: Rt=0.824 min; m/z=231.0).

### Example 5.3: Preparation of 5-(2-methyl-2-methylsulfonyl-propoxy)-2-(3-pyridyl)pyrazolo[4,3-b]pyridine I-D59

To a suspension of NaH (60% in mineral oil, 130 mg, 3.25 mmol) in THF (13 mL) at RT was added 2-methyl-2-methylsulfonyl-propan-1-ol (444 mg, 3.25 mmol). The mixture was stirred at RT for 30 min. 5-Chloro-2-(3-pyridyl)pyrazolo[4,3-b]pyridine hydrochloride (537 mg, 2.011 mmol) was added and the reaction was refluxed for 2h. The mixture was concentrated, diluted with CH₂Cl₂ (50 mL), washed with water (20 mL). The organic layer was dried over MgSO₄, filtered, and concentrated in vacuo. The residue was purified by silica gel chromatography (heptane/EtOAc) to afford 5-(2-methyl-2-methylsulfonyl-propoxy)-2-(3-pyridyl)pyrazolo[4,3-b]pyridine (420 mg, 56%) as a beige solid (HPLC/MS: Rt=0.790 min; m/z=347.1).

### Example 6: Preparation of 5-(2-ethoxyethylsulfanyl)-2-(3-pyridyl)pyrazolo[4,3-b]pyridine I-D16

To a suspension of NaH (60% in mineral oil, 140 mg, 3.50 mmol) in THF (15 mL) at RT was added 2-ethoxyethanethiol (280 mg, 2.64 mmol). The mixture was stirred at RT for 30 min. 5-Chloro-2-(3-pyridyl)pyrazolo[4,3-b]pyridine hydrochloride (200 mg, 0.749 mmol) was added and the reaction was refluxed for 2h. The mixture was concentrated, diluted with CH₂Cl₂ (50 mL), washed with water (20 mL). The organic layer was dried over MgSO₄, filtered, and concentrated in vacuo. The residue was purified by silica gel chromatography (heptane/EtOAc) to afford 5-(2-ethoxyethylsulfanyl)-2-(3-pyridyl)pyrazolo[4,3-b]pyridine (140 mg, 69%) as a white solid (HPLC/MS: Rt=0.899 min; m/z=301.0).

### Example 7: Preparation of 5-(2-ethoxyethylsulfinyl)-2-(3-pyridyl)pyrazolo[4,3-b]pyridine I-D39

To a solution of 5-(2-ethoxyethylsulfanyl)-2-(3-pyridyl)pyrazolo[4,3-b]pyridine (400 mg, 1.332 mmol) in CH₂CO₂ (25 mL) at 0° C. was added metachloroperbenzoic acid (77%, 300 mg, 1.339 mmol) and the clear solution was stirred at 0° C. for 30 min. The reaction mixture was diluted with CH₂CO₂ (50 mL), washed with sat. NaHCO₃ (20 mL) and brine (20 mL). The organic layer was dried over MgSO₄, filtered, and concentrated in vacuo. The residue was purified by silica gel chromatography (cyclohexane/EtOAc) to afford 5-(2-ethoxyethylsulfinyl)-2-(3-pyridyl)pyrazolo[4,3-b]pyridine (375 mg, 89%) as a beige solid (HPLC/MS: Rt=0.742 min; m/z=317.0).

### Example 8: Preparation of 5-(2-ethoxyethylsulfonyl)-2-(3-pyridyl)indazole

To a solution of 5-(2-ethoxyethylsulfinyl)-2-(3-pyridyl)pyrazolo[4,3-b]pyridine (200 mg, 0.634 mmol) in CH₂CO₂ (12 mL) at 0° C. was added metachloroperbenzoic acid (77%, 150 mg, 0.670 mmol) and the clear solution was stirred at 0° C. for 30 min. The reaction mixture was diluted with CH₂CO₂ (30 mL), washed with sat. NaHCO₃ (15 mL) and brine (15 mL). The organic layer was dried over MgSO₄, filtered, and concentrated in vacuo. The residue was purified by silica gel chromatography (cyclohexane/EtOAc) to afford 5-(2-ethoxyethylsulfonyl)-2-(3-pyridyl)pyrazolo[4,3-b]pyridine (193 mg, 92%) as a beige solid (HPLC/MS: Rt=0.742 min; m/z=317.0).

### A.4 Preparation Examples for Compounds of Formula I-E

### Example 9.1: Preparation of 6-chloro-2-(3-pyridyl)imidazo[1,2-b]pyridazine

6-chloropyridazin-3-amine (3.0 g, 23.16 mmol) and 2-bromo-1-(3-pyridyl)ethanone hydrobromide (6.5 g, 23.14 mmol) were suspended in EtOH (85 mL). Triethylamine (7.0 mL, 50.25 mmol) was added and the reaction mixture was refluxed under N₂ for 3h and stirred at RT for 16h. The reaction mixture was poored on water/ice (ca. 300 mL), adjusted to pH 6 with sat. ammonium chloride. The brown solid was filtered and purified by silica gel chromatography (CH₂CO₂/MeOH) to afford 6-chloro-2-(3-pyridyl)imidazo[1,2-b]pyridazine (1.0 g, 19%) as a off-white solid (HPLC/MS: Rt=0.664 min; m/z=231.0).

### Example 9.2: Preparation of 2-(3-pyridyl)-6-(2,2,2-trifluoroethoxy)imidazo[1,2-b]pyridazine I-E14

To a suspension of NaH (60% in mineral oil, 160 mg, 4.0 mmol) in THF (15 mL) at RT was added 2,2,2-trifluoroethanol (300 mg, 3.0 mmol). The mixture was stirred at RT for 30 min. 6-chloro-2-(3-pyridyl)imidazo[1,2-b]pyridazine (400 mg, 1.73 mmol) was added and the reaction was refluxed for 15h. The mixture was diluted with MTBE (50 mL), washed with sat. ammonium chloride (20 mL) and water (20 mL). The organic layer was dried over MgSO₄, filtered, and concentrated in vacuo. The residue was suspended in Et₂O (7 mL), cooled to 0° C. and stirred for 2h. Filtration afforded 2-(3-pyridyl)-6-(2,2,2-trifluoroethoxy)imidazo[1,2-b]pyridazine (310 mg, 61%) as a white solid (HPLC/MS: Rt=0.779 min; m/z=295.1).

### A.5 Preparation Examples for Compounds of Formula I-H

### Example 10.1: Preparation of N-(2-chloro-6-methoxy-3-pyridyl)pyridine-3-carboxamide

To a solution of 2-chloro-6-methoxy-pyridin-3-amine (890 mg, 5.61 mmol) and triethylamine (2.35 mL, 16.84 mmol) in CH₂Cl₂ (20 mL) at 0° C. was added pyridine-3-carbonyl chloride hydrochloride (1.50 g, 8.42 mmol) and the mixture was allowed to reach RT over 1h and was further stirred at RT for 16h. The reaction mixture was diluted with CH₂Cl₂ (30 mL) washed with water (20 mL). The organic layer was dried over MgSO₄, filtered, and concentrated in vacuo. The residue was purified by silica gel chromatography CH₂Cl₂/MeOH) to afford N-(2-chloro-6-methoxy-3-pyridyl)pyridine-3-carboxamide (1.06 g, 72%) as a beige solid (HPLC/MS: Rt=0.699 min; m/z=263.8).

### Example 10.2: Preparation of 5-methoxy-2-(3-pyridyl)thiazolo[5,4-b]pyridine I-H4

N-(2-chloro-6-methoxy-3-pyridyl)pyridine-3-carboxamide (300 mg, 1.14 mmol) was dissolved in pyridine (5 mL) under N₂ at RT. Phosphorus sulfide (250 mg, 1.14 mmol) was added and the reaction mixture was stirred at 110° C. for 18h. After cooling to RT, the reaction mixture was diluted with CH₂Cl₂ (50 mL), washed with sat. NaHCO₃ (20 mL), sat. ammonium chloride (20 mL) and brine (20 mL). The organic layer was dried over MgSO₄, filtered, and concentrated in vacuo. The residue was purified by silica gel chromatography (cyclohexane/EtOAc) to afford 5-methoxy-2-(3-pyridyl)thiazolo[5,4-b]pyridine (70 mg, 25%) as a beige solid (HPLC/MS: Rt=0.842 min; m/z=244.3).

A.6 Preparation Examples for Compounds of Formula I-I

### Example 11: Preparation of 6-methoxy-2-(3-pyridyl)-1,3-benzoxazole I-11

Nicotinic acid (500 mg, 4.06 mmol) and 2-amino-5-methoxyphenol (780 mg, 5.60 mmol) were dissolved in 1,2-dichlorobenzen (20 mL) at 120° C. Hexamethyldisiloxane (5.23 g, 32.3 mmol) and P₂O₅ (1.21 g, 8.53 mmol) were added and the reaction mixture was stirred at 180° C. under microwave irradiation for 2h. The reaction mixture was concentrated in vacuo and purified by silica gel chromatography (cyclohexane/EtOAc) to afford 6-methoxy-2-(3-pyridyl)-1,3-benzoxazole (477 mg, 52%) as a beige solid (HPLC/MS: Rt=0.867 min; m/z=227.0).

A.7 Preparation Examples for Compounds of Formula I-K

### Example 12: Preparation of 5-methoxy-2-(3-pyridyl)oxazolo[5,4-b]pyridine I-K1

To a solution of N-(2-chloro-6-methoxy-3-pyridyl)pyridine-3-carboxamide (300 mg, 1.14 mmol) in DME (10 mL) at RT under N₂ was added Cs₂CO₃ (560 mg, 1.71 mmol), CuI (20 mg, 0.11 mmol) and 1,10-phenantroline (20 mg, 0.11 mmol). The reaction mixture was stirred at 80° C. for 16h. After cooling to RT, the reaction mixture was diluted with CH₂Cl₂ (50 mL) and washed with water (20 mL). The organic layer was dried over MgSO₄, filtered, and concentrated in vacuo. The residue was purified by silica gel chromatography CH₂Cl₂/MeOH) to afford 6-methoxy-2-(3-pyridyl)-1,3-benzoxazole (180 mg, 70%) as a beige solid (HPLC/MS: Rt=0.814 min; m/z=227.9).

The following compounds of formula I-B, wherein D and R¹ are in each case H, were obtained in the same manner as described in the Preparation Examples A.1 for compounds of formula I-B:

TABLE I-B

| | | | Physical data | | |
|---|---|---|---|---|---|
| | | | (HPLC-MS) | | |
| Comp. | Y | B | Rt [min] | M/z [MH]⁺ | Method |
| I-B1 | O | B¹ | 0.706 | 289.0 | A |
| I-B2 | SO | CH₂CH₂OCH₂CH₃ | 0.743 | 316.0 | A |
| I-B3 | O | B² | 0.692 | 303.1 | A |
| I-B4 | O | CH₃ | 0.879 | NA | A |
| I-B5 | S | CH₂CH₂OCH₂CH₃ | 0.998 | 299.9 | A |
| I-B6 | S | CH₂CH₂C(O)OCH₃ | 0.936 | 314.1 | A |

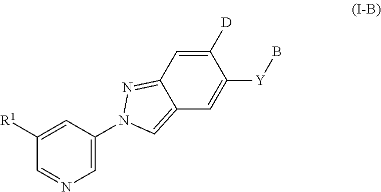

The following compounds of formula I-C, wherein D and R¹ are in each case H, were obtained in the same manner as described in the Preparation Examples A.2 for compounds of formula I-C:

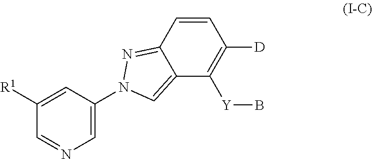

TABLE I-C

| | | | Physical data | | |
|---|---|---|---|---|---|
| | | | (HPLC-MS) | | |
| Comp. | Y | B | Rt [min] | M/z [MH]⁺ | Method |
| I-C1 | S | CH₂CH₂OCH₂CH₃ | 1.025 | 299.8 | A |
| I-C2 | O | CH₃ | 0.779 | 225.8 | A |
| I-C3 | SO | CH₂CH₂OCH₂CH₃ | 0.802 | 315.7 | A |
| I-C4 | SO₂ | CH₂CH₂OCH₂CH₃ | 0.879 | 332.0 | A |

The following compounds of formula I-D, wherein D and R¹ are in each case H, were obtained in the same manner as described in the Preparation Examples A.3 for compounds of formula I-D:

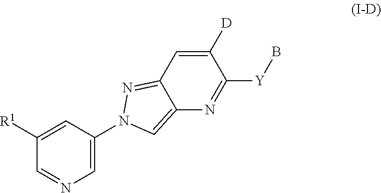

TABLE I-D

| | | | Physical data | | |
|---|---|---|---|---|---|
| | | | (HPLC-MS) | | |
| Comp. | Y | B | Rt [min] | M/z [MH]⁺ | Method |
| I-D1 | O | CH₂CH₃ | 0.805 | 241.1 | A |
| I-D2 | O | CH₂CH₂SCH₃ | 0.940 | 287.1 | A |
| I-D3 | O | CH₂CH₂CH₂S(O)CH₂CH₂CF₃ | 1.243 | 399.3 | A |
| I-D4 | SO₂ | CH₂CH₂CH₂CH₂CH₃ | 2.167 | 315.0 | B |
| I-D5 | S | B³ | 3.260 | 387.0 | B |
| I-D6 | O | B⁴ | 0.920 | 285.0 | A |
| I-D7 | O | CH₂—c-C₃H₅ | 0.831 | 267.0 | A |
| I-D8 | O | CH₂CH₂OCH₃ | 0.730 | 271.1 | A |
| I-D9 | O | CH₂C≡CH | 0.884 | 251.0 | A |
| I-D10 | S | CH₂CH═CH₂ | 1.000 | 268.8 | A |
| I-D11 | O | CH(CH₂OCH₂CF₃)CH₂CH₃ | 1.122 | 367.1 | A |
| I-D12 | S | CH₂CF₃ | 1.055 | 311.0 | A |
| I-D13 | O | c-C₅H₉ | 1.076 | 281.1 | A |
| I-D14 | O | CH₂CF₂CF₂H | 1.005 | 327.0 | A |
| I-D15 | O | CH₂CH(OCH₂CH₃)₂ | 0.998 | 329.2 | A |
| I-D16 | S | CH₂CH₂OCH₂CH₃ | 0.899 | 301.0 | A |
| I-D17 | S | CH(CH₃)₂ | 1.609 | 271.2 | B |
| I-D18 | O | CH(CH₃)(CH₂CH₃) | 1.035 | 269.1 | A |
| I-D19 | O | CH(CH₃)C≡CH | 0.942 | 265.2 | A |
| I-D20 | S | CH₃ | 0.868 | 243.1 | A |
| I-D21 | SO | CH(CH₃)₂ | 0.747 | 287.0 | A |
| I-D22 | O | B⁵ | 0.897 | 353.1 | A |
| I-D23 | S | CH(CH₃)CH(CH₃)₂ | 1.843 | 299.3 | B |
| I-D24 | S | c-C₅H₉ | 1.769 | 297.3 | B |
| I-D25 | S | CH₂CH₃ | 1.483 | 257.2 | B |
| I-D26 | O | CH₂CH₂S(O)CH₃ | 0.685 | 303.0 | A |
| I-D27 | O | CH₂CH₂SCH(CH₃)₂ | 2.880 | 315.1 | B |
| I-D28 | S | CH₂CH₂CH(CH₃)₂ | 1.869 | 299.3 | B |
| I-D29 | O | CH₂CH₂Si(CH₃)₃ | 3.393 | 313.2 | B |
| I-D30 | O | CH₂CH₂S(O)₂CH₃ | 0.699 | 319.1 | A |
| I-D31 | O | B⁶ | 1.192 | 417.0 | A |
| I-D32 | S | B⁷ | 3.673 | 375.0 | B |
| I-D33 | O | B⁸ | 0.868 | 292.1 | A |
| I-D34 | O | B⁹ | 1.074 | 335.1 | A |
| I-D35 | O | B¹⁰ | 1.589 | 309.2 | B |
| I-D36 | O | CH₂CH₂CH₂SCH₂CH₂CF₃ | 1.703 | 383.3 | B |
| I-D37 | O | CH₂CH₂CH₂S(O)CH₃ | 1.620 | 317.0 | B |
| I-D38 | O | CH(CH₃)₂ | 0.991 | 255.1 | A |
| I-D39 | SO | CH₂CH₂OCH₂CH₃ | 0.742 | 317.0 | A |
| I-D40 | O | CH₂CH₂SCF₃ | 1.110 | 341.0 | A |
| I-D41 | O | CH₂CHF₂ | 0.909 | 277.0 | A |
| I-D42 | O | B¹¹ | 0.990 | 303.0 | A |
| I-D43 | O | CH₂CH═CH₂ | 0.934 | 253.0 | A |
| I-D44 | O | CH₂CF₃ | 0.994 | 295.0 | A |
| I-D45 | O | c-C₄H₇ | 1.006 | 267.0 | A |
| I-D46 | SO₂ | CH₂CH₂OCH₂CH₃ | 0.805 | 333.0 | A |
| I-D47 | S | CH(CH₃)₂ | 1.035 | 271.0 | A |
| I-D48 | O | CH(CH₃)CH₂OCH═CH₂ | 0.996 | 297.0 | A |
| I-D49 | O | B¹² | 0.969 | 325.1 | A |
| I-D50 | O | B¹³ | 0.767 | 269.0 | A |
| I-D51 | O | B¹⁴ | 0.770 | 313.2 | A |
| I-D52 | O | CH(CH₃)CH₂(OCH₃) | 0.866 | 285.1 | A |
| I-D53 | S | CH(C₆H₅)CH₃ | 1.763 | 333.3 | B |
| I-D54 | SO | CH₂CH₂CH(CH₃)₂ | 2.140 | 315.0 | B |
| I-D55 | O | CH₂CH₂S(O)CH(CH₃)₂ | 1.787 | 331.1 | B |
| I-D56 | O | CH₂CH₂N[C(O)CF₃]S(O)₂CH₃ | 0.907 | 414.2 | A |
| I-D57 | O | CH₃ | 0.772 | 227.0 | A |
| I-D58 | O | CH(CH₃)CF₃ | 1.033 | 309.2 | A |
| I-D59 | O | CH₂C(CH₃)₂(S(O)₂CH₃) | 0.814 | 347.0 | A |
| I-D60 | O | CH₂CH₂CH₂SCH₃ | 2.613 | 301.2 | B |
| I-D61 | S | CH₂CH₂CH₂CH₂CH₃ | 1.889 | 299.3 | B |
| I-D62 | O | B¹⁵ | 3.220 | 387.2 | B |
| I-D63 | SO₂ | CH(CH₃)₂ | 0.807 | 303.0 | A |
| I-D64 | SO | CH₂CF₃ | 0.816 | 327.0 | A |
| I-D65 | O | B¹⁶ | 1.052 | 313.1 | A |
| I-D66 | O | CH₂CH(CH₃)CH₂SCH₃ | 1.649 | 315.2 | B |
| I-D67 | O | B¹⁷ | 1.289 | 297.4 | B |
| I-D68 | O | CH₂CH(CH₃)CH₂S(O)CH₃ | 1.103 | 331.2 | B |

The following compounds of formula I-E, wherein D and R¹ are in each case H, were obtained in the same manner as described in the Preparation Examples A.4 for compounds of formula I-E:

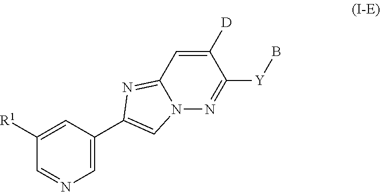

TABLE I-E

| | | | Physical data | | |
|---|---|---|---|---|---|
| | | | (HPLC-MS) | | |
| | | | Rt | M/z | |
| Comp. | Y | B | [min] | [MH]⁺ | Method |
| I-E1 | S | CH₂CH₃ | 0.789 | 257.1 | A |
| I-E2 | O | CH₂CH(OCH₃)CH₃ | 0.702 | 285.1 | A |
| I-E3 | O | CH₂CH₂SCH₃ | 0.750 | 287.1 | A |
| I-E4 | O | CH₂CH₂CH═CH₂ | 0.796 | 267.1 | A |
| I-E5 | SO | CH₂CH₃ | 0.551 | 273.0 | A |
| I-E6 | O | B¹⁰ | 0.827 | 309.0 | A |
| I-E7 | O | CH₂—c-C₃H₅ | 0.787 | 267.1 | A |
| I-E8 | S | CH₂C(O)NH(CH₃) | 0.566 | 300.0 | A |
| I-E9 | O | CH₂C(O)NHS(O)₂CH₃ | 0.548 | 348.0 | A |
| I-E10 | S | CH₂CH₂OCH₃ | 0.715 | 287.0 | A |
| I-E11 | O | B¹¹ | 0.774 | 302.9 | A |
| I-E12 | S | CH₂CH═CH₂ | 0.799 | 268.8 | A |
| I-E13 | S | CH₂C(CH═NOH)CH₃ | 0.680 | 300.0 | A |
| I-E14 | O | CH₂CF₃ | 0.780 | 294.7 | A |
| I-E15 | SO | CH₂CH₂OCH₂CH₃ | 0.631 | 317.1 | A |
| I-E16 | SO | CH₂CF₃ | 0.870 | 326.7 | A |
| I-E17 | S | CH₂CH₂CH₂SCH₂CH═CH₂ | 0.956 | 342.7 | A |
| I-E18 | O | CH₃ | 0.832 | 227.0 | B |
| I-E19 | S | CHCH₃C(O)OCH₂CH₃ | 0.806 | 329.0 | A |
| I-E20 | O | CH(CH₃)₂ | 0.863 | 255.0 | B |
| I-E21 | O | c-C₅H₉ | 0.856 | 281.1 | A |
| I-E22 | O | CH₂CH(CH₃)₂ | 0.847 | 269.1 | A |
| I-E23 | O | CH₂C≡CH | 0.684 | 251.1 | A |
| I-E24 | O | B¹⁸ | 0.779 | 338.1 | A |
| I-E25 | S | B¹⁹ | 0.662 | 307.0 | A |
| I-E26 | S | CH₂CH₂OCH₂CH₃ | 0.774 | 301.0 | A |
| I-E27 | O | B⁹ | 0.863 | 335.0 | A |
| I-E28 | O | CH₂CF₂CF₂H | 0.798 | 327.0 | A |
| I-E29 | O | CH₂CH₂OH | 0.565 | 257.0 | A |
| I-E30 | O | CH₂CH₂OC(O)c—C₃H₅ | 0.768 | 325.1 | A |
| I-E31 | SO₂ | CH₂CF₃ | 0.691 | 342.7 | A |
| I-E32 | S | CH₂CH₂C(O)OCH₂CH₃ | 0.806 | 329.0 | A |
| I-E33 | O | CH₂CH₃ | 0.727 | 241.1 | A |
| I-E34 | S | CH(CH₃)₂ | 0.851 | 271.1 | A |
| I-E35 | O | CH₂CH₂CH₃ | 0.798 | 254.8 | A |
| I-E36 | O | B¹⁷ | 0.695 | 296.8 | A |
| I-E37 | S | CH₂CF₃ | 0.812 | 310.7 | A |
| I-E38 | O | B²⁰ | 0.636 | 305.0 | A |
| I-E39 | SO | CH₂CH₂OCH₃ | 0.578 | 302.8 | A |
| I-E40 | O | B⁸ | 0.695 | 292.1 | A |
| I-E41 | O | CH₂CH₂F | 0.668 | 259.1 | A |

The following compounds of formula I-H, wherein D and R¹ are in each case H, were obtained in the same manner as described in the Preparation Examples A.5 for compounds of formula I-H:

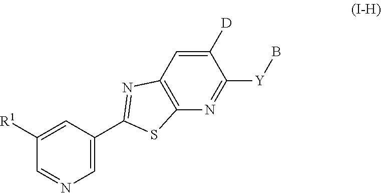

TABLE I-H

| | | | Physical data | | |
|---|---|---|---|---|---|
| | | | (HPLC-MS) | | |
| Comp. | Y | B | Rt [min] | M/z [MH]⁺ | Method |
| I-H1 | O | CH(CH₃)₂ | 0.897 | 272.1 | A |
| I-H2 | O | CH₂CH₃ | 0.875 | 258.0 | A |
| I-H3 | O | CH₂CH₂SCH₃ | 0.799 | 303.9 | A |
| I-H4 | O | CH₃ | 0.842 | 244.3 | A |

The following compounds of formula I-I, wherein D and R¹ are in each case H, were obtained in the same manner as described in the Preparation Examples A.6 for compounds of formula I-I:

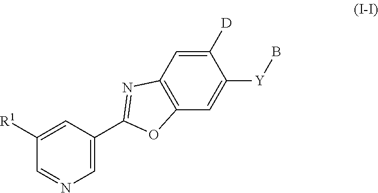

TABLE I-I

| | | | Physical data | | |
|---|---|---|---|---|---|
| | | | (HPLC-MS) | | |
| Comp. | Y | B | Rt [min] | M/z [MH]⁺ | Method |
| I-11 | O | CH₃ | 0.746 | 227.0 | A |

The following compounds of formula I-K, wherein D and R¹ are in each case H, were obtained in the same manner as described in the Preparation Examples A.7 for compounds of formula I-K:

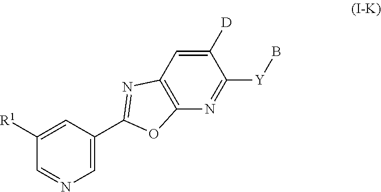

TABLE I-K

| | | | Physical data | | |
|---|---|---|---|---|---|
| | | | (HPLC-MS) | | |
| Comp. | Y | B | Rt [min] | M/z [MH]⁺ | Method |
| I-K1 | O | CH₃ | 0.814 | 227.9 | A |

In the above tables I-B, I-C, I-D, I-E, I-H, I-I, and I-K, the groups B¹ to B²⁰ have the following meanings, wherein § designates the bond to the Y-group of the compounds of formulae I-B, I-C, I-D, I-E, I-H, I-I, and I-K, respectively.

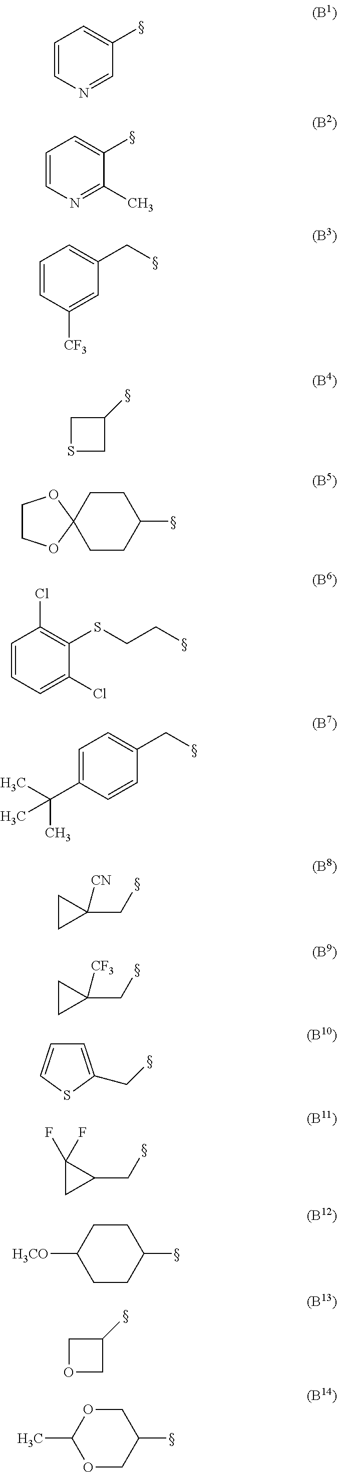

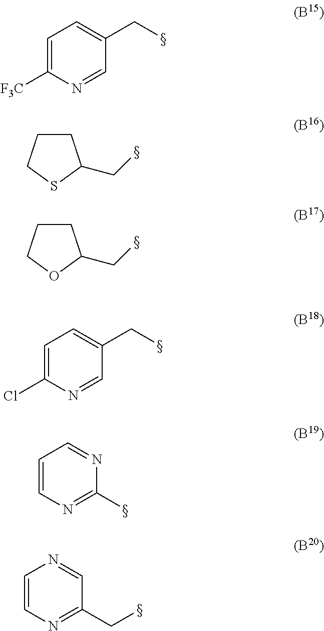

### B. Biological Examples

The activity of the compounds of formula I of the present invention could be demonstrated and evaluated in biological tests described in the following.

If not otherwise specified the test solutions were prepared as follows:

The active compound was dissolved at the desired concentration in a mixture of 1:1 (vol:vol) distilled water: acetone. The test solution was prepared at the day of use and in general at concentrations of ppm (wt/vol).

### B.1 Cowpea Aphid (Aphis craccivora)

Potted cowpea plants colonized with 100-150 aphids of various stages were sprayed after the pest population had been recorded. Population reduction was assessed after 24, 72, and 120 hours.

In this test, the compounds I-K1, I-1, I-E1, I-C1, I-B1, I-B2, I-B3, I-C3, I-H4, I-C4, I-B5, I-E5, I-B6, I-E8, I-D9, I-E10, I-E12, I-D13, I-E13, I-D14, I-E15, I-D15, I-E16, I-E17, I-D17, I-E18, I-E18, I-D19, I-E20, I-D22, I-D23, I-D24, I-D25, I-D26, I-E26, I-E30, I-D30, I-D31, I-E31, I-E33, I-E34, I-E37, I-E39, I-D40, I-D41, I-D43, I-D44, I-D45, I-D48, I-D49, I-D51, I-D52, I-D53, I-D57, I-D58, I-D59 and I-D65, respectively, at 500 ppm showed a mortality of at least 75% in comparison with untreated controls.

### B.2 Cotton Aphid (Aphis gossypii, Mixed Life Stages)

The active compounds were formulated in cyclohexanone as a 10,000 ppm solution supplied in 1.3 ml ABgene® tubes. These tubes were inserted into an automated electrostatic sprayer equipped with an atomizing nozzle and they served as stock solutions for which lower dilutions were made in 1:1 (vol:vol) water: acetone. A nonionic surfactant (Kinetic®) was included in the solution at a volume of 0.01% (v/v).

Cotton plants at the cotyledon stage were infested with aphids prior to treatment by placing a heavily infested leaf from the main aphid colony on top of each cotyledon. Aphids were allowed to transfer overnight to accomplish an infestation of 80-100 aphids per plant and the host leaf was removed. The infested plants were then sprayed by an automated electrostatic plant sprayer equipped with an atomizing spray nozzle. The plants were dried in the sprayer fume hood, removed from the sprayer, and then maintained in a growth room under fluorescent lighting in a 24-hr photoperiod at 25° C. and 20-40% relative humidity. Aphid mortality on the treated plants, relative to mortality on untreated control plants, was determined after 5 days.

In this test, the compounds 1-K1, I-H1, I-E1, I-D1, I-C1, I-B1, I-D2, I-E2, I-H2, I-B2, I-E3, I-E4, I-E5, I-B6, I-D7, I-E7, I-D8, I-E8, I-D9, I-E10, I-D11, I-E11, I-D12, I-D13, I-D14, I-E14, I-D15, I-D16, I-E17, I-D17, I-E18, I-D18, I-D19, I-D20, I-E21, I-D22, I-E22, I-D23, I-E23, I-D24, I-D25, I-D26, I-E26, I-D30, I-E33, I-E36, I-E37, I-D38, I-E39, I-D39, I-D40, I-D41, I-D42, I-D43, I-D44, I-D45, I-D46, I-D47, I-D48, I-D49, I-D50, I-D51, I-D52, I-D57, I-D58 and I-D59, respectively, at 300 ppm showed a mortality of at least 75% in comparison with untreated controls.

### B.3 Silverleaf Whitefly (Bemisia argentifolii, Adult)

The active compounds were formulated in cyclohexanone as a 10,000 ppm solution supplied in 1.3 ml ABgene® tubes. These tubes were inserted into an automated electrostatic sprayer equipped with an atomizing nozzle and they served as stock solutions for which lower dilutions were made in 1:1 (vol:vol) water: acetone. A nonionic surfactant (Kinetic®) was included in the solution at a volume of 0.01% (v/v).

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and 10 to 12 whitefly adults (approximately 3-5 days 2) were introduced. The insects were collected using an aspirator and 0.6 cm, nontoxic Tygon® tubing (R-3603) connected to a barrier pipette tip. The tip, containing the collected insects, was then gently inserted into the soil containing the treated plant, allowing insects to crawl out of the tip to reach the foliage for feeding. Cups were covered with a reusable screened lid (150-micron mesh polyester screen PeCap from Tetko, Inc.). Test plants were maintained in a growth room at 25° C. and 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, the compounds I-K1, I-H1, I-E1, I-D1, I-C1, I-B1, I-D2, I-E2, I-H2, I-B2, I-C2, I-E3, I-C3, I-C4, I-E4, I-B4, I-E5, I-E6, I-B6, I-D7, I-D8, I-D9, I-E9, I-D10, I-D11, I-E11, 1-E12, I-D12, I-D13, I-D14, I-E14, I-D15, I-E16, I-E17, I-D17, I-E18, I-D18, I-D19, I-D20, I-D21, I-E21, I-D22, I-E22, I-D23, I-E23, I-D24, I-E25, I-D26, I-E26, I-D31, I-E33, I-E34, I-E35, I-E36, I-E37, I-D38, I-D39, I-D40, I-D41, I-D42, I-D43, I-D44, I-D45, I-D46, I-D47, I-D48, I-D49, I-D50, I-D51, I-D52, I-D53, I-D57, I-D58, I-D59, I-D63 and I-D64, respectively, at 300 ppm showed a mortality of at least 75% in comparison with untreated controls.

### B.4 Vetch Aphid (Megoura viciae)

The active compounds were formulated in 3:1 (vol:vol) water: DMSO with different concentrations of formulated compounds.

Bean leaf disks were placed into microtiterplates filled with 0.8% agar-agar and 2.5 ppm OPUS™. The leaf disks were sprayed with 2.5 μl of the test solution and 5 to 8 adult aphids were placed into the microtiter plates which were then closed and kept at 23±1° C. and 50±5% relative humidity under fluorescent light for 6 days. Mortality was assessed on the basis of vital, reproduced aphids. Aphid mortality and fecundity was then visually assessed.

In this test, the compounds 1-K1, I-H1, I-E1, I-D1, I-C1, I-B1, I-D2, I-E2, I-H2, I-B2, I-C2, I-H3, I-B3, I-E3, I-C3, I-C4, I-B5, I-E5, I-D7, I-E7, I-D8, I-E8, I-D9, I-D10, I-E10, I-D11, I-E11, 1-E12, I-D12, I-D13, I-D14, I-E14, I-E15, I-D15, I-E16, I-D16, I-E17, I-D17, I-E18, I-D18, I-D19, I-D20, I-E20, I-D21, I-D22, I-E22, I-D23, I-E23, I-D24, I-D25, I-D26, I-E26, I-D27, I-E27, I-D28, I-E28, I-D29, I-E29, I-D30, I-D31, I-E31, I-D32, I-D33, I-E33, I-E34, I-D34, I-D35, I-E35, I-D36, I-E37, I-D38, I-D39, I-E40, I-D40, I-D41, I-E41, I-D42, I-D43, I-D44, I-D45, I-D46, I-D47, I-D48, I-D49, I-D50, I-D51, I-D52, I-D53, I-D57, I-D58, I-D59, I-D60, I-D61, I-D62, I-D63, I-D64, I-D65, I-D66, I-D67 and I-D68, respectively, at 800 ppm showed a mortality of at least 75% in comparison with untreated controls.

### B.5 Green Peach Aphid (Myzus persicae)

The active compounds were formulated in cyclohexanone as a 10,000 ppm solution supplied in 1.3 ml ABgene® tubes. These tubes were inserted into an automated electrostatic sprayer equipped with an atomizing nozzle and they served as stock solutions for which lower dilutions were made in 1:1 (vol:vol) water: acetone. A nonionic surfactant (Kinetic®) was included in the solution at a volume of 0.01% (v/v).

Bell pepper plants at the first true-leaf stage were infested prior to treatment by placing heavily infested leaves from the main colony on top of the treatment plants. Aphids were allowed to transfer overnight to accomplish an infestation of 30-50 aphids per plant and the host leaves were removed. The infested plants were then sprayed by an automated electrostatic plant sprayer equipped with an atomizing spray nozzle. The plants were dried in the sprayer fume hood, removed, and then maintained in a growth room under fluorescent lighting in a 24 hour photoperiod at 25° C. and 20-40% relative humidity. Aphid mortality on the treated plants, relative to mortality on untreated control plants, was determined after 5 days.

In this test, the compounds I-K1, I-H1, I-E1, I-D1, I-C1, I-B1, I-D2, I-E2, I-H2, I-B2, I-H3, I-E3, I-C3, I-E4, I-E5, I-C6, I-D7, I-E7, I-D8, I-E8, I-D9, I-E10, I-D11, I-E11, 1-E12, I-D12, I-E13, I-D14, I-E14, I-D15, I-D16, I-E17, I-D17, I-E18, I-D18, I-E19, I-D19, I-D20, I-E20, I-D21, I-E21, I-D22, I-E22, I-D23, I-E23, I-D24, I-D25, I-D26, I-E26, I-E29, I-D30, I-E33, I-E34, I-E35, I-E36, I-E37, I-D38, I-E38, I-E39, I-D39, I-D40, I-D41, I-D42, I-D43, I-D44, I-D45, I-D46, I-D47, I-D48, I-D49, I-D50, I-D51, I-D52, I-D53, I-D57, I-D58 and I-D59, respectively, at 300 ppm showed a mortality of at least 75% in comparison with untreated controls.

### B.6 Boll Weevil (Anthonomus grandis)

The compounds were formulated in 3:1 (vol:vol) water: DMSO.

For evaluating control of boll weevil (Anthonomus grandis) the test unit consisted of 24-well-microtiter plates containing an insect diet and 20-30 A. grandis eggs. Different concentrations of formulated compounds were sprayed onto the insect diet at 20 μl, using a custom built micro atomizer, at two replications. After application, the microtiter plates were incubated at 23±1° C. and 50±5% relative humidity for 5 days. Egg and larval mortality was then visually assessed.

In this test, the compounds 1-D11, I-E11, I-E23, I-D30, I-E31, I-D45 and I-D47, respectively, at 800 ppm showed a mortality of at least 75% in comparison with untreated controls.

### B.7 Orchid thrips (Dichromothrips corbetti)

The active compounds were formulated as a 1:1 (vol:vol) water: acetone solution. Surfactant (Alkamuls EL 620) was added at the rate of 0.1% (vol/vol). Vanda orchids petals were cleaned, washed and air dried prior to spraying. Petals were dipped into the test solution for 3 seconds, air dried, placed inside a resealable plastic and inoculated with 20 adults. The treated petals were kept inside the h2ing room at 28-29° C. and relative humidity of 50-60%. Percent mortality was recorded after 72 hours.

In this test, the compounds O-E18, I-D24, I-D25, I-D26, I-D59 and I-D65, respectively, at 500 ppm showed a mortality of at least 75% in comparison with untreated controls.

The present invention is further illustrated by the following embodiments:
1. Use of a substituted 3-pyridyl heterobicyclic compound of formula I or a salt, stereoisomer, tautomer, or N-oxide thereof

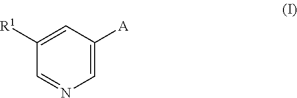

as an agrochemical pesticide,
wherein
R¹ is H, CN or halogen; and

A is a moiety selected from the group consisting of

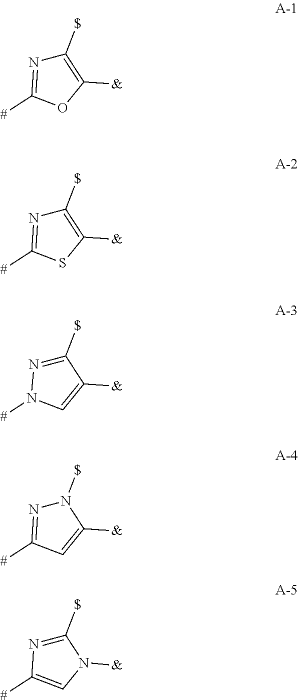

wherein
# denotes the bond to the pyridine ring of formula I; and
$ and & mark the connection to a C₃-C₄-alkylene or C₃-C₄-alkenylene chain, of which 1, 2, or 3 C-atoms may be replaced by a heteroatom independently selected from the group consisting of O, N, and S, and which forms together with the atoms to which it is bonded an annulated saturated, partially unsaturated, or aromatic 5 or 6-membered carbocyclic or heterocyclic ring, wherein the N and/or S atoms of the chain, independently of one another, may be oxidized, and wherein the C-atoms and/or the heteroatoms of the chain may be substituted by one or more, e.g. 1, 2, 3, 4 or 5 substituents R², said substituents R² being identical or different from one another if more than one substituent R² is present; wherein
R² is
   (i) halogen, CN, NO₂, C₁-Coo-alkyl, C₃-C₈-cycloalkyl, C₂-C₁₀-alkenyl, C₃-C₈-cycloalkenyl, C₂-C₁₀-alkynyl, wherein the aforementioned aliphatic and cycloaliphatic radicals each independently may be substituted with 1 to 10 substituents R¹¹, said substituents R¹¹ being identical or different from one another if more than one substituent R¹¹ is present;
   OR¹², NR^{13a}R^{13b}, S(O)ₙR¹², S(O)ₙNR^{13a}R^{13b}, Si(R¹⁵)₂R¹⁶;
   phenyl which may be substituted with one or more, e.g. 1, 2, 3, 4, or 5 substituents R¹⁴, said substituents R¹⁴ being identical or different from one another if more than one substituent R¹⁴ is present;
   or a 3-, 4-, 5-, 6- or 7-membered saturated, partially unsaturated or fully unsaturated heterocyclic ring wherein said heterocyclic ring comprises 1, 2 or 3 heteroatoms independently selected from the group consisting of O, N, and S, and may be substituted with one or more, e.g. 1, 2, 3, 4, or 5 substituents R¹⁴, said substituents R¹⁴ being identical or different from one another if more than one substituent R¹⁴ is present, and wherein said N and S atoms, independently of one another, may be oxidized;
   with the proviso that R² is not halogen, if R² is bonded to a heteroatom; or
   (ii) a substituent

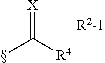

wherein
§ denotes the bond to the atom on which R² is present;
X is NR³, O, or S; and
R⁴ is H, CR⁵R⁶R⁷, NR⁸R⁹, OR¹⁰, or SR¹⁰; or
   (iii) two R² present on one C or S atom may together be ═O, ═S, or ═NR³; and wherein
R³ is H, CN, NO₂, C₁-C₁₀-alkyl, C₃-C₅-cycloalkyl, C₂-C₁₀-alkenyl, C₂-C₁₀-alkynyl, wherein the aforementioned aliphatic and cycloaliphatic radicals each independently may be substituted with 1 to 10 substituents R¹¹, said substituents R¹¹ being identical or different from one another if more than one substituent R¹¹ is present; OR¹², NR^{13a}R^{13b}, S(O)ₙR¹², S(O)ₙNR^{13a}R^{13b}, C(═O)R¹¹, C(═O)NR^{13a}R^{13b}, C(═O)OR¹², C(═S)R¹¹, C(═S)NR^{13a}R^{13b}, C(═S)OR¹², C(═S)SR¹², C(═NR^{13a})R¹¹, C(═NR^{13a})NR^{13a}R^{13b};
   phenyl which may be substituted with one or more, e.g. 1, 2, 3, 4, or 5 substituents R¹⁴, said substituents R¹⁴ being identical or different from one another if more than one substituent R¹⁴ is present;
   or a 3-, 4-, 5-, 6- or 7-membered saturated, partially unsaturated or fully unsaturated, or aromatic heterocyclic ring wherein said heterocyclic ring comprises 1, 2 or 3 heteroatoms independently selected from the group consisting of O, N, and S, and may be substituted with one or more, e.g. 1, 2, 3, 4, or 5, substituents R¹⁴, said substituents R¹⁴ being identical or different from one another if more than one substituent R¹⁴ is present, and wherein said N and/or S atoms, independently of one another, may be oxidized;
R⁵, R⁶, R⁷ are selected independently of one another from the group consisting of H, halogen, CN, NO₂, —SCN, C₁-C₆-alkyl, C₁-C₆-haloalkyl, C₁-C₆-alkoxy, C₁-C₆-haloalkoxy, C₁-C₆-alkyl-S(O)ₙ—, C₁-C₆-halo-S—, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, Si(R¹⁵)₂R¹⁶, OR¹², OSO₂R¹², S(O)ₙR¹², S(O)ₙNR^{13a}R^{13b}, NR^{13a}R^{13b}, C(═O)NR^{13a}R^{13b}, C(═S)NR^{13a}R^{13b}, C(═O)OR¹²;
   phenyl, optionally substituted with one or more, e.g. 1, 2, 3, 4 or 5, substituents R¹⁴ which are independently selected from one another;
   a 3-, 4-, 5-, 6- or 7-membered saturated, partially unsaturated or fully unsaturated, or aromatic heterocyclic ring comprising 1, 2 or 3 heteroatoms selected from O, N and S, optionally substituted with one or more, e.g. 1, 2, 3 or 4, substituents R¹⁴, selected independently from one another, and wherein the N and/or S atoms of the heterocyclic ring may optionally be oxidized;
   or R⁵ and R⁶ may together form ═O, ═CR¹¹R¹⁷, ═S, ═S(O)ₙR¹²; ═S(O)ₙNR^{13a}R^{13b}, ═NR^{13a}, ═NOR¹², ═NNR^{13a}, and R⁷ is selected from the group above;
   or R⁵ and R⁶ may together form a 3-, 4-, 5-, 6-, 7- or 8-membered saturated or partially unsaturated carbocyclic or heterocyclic ring together with the C-atom to which they are bonded to, and R⁷ is selected from the group above;
R⁸, R⁹ are selected independently of one another from the group consisting of H, CN, C₁-C₁₀-alkyl, C₃-C₈-cycloalkyl, C2-C₁₀-alkenyl, C₂-C₁₀-alkynyl, wherein the aforementioned aliphatic and cycloaliphatic radicals may be substituted with 1 to 10 substituents R¹¹ and wherein said substituents R¹¹ are selected independently from one another;
   OR¹², NR^{13a}R^{13b}, S(O)ₙNR^{13a}R^{13b}, C(═O)R¹¹, C(═O)NR^{13a}R^{13b}, C(═O)OR¹², C(═S)R¹¹, C(═S)NR^{13a}R^{13b}, C(═S)OR¹², C(═S)SR¹², C(═NR^{13a})R¹¹, C(═NR^{13a})NR^{13a}R^{13b}, Si(R¹⁵)₂R¹⁶;
   phenyl, which may be substituted with one or more, e.g. 1, 2, 3, 4, or 5 substituents R¹⁴, wherein said substituents R¹⁴ are selected independently from one another if more than one substituent R¹⁴ is present;
   a 3-, 4-, 5-, 6- or 7-membered saturated, partially unsaturated or fully unsaturated, or aromatic heterocyclic ring, wherein said heterocyclic ring comprises 1, 2, 3, or 4 heteroatoms independently selected from the group consisting of O, N and S, and may be substituted with one or more, e.g. 1, 2, 3, 4, or 5 substituents R¹⁴, said substituents R¹⁴ being identical or different from one another if more than one substituent R¹⁴ is present, and wherein said N and/or S atoms, independently of one another, may be oxidized;
   or R⁸ and R⁹ together are part of a C₂-C₇-alkylene, C₂-C₇-alkenylene or C₂-C₇-alkynylene chain and form a 3-, 4-, 5-, 6-, 7- or 8-membered saturated, partially unsaturated or fully unsaturated, or aromatic heterocyclic ring together with the N-atom they are bonded to, wherein 1 to 4 of any of the CH₂ groups in the C₂-C₇-alkylene chain or 1 to 4 of any of the CH₂ or CH groups in the C₂-C₇-alkenylene chain or 1 to 4 of any of the CH₂, CH or C groups in the C₂-C₇-alkynylene chain may be replaced by 1 to 4 groups independently selected from the group consisting of C═O, C═S, O, N and NH, and
   wherein the C- and/or N-atoms in the C₂-C₇-alkylene, C₂-C₇-alkenylene or C₂-C₇-alkynylene chain may be substituted with 1 to 5 substituents independently selected from the group consisting of halogen, CN, C₁-C₆-alkyl, C₁-C₆-haloalkyl, C₁-C₆-alkoxy, C₁-C₆-haloalkoxy, C₁-C₆-alky-S—, C₁-C₆-haloalkyl-S—, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl and phenyl, which may be substituted with 1 to 5 substituents R¹¹, said substituents R¹ being identical or different from one another if more than one substituent R¹¹ is present, and
   wherein the S- and/or N-atoms in the C₂-C₇-alkylene, C₂-C₇-alkenylene or C₂-C₇-alkynylene chain, independently of one another, may be oxidized;
   or R⁸ and R⁹ together may form a ═CHR¹⁷, ═CR¹¹R¹⁷, ═NR^{13a} or ═NOR¹² radical;
R¹⁰ is H, CN, C₁-C₆-alkyl, C₁-C₆-haloalkyl, C₁-C₆-alkoxy, C₁-C₆-haloalkoxy, C₁-C₆-alkylS(O)ₙ—, C₁-C₆-haloalkyl-S—, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁵)₂R¹⁶, S(O)ₙR¹², S(O)ₙNR^{13a}R^{13b}, NR^{13a}R^{13b}, —N═CR¹¹R¹⁷, —C(═O)R¹¹, C(═O)NR^{13a}R^{13b}, C(═S)NR^{13a}R^{13b}, C(═O)OR¹²;
   phenyl, optionally substituted with one or more substituents R¹⁴; which are selected independently from one another, or a 3-, 4-, 5-, 6- or 7-membered saturated, partially unsaturated or fully unsaturated,
   or aromatic heterocyclic ring comprising 1, 2 or 3 heteroatoms selected from O, N and S, optionally substituted with one or more, e.g. 1, 2, 3 or 4, substituents R¹⁴, selected independently from one another, and wherein the N and/or S atoms of the heterocyclic ring may optionally be oxidized;
   and wherein
R¹¹ is H, halogen, CN, NO₂, —SCN, C₁-C₆-alkyl, C₁-C₆-haloalkyl, C₁-C₆-alkoxy, C₁-C₆-haloalkoxy, C₁-C₆-alkyl-S(O)ₙ—, C₁-C₆-haloalkyl-S—, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, Si(R¹⁵)₂R¹⁶, OR²⁰, OSO₂R²⁰, S(O)ₙR²⁰, S(O)ₙNR^{21a}R^{21b}, NR^{21a}R^{21b}, C(═O)NR^{21a}R^{21b}C(═S)NR^{21a}R^{21b}, C(═O)OR²⁰;
   phenyl, optionally substituted with one or more, e.g. 1, 2, 3, 4 or 5 substituents R²² which are independently selected from one another,
   or a 3-, 4-, 5-, 6- or 7-membered saturated, partially unsaturated or fully unsaturated, or aromatic heterocyclic ring comprising 1, 2 or 3 heteroatoms selected from O, N and S, optionally substituted with one or more, e.g. 1, 2, 3 or 4, substituents R²², selected independently from one another, and wherein the N and/or S atoms of the heterocyclic ring may optionally be oxidized;
or two R¹¹ present on one C-atom may together form ═O, ═CR¹⁷R¹⁸, ═S, ═S(O)ₙR²⁰; ═S(O)ₙNR^{21a}R^{21b}, ═NR^{21a}, ═NOR²⁰, ═NNR^{21a};
   or two R¹¹ may form a 3-, 4-, 5-, 6-, 7- or 8-membered saturated or partly unsaturated carbocyclic or heterocyclic ring together with the C-atoms to which the two R¹¹ are bonded to;
R¹² is H, CN, C₁-C₆-alkoxy, C₁-C₆-haloalkoxy, C₁-C₆-alkyl-S(O)ₙ, C₁-C₆-haloalkyl-S;
C₁-C₆-alkyl, which may be substituted with CN, C₁-C₆-alkoxy, C₁-C₆-haloalkoxy, C₁-C₆-alkyl-S(O)ₙ, C₁-C₆-haloalkyl-S(O)ₙ, C₁-C₆-hetaryl;
   C₁-C₆-haloalkyl, which may be substituted with C₁-C₆-alkoxy, C₁-C₆-haloalkoxy, C₁-C₆-alkyl-S(O)ₙ, C₁-C₆-haloalkyl-S(O)ₙ;
   C₃-C₈-cycloalkyl-C₁-C₄-alkyl, which may be substituted with halogen, CN;
   C₁-C₄-alkyl-C₃-C₈-cycloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁵)₂R¹⁶, S(O)ₙR²⁰, S(O)ₙNR^{21a}R^{21b}, NR^{21a}R^{21b}, —N═CR¹⁷R¹⁸, —C(═O)R¹⁹, C(═O)NR^{21a}R^{21b}C(═S)NR^{21a}R^{21b}, C(═O)OR²⁰,
   phenyl, optionally substituted with one or more substituents R²²; which are selected independently from one another,
   or a 3-, 4-, 5-, 6- or 7-membered saturated, partially unsaturated or fully unsaturated, or aromatic heterocyclic ring comprising 1, 2 or 3 heteroatoms selected from O, N and S, optionally substituted with one or more, e.g. 1, 2, 3 or 4, substituents R²², selected independently from one another, and wherein the N and/or S atoms of the heterocyclic ring may optionally be oxidized;
R^{13a}, R^{13b} are each independently from one another selected from the group consisting of H, C₁-C₆-alkyl, C₁-C₆-haloalkyl, C₁-C₆-alkoxy, C₁-C₆-haloalkoxy, C₁-C₆-alkyl-S—, C₁-C₆-haloalky-S—, C₃-C₈-cycloalkyl, C₃-C₈-cycloalkyl-C₁-C₆-alkyl, C₃-C₈-halocycloalkyl, C₂-C₆-alkenyl, C₂-C₆-haloalkenyl, C₂-C₆-alkynyl, C₂-C₆ haloalkynyl, —S(O)ₙNR^{21a}R^{21b}C(═O)R¹⁹, C(═O)OR²⁰, C(═O)NR^{21a}R^{21b}, C(═S)R¹⁹, C(═S)SR²⁰, C(═S)NR^{21a}R^{21b}, C(═NR^{21a})R¹⁹;
   phenyl, optionally substituted with one or more, e.g. 1, 2, 3 or 4, substituents R²², which are selected independently from one another;
   a 3-, 4-, 5-, 6- or 7-membered saturated, partially unsaturated or fully unsaturated, or aromatic heterocyclic ring comprising 1, 2, 3 or 4 heteroatoms selected from O, N and S, optionally substituted with one or more, e.g. 1, 2, 3 or 4, substituents R²², selected independently from one another, and wherein the N and/or S atoms of the heterocyclic ring may optionally be oxidized;
   or R^{13a} and R^{13b} are together a C₂-C₇ alkylene or C₂-C₇ alkenylene chain and form a 3-, 4-, 5-, 6-, 7- or 8-membered saturated, partially unsaturated or fully unsaturated, or aromatic ring together with the N-atom they are bonded to,
   wherein the alkylene chain or alkenylene chain may contain one or two heteratoms selected from O, S and N, and may optionally be substituted with halogen, C₁-C₆-alkyl, C₁-C₆-haloalkyl, C₁-C₆-alkoxy, C₁-C₆-haloalkoxy, C₁-C₆-alkyl-S—, C₁-C₆-haloalkyl-S, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl;
   phenyl, optionally substituted with one or more substituents R²²; which are selected independently from one another;
   or a 3-, 4-, 5-, 6,- or 7-membered saturated, partially unsaturated or fully unsaturated, or aromatic heterocyclic ring comprising 1, 2 or 3 heteroatoms selected from O, S, and N, optionally substituted with one or more substituents R²², selected independently from one another, and wherein the N and/or S atoms of the heterocyclic ring may optionally be oxidized;
or R^{13a} and R^{13b} together may form a ═CR¹⁷R¹⁸, ═NR²¹ or ═NOR²⁰ radical; R¹⁴ is H, halogen, CN, NO₂, SCN, SF₅, C₁-C₁₀-alkyl, C₃-C₈-cycloalkyl, C₂-C₁₀-alkenyl, C₂-C₁₀-alkynyl, wherein the C-atoms of the aforementioned aliphatic and cyclo-aliphatic radicals may optionally be substituted with one or more R¹⁹, which are selected independently from one another;
   Si(R¹⁵)₂R¹⁶, OR²⁰, OS(O)ₙR²⁰, —S(O)ₙR²⁰, S(O)ₙNR^{21a}R^{21b}, NR^{21a}R^{21b}, C(═O)R¹⁹, C(═O)OR²⁰, —C(═NR^{21a})R¹⁹, C(═O)NR^{21a}R^{21b}, C(═S)NR^{21a}R^{21b};
   phenyl, optionally substituted with halogen, CN, NO₂, C₁-C₆-alkyl, C₁-C₆-haloalkyl, C₁-C₆-alkoxy or C₁-C₆-haloalkoxy;
   or a 3-, 4-, 5-, 6- or 7-membered saturated, partially unsaturated or fully unsaturated, or aromatic heterocyclic ring comprising 1, 2 or 3 heteroatoms selected from O, N and S, optionally substituted with one or more substituents selected independently from one another from halogen, CN, NO₂, C₁-C₆-alkyl, C₁-C₆-haloalkyl, C₁-C₆-alkoxy or C₁-C₆-haloalkoxy, and wherein the N and/or S atoms of the heterocyclic ring may optionally be oxidized;
   or two R¹⁴ present together on one atom of a partly saturated heterocycle may be ═O, ═CR¹⁷R¹⁸, ═NR^{21a}, ═NOR²⁰ or ═NNR^{21a};
   or two R¹⁴ on adjacent C-atoms may be a bridge selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{21a}, CH₂CH═N, CH═CHNR^{21a}, OCH═N, SCH═N and form together with the C-atoms to which the two R¹⁴ are bonded to a 5-membered or 6-membered saturated, partially unsaturated or fully unsaturated, or aromatic carbocyclic or heteocyclic ring, wherein the ring may optionally be substituted with one or two substituents selected from ═O, OH, CH₃, OCH₃, halogen, CN, halomethyl or halomethoxy;
R¹⁵, R¹⁶ are each independently from one another selected from the group consisting of H, C₁-C₆ alkyl, C₁-C₆ haloalkyl, C₁-C₆ alkoxy, C₁-C₄-alkoxy-C₁-C₄-alkyl, C₃-C₈ cycloalkyl, C₃-C₈ halocycloalkyl, C₁-C₄-haloalkoxy-C₁-C₄-alkyl, and phenyl;
R¹⁷, R¹⁸ are each independently from one another selected from the group consisting of H, C₁-C₄ alkyl, C₁-C₆ cycloalkyl, C₁-C₂-alkoxy-C₁-C₂-alkyl, phenyl and benzyl;
R¹⁹ is H, halogen, CN, NO₂, OH, SH, SCN, SF₅, C₁-C₆-alkoxy, C₁-C₆-haloalkoxy, C₁-C₆-alkyl-S(O)ₙ—, C₁-C₆-haloalkyl-S—, trimethylsilyl, triethylsilyl, tert-butyldimethylsilyl, C₁-C₆-alkyl, C₂-C₆-alkenyl, C₂-C₆-alkynyl, C₃-C₈-cycloalkyl, wherein the aliphatic and cyclo-aliphatic radicals may be unsubstituted, partially or fully halogenated and/or oxgenated and/or may carry 1 or 2 radicals selected from C₁-C₄ alkoxy;
   phenyl, benzyl, pyridyl, or phenoxy, wherein the radicals may be unsubstituted, partially or fully halogenated and/or to carry 1, 2 or 3 substituents selected from C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino or di-(C₁-C₆-alkyl)amino;
   or two R¹⁹ present on the same C-atom may together be ═O, ═CH(C₁-C₄-alkyl), ═C(C₁-C₄-alkyl)C₁-C₄-alkyl, ═N(C₁-C₆-alkyl) or ═NO(C₁-C₆-alkyl);
R²⁰ is H, CN, C₁-C₆-alkoxy, C₁-C₆-haloalkoxy, C₁-C₆-alkyl-S(O)ₙ, C₁-C₆-haloalkyl-S—, trimethylsilyl, triethylsilyl, tert-butyldimethylsilyl, C₁-C₆-alkyl, C₂-C₆-alkenyl, C₂-C₆-alkynyl, C₃-C₈-cycloalkyl, wherein the aliphatic and cycloaliphatic radicals may be unsubstituted, partially or fully halogenated and/or oxygenated and/or may carry 1 or 2 radicals selected from C₁-C₄ alkoxy, phenyl, benzyl, pyridyl, or phenoxy, wherein the radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl;
R^{21a}, R^{21b} are each independently from one another selected from the group consisting of H, CN, C₁-C₆-alkoxy, C₁-C₆-haloalkoxy, C₁-C₆-alkyl-S(O)ₙ—, trimethylsilyl, triethylsilyl, tert-butyldimethylsilyl, C₁-C₆-alkyl, C₂-C₆-alkenyl, C₂-C₆-alkynyl, C₃-C₈-cycloalkyl, C₃-C₈-cycloalkyl-C₁-C₆-alkyl, C₁-C₆-alkyl-C₃-C₈-cycloalkyl, wherein the aliphatic and cyclo-aliphatic radicals may be unsubstituted, partially or fully halogenated and/or oxygenated and/or may carry 1 or 2 radicals selected from C₁-C₄-alkoxy,
   phenyl, benzyl, pyridyl, and phenoxy, wherein the radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl; or R^{21a} and R^{21b} may together be a C₂-C₆ alkylene chain forming a 3- to 7-membered saturated, partly saturated or unsaturated ring together with the N-atom R^{21a} and R^{21b} are bonded to, wherein the alkylene chain may contain 1 or 2 heteroatoms selected from O, S, and N, and may optionally be substituted with halogen, C₁-C₄-haloalkyl, C₁-C₄-alkoxy or C₁-C₄-haloalkoxy, and wherein the N and/or S atoms of the heterocyclic ring may optionally be oxidized;
R²² is H, halogen, NO₂, CN, OH, SH, C₁-C₆-alkoxy, C₁-C₆-haloalkoxy, C₁-C₆-alkyl-S(O)ₙ, C₁-C₆-haloalkyl-S—, trimethylsilyl, triethylsilyl, tert-butyldimethylsilyl, C₁-C₆-alkyl, C₂-C₆-alkenyl, C₂-C₆-alkynyl, C₃-C₈-cycloalkyl, wherein the aliphatic and cyclo-aliphatic radicals may be unsubstituted, partially or fully halogenated and/or oxygenated and/or may carry 1 or 2 radicals selected from C₁-C₄-alkoxy,
   phenyl, benzyl, pyridyl, or phenoxy, wherein the radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy, (C₁-C₆-alkoxy)carbonyl; or two R²² present together on one atom may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
   or two R²² on two adjacent C-atoms may be together a C₂-C₆ alkylene chain or C₂-C₆ alkenylene chain, which form together with the C-atom they are bonded to a 3-, 4-, 5-, 6- or 7-membered saturated, partially unsaturated or fully unsaturated, or aromatic ring, wherein the alkylene or alkenylene chain may contain 1 or 2 heteroatoms selected from O, S, and N, and may optionally be substituted with halogen, C₁-C₄-haloalkyl, C₁-C₄-alkoxy or C₁-C₄-haloalkoxy, and wherein the N and/or S atoms of the heterocyclic ring may optionally be oxidized;
n is 0, 1 or 2.
2. The use of compounds of formula I according to embodiment 1, wherein
   A is a moiety selected from A-1, A-2, A-3, A-4, and A-5, wherein
   # denotes the bond to the pyridine ring of formula I; and
   $ and & mark the connection to a C₄-alkylene or C₄-alkenylene chain of which 1 C-atom may be replaced by N, and which forms together with the atoms to which it is bonded an annulated saturated, partially unsaturated, or aromatic 6-membered carbocyclic or heterocyclic ring,
   wherein the C-atoms and/or the N-atom of the chain may be substituted by 1, 2, or 3 substituents R², said substituents R² being identical or different from one another if more than one substituent R² is present.
3. The use of compounds of formula I according to any one of embodiments 1 to 2, wherein
   A is a moiety selected from A-1, A-2, A-3, A-4, and A-5, wherein
   # denotes the bond to the pyridine ring of formula I; and
   $ and & mark the connection to a chain, which is selected from the group consisting of

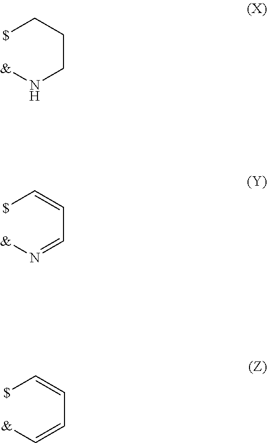

wherein in each case $ marks the connection to the $-position of the moiety A and &
marks the connection to the &-position of the moiety A,
and wherein the C-atoms and/or the N-atom of the chains may be substituted by 1, 2, or 3 substituents R², said substituents R² being identical or different from one another if more than one substituent R² is present.
4. The use of compounds of formula I according to any one of embodiments 1 to 3, wherein
   A is a moiety selected from A-1, A-2, A-3, A-4, and A-5, wherein
   # denotes the bond to the pyridine ring of formula I; and
   $ and & mark the connection to a chain, which is selected from the group consisting of

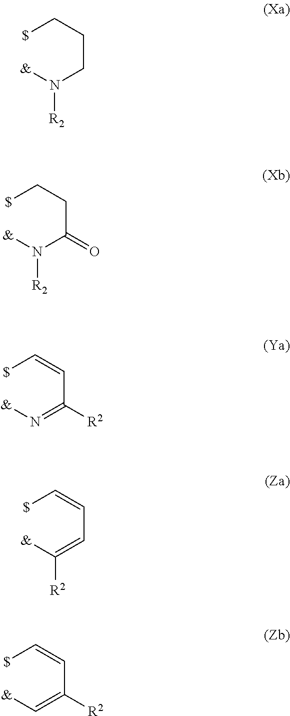

wherein in each case $ marks the connection to the $-position of the moiety A and & marks the connection to the &-position of the moiety A.
5. The use of compounds of formula I according to any one of embodiments 1 to 4, wherein
   A is a moiety selected from the group consisting of

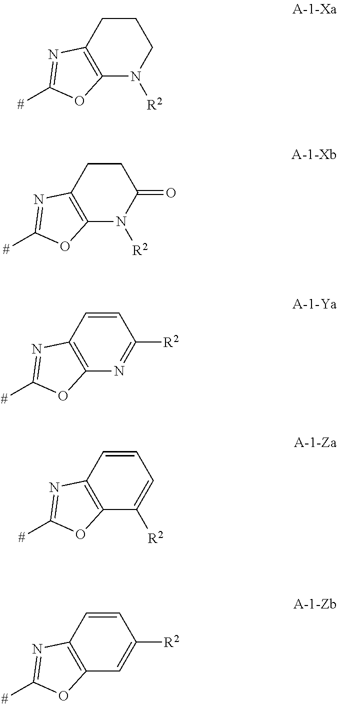

or
A is a moiety selected from the group consisting of

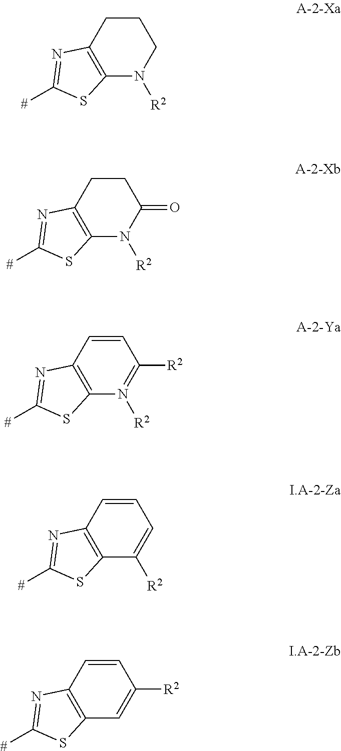

or
A is a moiety selected from the group consisting of

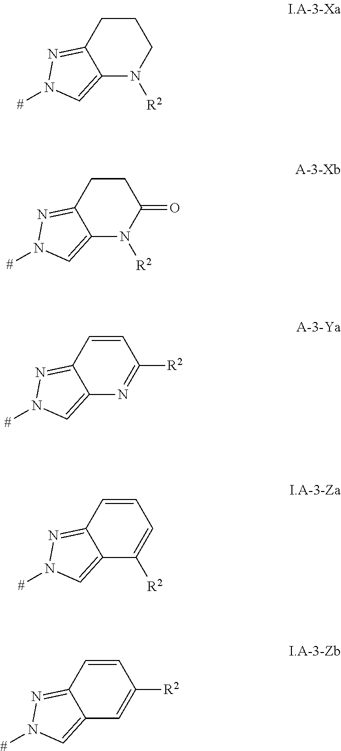

or
A is a moiety selected from the group consisting of

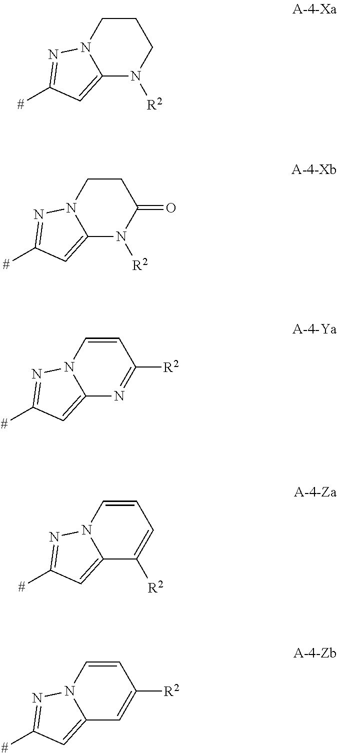

or
A is a moiety selected from the group consisting of

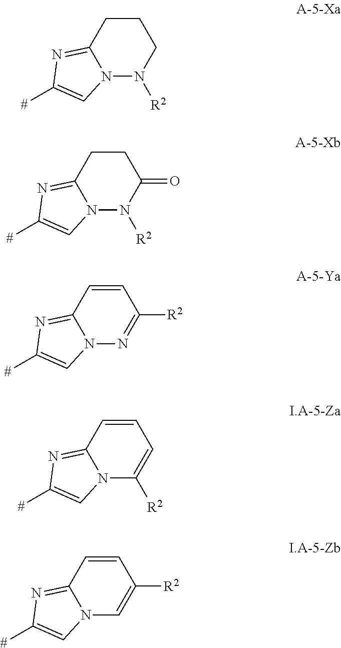

6. A substituted 3-pyridyl heterobicyclic compound of formula I*, corresponding to formula I, or a salt, stereoisomer, tautomer, or N-oxide thereof

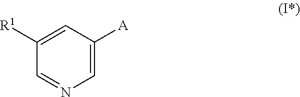

wherein
A is a moiety selected from the group consisting of

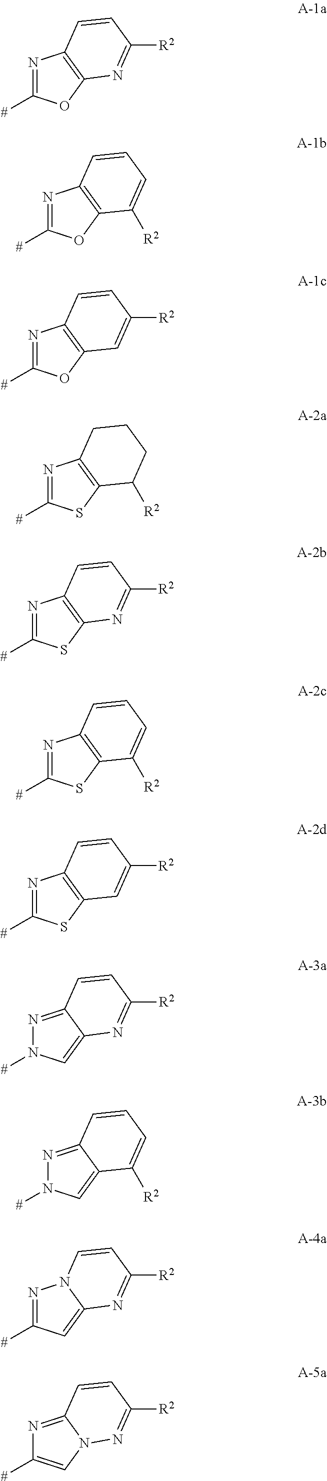

and wherein
R¹ and R² are as defined in embodiment 1.
7. The use of compounds of formula I according to any one of embodiments 1 to 5, or the compound of formula I* according to embodiment 6, wherein R¹ is H or F, preferably H.
8. The use of compounds I according to any one of embodiments 1 to 5, or the compounds I* according to any one of embodiments 6 or 7, wherein
   R¹² is H, CN, C₁-C₆-alkyl, C₁-C₆-haloalkyl, C₁-C₆-alkoxy, C₁-C₆-haloalkoxy, C₁-C₆-alkyl-S(O)ₙ, C₁-C₆-haloalky-S, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁵)₂R¹⁶, S(O)ₙR²⁰, S(O)ₙNR^{21a}R^{21b}, NR^{21a}R^{21b}, —N═CR¹⁷R¹⁸, —C(═O)R¹⁹, C(═O)NR^{21a}R^{21b}, C(═S)NR^{21a}R^{21b}, C(═O)OR²⁰,
      phenyl, optionally substituted with one or more substituents R²²; which are selected independently from one another, or a 3-, 4-, 5-, 6- or 7-membered saturated, partially unsaturated or fully unsaturated,
      or aromatic heterocyclic ring comprising 1, 2 or 3 heteroatoms selected from O, N and S, unsubstituted or substituted with one or more, e.g. 1, 2, 3 or 4, substituents R²², selected independently from one another, and wherein the N and/or S atoms of the heterocyclic ring may optionally be oxidized,
   R^{13a}, R^{13b} are each independently from one another selected from the group consisting of H, C₁-C₆-alkyl, C₁-C₆-haloalkyl, C₁-C₆-alkoxy, C₁-C₆-haloalkoxy, C₁-C₆-alkyl-S—, C₁-C₆-haloalky-S—, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, —S(O)ₙNR^{21a}R^{21b}, C(═O)R¹⁹, C(═O)OR²⁰, C(═O)NR^{21a}R^{21b}, C(═S)R¹⁹, C(═S)SR²⁰, C(═S)NR^{21a}R^{21b}, C(═NR^{21a})R¹⁹;
      phenyl, optionally substituted with one or more, e.g. 1, 2, 3 or 4, substituents R²², which are selected independently from one another;
      a 3-, 4-, 5-, 6- or 7-membered saturated, partially unsaturated or fully unsaturated, or aromatic heterocyclic ring comprising 1, 2, 3 or 4 heteroatoms selected from O, N and S, optionally substituted with one or more, e.g. 1, 2, 3 or 4, substituents R²², selected independently from one another, and wherein the N and/or S atoms of the heterocyclic ring may optionally be oxidized;
      or R^{13a} and R^{13b} are together a C₂-C₇ alkylene or C₂-C₇ alkenylene chain and form a 3-, 4-, 5-, 6-, 7- or 8-membered saturated, partially unsaturated or fully unsaturated, or aromatic ring together with the N-atom they are bonded to,
      wherein the alkylene chain or alkenylene chain may contain one or two heteratoms selected from O, S and N, and may optionally be substituted with halogen, C₁-C₆-alkyl, C₁-C₆-haloalkyl, C₁-C₆-alkoxy, C₁-C₆-haloalkoxy, C₁-C₆-alkyl-S—, C₁-C₆-haloalkyl-S, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl;
      phenyl, optionally substituted with one or more substituents R²²; which are selected independently from one another;
      or a 3-, 4-, 5-, 6,- or 7-membered saturated, partially unsaturated or fully unsaturated, or aromatic heterocyclic ring comprising 1, 2 or 3 heteroatoms selected from O, S, and N, optionally substituted with one or more substituents R²², selected independently from one another, and wherein the N and/or S atoms of the heterocyclic ring may optionally be oxidized;
   R^{21a}, R^{21b} are each independently from one another selected from the group consisting of H, CN, C₁-C₆-alkoxy, C₁-C₆-haloalkoxy, C₁-C₆-alkyl-S(O)ₙ—, trimethylsilyl, triethylsilyl, tert-butyldimethylsilyl,
      C₁-C₆-alkyl, C₂-C₆-alkenyl, C₂-C₆-alkynyl, C₃-C₈-cycloalkyl, C₃-C₈-cycloalkyl-C₁-C₆-alkyl, C₁-C₆-alkyl-C₃-C₈-cycloalkyl, wherein the aliphatic and cyclo-aliphatic radicals may be unsubstituted, partially or fully halogenated and/or oxygenated and/or may carry 1 or 2 radicals selected from C₁-C₄-alkoxy,
      phenyl, benzyl, pyridyl, and phenoxy, wherein the radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl;
      or R^{21a} and R^{21b} may together be a C₂-C₆ alkylene chain forming a 3- to 7-membered saturated, partly saturated or unsaturated ring together with the N-atom R^{21a} and R^{21b} are bonded to, wherein the alkylene chain may contain 1 or 2 heteroatoms selected from O, S, and N, and may optionally be substituted with halogen, C₁-C₄-haloalkyl, C₁-C₄-alkoxy or C₁-C₄-haloalkoxy, and wherein the N and/or S atoms of the heterocyclic ring may optionally be oxidized;
   and wherein all other substituents have the meaning as defined in embodiment 1.
9. The use of compounds of formula I according to any one of embodiments 1 to 5, or the compounds I* according to any one of embodiments 6 or 7, wherein
   R² is OR¹², S(O)ₙR¹², S(O)ₙNR^{13a}R^{13b}, preferably OR¹²;
   R¹² is C₁-C₆-alkyl, which may be substituted with CN, C₁-C₆-alkoxy, C₁-C₆-haloalkoxy, C₁-C₆-alkyl-S(O)ₙ, C₁-C₆-haloalkyl-S(O)ₙ, C₁-C₆-hetaryl;
      C₁-C₆-haloalkyl, which may be substituted with C₁-C₆-alkoxy, C₁-C₆-haloalkoxy, C₁-C₆-alkyl-S(O)ₙ, C₁-C₆-haloalkyl-S(O)ₙ;
      C₃-C₈-cycloalkyl-C₁-C₄-alkyl, which may be substituted with halogen, CN;
      C₁-C₄-alkyl-C₃-C₈-cycloalkyl, C₃-C₈-cycloalkyl, C₃-C₈-halocycloalkyl, C₂-C₆-alkenyl, C₂-C₆-alkynyl, NR^{21a}R^{21b};
   R^{13a}, R^{13b} are each independently from one another selected from the group consisting of C₁-C₆-alkyl, C₃-C₈-cycloalkyl, C₃-C₈-cycloalkyl-C₁₋C₆-alkyl, C(═O)R¹⁹;
   R^{21a}, R^{21b} are each independently from one another selected from the group consisting of H, C₁-C₆-alkyl, C₃-C₈-cycloalkyl, C₃-C₈-cycloalkyl-C₁-C₆-alkyl, C₁-C₆-alkyl-C₃-C₈-cycloalkyl, wherein the aliphatic and cyclo-aliphatic radicals may be unsubstituted, partially or fully halogenated and/or oxygenated and/or may carry 1 or 2 radicals selected from C₁-C₄-alkoxy,
   and wherein all other substituents have the meaning as defined in embodiment 1.
10. The use of compounds of formula I according to any one of embodiments 1 to 5, or the compounds I* according to any one of embodiments 6 to 8, wherein
   R² is a substituent

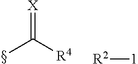

wherein
§ denotes the bond to the atom on which R² is present;
X is NR³, O, or S; and
R⁴ is H, CR⁵R⁶R⁷, NR⁸R⁹, OR¹⁰, or SR¹⁰,
wherein R⁵, R⁶, R⁷, R⁸, R⁹, and R¹⁰ are defined as in embodiment 1.
11. An agricultural or veterinary composition comprising at least a compound of formula I or formula I* as defined in any one of embodiments 1 to 10.
12. A method for combating or controlling invertebrate pests comprising contacting the invertebrate pests, or their food supply, habitat or breeding grounds with a pesticidally effective amount of a compound of formula I or formula I* as defined in any one of embodiments 1 to 10, or a composition according to embodiment 11.
13. A method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a compound of formula I or formula I* as defined in any one of embodiments 1 to 10, or a composition according to embodiment 11.
14. A compound of formula I or formula I* as defined in any of embodiments 1 to 10, or a composition according to embodiment 11 for use in treating or protecting animals against attack, infestation or infection by parasites, wherein the compound or composition is preferably administered or applied orally, topically or parenterally to the animals.
15. Seed comprising a compound of formula I or formula I* as defined in any one of embodiments 1 to 10.

1-15. (canceled)

16: A method for protecting crops, plants, plant propagation material, and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a substituted 3-pyridyl heterobicyclic compound of formula I or a salt, stereoisomer, tautomer, or N-oxide thereof 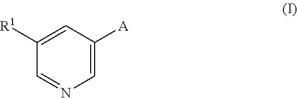
wherein
R¹ is H, CN or halogen; and
A is a moiety selected from 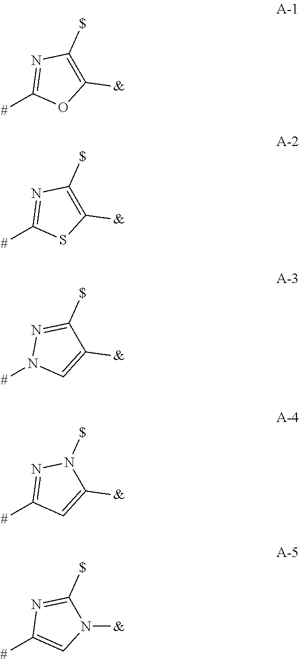
wherein
# denotes the bond to the pyridine ring of formula I; and
$ and & mark the connection to a C₃-C₄-alkylene, or C₃-C₄-alkenylene chain, of which none, or one or more C-atoms are independently replaced by a heteroatom O, N, or S, and which forms together with the atoms to which it is bonded an annulated saturated, partially unsaturated, or fully unsaturated carbocyclic, or heterocyclic ring;
wherein the N- and/or S-atoms of the chain are independently oxidized, or non-oxidized, and wherein the C-atoms and/or the heteroatoms of the chain are unsubstituted, or substituted by one or more, same or different substituents R²;
wherein
R² is
(i) halogen, CN, NO₂, C₁-C₁₀-alkyl, C₃-C₈-cycloalkyl, C₂-C₁₀-alkenyl, C₃-C₈-cycloalkenyl, C₂-C₁₀-alkynyl, which aliphatic and cycloaliphatic radicals are in each case unsubstituted or substituted with one or more, same or different substituents R¹¹;
OR¹², NR^{13a}R^{13b}, S(O)ₙR¹², S(O)ₙNR^{13a}R^{13b}, Si(R¹⁵)₂R¹⁶;
phenyl, which is unsubstituted, or substituted with one or more, same or different substituents R¹⁴;
or a 3- to 7-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents R¹⁴, and wherein said N- and S-atoms are independently oxidized, or non-oxidized;
with the proviso that R² is not halogen, if R² is bonded to a heteroatom; or
(ii) a substituent 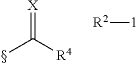
wherein
§ denotes the bond to the atom on which R² is present;
X is NR³, O, or S; and
R⁴ is H, CR⁵R⁶R⁷, NR⁸R⁹, OR¹⁰, or SR¹⁰; or
(iii) two R² present on one C- or S-atom are together ═O, ═S, or ═NR³;
and wherein
R³ is H, CN, NO₂, C₁-C₁₀-alkyl, C₃-C₈-cycloalkyl, C₂-C₁₀-alkenyl, C₂-C₁₀-alkynyl, which aliphatic and cycloaliphatic radicals are in each case unsubstituted, or substituted with one or more, same or different substituents R¹¹;
OR¹², NR^{13a}R^{13b}, S(O)ₙR¹², S(O)ₙNR^{13a}R^{13b}, C(═O)R¹¹, C(═O)NR^{13a}R^{13b}, C(═O)OR¹², C(═S)R¹¹, C(═S)NR^{13a}R^{13b}, C(═S)OR¹², C(═S)SR¹², C(═NR^{13a})R¹¹, C(═NR^{13a})NR^{13a}R^{13b};
Phenyl, which is unsubstituted, or substituted with one or more, same or different substituents R¹⁴;
or a 3- to 7-membered saturated, partially unsaturated, or fully unsaturated heterocyclic ring wherein said heterocyclic ring comprises one or more, same or different heteroatoms O, N, or S, and is unsubstituted, or substituted with one or more, same or different substituents R¹⁴, and wherein said N- and/or S-atoms, independently of one another, are oxidized, or non-oxidized;
R⁵, R⁶, R⁷ are independently H, halogen, CN, NO₂, —SCN, C₁-C₆-alkyl, C₁-C₆-haloalkyl, C₁-C₆-alkoxy, C₁-C₆-haloalkoxy, C₁-C₆-alkyl-S(O)ₙ—, C₁-C₆-haloalkyl-S(O)ₙ—, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl;
Si(R¹⁵)₂R¹⁶, OR¹², OSO₂R¹², S(O)ₙR¹², S(O)ₙNR^{13a}R^{13b}, NR^{13a}R^{13b}, C(═O)NR^{13a}R^{13b}, C(═S)NR^{13a}R^{13b}, C(═O)OR¹²;
phenyl, which is unsubstituted, or substituted with one or more, same or different substituents R¹⁴;
a 3- to 7-membered saturated, partially unsaturated, or fully unsaturated heterocyclic ring comprising one or more, same or different heteroatoms O, N or S, wherein said heterocyclic ring is unsubstituted, or substituted with one or more, same or different substituents R¹⁴, and wherein the N- and/or S-atoms of the heterocyclic ring are oxidized, or non-oxidized;
or R⁵ and R⁶ together form ═O, ═CR¹¹R¹⁷, ═S, ═S(O)ₙR¹²; ═S(O)ₙNR^{13a}R^{13b}, ═NR^{13a}, ═NOR¹², ═NNR^{13a}R^{13b}, and R⁷ is selected from the group above;
or R⁵ and R⁶ together form a 3- to 8-membered saturated or partially unsaturated carbocyclic or heterocyclic ring together with the C-atom to which they are bonded to, and R⁷ is selected from the group above;
R⁸, R⁹ are independently H, CN, C₁-C₁₀-alkyl, C₃-C₈-cycloalkyl, C₂-C₁₀-alkenyl, C₂-C₁₀-alkynyl, which aliphatic and cycloaliphatic radicals are unsubstituted or substituted with one or more substituents R¹¹;
OR¹², NR^{13a}R^{13b}, S(O)ₙNR^{13a}R^{13b}, C(═O)R¹¹, C(═O)NR^{13a}R^{13b}, C(═O)OR¹², C(═S)R¹¹, C(═S)NR^{13a}R^{13b}, C(═S)OR¹², C(═S)SR¹², C(═NR^{13a})R¹¹, C(═NR^{13a})NR^{13a}R^{13b}, Si(R¹⁵)₂R¹⁶;
phenyl, which is unsubstituted or substituted with one or more, same or different substituents R¹⁴;
a 3- to 7-membered saturated, partially unsaturated, or fully unsaturated heterocyclic ring, wherein said heterocyclic ring comprises one or more, same or different heteroatoms O, N, or S, and is unsubstituted or substituted with one or more, same or different substituents R¹⁴, and wherein said N- and/or S-atoms, independently of one another, are oxidized or non-oxidized; or
R⁸ and R⁹ together are part of a C₂-C₇-alkylene, C₂-C₇-alkenylene, or C₂-C₇-alkynylene chain and form a 3- to 8-membered saturated, partially unsaturated, or fully unsaturated heterocyclic ring together with the N-atom they are bonded to, wherein none, one or more of the CH₂, CH, or C groups are replaced by one or more, same or different groups C═O, C═S, O, N or NH, and wherein the C-, and/or N-atoms in the C₂-C₇-alkylene, C₂-C₇-alkenylene, or C₂-C₇-alkynylene chain are unsubstituted, or substituted by one or more, same or different substituents selected from halogen, CN, C₁-C₆-alkyl, C₁-C₆-haloalkyl, C₁-C₆-alkoxy, C₁-C₆-haloalkoxy, C₁-C₆-alky-S(O)ₙ—, C₁-C₆-haloalkyl-S(O)ₙ—, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, and phenyl, which groups are unsubstituted, or substituted with one or more, same or different substituents R¹¹, and wherein the S—, and/or N-atoms in the C₂-C₇-alkylene, C₂-C₇-alkenylene or C₂-C₇-alkynylene chain, independently of one another, are oxidized, or non-oxidized;
or R⁸ and R⁹ together form a ═CHR¹⁷, ═CR¹¹R¹⁷, ═NR^{13a} or ═NOR¹² radical;
R¹⁰ is H, CN, C₁-C₆-alkyl, C₁-C₆-haloalkyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl;
Si(R¹⁵)₂R¹⁶, S(O)ₙR¹², S(O)ₙNR^{13a}R^{13b}, NR^{13a}R^{13b}, —N═CR¹¹R¹⁷, C(═O)R¹¹, C(═O)NR^{13a}R^{13b}, C(═S)NR^{13a}R^{13b}, C(═O)OR¹²;
phenyl, which is unsubstituted, or substituted with one or more, same or different substituents R¹⁴;
or a 3- to 7-membered saturated, partially unsaturated, or fully unsaturated heterocyclic ring comprising one or more, same or different heteroatoms O, N or S, wherein the heterocyclic ring is unsubstituted or substituted with one or more, same or different substituents R¹⁴, and wherein the N- and/or S-atoms of the heterocyclic ring are oxidized, or non-oxidized;
and wherein
R¹¹ is H, halogen, CN, NO₂, —SCN, C₁-C₆-alkyl, C₁-C₆-haloalkyl, C₁-C₆-alkoxy, C₁-C₆-haloalkoxy, C₁-C₆-alkyl-S(O)ₙ—, C₁-C₆-haloalkyl-S(O)ₙ—, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl;
Si(R¹⁵)₂R¹⁶, OR²⁰, OSO₂R²⁰, S(O)ₙR²⁰, S(O)ₙNR^{21a}R^{21b}, NR^{21a}R^{21b}C(═O)NR^{21a}R^{21b}, C(═S)NR^{21a}R^{21b}, C(═O)OR²⁰;
phenyl, which is unsubstituted, or substituted with one or more, same or different substituents R²²,
or a 3- to 7-membered saturated, partially unsaturated, or fully unsaturated heterocyclic ring comprising one or more, same or different heteroatoms O, N or S, wherein the heterocyclic ring is unsubstituted, or substituted with one or more, same or different substituents R²², and wherein the N- and/or S-atoms of the heterocyclic ring are oxidized, or non-oxidized;
or two R¹¹ present on one C-atom together form ═O, ═CR¹⁷R¹⁸, ═S, ═S(O)ₙR²⁰; ═S(O)ₙNR^{21a}R^{21b}, ═NR^{21a}═NOR²⁰, ═NNR^{21a};
or two R¹¹ form a 3- to 8-membered saturated, or partially unsaturated carbocyclic, or heterocyclic ring together with the C-atoms to which the two R¹¹ are bonded to;
R¹² is H, CN;
C₁-C₆-alkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
C₁-C₆-haloalkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
C₂-C₆-alkenyl, C₂-C₆-haloalkenyl, C₂-C₆-alkynyl, or C₂-C₆-haloalkynyl;
C₃-C₈-cycloalkyl, C₃-C₈-halocycloalkyl, C₃-C₈-cycloalkyl-C₁-C₄-alkyl, or C₃-C₈-halocycloalkyl-C₁-C₄-alkyl, wherein the cyclic moieties are unsubstituted, or substituted with one or more, same or different substituents selected from halogen, CN, C₁-C₄-alkyl, C₁-C₄-haloalkyl, C₁-C₄-alkoxy, C₁-C₄-haloalkoxy, ═O, and acetal groups thereof based on alcohols, or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH;
Si(R¹⁵)₂R¹⁶, S(O)ₙR²⁰, S(O)ₙNR^{21a}R^{21b} NR^{21a}R^{21b}, N═CR¹⁷R¹⁸, C(═O)R¹⁹, C(═O)NR^{21a}R^{21b}, C(═S)NR^{21a}R^{21b}, C(═O)OR²⁰;
phenyl, or phenyl-C₁-C₄-alkyl, wherein the aromatic rings are unsubstituted, or substituted with one or more, same or different substituents R²²;
or a 3- to 7-membered saturated, partially unsaturated, or fully unsaturated heterocyclic ring comprising one or more, same or different heteroatoms O, N or S, wherein the heterocyclic ring is directly bonded to the remainder of the molecule, or bonded to the remainder of the molecule via a C₁-C₄-alkyl group, and wherein the heterocyclic ring is unsubstituted, or substituted with one or more, same or different substituents R²², and wherein the N- and/or S-atoms of the heterocyclic ring are oxidized, or non-oxidized;
R^{13a}, R^{13b} are independently H, C₁-C₆-alkyl, C₁-C₆-haloalkyl, C₁-C₆-alkoxy, C₁-C₆-haloalkoxy, C₁-C₆-alkyl-S—, C₁-C₆-haloalky-S—, C₃-C₈-cycloalkyl, C₃-C₈-cycloalkyl-C₁-C₆-alkyl, C₃-C₈-halocycloalkyl, C₂-C₆-alkenyl, C₂-C₆-haloalkenyl, C₂-C₆-alkynyl, C₂-C₆ haloalkynyl, —S(O)ₙNR^{21a}R^{21b}, C(═O)R¹⁹, C(═O)OR²⁰, C(═O)NR^{21a}R^{21b}, C(═S)R¹⁹, C(═S)SR²⁰, C(═S)NR^{21a}R^{21b} C(═NR^{21a})R¹⁹;
phenyl, which is unsubstituted, or substituted with one or more, same or different substituents R²²;
a 3- to 7-membered saturated, partially unsaturated, or fully unsaturated heterocyclic ring comprising one or more, same or different heteroatoms O, N or S, wherein the heterocyclic ring is unsubstituted, or substituted with one or more, same or different substituents R²², and wherein the N- and/or S-atoms of the heterocyclic ring are oxidized, or non-oxidized;
or R^{13a} and R^{13b} are together a C₂-C₇ alkylene, or C₂-C₇ alkenylene chain, and form a 3- to 8-membered saturated, partially unsaturated, or fully unsaturated ring together with the N-atom they are bonded to,
wherein the C₂-C₇ alkylene chain or C₂-C₇ alkenylene chain contains none, one or more, same or different heteratoms O, S or N, and is substituted or unsubstituted with one or more, same or different substituents selected from halogen, C₁-C₆-alkyl, C₁-C₆-haloalkyl, C₁-C₆-alkoxy, C₁-C₆-haloalkoxy, C₁-C₆-alkyl-S—, C₁-C₆-haloalkyl-S, C₃-C₈-cycloalkyl, C₃-C₈-halocycloalkyl, C₂-C₆-alkenyl, C₂-C₆-haloalkenyl, C₂-C₆-alkynyl, and C₂-C₆ haloalkynyl;
phenyl, which is unsubstituted, or substituted with one or more, same or different substituents R²²;
or a 3- to 7-membered saturated, partially unsaturated, or fully unsaturated heterocyclic ring comprising one or more, same or different heteroatoms O, S, or N, and is substituted or unsubstituted with one or more, same or different substituents R²², and wherein the N- and/or S-atoms of the heterocyclic ring are oxidized or non-oxidized;
or R^{13a} and R¹³b together form a ═CR¹⁷R¹⁸, ═NR²¹, or ═NOR²⁰ radical;
R¹⁴ is H, halogen, CN, NO₂, SCN, SF₅, C₁-C₁₀-alkyl, C₃-C₈-cycloalkyl, C₂-C₁₀-alkenyl, C₂-C₁₀-alkynyl, wherein the aliphatic and cycloaliphatic radicals are unsubstituted or substituted with one or more, same or different substituents R¹⁹; Si(R¹⁵)₂R¹⁶, OR²⁰, OS(O)ₙR²⁰, —S(O)ₙR²⁰, S(O)ₙNR^{21a}R^{21b}, NR^{21a}R^{21b}, C(═O)R¹⁹, C(═O)OR²⁰, —C(═NR^{21a})R¹⁹, C(═O)NR^{21a}R^{21b}, C(═S)NR^{21a}R^{21b}b;
phenyl, which is unsubstituted or substituted with one or more, same or different substituents selected from halogen, CN, NO₂, C₁-C₆-alkyl, C₁-C₆-haloalkyl, C₁-C₆-alkoxy, and C₁-C₆-haloalkoxy;
or a 3- to 7-membered saturated, partially unsaturated, or fully unsaturated, or aromatic heterocyclic ring comprising one or more, same or different heteroatoms O, N, or S, which is unsubstituted or substituted with one or more, same or different substituents selected from halogen, CN, NO₂, C₁-C₆-alkyl, C₁-C₆-haloalkyl, C₁-C₆-alkoxy, and C₁-C₆-haloalkoxy, and wherein the N- and/or S-atoms of the heterocyclic ring are oxidized or non-oxidized;
or two R¹⁴ present together on one atom of a partly saturated heterocycle are ═O, ═CR¹⁷R¹⁸, ═NR^{21a}, ═NOR²⁰, or ═NNR^{21a};
or two R¹⁴ on adjacent C-atoms form a bridge CH₂CH₂CH₂CH₂, CH═CHCH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{21a}, CH₂CH═N, CH═CH—NR^{21a}, OCH═N, SCH═N, wherein the bridge is substituted or unsubstituted with one or more, same or different substituents selected from ═O, OH, CH₃, OCH₃, halogen, CN, halomethyl, or halomethoxy;
R¹⁵, R¹⁶ are independently H, C₁-C₆-alkyl, C₁-C₆-haloalkyl, C₁-C₆-alkoxy, C₁-C₄-alkoxy-C₁-C₄-alkyl, C₃-C₈-cycloalkyl, C₃-C₈-halocycloalkyl, C₁-C₄-haloalkoxy-C₁-C₄-alkyl, or phenyl;
R¹⁷, R¹⁸ are independently H, C₁-C₄-alkyl, C₁-C₆-cycloalkyl, C₁-C₂-alkoxy-C₁-C₂-alkyl, phenyl, or benzyl;
R¹⁹ is H, halogen, CN, NO₂, OH, SH, SCN, SF₅, C₁-C₆-alkoxy, C₁-C₆-haloalkoxy, C₁-C₆-alkyl-S(O)ₙ—, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, C₃-C₈-cycloalkyl, wherein the aliphatic and cyclo-aliphatic radicals are unsubstituted, partially or fully halogenated and/or oxgenated and/or are unsubstituted or substituted by one or more, same or different radicals selected from C₁-C₄ alkoxy;
phenyl, benzyl, pyridyl, or phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino, and di-(C₁-C₆-alkyl)amino;
or two R¹⁹ present on the same C-atom are together ═O, ═CH(C₁-C₄-alkyl), ═C(C₁-C₄-alkyl)C₁-C₄-alkyl, ═N(C₁-C₆-alkyl), or ═NO(C₁-C₆-alkyl);
R²⁰ is H, CN, C₁-C₆-alkyl-S(O)ₙ, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, C₃-C₈-cycloalkyl, wherein the aliphatic and cycloaliphatic radicals are unsubstituted, partially or fully halogenated and/or oxygenated and/or substituted by one or more, same or different radicals selected from C₁-C₄-alkoxy;
phenyl, benzyl, pyridyl, or phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
R^{21a}, R^{21b} are independently H, CN, C₁-C₆-alkoxy, C₁-C₆-haloalkoxy, C₁-C₆-alkylS(O)ₙ—, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, C₃-C₈-cycloalkyl, C₃-C₈-cycloalkyl-C₁-C₆-alkyl, C₁-C₆-alkyl-C₃-C₈-cycloalkyl, wherein the aliphatic and cycloaliphatic radicals are unsubstituted, partially or fully halogenated and/or oxygenated and/or substituted by one or more, same or different radicals selected from C₁-C₄-alkoxy;
phenyl, benzyl, pyridyl, and phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
or R^{21a} and R^{21b} together represent a C₂-C₆ alkylene chain forming a 3- to 7-membered saturated, partially unsaturated, or fully unsaturated heterocyclic ring together with the N-atom R^{21a} and R^{21b} are bonded to, wherein the alkylene chain contains none, or one or more, same or different heteroatoms O, S, or N, and is unsubstituted or substituted with one or more, same or different substituents selected from halogen, C₁-C₄-haloalkyl, C₁-C₄-alkoxy, and C₁-C₄-haloalkoxy, and wherein the N- and/or S-atoms of the heterocyclic ring are oxidized or non-oxidized;
R²² is H, halogen, NO₂, CN, OH, SH, C₁-C₆-alkoxy, C₁-C₆-haloalkoxy, C₁-C₆-alkylS(O)ₙ, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, or C₃-C₈-cycloalkyl, wherein the aliphatic and cyclo-aliphatic radicals are unsubstituted, partially or fully halogenated and/or oxygenated and/or are substituted by one or more, same or different radicals selected from C₁-C₄-alkoxy;
phenyl, benzyl, pyridyl, or phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
or two R²² present together on one atom are ═O, ═S, ═N(C₁-C₆-alkyl), ═NO(C₁-C₆-alkyl), ═CH(C₁-C₄-alkyl), or ═C(C₁-C₄-alkyl)C₁-C₄-alkyl;
or two R²² on two adjacent C-atoms are together a C₂-C₆ alkylene chain or C₂-C₆ alkenylene chain, which form together with the C-atom they are bonded to a 3- to 7-membered saturated, partially unsaturated, or fully unsaturated ring, wherein the alkylene or alkenylene chain contains none, or one or more, same or different heteroatoms selected from O, S, or N, and is unsubstituted or substituted with halogen, C₁-C₄-haloalkyl, C₁-C₄-alkoxy, or C₁-C₄-haloalkoxy, and wherein the N- and/or S-atoms of the heterocyclic ring are oxidized or non-oxidized;
n is 0, 1, or 2.

17: The method of claim 16, wherein
A is a moiety selected from A-1, A-2, A-3, A-4, and A-5, wherein
# denotes the bond to the pyridine ring of formula I; and
$ and & mark the connection to a C₄-alkylene or C₄-alkenylene chain of which none, or
one C-atom is replaced by N, and which forms together with the atoms to which it is
bonded an annulated saturated, partially unsaturated, or fully unsaturated 6-membered
carbocyclic or heterocyclic ring,
wherein the C-atoms and/or the N-atom of the chain are unsubstituted, or substituted by
one or more, same or different substituents R².

18: The method of claim 16, wherein
A is a moiety A-1, A-2, A-3, A-4, or A-5, wherein
# denotes the bond to the pyridine ring of formula I; and
$ and & mark the connection to a chain, which is selected from 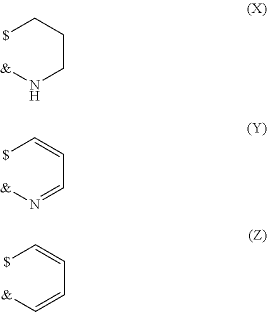
wherein in each case $ marks the connection to the $-position of the moiety A and &
marks the connection to the &-position of the moiety A;
and wherein the C-atoms and/or the N-atom of the chains are unsubstituted or
substituted by one or more, same or different substituents R².

19: The method of claim 16, wherein
A is a moiety A-1, A-2, A-3, A-4, or A-5; wherein
# denotes the bond to the pyridine ring of formula I; and
$ and & mark the connection to a chain, which is selected from 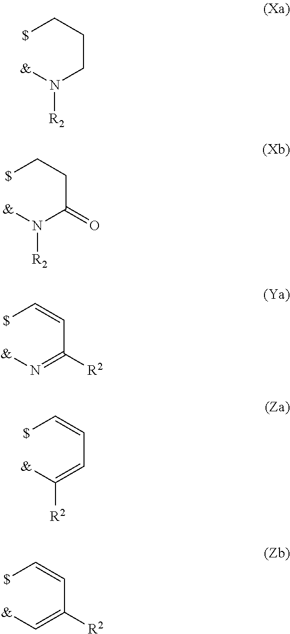
wherein in each case $ marks the connection to the $-position of the moiety A, and &
marks the connection to the &-position of the moiety A.

20: The method of claim 16, wherein
A is a moiety selected from 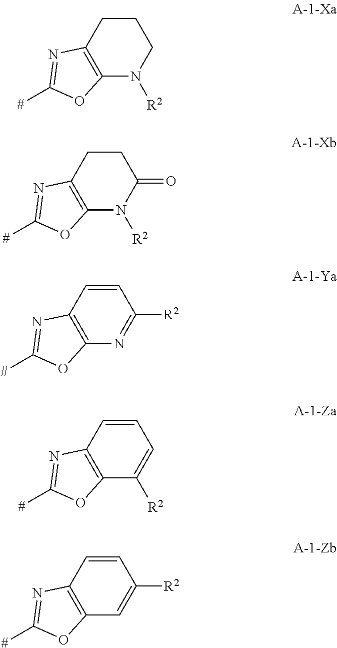
or
A is a moiety selected from 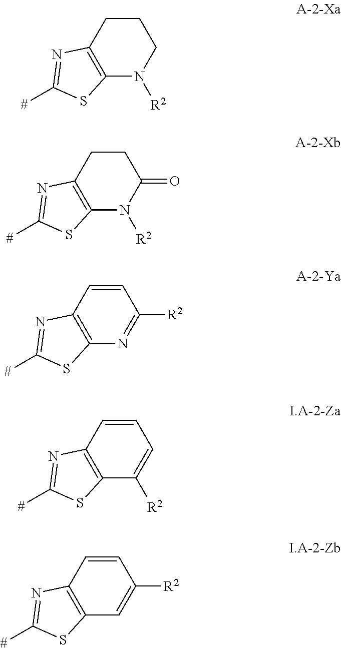
or
A is a moiety selected from 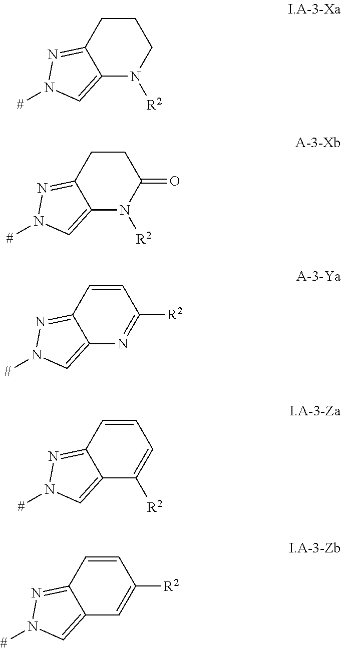
or
A is a moiety selected from 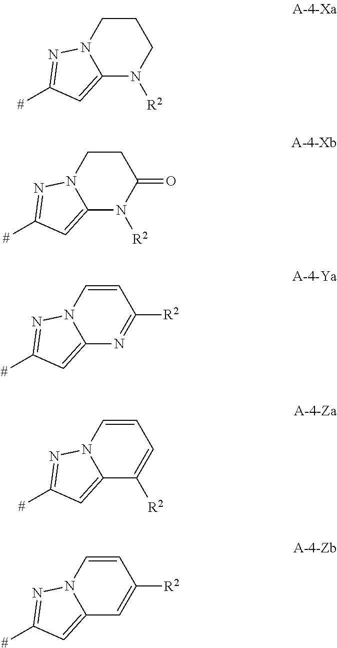
or
A is a moiety selected from

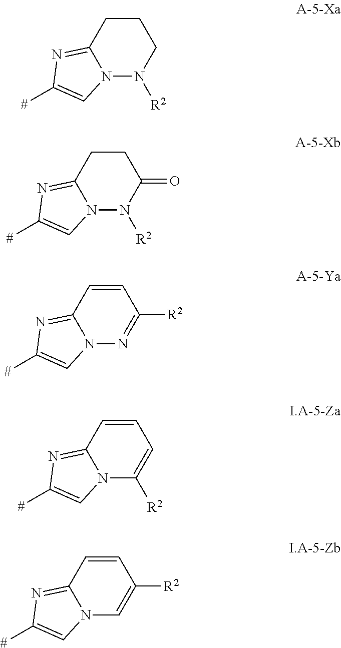

21: A compound of formula (I*), or a salt, stereoisomer, tautomer, or N-oxide thereof 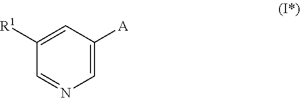
wherein
A is a moiety selected from 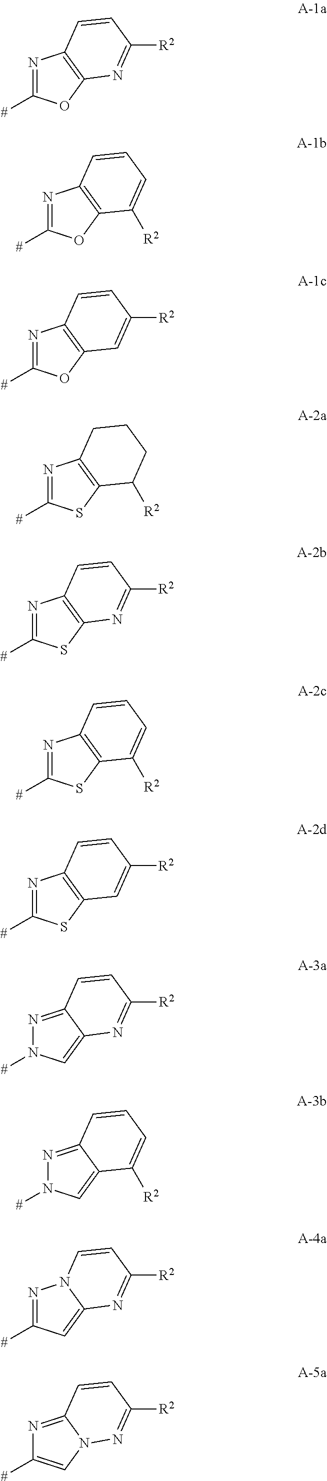
and wherein
R¹ is H, CN or halogen; and
R² is
(i) halogen, CN, NO₂, C₁-C₁₀-alkyl, C₃-C₈-cycloalkyl, C₂-C₁₀-alkenyl, C₃-C₈-cycloalkenyl, C₂-C₁₀-alkynyl, which aliphatic and cycloaliphatic radicals are in each case unsubstituted or substituted with one or more, same or different substituents R¹¹;
OR¹², NR^{13a}R^{13b}, S(O)ₙR¹², S(O)ₙNR^{13a}R^{13b}, Si(R¹⁵)₂R¹⁶;
phenyl, which is unsubstituted, or substituted with one or more, same or different substituents R¹⁴;
or a 3- to 7-membered saturated, partially unsaturated, or fully unsaturated heterocyclic ring, wherein said heterocyclic ring comprises one or more, same or different heteroatoms O, N, or S, and is unsubstituted, or substituted with one or more, same or different substituents R¹⁴, and wherein said N- and S-atoms are independently oxidized, or non-oxidized;
with the proviso that R² is not halogen, if R² is bonded to a heteroatom; or
(ii) a substituent 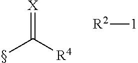
wherein
§ denotes the bond to the atom on which R² is present;
X is NR³, O, or S; and
R⁴ is H, CR⁵R⁶R⁷, NR⁸R⁹, OR¹⁰, or SR¹⁰; or
(iii) two R² present on one C- or S-atom are together ═O, ═S, or ═NR³;
and wherein
R³ is H, CN, NO₂, C₁-C₁₀-alkyl, C₃-C₈-cycloalkyl, C₂-C₁₀-alkenyl, C₂-C₁₀-alkynyl, which aliphatic and cycloaliphatic radicals are in each case unsubstituted, or substituted with one or more, same or different substituents R¹¹;
OR¹², NR^{13a}R^{13b}, S(O)ₙR¹², S(O)ₙNR^{13a}R^{13b}, C(═O)R¹¹, C(═O)NR^{13a}R^{13b}, C(═O)OR¹², C(═S)R¹¹, C(═S)NR^{13a}R^{13b}, C(═S)OR¹², C(═S)SR¹², C(═NR^{13a})R¹¹, C(═NR^{13a})NR^{13a}R^{13b};
Phenyl, which is unsubstituted, or substituted with one or more, same or different substituents R¹⁴;
or a 3- to 7-membered saturated, partially unsaturated, or fully unsaturated heterocyclic ring wherein said heterocyclic ring comprises one or more, same or different heteroatoms O, N, or S, and is unsubstituted, or substituted with one or more, same or different substituents R¹⁴, and wherein said N- and/or S-atoms, independently of one another, are oxidized, or non-oxidized;
R⁵, R⁶, R⁷ are independently H, halogen, CN, NO₂, —SCN, C₁-C₆-alkyl, C₁-C₆-haloalkyl, C₁-C₆-alkoxy, C₁-C₆-haloalkoxy, C₁-C₆-alkyl-S(O)ₙ—, C₁-C₆-haloalkyl-S(O)ₙ—, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl;
Si(R¹⁵)₂R¹⁶, OR¹², OSO₂R¹², S(O)ₙR¹², S(O)ₙNR^{13a}R^{13b}, NR^{13a}R^{13b}, C(═O)NR^{13a}R^{13b}, C(═S)NR^{13a}R^{13b}, C(═O)OR¹²;
phenyl, which is unsubstituted, or substituted with one or more, same or different substituents R¹⁴;
a 3- to 7-membered saturated, partially unsaturated, or fully unsaturated heterocyclic ring comprising one or more, same or different heteroatoms O, N or S, wherein said heterocyclic ring is unsubstituted, or substituted with one or more, same or different substituents R¹⁴, and wherein the N- and/or S-atoms of the heterocyclic ring are oxidized, or non-oxidized;
or R⁵ and R⁶ together form ═O, ═CR¹¹R¹⁷, ═S, ═S(O)ₙR¹²; ═S(O)ₙNR^{13a}R^{13b}, ═NR^{13a}, ═NOR¹², ═NNR^{13a}R^{13b}, and R⁷ is selected from the group above;
or R⁵ and R⁶ together form a 3- to 8-membered saturated or partially unsaturated carbocyclic or heterocyclic ring together with the C-atom to which they are bonded to, and R⁷ is selected from the group above;
R⁸, R⁹ are independently H, CN, C₁-C₁₀-alkyl, C₃-C₈-cycloalkyl, C₂-C₁₀-alkenyl, C₂-C₁₀-alkynyl, which aliphatic and cycloaliphatic radicals are unsubstituted or substituted with one or more substituents R¹¹;
OR¹², NR^{13a}R^{13b}, S(O)ₙNR^{13a}R^{13b}, C(═O)R¹¹, C(═O)NR^{13a}R^{13b}, C(═O)OR¹², C(═S)R¹¹, C(═S)NR^{13a}R^{13b}, C(═S)OR¹², C(═S)SR¹², C(═NR^{13a})R¹¹, C(═NR^{13a})NR^{13a}R^{13b}, Si(R¹⁵)₂R¹⁶;
phenyl, which is unsubstituted or substituted with one or more, same or different substituents R¹⁴;
a 3- to 7-membered saturated, partially unsaturated, or fully unsaturated heterocyclic ring, wherein said heterocyclic ring comprises one or more, same or different heteroatoms O, N, or S, and is unsubstituted or substituted with one or more, same or different substituents R¹⁴, and wherein said N- and/or S-atoms, independently of one another, are oxidized or non-oxidized; or
R⁸ and R⁹ together are part of a C₂-C₇-alkylene, C₂-C₇-alkenylene, or C₂-C₇-alkynylene chain and form a 3- to 8-membered saturated, partially unsaturated, or fully unsaturated heterocyclic ring together with the N-atom they are bonded to, wherein none, one or more of the CH₂, CH, or C groups are replaced by one or more, same or different groups C═O, C═S, O, N or NH, and wherein the C-, and/or N-atoms in the C₂-C₇-alkylene, C₂-C₇-alkenylene, or C₂-C₇-alkynylene chain are unsubstituted, or substituted by one or more, same or different substituents selected from halogen, CN, C₁-C₆-alkyl, C₁-C₆-haloalkyl, C₁-C₆-alkoxy, C₁-C₆-haloalkoxy, C₁-C₆-alky-S(O)ₙ—, C₁-C₆-haloalkyl-S(O)ₙ—, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, and phenyl, which groups are unsubstituted, or substituted with one or more, same or different substituents R¹¹, and wherein the S—, and/or N-atoms in the C₂-C₇-alkylene, C₂-C₇-alkenylene or C₂-C₇-alkynylene chain, independently of one another, are oxidized, or non-oxidized;
or R⁸ and R⁹ together form a ═CHR¹⁷, ═CR¹¹R¹⁷, ═NR^{13a} or ═NOR¹² radical;
R¹⁰ is H, CN, C₁-C₆-alkyl, C₁-C₆-haloalkyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl;
Si(R¹⁵)₂R¹⁶, S(O)ₙR¹², S(O)ₙNR^{13a}R^{13b}, NR^{13a}R^{13b}, — N═CR¹¹R₁₇, C(═O)R₁₁, C(═O)NR^{13a}R^{13b}, C(═S)NR^{13a}R^{13b}, C(═O)OR¹²;
phenyl, which is unsubstituted, or substituted with one or more, same or different substituents R¹⁴;
or a 3- to 7-membered saturated, partially unsaturated, or fully unsaturated heterocyclic ring comprising one or more, same or different heteroatoms O, N or S, wherein the heterocyclic ring is unsubstituted or substituted with one or more, same or different substituents R¹⁴, and wherein the N- and/or S-atoms of the heterocyclic ring are oxidized, or non-oxidized;
and wherein
R¹¹ is H, halogen, CN, NO₂, —SCN, C₁-C₆-alkyl, C₁-C₆-haloalkyl, C₁-C₆-alkoxy, C₁-C₆-haloalkoxy, C₁-C₆-alkyl-S(O)ₙ—, C₁-C₆-haloalkyl-S(O)ₙ—, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl;
Si(R¹⁵)₂R¹⁶, OR²⁰, OSO₂R²⁰, S(O)ₙR²⁰, S(O)ₙNR^{21a}R^{21b}, NR^{21a}R^{21b}C(═O)NR^{21a}R^{21b}, C(═S)NR^{21a}R^{21b}, C(═O)OR²⁰;
phenyl, which is unsubstituted, or substituted with one or more, same or different substituents R²²,
or a 3- to 7-membered saturated, partially unsaturated, or fully unsaturated heterocyclic ring comprising one or more, same or different heteroatoms O, N or S, wherein the heterocyclic ring is unsubstituted, or substituted with one or more, same or different substituents R²², and wherein the N- and/or S-atoms of the heterocyclic ring are oxidized, or non-oxidized;
or two R¹¹ present on one C-atom together form ═O, ═CR¹⁷R¹⁸, ═S, ═S(O)ₙR²⁰;
═S(O)ₙNR^{21a}R^{21b}, ═NR^{21a}, ═NOR²⁰, ═NNR^{21a};
or two R¹¹ form a 3- to 8-membered saturated, or partially unsaturated carbocyclic, or heterocyclic ring together with the C-atoms to which the two R¹¹ are bonded to;
R¹² is H, CN;
C₁-C₆-alkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
C₁-C₆-haloalkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
C₂-C₆-alkenyl, C₂-C₆-haloalkenyl, C₂-C₆-alkynyl, or C₂-C₆-haloalkynyl;
C₃-C₈-cycloalkyl, C₃-C₈-halocycloalkyl, C₃-C₈-cycloalkyl-C₁-C₄-alkyl, or C₃-C₈-halocycloalkyl-C₁-C₄-alkyl, wherein the cyclic moieties are unsubstituted, or substituted with one or more, same or different substituents selected from halogen, CN, C₁-C₄-alkyl, C₁-C₄-haloalkyl, C₁-C₄-alkoxy, C₁-C₄-haloalkoxy, ═O, and acetal groups thereof based on alcohols, or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH;
Si(R¹⁵)₂R¹⁶, S(O)ₙR²⁰, S(O)ₙNR^{21a}R^{21b} NR^{21a}R^{21b}, N═CR¹⁷R¹⁸, C(═O)R¹⁹, C(═O)NR^{21a}R^{21b}, C(═S)NR^{21a}R^{21b}, C(═O)OR²⁰;
phenyl, or phenyl-C₁-C₄-alkyl, wherein the aromatic rings are unsubstituted, or substituted with one or more, same or different substituents R²²;
or a 3- to 7-membered saturated, partially unsaturated, or fully unsaturated heterocyclic ring comprising one or more, same or different heteroatoms O, N or S, wherein the heterocyclic ring is directly bonded to the remainder of the molecule, or bonded to the remainder of the molecule via a C₁-C₄-alkyl group, and wherein the heterocyclic ring is unsubstituted, or substituted with one or more, same or different substituents R²², and wherein the N- and/or S-atoms of the heterocyclic ring are oxidized, or non-oxidized;
R^{13a}, R^{13b} are independently H, C₁-C₆-alkyl, C₁-C₆-haloalkyl, C₁-C₆-alkoxy, C₁-C₆-haloalkoxy, C₁-C₆-alkyl-S—, C₁-C₆-haloalky-S—, C₃-C₈-cycloalkyl, C₃-C₈-cycloalkyl-C₁-C₆-alkyl, C₃-C₈-halocycloalkyl, C₂-C₆-alkenyl, C₂-C₆-haloalkenyl, C₂-C₆-alkynyl, C₂-C₆ haloalkynyl, —S(O)ₙNR^{21a}R^{21b}, C(═O)R¹⁹, C(═O)OR²⁰, C(═O)NR^{21a}R^{21b}, C(═S)R¹⁹, C(═S)SR²⁰, C(═S)NR^{21a}R^{21b} C(═NR^{21a})R¹⁹;
phenyl, which is unsubstituted, or substituted with one or more, same or different substituents R²²;
a 3- to 7-membered saturated, partially unsaturated, or fully unsaturated heterocyclic ring comprising one or more, same or different heteroatoms O, N or S, wherein the heterocyclic ring is unsubstituted, or substituted with one or more, same or different substituents R²², and wherein the N- and/or S-atoms of the heterocyclic ring are oxidized, or non-oxidized;
or R^{13a} and R^{13b} are together a C₂-C₇ alkylene, or C₂-C₇ alkenylene chain, and form a 3- to 8-membered saturated, partially unsaturated, or fully unsaturated ring together with the N-atom they are bonded to,
wherein the C₂-C₇ alkylene chain or C₂-C₇ alkenylene chain contains none, one or more, same or different heteratoms O, S or N, and is substituted or unsubstituted with one or more, same or different substituents selected from halogen, C₁-C₆-alkyl, C₁-C₆-haloalkyl, C₁-C₆-alkoxy, C₁-C₆-haloalkoxy, C₁-C₆-alkyl-S—, C₁-C₆-haloalkyl-S, C₃-C₈-cycloalkyl, C₃-C₈-halocycloalkyl, C₂-C₆-alkenyl, C₂-C₆-haloalkenyl, C₂-C₆-alkynyl, and C₂-C₆ haloalkynyl;
phenyl, which is unsubstituted, or substituted with one or more, same or different substituents R²²;
or a 3- to 7-membered saturated, partially unsaturated, or fully unsaturated heterocyclic ring comprising one or more, same or different heteroatoms O, S, or N, and is substituted or unsubstituted with one or more, same or different substituents R²², and wherein the N- and/or S-atoms of the heterocyclic ring are oxidized or non-oxidized;
or R^{13a} and R^{13b} together form a ═CR¹⁷R¹⁸, ═NR²¹, or ═NOR²⁰ radical;
R¹⁴ is H, halogen, CN, NO₂, SCN, SF₅, C₁-C₁₀-alkyl, C₃-C₈-cycloalkyl, C₂-C₁₀-alkenyl, C₂-C₁₀-alkynyl, wherein the aliphatic and cycloaliphatic radicals are unsubstituted or substituted with one or more, same or different substituents R¹⁹;
Si(R¹⁵)₂R¹⁶, OR²⁰, OS(O)ₙR²⁰, —S(O)ₙR²⁰, S(O)ₙNR^{21a}R^{21b}, NR^{21a}R^{21b}, C(═O)R¹⁹, C(═O)OR²⁰, —C(═NR^{21a})R¹⁹, C(═O)NR^{21a}R^{21b}, C(═S)NR^{21a}R^{21b};
phenyl, which is unsubstituted or substituted with one or more, same or different substituents selected from halogen, CN, NO₂, C₁-C₆-alkyl, C₁-C₆-haloalkyl, C₁-C₆-alkoxy, and C₁-C₆-haloalkoxy;
or a 3- to 7-membered saturated, partially unsaturated, or fully unsaturated, or aromatic heterocyclic ring comprising one or more, same or different heteroatoms O, N, or S, which is unsubstituted or substituted with one or more, same or different substituents selected from halogen, CN, NO₂, C₁-C₆-alkyl, C₁-C₆-haloalkyl, C₁-C₆-alkoxy, and C₁-C₆-haloalkoxy, and wherein the N- and/or S-atoms of the heterocyclic ring are oxidized or non-oxidized;
or two R¹⁴ present together on one atom of a partly saturated heterocycle are ═O, ═CR¹⁷R¹⁸, ═NR^{21a}, ═NOR²⁰, or ═NNR^{21a};
or two R¹⁴ on adjacent C-atoms form a bridge CH₂CH₂CH₂CH₂, CH═CHCH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{21a}, CH₂CH═N, CH═CH—NR^{21a}, OCH═N, SCH═N, wherein the bridge is substituted or unsubstituted with one or more, same or different substituents selected from ═O, OH, CH₃, OCH₃, halogen, CN, halomethyl, or halomethoxy;
R¹⁵, R¹⁶ are independently H, C₁-C₆-alkyl, C₁-C₆-haloalkyl, C₁-C₆-alkoxy, C₁-C₄-alkoxy-C₁-C₄-alkyl, C₃-C₈-cycloalkyl, C₃-C₈-halocycloalkyl, C₁-C₄-haloalkoxy-C₁-C₄-alkyl, or phenyl;
R¹⁷, R¹⁸ are independently H, C₁-C₄-alkyl, C₁-C₆-cycloalkyl, C₁-C₂-alkoxy-C₁-C₂-alkyl, phenyl, or benzyl;
R¹⁹ is H, halogen, CN, NO₂, OH, SH, SCN, SF₅, C₁-C₆-alkoxy, C₁-C₆-haloalkoxy, C₁-C₆-alkyl-S(O)ₙ—, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, C₃-C₈-cycloalkyl, wherein the aliphatic and cyclo-aliphatic radicals are unsubstituted, partially or fully halogenated and/or oxgenated and/or are unsubstituted or substituted by one or more, same or different radicals selected from C₁-C₄ alkoxy;
phenyl, benzyl, pyridyl, or phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino, and di-(C₁-C₆-alkyl)amino;
or two R¹⁹ present on the same C-atom are together ═O, ═CH(C₁-C₄-alkyl), ═C(C₁-C₄-alkyl)C₁-C₄-alkyl, ═N(C₁-C₆-alkyl), or ═NO(C₁-C₆-alkyl);
R²⁰ is H, CN, C₁-C₆-alkyl-S(O)ₙ, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, C₃-C₈-cycloalkyl,
wherein the aliphatic and cycloaliphatic radicals are unsubstituted, partially or fully halogenated and/or oxygenated and/or substituted by one or more, same or different radicals selected from C₁-C₄-alkoxy;
phenyl, benzyl, pyridyl, or phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
R^{21a}, R^{21b} are independently H, CN, C₁-C₆-alkoxy, C₁-C₆-haloalkoxy, C₁-C₆-alkylS(O)ₙ—, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, C₃-C₈-cycloalkyl, C₃-C₈-cycloalkyl-C₁-C₆-alkyl, C₁-C₆-alkyl-C₃-C₈-cycloalkyl, wherein the aliphatic and cycloaliphatic radicals are unsubstituted, partially or fully halogenated and/or oxygenated and/or substituted by one or more, same or different radicals selected from C₁-C₄-alkoxy;
phenyl, benzyl, pyridyl, and phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
or R^{21a} and R^{21b} together represent a C₂-C₆ alkylene chain forming a 3- to 7-membered saturated, partially unsaturated, or fully unsaturated heterocyclic ring together with the N-atom R^{21a} and R^{21b} are bonded to, wherein the alkylene chain contains none, or one or more, same or different heteroatoms O, S, or N, and is unsubstituted or substituted with one or more, same or different substituents selected from halogen, C₁-C₄-haloalkyl, C₁-C₄-alkoxy, and C₁-C₄-haloalkoxy, and wherein the N- and/or S-atoms of the heterocyclic ring are oxidized or non-oxidized;
R²² is H, halogen, NO₂, CN, OH, SH, C₁-C₆-alkoxy, C₁-C₆-haloalkoxy, C₁-C₆-alkylS(O)ₙ, C₁-C₆-haloalkyl-S(O)ₙ—, (CH₃)₃Si, (CH₃CH₂)₃Si, ((CH₃)₃C)(CH₃)₂Si, C₁-C₆-alkyl, C₂-C₆-alkenyl, C₂-C₆-alkynyl, or C₃-C₈-cycloalkyl, wherein the aliphatic and cyclo-aliphatic radicals are unsubstituted, partially or fully halogenated and/or oxygenated and/or are substituted by one or more, same or different radicals selected from C₁-C₄-alkoxy;
phenyl, benzyl, pyridyl, or phenoxy, wherein the radicals are unsubstituted, partially or fully halogenated and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
or two R²² present together on one atom are ═O, ═S, ═N(C₁-C₆-alkyl), ═NO(C₁-C₆-alkyl), ═CH(C₁-C₄-alkyl), or ═C(C₁-C₄-alkyl)C₁-C₄-alkyl;
or two R²² on two adjacent C-atoms are together a C₂-C₆ alkylene chain or C₂-C₆ alkenylene chain, which form together with the C-atom they are bonded to a 3- to 7-membered saturated, partially unsaturated, or fully unsaturated ring, wherein the alkylene or alkenylene chain contains none, or one or more, same or different heteroatoms selected from O, S, or N, and is unsubstituted or substituted with halogen, C₁-C₄-haloalkyl, C₁-C₄-alkoxy, or C₁-C₄-haloalkoxy, and wherein the N- and/or S-atoms of the heterocyclic ring are oxidized or non-oxidized;
n is 0, 1, or 2.

22: A method for protecting crops, plants, plant propagation material, and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with the compound of claim 21, wherein R¹ is H or F.

23: The method of claim 16, wherein at least one substituent R² is present, and wherein said at least one substituent R² is OR¹², S(O)ₙR¹², S(O)ₙNR^{13a}R^{13b}, and wherein
R¹² is H;
C₁-C₆-alkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
C₁-C₆-haloalkyl, which is unsubstituted, or substituted with one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, OR²⁰, S(O)ₙR²⁰, Si(R¹⁵)₂R¹⁶, C(═O)OR²⁰, OC(═O)R¹⁹, C(═O)NR^{21a}R^{21b}, CR¹⁹═NOR²⁰, N[C(O)R¹⁹]S(O)ₙR²⁰, phenyl, and C₅-C₆-hetaryl;
C₃-C₈-cycloalkyl, C₃-C₈-cycloalkyl-C₁-C₄-alkyl, C₃-C₈-halocycloalkyl, C₃-C₈-cycloalkyl-C₁-C₄-alkyl, wherein the cyclic moieties are unsubstituted, or substituted with one or more, same or different substituents selected from halogen, CN, C₁-C₄-alkyl, C₁-C₄-haloalkyl, C₁-C₄-alkoxy, C₁-C₄-haloalkoxy, ═O, and acetal groups thereof based on alcohols, or thiols selected from HOCH₂CH₂OH, HOCH₂CH₂CH₂OH, HOCH₂CH₂SH, HOCH₂CH₂CH₂SH, HSCH₂CH₂SH, HSCH₂CH₂CH₂SH;
C₂-C₆-alkenyl, C₂-C₆-haloalkenyl, C₂-C₆-alkynyl, C₂-C₆-haloalkynyl;
Phenyl, or phenyl-C₁-C₄-alkyl, wherein the aromatic rings are in each case unsubstituted, or substituted with one or more, same or different substituents R²²;
or a 3- to 7-membered saturated, partially unsaturated, or fully unsaturated heterocyclic ring comprising one or more, same or different heteroatoms O, N or S, wherein the heterocyclic ring is directly bonded to the remainder of the molecule, or bonded to the remainder of the molecule via a C₁-C₄-alkyl group, and wherein the heterocyclic ring is unsubstituted, or substituted with one or more, same or different substituents R²², and wherein the N- and/or S-atoms of the heterocyclic ring are oxidized, or non-oxidized,
R^{13a}, R^{13b} are H, C₁-C₆-alkyl, C₁-C₆-haloalkyl, C₁-C₆-alkoxy, C₁-C₆-haloalkoxy, C₁-C₆-alkyl-S—, C₁-C₆-haloalky-S—, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, —S(O)ₙNR^{21a}R^{21b}C(═O)R¹⁹, C(═O)OR²⁰, C(═O)NR^{21a}R^{21b}, C(═S)R¹⁹, C(═S)SR²⁰, C(═S)NR^{21a}R^{21b}, C(═NR^{21a})R¹⁹;
phenyl, which is unsubstituted, or substituted with one or more, same or different substituents R²²;
a 3- to 7-membered saturated, partially unsaturated or fully unsaturated heterocyclic ring comprising one or more, same or different heteroatoms O, N, or S, wherein the heterocyclic ring is unsubstituted, or substituted with one or more, same or different substituents R²², and wherein the N- and/or S-atoms of the heterocyclic ring are oxidized, or non-oxidized;
or R^{13a} and R^{13b} are together a C₂-C₇ alkylene, or C₂-C₇ alkenylene chain, and form a 3- to 8-membered saturated, partially unsaturated, or fully unsaturated ring together with the N-atom they are bonded to,
wherein the C₂-C₇ alkylene chain or C₂-C₇ alkenylene chain contains none, or one or more, same or different heteratoms O, S, or N, and is unsubstituted, or substituted with halogen, C₁-C₆-alkyl, C₁-C₆-haloalkyl, C₁-C₆-alkoxy, C₁-C₆-haloalkoxy, C₁-C₆-alkyl-S—, C₁-C₆-haloalkyl-S, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl;
phenyl, which is unsubstituted, or substituted with one or more, same or different substituents R²²;
or a 3- to 7-membered saturated, partially unsaturated, or fully unsaturated heterocyclic ring comprising one or more, same or different heteroatoms O, S, or N, wherein the heterocyclic ring is unsubstituted, or substituted with one or more, same or different substituents R²², and wherein the N- and/or S-atoms of the heterocyclic ring are oxidized, or non-oxidized;
and wherein all other substituents have the meanings as defined in claim 16.

24: The method of claim 16, wherein
R¹⁵, R¹⁶ are independently C₁-C₆ alkyl, or phenyl;
R¹⁹ is H, C₁-C₆-alkoxy, C₁-C₆-haloalkoxy, C₁-C₆-alkyl-S(O)ₙ—, C₁-C₆-haloalkyl-S(O)ₙ—, C₁-C₆-alkyl, C₂-C₆-alkenyl, C₂-C₆-alkynyl, C₃-C₈-cycloalkyl, wherein the aliphatic and cycloaliphatic radicals are unsubstituted, partially, or fully halogenated, and/or oxgenated, and/or substituted by one or more, same or different radicals selected from C₁-C₄-alkoxy;
phenyl, benzyl, pyridyl, or phenoxy, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino, and di-(C₁-C₆-alkyl)amino;
R²⁰ is H, C₁-C₆-alkyl, C₂-C₆-alkenyl, C₂-C₆-alkynyl, or C₃-C₈-cycloalkyl, wherein the aliphatic and cycloaliphatic radicals are unsubstituted, partially, or fully halogenated, and/or oxygenated, and/or substituted by one or more, same or different substituents selected from C₁-C₄-alkoxy;
phenyl, benzyl, or pyridyl, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
R^{21a}, R^{21b} are independently H, C₁-C₆-alkoxy, C₁-C₆-haloalkoxy, C₁-C₆-alkyl-S(O)ₙ—, C₁-C₆-haloalkyl-S(O)ₙ—, C₁-C₆-alkyl, C₂-C₆-alkenyl, C₂-C₆-alkynyl, C₃-C₈-cycloalkyl, C₃-C₈-cycloalkyl-C₁-C₆-alkyl, wherein the aliphatic and cycloaliphatic radicals are unsubstituted, partially, or fully halogenated, and/or oxygenated, and/or substituted by one or more, same or different substituents selected from C₁-C₄-alkoxy;
phenyl, benzyl, pyridyl, and phenoxy, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
or R^{21a} and R^{21b} are together a C₂-C₆ alkylene chain forming a 3- to 7-membered saturated, partially unsaturated, or fully unsaturated ring together with the N-atom they are bonded to, wherein the alkylene chain contains none, one or more, same or different heteroatoms O, S, or N, and is unsubstituted, or substituted with one or more, same or different substituents selected from halogen, C₁-C₄-haloalkyl, C₁-C₄-alkoxy, and C₁-C₄-haloalkoxy, and wherein the N- and/or S-atoms of the heterocyclic ring are oxidized, or non-oxidized; and
R²² is H, halogen, NO₂, CN, OH, SH, C₁-C₆-alkoxy, C₁-C₆-haloalkoxy, C₁-C₆-alkylS(O)ₙ, C₁-C₆-haloalkyl-S(O)ₙ—, C₁-C₆-alkyl, C₂-C₆-alkenyl, C₂-C₆-alkynyl, C₃-C₈-cycloalkyl, wherein the aliphatic and cycloaliphatic radicals are unsubstituted, partially, or fully halogenated, and/or oxygenated, and/or substituted by one or more, same or different substituents selected from C₁-C₄-alkoxy,
phenyl, benzyl, pyridyl, or phenoxy, wherein the radicals are unsubstituted, partially, or fully halogenated, and/or substituted by one or more, same or different substituents selected from C₁-C₆-alkyl, C₁-C₆-haloalkyl, C₁-C₆-alkoxy, C₁-C₆ haloalkoxy, and (C₁-C₆-alkoxy)carbonyl;
or two R²² present together on one atom are ═O, ═S, ═N(C₁-C₆-alkyl), ═NO(C₁-C₆-alkyl), ═CH(C₁-C₄-alkyl), or ═C(C₁-C₄-alkyl)C₁-C₄-alkyl;
or two R²² on two adjacent C-atoms are together a C₂-C₆ alkylene chain, or C₂-C₆-alkenylene chain, which form together with the C-atom they are bonded to a 3- to 7-membered saturated, partially unsaturated, or fully unsaturated ring, wherein the alkylene, or alkenylene chain contains one or more, same or different heteroatoms O, S, or N, and is unsubstituted, or substituted with one or more, same or different substituents selected from halogen, C₁-C₄-haloalkyl, C₁-C₄-alkoxy, and C₁-C₄-haloalkoxy, and wherein the N- and/or S-atoms of the heterocyclic ring are oxidized, or non-oxidized.

25: The method of claim 16, wherein at least one substituent R² is present, and wherein said at least one R² is OR¹², S(O)ₙR¹², S(O)ₙNR^{13a}R^{13b};
and wherein
R¹² is C₁-C₆-alkyl, which is unsubstituted, or substituted by one or more, same or different substituents selected from CN, C₁-C₆-alkoxy, C₁-C₆-haloalkoxy, C₁-C₆-alkyl-S(O)ₙ, C₁-C₆-haloalkyl-S(O)ₙ, and C₁-C₆-hetaryl;
C₁-C₆-haloalkyl, which is unsubstituted or substituted by one or more, same or different substituents selected from C₁-C₆-alkoxy, C₁-C₆-haloalkoxy, C₁-C₆-alkylS(O)ₙ, and C₁-C₆-haloalkyl-S(O)ₙ;
C₃-C₈-cycloalkyl-C₁-C₄-alkyl, which is unsubstituted, or substituted with one or more, same or different substituents selected from halogen, and CN;
C₁-C₄-alkyl-C₃-C₈-cycloalkyl, C₃-C₈-cycloalkyl, C₃-C₈-halocycloalkyl, C₂-C₆-alkenyl, C₂-C₆-alkynyl, NR^{21a}R^{21b};
R^{13a}, R^{13b} are independently C₁-C₆-alkyl, C₃-C₈-cycloalkyl, C₃-C₈-cycloalkyl-C₁-C₆-alkyl, or C(═O)R¹⁹;
R^{21a}, R^{21b} are independently H, C₁-C₆-alkyl, C₃-C₈-cycloalkyl, C₃-C₈-cycloalkyl-C₁-C₆-alkyl, or C₁-C₆-alkyl-C₃-C₈-cycloalkyl, wherein the aliphatic and cyclo-aliphatic radicals are unsubstituted, partially or fully halogenated, and/or oxygenated, and/or substituted by one or more, same or different substituents selected from C₁-C₄-alkoxy.

26: An agricultural or veterinary composition comprising a compound of claim 21.

27: Method for combating, or controlling invertebrate pests comprising contacting the invertebrate pests, or their food supply, habitat or breeding grounds with a pesticidally effective amount of a compound I of claim 21.

28: Method for protecting crops, plants, plant propagation material, and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a compound of claim 21.

29: Seed treated with a compound of claim 21.
